SEQUENCE LISTING (178 sequences)

SEQ ID NO: 1 - DNA, 14 bp, synthetic construct
  misc_feature 1..14: note = Synthetic Construct
  modified_base 1: mod_base = OTHER; note = 2' deoxy corresponding nucleotide
  modified_base 2: mod_base = OTHER; note = 2'-deoxyuridine
  modified_base 3..14: mod_base = OTHER; note = 2' deoxy corresponding nucleotide
  source 1..14: mol_type = other DNA
ttcgtcgtgacgtt

SEQ ID NO: 2 - DNA, 13 bp, synthetic construct
  misc_feature 1..13: note = Synthetic Construct
  modified_base 1: mod_base = OTHER; note = 2'-deoxyuridine
  modified_base 2..13: mod_base = OTHER; note = 2' deoxy corresponding nucleotide
  source 1..13: mol_type = other DNA
tcgtcgtgtcgtt

SEQ ID NO: 3 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-OMOE thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 4 - DNA, 13 bp, synthetic construct
  misc_feature 1..13: note = Synthetic Construct
  modified_base 1: mod_base = OTHER; note = 5-Bromo-2'-deoxyuridine
  modified_base 3: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 5 - DNA, 13 bp, synthetic construct
  misc_feature 1..13: note = Synthetic Construct
  modified_base 1: mod_base = OTHER; note = 5-Bromo-2'-deoxyuridine
  modified_base 6: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 6 - DNA, 13 bp, synthetic construct
  misc_feature 1..13: note = Synthetic Construct
  modified_base 1: mod_base = OTHER; note = 5-Bromo-2'-deoxyuridine
  modified_base 8: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 7 - DNA, 13 bp, synthetic construct
  misc_feature 1..13: note = Synthetic Construct
  modified_base 1: mod_base = OTHER; note = 5-Bromo-2'-deoxyuridine
  modified_base 11: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 8 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 9 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 10 - DNA, 15 bp, synthetic construct
  misc_feature 1..15: note = Synthetic Construct
  modified_base 1: mod_base = OTHER; note = 5-Bromo-2'-deoxyuridine
  modified_base 13..15: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..15: mod_base = OTHER; note = phosphorothioate linkages
  misc_feature 15: note = 3' spacer C3
  source 1..15: mol_type = other DNA
tcgtcgtgtcgtttt

SEQ ID NO: 11 - DNA, 13 bp, synthetic construct
  misc_feature 1..13: note = Synthetic Construct
  modified_base 1: mod_base = OTHER; note = 5-Bromo 2'-O-Methyl-uridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 12 - DNA, 14 bp, synthetic construct
  misc_feature 1..14: note = Synthetic Construct
  modified_base 1: mod_base = OTHER; note = 5-Bromo 2'-O-Methyl-uridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 13 - DNA, 14 bp, synthetic construct
  misc_feature 1..14: note = Synthetic Construct
  modified_base 1: mod_base = OTHER; note = 5-Bromo 2'-O-Methyl-uridine
  modified_base 2: mod_base = cm
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 14 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 15 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 16 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 17 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5^6: mod_base = OTHER; note = phosphorothioate linkage
  modified_base 6^7: mod_base = OTHER; note = phosphorodithioate linkage
  modified_base 7..13: mod_base = OTHER; note = phosphorothioate linkages
  misc_feature 13: note = 3' spacer C3
  source 1..13: mol_type = other DNA
tcgtcgtgtcgtt

SEQ ID NO: 18 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..7: mod_base = OTHER; note = phosphorothioate linkages
  modified_base 7^8: mod_base = OTHER; note = phosphorodithioate linkage
  modified_base 8..13: mod_base = OTHER; note = phosphorothioate linkages
  misc_feature 13: note = 3' spacer C3
  source 1..13: mol_type = other DNA
tcgtcgtgtcgtt

SEQ ID NO: 19 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..8: mod_base = OTHER; note = phosphorothioate linkages
  modified_base 8^9: mod_base = OTHER; note = phosphorodithioate linkage
  modified_base 9..13: mod_base = OTHER; note = phosphorothioate linkages
  misc_feature 13: note = 3' spacer C3
  source 1..13: mol_type = other DNA
tcgtcgtgtcgtt

SEQ ID NO: 20 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..9: mod_base = OTHER; note = phosphorothioate linkages
  modified_base 9^10: mod_base = OTHER; note = phosphorodithioate linkage
  modified_base 10..13: mod_base = OTHER; note = phosphorothioate linkages
  misc_feature 13: note = 3' spacer C3
  source 1..13: mol_type = other DNA
tcgtcgtgtcgtt

SEQ ID NO: 21 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..10: mod_base = OTHER; note = phosphorothioate linkages
  modified_base 10^11: mod_base = OTHER; note = phosphorodithioate linkage
  modified_base 11..13: mod_base = OTHER; note = phosphorothioate linkages
  misc_feature 13: note = 3' spacer C3
  source 1..13: mol_type = other DNA
tcgtcgtgtcgtt

SEQ ID NO: 22 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..11: mod_base = OTHER; note = phosphorothioate linkages
  modified_base 11^12: mod_base = OTHER; note = phosphorodithioate linkage
  modified_base 12^13: mod_base = OTHER; note = phosphorothioate linkage
  misc_feature 13: note = 3' spacer C3
  source 1..13: mol_type = other DNA
tcgtcgtgtcgtt

SEQ ID NO: 23 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..12: mod_base = OTHER; note = phosphorothioate linkages
  modified_base 12^13: mod_base = OTHER; note = phosphorodithioate linkage
  misc_feature 13: note = 3' spacer C3
  source 1..13: mol_type = other DNA
tcgtcgtgtcgtt

SEQ ID NO: 24 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = um
  modified_base 1..4: mod_base = OTHER; note = phosphorothioate linkages
  misc_feature 4^5: note = phosphotriester linker
  modified_base 5..13: mod_base = OTHER; note = phosphorothioate linkages
  modified_base 13: mod_base = OTHER; note = phosphorodithioate linkage
  misc_feature 13: note = 3' spacer C3
  source 1..13: mol_type = other DNA
tcgtcgtgtcgtt

SEQ ID NO: 25 - DNA, 13 bp, synthetic construct
  misc_feature 1..13: note = Synthetic Construct
  modified_base 1: mod_base = OTHER; note = 5-Bromo-2'-deoxyuridine
  modified_base 13: mod_base = um
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker
  modified_base 5..13: mod_base = OTHER; note = phosphorothioate linkages
  modified_base 13: mod_base = OTHER; note = phosphorodithioate linkage
  misc_feature 13: note = 3' spacer C3
  source 1..13: mol_type = other DNA
tcgtcgtgtcgtt

SEQ ID NO: 26 - DNA, 14 bp, synthetic construct
  misc_feature 1..14: note = Synthetic Construct
  modified_base 1: mod_base = OTHER; note = 5-Bromo 2'-O-Methyl-uridine
  modified_base 2: mod_base = cm
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 27 - DNA, 14 bp, synthetic construct
  misc_feature 1..14: note = Synthetic Construct
  modified_base 1: mod_base = OTHER; note = 5-Bromo 2'-O-Methyl-uridine
  modified_base 2: mod_base = cm
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 28 - DNA, 14 bp, synthetic construct
  misc_feature 1..14: note = Synthetic Construct
  modified_base 1: mod_base = OTHER; note = 5-Bromo 2'-O-Methyl-uridine
  modified_base 2: mod_base = cm
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 29 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 30 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..8: mod_base = OTHER; note = phosphorothioate linkages
  modified_base 8^9: mod_base = OTHER; note = phosphorodithioate linkage
  modified_base 9..14: mod_base = OTHER; note = phosphorothioate linkages
  misc_feature 14: note = 3' spacer C3
  source 1..14: mol_type = other DNA
tcgtcgtgtcgttt

SEQ ID NO: 31 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..8: mod_base = OTHER; note = phosphorothioate linkages
  modified_base 8^9: mod_base = OTHER; note = phosphorodithioate linkage
  modified_base 9..14: mod_base = OTHER; note = phosphorothioate linkages
  misc_feature 14: note = 3' spacer C3
  source 1..14: mol_type = other DNA
tcgtcgtgtcgttt

SEQ ID NO: 32 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5^6: mod_base = OTHER; note = phosphorodithioate linkage
  modified_base 6..14: mod_base = OTHER; note = phosphorothioate linkages
  misc_feature 14: note = 3' spacer C3
  source 1..14: mol_type = other DNA
tcgtcgtgtcgttt

SEQ ID NO: 33 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5^6: mod_base = OTHER; note = phosphorothioate linkage
  modified_base 6^7: mod_base = OTHER; note = phosphorodithioate linkage
  modified_base 7..14: mod_base = OTHER; note = phosphorothioate linkages
  misc_feature 14: note = 3' spacer C3
  source 1..14: mol_type = other DNA
tcgtcgtgtcgttt

SEQ ID NO: 34 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..7: mod_base = OTHER; note = phosphorothioate linkages
  modified_base 7^8: mod_base = OTHER; note = phosphorodithioate linkage
  modified_base 8..14: mod_base = OTHER; note = phosphorothioate linkages
  misc_feature 14: note = 3' spacer C3
  source 1..14: mol_type = other DNA
tcgtcgtgtcgttt

SEQ ID NO: 35 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 36 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 37 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 38 - DNA, 14 bp, synthetic construct
  misc_feature 1..14: note = Synthetic Construct
  modified_base 1: mod_base = OTHER; note = 5-Bromo-2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..10: mod_base = OTHER; note = phosphorothioate linkages
  modified_base 10^11: mod_base = OTHER; note = phosphorodithioate linkage
  modified_base 11..14: mod_base = OTHER; note = phosphorothioate linkages
  misc_feature 14: note = 3' spacer C3
  source 1..14: mol_type = other DNA
tcgtcgtgtcgttt

SEQ ID NO: 39 - protein, 11 aa, synthetic construct
  REGION 1..11: note = Synthetic Construct
LSLSPGLLQGG

SEQ ID NO: 40 - protein, 7 aa, synthetic construct
  REGION 1..7: note = Synthetic Construct
WPAQGPT

SEQ ID NO: 41 - protein, 6 aa, synthetic construct
  REGION 1..6: note = Synthetic Construct
WPQGPT

SEQ ID NO: 42 - protein, 7 aa, synthetic construct
  REGION 1..7: note = Synthetic Construct
WAPQGPT

SEQ ID NO: 43 - protein, 6 aa, synthetic construct
  REGION 1..6: note = Synthetic Construct
WAQGPT

SEQ ID NO: 44 - protein, 7 aa, synthetic construct
  REGION 1..7: note = Synthetic Construct
TPGQAPW

SEQ ID NO: 45 - protein, 7 aa, synthetic construct
  REGION 1..7: note = Synthetic Construct
PNPQLPF

SEQ ID NO: 46 - protein, 5 aa, synthetic construct
  REGION 1..5: note = Synthetic Construct
RPQQF

SEQ ID NO: 47 - protein, 5 aa, synthetic construct
  REGION 1..5: note = Synthetic Construct
RPQGF

SEQ ID NO: 48 - protein, 7 aa, synthetic construct
  REGION 1..7: note = Synthetic Construct
RPQGFPP

SEQ ID NO: 49 - protein, 8 aa, synthetic construct
  REGION 1..8: note = Synthetic Construct
RPQGFGPP

SEQ ID NO: 50 - protein, 7 aa, synthetic construct
  REGION 1..7: note = Synthetic Construct
RPRPQQF

SEQ ID NO: 51 - protein, 8 aa, synthetic construct
  REGION 1..8: note = Synthetic Construct
LSQSKVLG

SEQ ID NO: 52 - protein, 6 aa, synthetic construct
  REGION 1..6: note = Synthetic Construct
WGGQLL

SEQ ID NO: 53 - protein, 12 aa, synthetic construct
  REGION 1..12: note = Synthetic Construct
WALQRPHYSYPD

SEQ ID NO: 54 - protein, 12 aa, synthetic construct
  REGION 1..12: note = Synthetic Construct
WALQRPYTLTES

SEQ ID NO: 55 - protein, 12 aa, synthetic construct
  REGION 1..12: note = Synthetic Construct
WALQGPYTLTES

SEQ ID NO: 56 - protein, 5 aa, synthetic construct
  REGION 1..5: note = Synthetic Construct
SNAMS

SEQ ID NO: 57 - protein, 17 aa, synthetic construct
  REGION 1..17: note = Synthetic Construct
GISAGGSDTYYPASVKG

SEQ ID NO: 58 - protein, 9 aa, synthetic construct
  REGION 1..9: note = Synthetic Construct
ETWNHLFDY

SEQ ID NO: 59 - protein, 11 aa, synthetic construct
  REGION 1..11: note = Synthetic Construct
SGGSYSSYYYA

SEQ ID NO: 60 - protein, 7 aa, synthetic construct
  REGION 1..7: note = Synthetic Construct
SDDKRPS

SEQ ID NO: 61 - protein, 11 aa, synthetic construct
  REGION 1..11: note = Synthetic Construct
GGYDQSSYTNP

SEQ ID NO: 62 - protein, 118 aa, synthetic construct
  REGION 1..118: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSS

SEQ ID NO: 63 - protein, 108 aa, synthetic construct
  REGION 1..108: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTQLTVL

SEQ ID NO: 64 - protein, 108 aa, synthetic construct
  REGION 1..108: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVL

SEQ ID NO: 65 - protein, 108 aa, synthetic construct
  REGION 1..108: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTELTVL

SEQ ID NO: 66 - protein, 452 aa, synthetic construct
  REGION 1..452: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRPQGFGPP

SEQ ID NO: 67 - protein, 455 aa, synthetic construct
  REGION 1..455: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 68 - protein, 455 aa, synthetic construct
  REGION 1..455: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 69 - protein, 106 aa, synthetic construct
  REGION 1..106: note = Synthetic Construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 70 - protein, 106 aa, synthetic construct
  REGION 1..106: note = Synthetic Construct
GQPKANPTVTLFPPSSEELQANKATLVCLISDFYPGAVTVAWKADGSPVKAGVETTKPSKQSSDKYAASSYLSLTPEQWKSHRSYSCQVTHEGSTVEKTVAPTECS

SEQ ID NO: 71 - protein, 106 aa, synthetic construct
  REGION 1..106: note = Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTQLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 73 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 74 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTELTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 75 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTQLTVLGQPKANPTVTLFPPSSEELQANKATLVCLISDFYPGAVTVAWKADGSPVKAGVETTKPSKQSSDKYAASSYLSLTPEQWKSHRSYSCQVTHEGSTVEKTVAPTECS

SEQ ID NO: 76 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVLGQPKANPTVTLFPPSSEELQANKATLVCLISDFYPGAVTVAWKADGSPVKAGVETTKPSKQSSDKYAASSYLSLTPEQWKSHRSYSCQVTHEGSTVEKTVAPTECS

SEQ ID NO: 77 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTELTVLGQPKANPTVTLFPPSSEELQANKATLVCLISDFYPGAVTVAWKADGSPVKAGVETTKPSKQSSDKYAASSYLSLTPEQWKSHRSYSCQVTHEGSTVEKTVAPTECS

SEQ ID NO: 78 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - protein, 214 aa, synthetic construct
  REGION 1..214: note = Synthetic Construct
SYELTQPPSVSVSPGQTARITCSGGSYSSYYYAWYQQKPGQAPVTLIYSDDKRPSNIPERFSGSSSGTTVTLTISGVQAEDEADYYCGGYDQSSYTNPFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 81 - protein, 119 aa, synthetic construct
  REGION 1..119: note = Synthetic Construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 82 - protein, 118 aa, synthetic construct
  REGION 1..118: note = Synthetic Construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 83 - protein, 119 aa, synthetic construct
  REGION 1..119: note = Synthetic Construct
KELKVTQPEKSVSVAAGDSTVLNCTLTSLLPVGPIKWYRGVGQSRLLIYSFTGEHFPRVTNVSDATKRNNMDFSIRISNVTPEDAGTYYCVKFQKGPSEPDTEIQSGGGTEVYVLAKPS

SEQ ID NO: 84 - protein, 118 aa, synthetic construct
  REGION 1..118: note = Synthetic Construct
EEELQVIQPEKSVSVAAGESATLNCTATSLIPVGPIQWFRGVGPGRELIYHQKEGHFPRVTPVSDPTKRNNMDFSIRISNITPADAGTYYCVKFRKGSPDVELKSGAGTELSVRAKPS

SEQ ID NO: 85 - protein, 119 aa, synthetic construct
  REGION 1..119: note = Synthetic Construct
EDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPS

SEQ ID NO: 86 - protein, 119 aa, synthetic construct
  REGION 1..119: note = Synthetic Construct
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPS

SEQ ID NO: 87 - protein, 444 aa, synthetic construct
  REGION 1..444: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 88 - protein, 447 aa, synthetic construct
  REGION 1..447: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 447 aa, synthetic construct
  REGION 1..447: note = Synthetic Construct
EVQLVESGGGVVQPGGSLRLSCAASGFTFSSNAMSWVRQAPGKGLEWVAGISAGGSDTYYPASVKGRFTISRDNSKNTLYLQMNSLRAEDTAVYYCARETWNHLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 455 aa, synthetic construct
  REGION 1..455: note = Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGRPQGFGPP

SEQ ID NO: 91 - protein, 449 aa, synthetic construct
  REGION 1..449: note = Synthetic Construct
DVQLVESGGGVVRPGESLRLSCAASGFTFSSNAMSWVRQAPGKGLEWLAGISAGGSDTYYPASVKGRFTISRDNSKNTLYLQMNTLTAEDTAVYYCARETWNHLFDYWGLGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGRPQGFGPP

SEQ ID NO: 92 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 337 aa, synthetic construct
  REGION 1..337: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 96 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 100 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 101 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 102 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFASTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 103 - protein, 325 aa, synthetic construct
  REGION 1..325: note = Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 104 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 105 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 106 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 107 - protein, 326 aa, synthetic construct
  REGION 1..326: note = Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 108 - protein, 107 aa, Homo sapiens [sequence omitted]

SEQ ID NO: 109 - protein, 106 aa, Homo sapiens; identical to SEQ ID NO: 69

SEQ ID NO: 110 - protein, 106 aa, Homo sapiens; identical to SEQ ID NO: 71

SEQ ID NO: 111 - protein, 213 aa, synthetic construct
  REGION 1..213: note = Synthetic Construct
ALTQPASVSANPGETVKIACSGGDYYSYYYGWYQQKAPGSALVTVIYSDDKRPSDIPSRFSGSASGSTATLTITGVRAEDEAVYYCGGYDYSTYANAFGAGTTLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 112 - protein, 123 aa, synthetic construct
  REGION 1..123: note = Synthetic Construct
QVQLLESGGGVVQPGGSLRLSCAASGFAFSIYDMNWVRQAPGKGLEWVSAISSGGGTTYYADSVKGRFTISRDNAKNSLYLQMNSLRAEDTAVYYCARHSGYGTHWGVLFAYWGRGTLVTVSS

SEQ ID NO: 113 - protein, 107 aa, synthetic construct
  REGION 1..107: note = Synthetic Construct
DIQMTQSPSSLSASVGDRVTITCRASQDIHGYLNWYQQKPGKAPKLLIYYTSILHSGVPSRFSGSGSGTDFTLTISSLQPEDFATYFCQQGSTLPWTFGQGTKLEIK

SEQ ID NO: 114 - protein, 15 aa, Mus musculus
GLNDIFEAQKIEWHE

SEQ ID NO: 115 - protein, 342 aa, synthetic construct
  REGION 1..342: note = Synthetic Construct
KELKVTQADKSVSVAAGDSATLNCTVSSLTPVGPIKWFKGEGQNRSPIYSFIGGEHFPRITNVSDATKRNNMDFSICISNVTPEDAGTYYCVKFQKGIVEPDTEIKSGGGTTLYVLAKPSSPEVSGPDSRGSPGQTVNFTCKSYGFSPRNITLKWLKDGKELSHLETTISSKSNVSYNISSTVSVKLSPEDIHSRVICEVAHVTLEGRPLNGTANFSNIIRVSPTLKITQQPLTPASQVNLTCQVQKFYPKALQLNWLENGNLSRTDKPEHFTDNRDGTYNYTSLFLVNSSAHREDVVFTCQVEHDSQPAITENHTVRAFAHSSSGGSMETIPDNNAYYNWN

SEQ ID NO: 116 - protein, 442 aa, synthetic construct
  REGION 1..442: note = Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 117 - protein, 214 aa, synthetic construct
  REGION 1..214: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118-120 - skipped (3 entries)

SEQ ID NO: 121 - DNA, 14 bp, synthetic construct
  modified_base 2: mod_base = OTHER; note = iodo-uridine
  modified_base 1..5: mod_base = OTHER; note = phosphorothioate linkages
  misc_feature 5^6: note = phosphotriester linker
  modified_base 6..14: mod_base = OTHER; note = phosphorothioate linkages
  source 1..14: mol_type = other DNA
  misc_feature 14: note = 3' spacer C3
ttcgtcgtgacgtt

SEQ ID NO: 122-128 - skipped (7 entries)

SEQ ID NO: 129 - DNA, 24 bp, synthetic construct
  misc_feature 1..24: note = Synthetic Construct
  modified_base 1..24: mod_base = OTHER; note = 2' deoxy corresponding nucleotide
  source 1..24: mol_type = other DNA
tcgtcgttttgtcgttttgtcgtt

SEQ ID NO: 130 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-OMOE thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 131 - DNA, 13 bp, synthetic construct
  misc_feature 1..13: note = Synthetic Construct
  modified_base 1: mod_base = OTHER; note = 2'-deoxyuridine
  modified_base 3: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 132 - DNA, 13 bp, synthetic construct
  misc_feature 1..13: note = Synthetic Construct
  modified_base 1: mod_base = OTHER; note = 2'-deoxyuridine
  modified_base 6: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 133 - DNA, 13 bp, synthetic construct
  misc_feature 1..13: note = Synthetic Construct
  modified_base 1: mod_base = OTHER; note = 2'-deoxyuridine
  modified_base 8: mod_base = OTHER; note = 8-oxo-7,8-dihydro-2'-deoxyguanosine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 134 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 135 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 136 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 137 - DNA, 15 bp, synthetic construct
  misc_feature 1..15: note = Synthetic Construct
  modified_base 1: mod_base = OTHER; note = 2'-deoxyuridine
  modified_base 13..15: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..15: mod_base = OTHER; note = phosphorothioate linkages
  misc_feature 15: note = 3' spacer C3
  source 1..15: mol_type = other DNA
tcgtcgtgtcgtttt

SEQ ID NO: 138 - DNA, 13 bp, synthetic construct
  misc_feature 1..13: note = Synthetic Construct
  modified_base 1: mod_base = um
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 139 - DNA, 14 bp, synthetic construct
  misc_feature 1..14: note = Synthetic Construct
  modified_base 1: mod_base = um
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 140 - DNA, 14 bp, synthetic construct
  misc_feature 1..14: note = Synthetic Construct
  modified_base 1: mod_base = um
  modified_base 2: mod_base = cm
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 141 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 142 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker
  modified_base 5..13: mod_base = OTHER; note = phosphorothioate linkages
  misc_feature 13: note = 3' spacer C3
  source 1..13: mol_type = other DNA
tcgtcgtgtcgtt

SEQ ID NO: 143 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5^6: mod_base = OTHER; note = phosphorodithioate linkage
  modified_base 6..13: mod_base = OTHER; note = phosphorothioate linkages
  misc_feature 13: note = 3' spacer C3
  source 1..13: mol_type = other DNA
tcgtcgtgtcgtt

SEQ ID NO: 144 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5^6: mod_base = OTHER; note = phosphorothioate linkage
  modified_base 6^7: mod_base = OTHER; note = phosphorodithioate linkage
  modified_base 7..13: mod_base = OTHER; note = phosphorothioate linkages
  misc_feature 13: note = 3' spacer C3
  source 1..13: mol_type = other DNA
tcgtcgtgtcgtt

SEQ ID NO: 145 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..7: mod_base = OTHER; note = phosphorothioate linkages
  modified_base 7^8: mod_base = OTHER; note = phosphorodithioate linkage
  modified_base 8..13: mod_base = OTHER; note = phosphorothioate linkages
  misc_feature 13: note = 3' spacer C3
  source 1..13: mol_type = other DNA
tcgtcgtgtcgtt

SEQ ID NO: 146 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..8: mod_base = OTHER; note = phosphorothioate linkages
  modified_base 8^9: mod_base = OTHER; note = phosphorodithioate linkage
  modified_base 9..13: mod_base = OTHER; note = phosphorothioate linkages
  misc_feature 13: note = 3' spacer C3
  source 1..13: mol_type = other DNA
tcgtcgtgtcgtt

SEQ ID NO: 147 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..9: mod_base = OTHER; note = phosphorothioate linkages
  modified_base 9^10: mod_base = OTHER; note = phosphorodithioate linkage
  modified_base 10..13: mod_base = OTHER; note = phosphorothioate linkages
  misc_feature 13: note = 3' spacer C3
  source 1..13: mol_type = other DNA
tcgtcgtgtcgtt

SEQ ID NO: 148 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..10: mod_base = OTHER; note = phosphorothioate linkages
  modified_base 10^11: mod_base = OTHER; note = phosphorodithioate linkage
  modified_base 11..13: mod_base = OTHER; note = phosphorothioate linkages
  misc_feature 13: note = 3' spacer C3
  source 1..13: mol_type = other DNA
tcgtcgtgtcgtt

SEQ ID NO: 149 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..11: mod_base = OTHER; note = phosphorothioate linkages
  modified_base 11^12: mod_base = OTHER; note = phosphorodithioate linkage
  modified_base 12^13: mod_base = OTHER; note = phosphorothioate linkage
  misc_feature 13: note = 3' spacer C3
  source 1..13: mol_type = other DNA
tcgtcgtgtcgtt

SEQ ID NO: 150 - DNA, 13 bp, synthetic construct
  misc_feature 1..13: note = Synthetic Construct
  modified_base 1: mod_base = OTHER; note = 2'-deoxyuridine
  modified_base 13: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..12: mod_base = OTHER; note = phosphorothioate linkages
  modified_base 12^13: mod_base = OTHER; note = phosphorodithioate linkage
  misc_feature 13: note = 3' spacer C3
  source 1..13: mol_type = other DNA
tcgtcgtgtcgtt

SEQ ID NO: 151 - DNA, 13 bp, synthetic construct
  misc_feature 1..13: note = Synthetic Construct
  modified_base 1: mod_base = OTHER; note = 2'-deoxyuridine
  modified_base 13: mod_base = um
  modified_base 1..4: mod_base = OTHER; note = phosphorothioate linkages
  misc_feature 4^5: note = phosphotriester linker
  modified_base 5..13: mod_base = OTHER; note = phosphorothioate linkages
  modified_base 13: mod_base = OTHER; note = phosphorodithioate linkage
  misc_feature 13: note = 3' spacer C3
  source 1..13: mol_type = other DNA
tcgtcgtgtcgtt

SEQ ID NO: 152 - DNA, 13 bp, synthetic construct
  misc_feature 1..13: note = Synthetic Construct
  modified_base 1: mod_base = OTHER; note = 2'-deoxyuridine
  modified_base 13: mod_base = um
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker
  modified_base 5..13: mod_base = OTHER; note = phosphorothioate linkages
  modified_base 13: mod_base = OTHER; note = phosphorodithioate linkage
  misc_feature 13: note = 3' spacer C3
  source 1..13: mol_type = other DNA
tcgtcgtgtcgtt

SEQ ID NO: 153 - DNA, 14 bp, synthetic construct
  misc_feature 1..14: note = Synthetic Construct
  modified_base 1: mod_base = um
  modified_base 2: mod_base = cm
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 154 - DNA, 14 bp, synthetic construct
  misc_feature 1..14: note = Synthetic Construct
  modified_base 1: mod_base = um
  modified_base 2: mod_base = cm
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 155 - DNA, 14 bp, synthetic construct
  misc_feature 1..14: note = Synthetic Construct
  modified_base 1: mod_base = um
  modified_base 2: mod_base = cm
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 156 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 157 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..8: mod_base = OTHER; note = phosphorothioate linkages
  modified_base 8^9: mod_base = OTHER; note = phosphorodithioate linkage
  modified_base 9..14: mod_base = OTHER; note = phosphorothioate linkages
  misc_feature 14: note = 3' spacer C3
  source 1..14: mol_type = other DNA
tcgtcgtgtcgttt

SEQ ID NO: 158 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..8: mod_base = OTHER; note = phosphorothioate linkages
  modified_base 8^9: mod_base = OTHER; note = phosphorodithioate linkage
  modified_base 9..14: mod_base = OTHER; note = phosphorothioate linkages
  misc_feature 14: note = 3' spacer C3
  source 1..14: mol_type = other DNA
tcgtcgtgtcgttt

SEQ ID NO: 159 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5^6: mod_base = OTHER; note = phosphorodithioate linkage
  modified_base 6..14: mod_base = OTHER; note = phosphorothioate linkages
  misc_feature 14: note = 3' spacer C3
  source 1..14: mol_type = other DNA
tcgtcgtgtcgttt

SEQ ID NO: 160 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5^6: mod_base = OTHER; note = phosphorothioate linkage
  modified_base 6^7: mod_base = OTHER; note = phosphorodithioate linkage
  modified_base 7..14: mod_base = OTHER; note = phosphorothioate linkages
  misc_feature 14: note = 3' spacer C3
  source 1..14: mol_type = other DNA
tcgtcgtgtcgttt

SEQ ID NO: 161 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..7: mod_base = OTHER; note = phosphorothioate linkages
  modified_base 7^8: mod_base = OTHER; note = phosphorodithioate linkage
  modified_base 8..14: mod_base = OTHER; note = phosphorothioate linkages
  misc_feature 14: note = 3' spacer C3
  source 1..14: mol_type = other DNA
tcgtcgtgtcgttt

SEQ ID NO: 162 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 163 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 164 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1^2: mod_base = OTHER; note = phosphorothioate linkage
  modified_base 2^3: mod_base = OTHER; note = phosphorodithioate linkage
  modified_base 3^4: mod_base = OTHER; note = phosphorothioate linkage
  misc_feature 4^5: note = phosphotriester linker-PEG24-NH2
  modified_base 5..9: mod_base = OTHER; note = phosphorothioate linkages
  modified_base 9^10: mod_base = OTHER; note = phosphorodithioate linkage
  modified_base 10..14: mod_base = OTHER; note = phosphorothioate linkages
  misc_feature 14: note = 3' spacer C3
  source 1..14: mol_type = other DNA
tcgtcgtgtcgttt

SEQ ID NO: 165 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..3: mod_base = OTHER; note = phosphorothioate linkages
  modified_base 3^4: mod_base = OTHER; note = phosphorodithioate linkage
  misc_feature 4^5: note = phosphotriester linker-PEG24-NH2
  modified_base 5..14: mod_base = OTHER; note = phosphorothioate linkages
  misc_feature 14: note = 3' spacer C3
  source 1..14: mol_type = other DNA
tcgtcgtgtcgttt

SEQ ID NO: 166 - DNA, 14 bp, synthetic construct
  misc_feature 1..14: note = Synthetic Construct
  modified_base 1: mod_base = OTHER; note = 2'-deoxyuridine
  modified_base 13..14: mod_base = OTHER; note = 2'-O-Methyl-Thymidine
  modified_base 1..4: mod_base = OTHER; note = phosphorothioate linkages
  misc_feature 4^5: note = phosphotriester linker-PEG24-NH2
  modified_base 5..10: mod_base = OTHER; note = phosphorothioate linkages
  modified_base 10^11: mod_base = OTHER; note = phosphorodithioate linkage
  modified_base 11..14: mod_base = OTHER; note = phosphorothioate linkages
  misc_feature 14: note = 3' spacer C3
  source 1..14: mol_type = other DNA
tcgtcgtgtcgttt

SEQ ID NO: 167 - DNA, 24 bp, synthetic construct
  misc_feature 1..24: note = Synthetic Construct
  source 1..24: mol_type = other DNA
tcgtcgttttgtcgttttgtcgtt

SEQ ID NO: 168-171 - skipped (4 entries)

SEQ ID NO: 172 - protein, 5 aa, synthetic construct
  REGION 1..5: note = Synthetic Construct
LLQGG

SEQ ID NO: 173 - protein, 8 aa, synthetic construct
  REGION 1..8: note = Synthetic Construct
GGGLLQGG

SEQ ID NO: 174-175 - skipped (2 entries)

SEQ ID NO: 176 - DNA, 20 bp, synthetic construct
  misc_feature 1..20: note = Synthetic Construct
  source 1..20: mol_type = other DNA
tccatgacgttcctgacgtt

SEQ ID NO: 177 - skipped

SEQ ID NO: 178 - protein, 337 aa, synthetic construct
  REGION 1..337: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

(19) United States
(12) Patent Application Publication
(10) Pub. No.: US 2024/0374746 A1
(43) Pub. Date: Nov. 14, 2024

(54) SIRP-ALPHA ANTIBODIES AND CONJUGATES

(71) Applicant: Tallac Therapeutics, Inc., Burlingame, CA (US)

(72) Inventors: Min LI, Dublin, CA (US); Ons HARRABI, Burlingame, CA (US); Amy CHEN, Burlingame, CA (US); Emma Ruth SANGALANG, Oakland, CA (US); Tracy Chia-Chien KUO, San Mateo, CA (US); Bang Janet SIM, Brisbane, CA (US); Hong I. WAN, Foster City, CA (US); Jaume PONS, San Francisco, CA (US)

(21) Appl. No.: 18/583,813

(22) Filed: Feb. 21, 2024

Related U.S. Application Data
(63) Continuation of application No. PCT/US2022/075381, filed on Aug. 24, 2022.
(60) Provisional application No. 63/236,989, filed on Aug. 25, 2021.
(60) Provisional application No. 63/256,270, filed on Oct. 15, 2021.

(51) Int. Cl. A61K 47/68; A61P 35/00

(52) U.S. Cl. CPC A61K 47/6807; A61K 47/6849; A61K 47/6889; A61P 35/00

(57) ABSTRACT

The present disclosure provides anti-SIRP-α polypeptide antibodies and oligonucleotide conjugates thereof. Also provided are related methods of preparation thereof and methods of use thereof, including therapeutic uses.

### CROSS-REFERENCE TO RELATED APPLICATIONS

This application is a continuation of International Application No. PCT/US2022/075381, filed on Aug. 24, 2022, which claims the priority benefit of U.S. Provisional Application Ser. No. 63/236,989, filed on Aug. 25, 2021, and U.S. Provisional Application Ser. No. 63/256,270, filed on Oct. 15, 2021, each of which is hereby incorporated by reference in its entirety.

### REFERENCE TO AN ELECTRONIC SEQUENCE LISTING

The contents of the electronic sequence listing (186492000401SUBSEQLIST.xml; Size: 501,134 bytes; and Date of Creation: May 10, 2024) are incorporated herein by reference in their entirety.

### FIELD OF INVENTION

The present disclosure relates to anti-SIRP-α antibodies and conjugates thereof and uses of such, including therapeutic uses.

### BACKGROUND

Signal-regulatory protein alpha (SIRP-α) is part of a family of cell-surface receptors that plays critical roles in the regulation of the immune system (see, e.g., Barclay, A. N. and Brown, M. H. (2006) Nat. Rev. Immunol. 6:457-64). One of the major roles of SIRP-α is its regulation of the immune response through interactions with CD47. CD47 is expressed on the surface of a variety of cell types. When the IgSF domain of CD47 binds the extracellular domain (e.g., the D1 domain) of SIRP-α expressed on an immune cell (e.g., a macrophage), this transduces a SIRP-α-mediated signal in the immune cell that prevents phagocytosis of the CD47-expressing cell. Thus, CD47 serves to convey what has been termed a “don't eat me” signal to the immune system that prevents phagocytosis of healthy cells (see, e.g., WO2015/138600 and Weiskopf, K. et al. (2013) Science 341:88-91). However, CD47 has also been shown to be highly expressed by a variety of cancers, and its interaction with SIRP-α in this context is thought to allow tumors to mimic the healthy “don't eat me” signal in order to evade immune surveillance and phagocytosis by macrophages (see, e.g., Majeti, R. et al. (2009) Cell 138:286-99; Zhao, X. W. et al. (2011) Proc. Natl. Acad. Sci. 108:18342-7). As such, antibodies that block this interaction are highly desirable.

SIRP-α is expressed on the surface of various cells, including leukocytes such as dendritic cells, eosinophils, neutrophils, and macrophages. SIRP-α includes an extracellular domain that interacts with external stimuli such as ligands and an intracellular domain that mediates a variety of intracellular signals. SIRP-α is a myeloid inhibitory receptor that suppresses immune activation following binding of its ligand CD47. Blockade of CD47-SIRPα myeloid checkpoint pathway has been shown to promote myeloid-mediated anti-tumor functions leading to the induction of adaptive immunity (Kuo, T. et al. (2020) J. Hematol. Oncol. 13:160). Additionally, SIRPα is highly expressed in various tumor types including renal cell carcinoma and melanoma (Yanagita, T. et al. (2017) JCI Insight 2:e89140).

Pathogen-associated molecular patterns (PAMPs) are molecules associated with various pathogens and are recognized by toll-like receptors (TLRs) and other pattern recognition receptors (PRRs) activating innate immune responses. The ability of PAMPs to recruit immune system in the absence of pathogens provides a strategy for treating a variety of diseases involving cell destruction (e.g., anticancer therapy) through the use of innate immune system response. One class of PAMPs that has been investigated for a variety of therapeutic applications is immunostimulating oligonucleotides, such as unmethylated cytosine-guanine dinucleotide (CpG)-containing oligodeoxynucleotides (CpG ODNs) (e.g., agatolimod). It is thought that CpG ODNs mediate TLR9 dimerization in immune cells (e.g., B cells, monocytes and plasmacytoid dendritic cells (pDCs)) to upregulate cytokines (e.g., type I interferon and interleukins), thereby activating natural killer cells.

Toll-like receptor 9 (TLR9), also designated as CD289, is an important receptor expressed in immune system cells including dendritic cells (DCs), B lymphocytes, macrophages, natural killer cells, and other antigen presenting cells. TLR9 activation triggers intracellular signaling cascades, leading to activation, maturation, proliferation and cytokine productions in these immune cells, thus bridges the innate and adaptive immunity. Martinez-Campos et al., Viral Immunol. 2016, 30, 98-105; Notley et al., Sci. Rep. 2017, 7, 42204. Natural TLR-9 agonists include unmethylated cytosine-guanine dinucleotide (CpG)-containing oligodeoxynucleotides (CpG ODNs).

CpG ODNs may include, for example, oligodeoxynucleotides having poly-G tails with phosphorothioate backbones at 3′- and 5′-termini and a central palindromic sequence including a phosphate backbone and a CpG within its central palindrome sequence, or oligodeoxynucleotides having a fully phosphorothioate backbone, and a sequence at the 5′ end for TLR9 activation, or oligodeoxynucleotides having a fully phosphorothioate backbone with a 3′-end sequence enabling formation of a duplex. However, CpG ODNs are often susceptible to degradation in serum and thus pharmacokinetics of CpG ODNs may be one of the limiting factors in their development as therapeutics. Also CpG ODNs often exhibit uneven tissue distribution in vivo, with primary sites of accumulation being in liver, kidney, and spleen. Such distribution can elicit off-target activity and local toxicity associated with PAMPs.

All references cited herein, including patent applications, patent publications, and scientific literature, are herein incorporated by reference in their entirety, as if each individual reference were specifically and individually indicated to be incorporated by reference.

### BRIEF SUMMARY

As demonstrated herein, conjugates were found to integrate TLR9 activation with blockade of CD47-SIRP-α interaction of myeloid cells, leading to anti-tumor immune responses engaging both the innate and adaptive immune systems. Without wishing to be bound to theory, it is thought that anti-SIRP-α antibody-CpG oligonucleotide conjugates can have the additional benefit of preferential tumor cell targeting in SIRP-α-expressing cancers.

In one aspect, provided herein is a conjugate comprising (i) an antibody or antigen-binding fragment thereof that specifically binds an extracellular domain of a human SIRP-α polypeptide and (ii) one or more immunomodulating oligonucleotides (P), wherein the antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A):

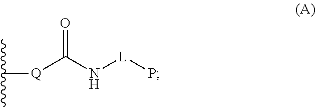

wherein [symbol] indicates the point of attachment of each Q to the antibody or antigen-binding fragment thereof (Ab);
wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO: 59), a CDR-L2 comprising the amino acid sequence of SDDKRPS (SEQ ID NO:60), and a CDR-L3 comprising the amino acid sequence of GGYDQSSYTNP (SEQ ID NO:61);
wherein each P is independently an immunomodulating oligonucleotide comprising the structure

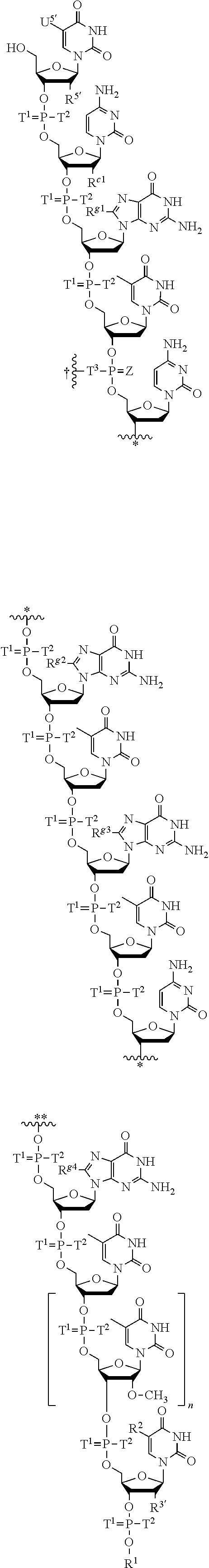

wherein
   [symbol] * and [symbol] ** indicate the points of attachment within the oligonucleotide, and wherein [symbol] † indicates the point of attachment to the linker L;
   each T¹ is independently O or S;
   each T² is S;
   T³ is a group

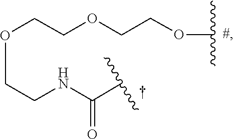 [SMILES: *OCCOCCOCCNC(*)=O]

wherein [symbol] † indicates the point of attachment to L and wherein [symbol] # indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2.

In another aspect, provided herein is a conjugate comprising (i) an antibody or antigen-binding fragment thereof that specifically binds an extracellular domain of a human SIRP-α polypeptide and (ii) one or more immunomodulating oligonucleotides (P); wherein the antibody or antigen-binding fragment is linked to one or more Q-tag peptides (Q); wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO: 61); and wherein each immunomodulating oligonucleotide is linked to a Q-tag peptide via an amide bond with the glutamine residue of the Q-tag peptide and a linker (L) as shown in Formula (A)

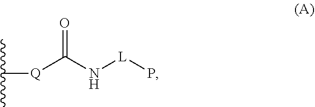

wherein:
   [symbol] indicates the point of attachment of each Q to the antibody or antigen-binding fragment thereof (Ab)
   each Q independently comprises a Q-tag peptide sequence RPQGF (SEQ ID NO: 47);
   each L is independently a bond or a linker moiety

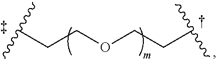

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and each P is independently an immunomodulating oligonucleotide comprising the structure

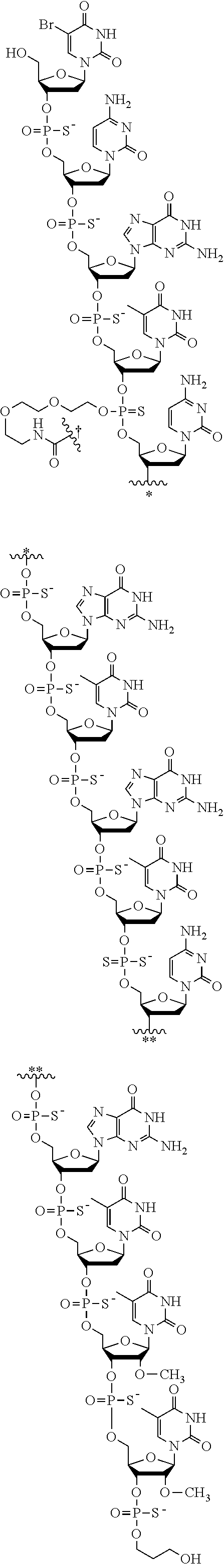

wherein [symbol] * and [symbol] ** indicate the points of attachment within the oligonucleotide, and wherein [symbol] † indicates the point of attachment to the linker L.

In another aspect, provided herein is a conjugate comprising (i) an antibody or antigen-binding fragment thereof that specifically binds an extracellular domain of a human SIRP-α polypeptide and (ii) one or more immunomodulating oligonucleotides (P); wherein the antibody or antigen-binding fragment is linked to one or more Q-tag peptides (Q); wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO: 61); and wherein each immunomodulating oligonucleotide is linked to a Q-tag peptide via an amide bond with the glutamine residue of the Q-tag peptide and a linker (L) as shown in Formula (A)

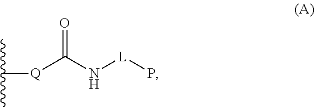

wherein:
   [symbol] indicates the point of attachment of each Q to the antibody or antigen-binding fragment thereof (Ab)
   each Q independently comprises a Q-tag peptide sequence RPQGF (SEQ ID NO: 47);
   each L is independently a bond or a linker moiety

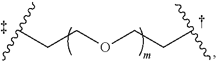

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue; and
each P is independently an immunomodulating oligonucleotide comprising the structure

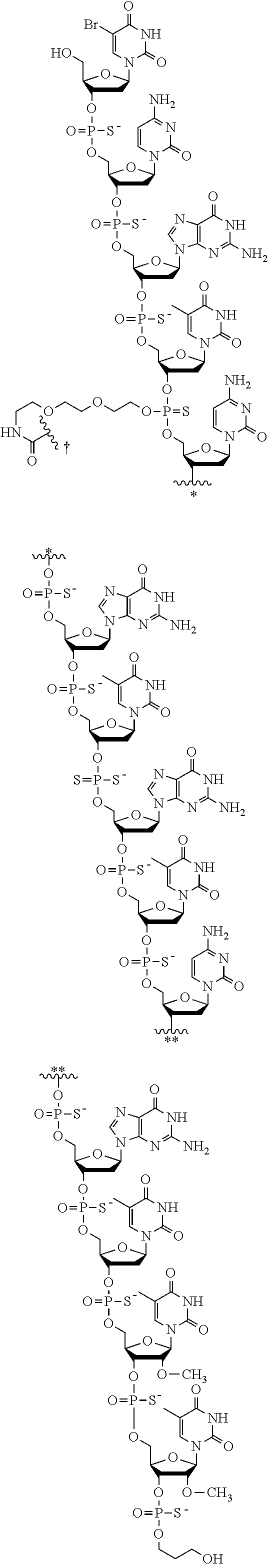

wherein [symbol] * and [symbol] ** indicate the points of attachment within the oligonucleotide, and wherein [symbol] † indicates the point of attachment to the linker L.

In another aspect, provided herein is a conjugate comprising (i) an antibody or antigen-binding fragment thereof that specifically binds an extracellular domain of a human SIRP-α polypeptide and (ii) one or more immunomodulating oligonucleotides (P), wherein the antibody or antigen-binding fragment is linked to one or more Q-tag peptides (Q) that comprise the amino acid sequence RPQGF (SEQ ID NO:47); wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61); and wherein each immunomodulating oligonucleotide (P) is linked to a Q-tag peptide via an amide bond with the glutamine residue of the Q-tag peptide and a linker (L) as shown in Formula (A):

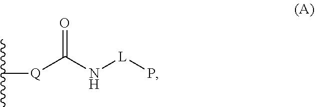

wherein [symbol] indicates the point of attachment of each Q to the antibody or antigen-binding fragment thereof (Ab).

In another aspect, provided herein is a conjugate comprising an antibody or antigen-binding fragment thereof (Ab) that specifically binds an extracellular domain of a human SIRP-α polypeptide, wherein the antibody comprises two antibody light chains that each comprise a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO: 61); two antibody heavy chains that each comprise a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58); and two Q-tag peptides comprising a peptide sequence RPQGF (SEQ ID NO:47); wherein each of the Q-tag peptides is linked to the C-terminus of one of the antibody heavy chains; and wherein at least one of the Q-tag peptides is linked to an immunomodulating oligonucleotide (P) via an amide bond with the glutamine residue of the Q-tag peptide and linker (L) as shown in FIG. 9A or FIG. 9B.

In another aspect, provided herein is a conjugate comprising (i) an antibody or antigen-binding fragment thereof that specifically binds an extracellular domain of a human SIRP-α polypeptide and (ii) one or more immunomodulating oligonucleotides (P), wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO: 61); wherein the antibody or antigen-binding fragment is linked to one or more Q-tag peptides (Q), wherein each immunomodulating oligonucleotide (P) is linked to a Q-tag peptide via an amide bond with the glutamine residue of the Q-tag peptide and a linker (L) as shown in Formula (A):

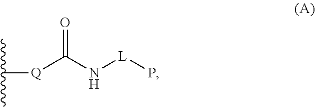

wherein [symbol] indicates the point of attachment of each Q to the antibody or antigen-binding fragment thereof (Ab); and wherein the antibody heavy chain comprises an Fc region (e.g., a human IgG1, IgG2, or IgG4 Fc region) comprising an N297A substitution, amino acid position numbering according to EU index. In another aspect, provided herein is a conjugate comprising (i) an antibody or antigen-binding fragment thereof that specifically binds an extracellular domain of a human SIRP-α polypeptide and (ii) one or more immunomodulating oligonucleotides (P); wherein the antibody or antigen-binding fragment is linked to one or more Q-tag peptides (Q); wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO: 59), a CDR-L2 comprising the amino acid sequence of SDDKRPS (SEQ ID NO:60), and a CDR-L3 comprising the amino acid sequence of GGYDQSSYTNP (SEQ ID NO:61); and wherein each immunomodulating oligonucleotide is linked to a Q-tag peptide via an amide bond with the glutamine residue of the Q-tag peptide and a linker (L) as shown in Formula (A)

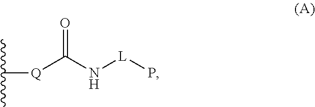

wherein:
[symbol] indicates the point of attachment of each Q to the antibody or antigen-binding fragment thereof (Ab)
each L is independently a bond or a linker moiety

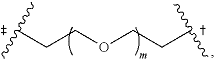

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue;
wherein each P is independently an immunomodulating oligonucleotide comprising the structure

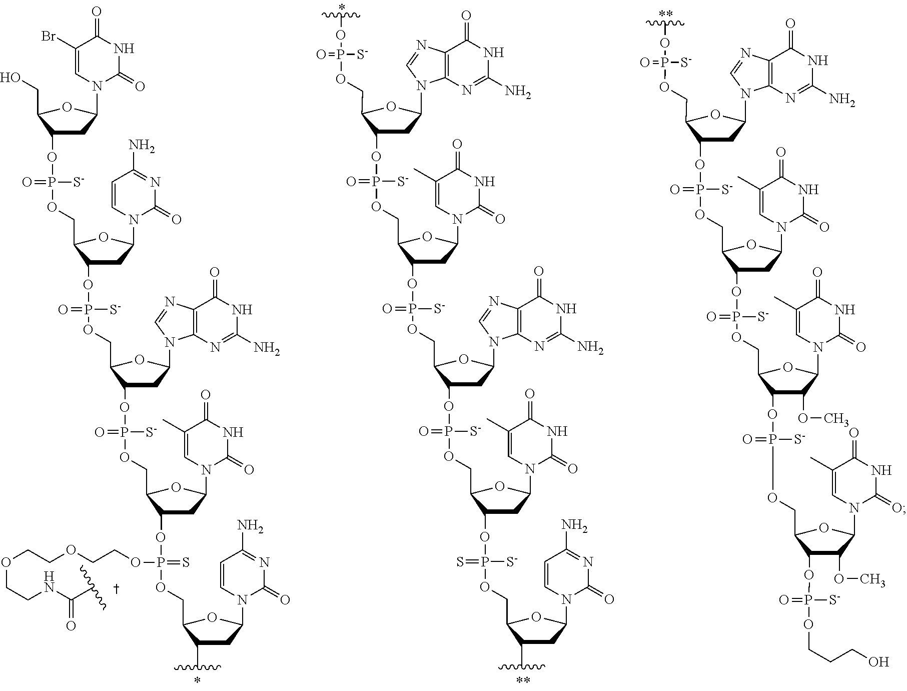

wherein [symbol] * and [symbol] ** indicate the points of attachment within the oligonucleotide, and
wherein [symbol] † indicates the point of attachment to the linker L; and
wherein the antibody heavy chain comprises an Fc region (e.g., a human IgG1, IgG2, or IgG4 Fc region) comprising an N297A substitution, amino acid position numbering according to EU index. In another aspect, provided herein is a conjugate comprising (i) an antibody or antigen-binding fragment thereof that specifically binds an extracellular domain of a human SIRP-α polypeptide and (ii) one or more immunomodulating oligonucleotides (P); wherein the antibody or antigen-binding fragment is linked to one or more Q-tag peptides (Q); wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61); and wherein each immunomodulating oligonucleotide is linked to a Q-tag peptide via an amide bond with the glutamine residue of the Q-tag peptide and a linker (L) as shown in Formula (A)

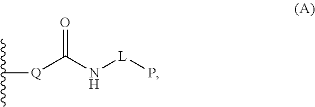

wherein:
[symbol] indicates the point of attachment of each Q to the antibody or antigen-binding fragment thereof (Ab);
each L is independently a bond or a linker moiety

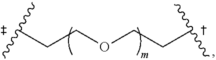

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue;
wherein each P is independently an immunomodulating oligonucleotide comprising the structure

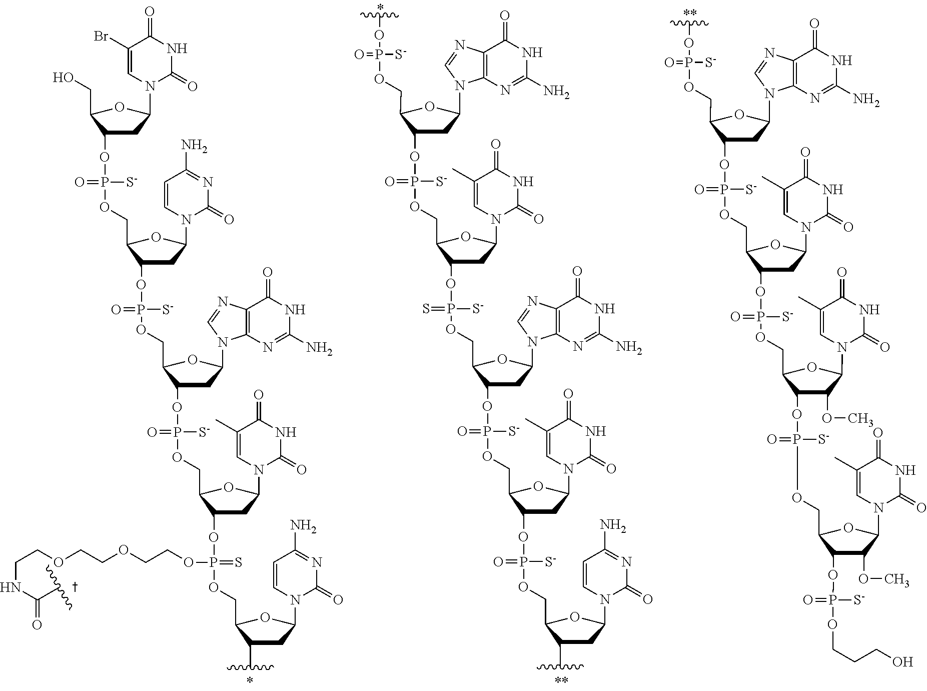

wherein [symbol] * and [symbol] ** indicate the points of attachment within the oligonucleotide, and
wherein [symbol] † indicates the point of attachment to the linker L; and
wherein the antibody heavy chain comprises an Fc region (e.g., a human IgG1, IgG2, or IgG4 Fc region) comprising an N297A substitution, amino acid position numbering according to EU index. In another aspect, provided herein is a conjugate comprising an antibody or antigen-binding fragment thereof (Ab) that specifically binds an extracellular domain of a human SIRP-α polypeptide, wherein the antibody comprises two antibody light chains that each comprise a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61); two antibody heavy chains that each comprise a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58); and two Q-tag peptides; wherein each of the Q-tag peptides is linked to the C-terminus of one of the antibody heavy chains or present in the Fc region of one of the antibody heavy chains; wherein the antibody heavy chain comprises an Fc region (e.g., a human IgG1, IgG2, or IgG4 Fc region) comprising an N297A substitution, amino acid position numbering according to EU index; and wherein at least one of the Q-tag peptides is linked to an immunomodulating oligonucleotide (P) via an amide bond with the glutamine residue of the Q-tag peptide and linker (L) as shown in FIG. 9A or FIG. 9B. In some embodiments, Q295 of the Fc region serves as the glutamine residue of the Q-tag peptide.

In another aspect, provided herein is a conjugate comprising an antibody (Ab) that specifically binds an extracellular domain of a human SIRP-α polypeptide, at least one Q tag peptide sequence comprising a glutamine residue, and at least one immunomodulatory oligonucleotide (P), wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61); wherein the Q-tag peptide sequence is naturally occurring or synthetic; wherein each immunomodulatory oligonucleotide is linked to a Q-tag via an amide bond with the glutamine residue and linker (L); and wherein at least one Q-tag peptide sequence is selected from the group consisting of SEQ ID NOs: 39-55.

In some embodiments according to any of the embodiments described herein, the VH domain comprises the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62). In some embodiments, the VH domain comprises the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62), and the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVL (SEQ ID NO:63). In some embodiments, the VH domain comprises the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62), and the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTKLTVL (SEQ ID NO:64). In some embodiments, the VH domain comprises the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62), and the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTELTVL (SEQ ID NO:65).

In some embodiments according to any of the embodiments described herein, the antibody is a monoclonal antibody. In some embodiments, the antibody is a Fab, F(ab′)2, Fab′-SH, Fv, or scFv antibody or antibody fragment. In some embodiments, the antibody is a humanized, human, or chimeric antibody or fragment thereof. In some embodiments, the antibody comprises an antibody heavy chain comprising a VH domain of the present disclosure and an Fc region. In some embodiments, the antibody heavy chain comprises a human IgG1, human IgG2, or human IgG4 Fc region. In some embodiments, the antibody heavy chain comprises a human IgG1 Fc region comprising L234A, L235A, and/or G237A substitutions, amino acid position numbering according to EU index. In some embodiments, the antibody heavy chain comprises a wild-type human IgG1 Fc region. In some embodiments, the antibody heavy chain comprises a human IgG1 Fc region comprising an N297A substitution, amino acid position numbering according to EU index. In some embodiments, the antibody heavy chain comprises a human IgG1 Fc region comprising a D265A substitution, amino acid position numbering according to EU index. In some embodiments, the antibody heavy chain comprises a wild-type human IgG2 Fc region. In some embodiments, the antibody heavy chain comprises a human IgG2 Fc region comprising an N297A substitution, amino acid position numbering according to EU index. In some embodiments, the antibody heavy chain comprises a human IgG4 Fc region comprising an S228P substitution, amino acid position numbering according to EU index. In some embodiments, the Fc region comprises an N297A substitution, amino acid position numbering according to EU index. In some embodiments, the conjugate further comprises an immunomodulating oligonucleotide P attached to the Q295 of the Fc region residue as shown in the following formula

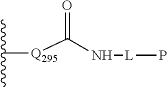

wherein L is a linker moiety connected to Q295 of the Fc region via an amide bond.

In some embodiments according to any of the embodiments described herein, the antibody comprises an antibody heavy chain, with C-terminal Q-tag peptide, comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLGRPQGFGPP (SEQ ID NO:66). In some embodiments, the antibody comprises an antibody heavy chain comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLG (SEQ ID NO:87). In some embodiments, the antibody comprises an antibody heavy chain comprising the amino acid sequence of SEQ ID NO: 87 and a Q-tag sequence of the present disclosure. In some embodiments, the antibody comprises an antibody heavy chain, with C-terminal Q-tag peptide, comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEA AGAPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGRPQGFGPP (SEQ ID NO:67). In some embodiments, the antibody comprises an antibody heavy chain comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEA AGAPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:88). In some embodiments, the antibody comprises an antibody heavy chain comprising the amino acid sequence of SEQ ID NO: 88 and a Q-tag sequence of the present disclosure. In some embodiments, the antibody comprises an antibody heavy chain, with C-terminal Q-tag peptide, comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIA VEWESNGQPENNYKTTPPVLDSDGSFFLYSKLT VDKSRWQQGNVFSCSVMHEALHNHYTQKSLSLSPGRPQGFGPP (SEQ ID NO:68). In some embodiments, the antibody comprises an antibody heavy chain comprising the amino acid sequence EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:89). In some embodiments, the antibody comprises an antibody heavy chain comprising the amino acid sequence of SEQ ID NO:89 and a Q-tag sequence of the present disclosure.

In some embodiments according to any of the embodiments described herein, the antibody comprises an antibody light chain comprising the VL domain and a light chain constant (CL) domain comprising the amino acid sequence GQPKANPTVTLFPPSSEELQANKATLVCLISDFYPGAVTVAWKADGSPVKAGVETTKPS KQSNNKYAASSYLSLTPEQWKSHRSYSCQVTHEGSTVEKTVAPTECS (SEQ ID NO:69). In some embodiments, the antibody comprises an antibody light chain comprising the VL domain and a light chain constant (CL) domain comprising the amino acid sequence GQPKANPTVTLFPPSSEELQANKATLVCLISDFYPGAVTVAWKADGSPVKAGVETTKPS KQSSDKYAASSYLSLTPEQWKSHRSYSCQVTHEGSTVEKTVAPTECS (SEQ ID NO:70). In some embodiments, the antibody comprises an antibody light chain comprising the VL domain and a light chain constant (CL) domain comprising the amino acid sequence GQPKAAPSVTLFPPSSEELQANKATLVCLISDFYPGAVTVAWKADSSPVKAGVETTTPS KQSNNKYAASSYLSLTPEQWKSHRSYSCQVTHEGSTVEKTVAPTECS (SEQ ID NO:71). In some embodiments, the antibody comprises an antibody light chain comprising an amino acid sequence selected from the group consisting of SEQ ID Nos: 72-80. In some embodiments, the antibody comprises an antibody heavy chain, with C-terminal Q-tag peptide, comprising the amino acid sequence of SEQ ID NO:68 and an antibody light chain comprising the amino acid sequence of SEQ ID NO:79. In some embodiments, the antibody comprises an antibody heavy chain, with C-terminal Q-tag peptide, comprising the amino acid sequence of SEQ ID NO:67 and an antibody light chain comprising the amino acid sequence of SEQ ID NO:79. In some embodiments, the antibody comprises an antibody heavy chain, with C-terminal Q-tag peptide, comprising the amino acid sequence of SEQ ID NO:66 and an antibody light chain comprising the amino acid sequence of SEQ ID NO:79. In some embodiments, the antibody comprises an antibody heavy chain, with C-terminal Q-tag peptide, comprising the amino acid sequence of SEQ ID NO:68 and an antibody light chain comprising the amino acid sequence of SEQ ID NO: 75. In some embodiments, the antibody comprises an antibody heavy chain, with C-terminal Q-tag peptide, comprising the amino acid sequence of SEQ ID NO:67 and an antibody light chain comprising the amino acid sequence of SEQ ID NO:75. In some embodiments, the antibody comprises an antibody heavy chain, with C-terminal Q-tag peptide, comprising the amino acid sequence of SEQ ID NO:66 and an antibody light chain comprising the amino acid sequence of SEQ ID NO:75. In some embodiments, the antibody comprises an antibody heavy chain, with C-terminal Q-tag peptide, comprising the amino acid sequence of SEQ ID NO:68 and an antibody light chain comprising the amino acid sequence of SEQ ID NO:73. In some embodiments, the antibody comprises an antibody heavy chain, with C-terminal Q-tag peptide, comprising the amino acid sequence of SEQ ID NO:67 and an antibody light chain comprising the amino acid sequence of SEQ ID NO:73. In some embodiments, the antibody comprises an antibody heavy chain, with C-terminal Q-tag peptide, comprising the amino acid sequence of SEQ ID NO:66 and an antibody light chain comprising the amino acid sequence of SEQ ID NO: 73. In some embodiments, the antibody comprises an antibody heavy chain comprising the amino acid sequence of SEQ ID NO:88 and an antibody light chain comprising the amino acid sequence of SEQ ID NO:79. In some embodiments, the antibody comprises an antibody heavy chain comprising the amino acid sequence of SEQ ID NO:87 and an antibody light chain comprising the amino acid sequence of SEQ ID NO:79. In some embodiments, the antibody comprises an antibody heavy chain comprising the amino acid sequence of SEQ ID NO:89 and an antibody light chain comprising the amino acid sequence of SEQ ID NO:75. In some embodiments, the antibody comprises an antibody heavy chain comprising the amino acid sequence of SEQ ID NO:88 and an antibody light chain comprising the amino acid sequence of SEQ ID NO:75. In some embodiments, the antibody comprises an antibody heavy chain comprising the amino acid sequence of SEQ ID NO:87 and an antibody light chain comprising the amino acid sequence of SEQ ID NO:75. In some embodiments, the antibody comprises an antibody heavy chain comprising the amino acid sequence of SEQ ID NO:89 and an antibody light chain comprising the amino acid sequence of SEQ ID NO:73. In some embodiments, the antibody comprises an antibody heavy chain comprising the amino acid sequence of SEQ ID NO: 88 and an antibody light chain comprising the amino acid sequence of SEQ ID NO:73. In some embodiments, the antibody comprises an antibody heavy chain comprising the amino acid sequence of SEQ ID NO:87 and an antibody light chain comprising the amino acid sequence of SEQ ID NO:73. In some embodiments, the antibody heavy and/or light chain further comprises a Q-tag peptide sequence of the present disclosure.

In some embodiments according to any of the embodiments described herein, each of the one or more Q-tag peptides (Q) comprises a peptide sequence having between 5 and 15 amino acid residues. In some embodiments, each of the one or more Q-tag peptides is naturally occurring. In some embodiments, each of the one or more Q-tag peptides comprises a sequence independently selected from the group consisting of SEQ ID NOs: 39-55. In some embodiments, each of the one or more Q-tag peptides comprises the peptide sequence RPQGF (SEQ ID NO: 47), RPQGFPP (SEQ ID NO:48), or RPQGFGPP (SEQ ID NO:49). In some embodiments, each of the one or more Q-tag peptides comprises the peptide sequence RPQGF (SEQ ID NO: 47). In some embodiments, the at least one Q-tag peptide sequence comprises the peptide sequence RPQGF (SEQ ID NO:47), RPQGFPP (SEQ ID NO:48), or RPQGFGPP (SEQ ID NO: 49). In some embodiments, the at least one Q-tag peptide sequence comprises the peptide sequence RPQGFGPP (SEQ ID NO:49). In some embodiments, the antibody comprises two antibody heavy chains and two antibody light chains, and wherein one or both heavy chains further comprises a Q-tag. In some embodiments, the Q-tag is fused to the C-terminus of one or both of the heavy chains. In some embodiments, the Q-tag is within the Fc domain. In some embodiments, the antibody comprises two antibody heavy chains and two antibody light chains, and wherein one or both light chains further comprises a Q-tag. In some embodiments, the conjugate induces activation of TLR9. In some embodiments, 1 or 2 Q-tags is/are linked to the antibody. In some embodiments, the conjugate has a DAR of 1 or 2.

In some embodiments, the linker L comprises a polyethylene glycol moiety. In some embodiments, the linker L is

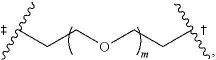

wherein m is an integer ranging from about 0 to about 50, and wherein [symbol] † indicates the point of attachment to T³, and [symbol] ‡ indicates the point of attachment to the rest of the conjugate. In some embodiments, Z is S. In some embodiments, the oligonucleotide P comprises at least one pair of geminal T¹ and T² wherein T¹ is S and T² is S⁻. In some embodiments, the at least one pair of geminal T¹ and T² wherein T¹ is S and T² is S is located at the 3′-position of nucleoside residue 1. In some embodiments, the at least one pair of geminal T¹ and T² wherein T¹ is S and T² is S is located at the 3′-position of nucleoside residue 2. In some embodiments, the at least one pair of geminal T¹ and T² wherein T¹ is S and T² is S is located at the 3′-position of nucleoside residue 3. In some embodiments, the at least one pair of geminal T¹ and T² wherein T¹ is S and T² is S is located at the 3′-position of nucleoside residue 5. In some embodiments, the at least one pair of geminal T¹ and T² wherein T¹ is S and T² is S is located at the 3′-position of nucleoside residue 6. In some embodiments, the at least one pair of geminal T¹ and T² wherein T¹ is S and T² is S is located at the 3′-position of nucleoside residue 7. In some embodiments, the at least one pair of geminal T¹ and T² wherein T¹ is S and T² is S is located at the 3′-position of nucleoside residue 8. In some embodiments, the at least one pair of geminal T¹ and T² wherein T¹ is S and T² is S is located at the 3′-position of nucleoside residue 9. In some embodiments, the at least one pair of geminal T¹ and T² wherein T¹ is S and T² is S is located at the 3′-position of nucleoside residue 10. In some embodiments, the at least one pair of geminal T¹ and T² wherein T¹ is S and T² is S is located at the 3′-position of nucleoside residue 11. In some embodiments, the at least one pair of geminal T¹ and T² wherein T¹ is S and T² is Sis located at the 3′-position of nucleoside residue 12. In some embodiments, the at least one pair of geminal T¹ and T² wherein T¹ is S and T² is S is located at the 3′-position of nucleoside residue 13. In some embodiments, the at least one pair of geminal T¹ and T² wherein T¹ is S and T² is S is located at the 3′-position of nucleoside residue 14. In some embodiments, the at least one pair of geminal T¹ and T² wherein T¹ is S and T² is S is located at the 3′-position of nucleoside residue 15. In some embodiments, the oligonucleotide P comprises at least two pairs of geminal T¹ and T² wherein T¹ is S and T² is S⁻. In some embodiments, R⁵′ is H. In some embodiments, R⁵′ is methoxy. In some embodiments, R^{c1} is H. In some embodiments, R^{c1} is methoxy. In some embodiments, R² is methyl. In some embodiments, R² is H. In some embodiments, U^{5′} is bromo. In some embodiments, U^{5′} is —H. In some embodiments, m is an integer from 20 to 25. In some embodiments, m is 24. In some embodiments, each P independently comprises an oligonucleotide selected from Table 2, Table 14, and Table 15. In some embodiments, each linker (L) and an each immunomodulating oligonucleotide (P) independently comprises an oligonucleotide selected from Table 2, Table 16 and Table 17. In some embodiments, each P independently comprises an oligonucleotide selected from SEQ ID NOS: 1-38 and 129-166. In some embodiments, each immunomodulating oligonucleotide P is independently

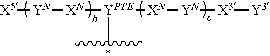

wherein
   b and c are each independently an integer from 1 to 25; with the proviso that the sum of b and c is at least 5;
   [symbol] * indicates the point of attachment of the immunomodulating oligonucleotide P to the rest of the conjugate;
X^{5′} is a 5′ terminal nucleoside comprising the structure

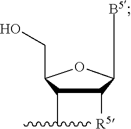

X^{3′} is a 3′ terminal nucleoside comprising the structure

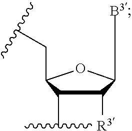

Y^{PTE} is an internucleoside phosphotriester comprising the structure

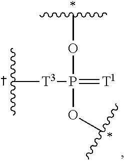

wherein * indicates the points of attachment to the rest of the oligonucleotide and [symbol] † indicates the point of attachment to the linker L, or, if L is absent, [symbol] † indicates the point of attachment to the Q tag peptide at the glutamine residue via an amide bond;
Y^{3′} is a terminal phosphotriester comprising the structure

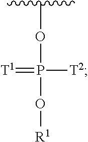

each X^{N} is independently a nucleoside comprising the structure

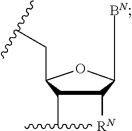

each Y^{N} is independently an internucleoside linker comprising the structure

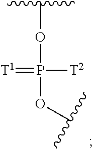

wherein each B^{N} is independently a modified or unmodified nucleobase;
   each R^{N} is independently —H or —O—C₁₋₄-alkyl, wherein the C₁₋₄-alkyl of the —O—C₁₋₄-alkyl is optionally further substituted by —O—C₁-C₄-alkyl;
   B^{5′} and B^{3′} are independently a modified or unmodified nucleobase;
   R^{5′} and R^{3′} are independently —H or —O—C₁-C₄-alkyl, wherein the C₁₋₄-alkyl of the —O—C₁₋₄-alkyl is optionally further substituted by —O—C₁₋₄-alkyl;
   each T₁ is independently O or S;
   each T₂ is independently O⁻ or S⁻; and
   T₃ is a group comprising an oligoethylene glycol moiety; and
   R¹ is C₁₋₄-alkylene-hydroxy.

In some embodiments according to any of the embodiments described herein, b is 3. In some embodiments, (i) P comprises at least one modified nucleoside X^{N}; (ii) P comprises at least one modified internucleoside linker Y^{N}, wherein at least one of T¹ or T² is S; or (iii) both (i) and (ii). In some embodiments, P comprises at least one phosphorodithioate or phosphorothioate internucleoside linker. In some embodiments, P comprises 0, 1, 2, or 3 phosphorodithioate internucleoside linkers. In some embodiments, P comprises a modified nucleoside selected from the group consisting of 2′-O-alkyl nucleoside, 2′-O-alkoxyalkyl nucleoside, 2′-deoxynucleoside and ribonucleoside. In some embodiments, the modified nucleoside is selected from the group consisting of 5-bromo-2′-O-methyluridine, 5-bromo-2′-deoxyuridine, 2′-O-methyluridine, 2′-deoxyuridine, 2′-O-methylthymidine, 2′-O-methylcytidine, 2′-O-(2-methoxyethyl) thymidine and 8-oxo-7,8-dihydro-2′-deoxyguanosine. In some embodiments, X^{5′} is a 5-bromo-2′-O-methyluridine, 5-bromo-2′-deoxyuridine, 2′-O-methyluridine or 2′-deoxyuridine. In some embodiments, Y^{3′} or the Y^{N} at the 3′ position of X^{5′} comprises an unsubstituted or substituted phosphorothioate. In some embodiments, Y^{PTE} is

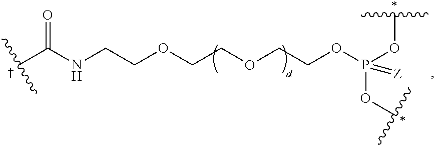

wherein Z is O or S; d is an integer from 0 to 95; the two [symbol] * on the right side of the structure indicate the points of attachment to the adjacent nucleosides X^{N} in the oligonucleotide P, and the [symbol] † on the left side of the structure indicates the point of attachment to the linker L. In some embodiments, Y^{PTE} is:

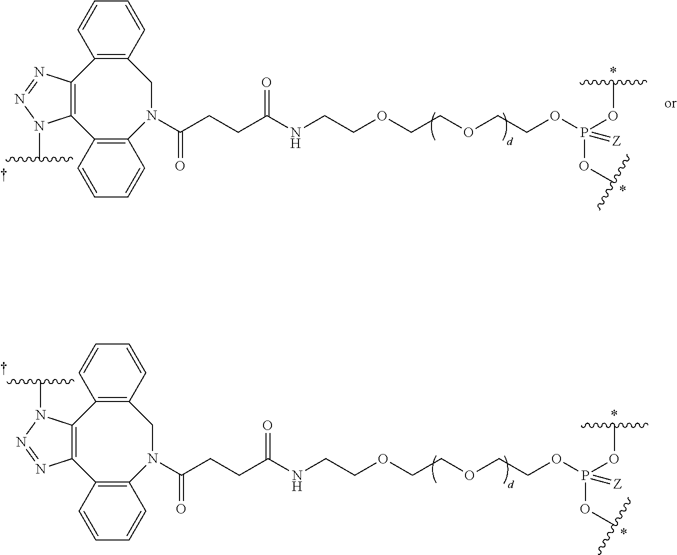

wherein Z is O or S; d is an integer from 0 to 95; the two [symbol] * on the right side of the structure indicate the points of attachment to the adjacent nucleosides X^{N} in the oligonucleotide P, and the one [symbol] † on the left side of the structure indicates the point of attachment to the linker L. In some embodiments, Z is S. In some embodiments, d is an integer from 1 to 25. In some embodiments, the linker L comprises a polyethylene glycol moiety. In some embodiments, the linker L is

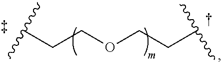

wherein m is an integer ranging from about 0 to about 50, and wherein [symbol] † indicates the point of attachment to Y^{PTE}, and [symbol] ‡ indicates the point of attachment to the rest of the conjugate. In some embodiments, P comprises one or more CpG sites. In some embodiments, the antibody comprises two antibody light chains, two antibody heavy chains, and two Q-tag peptides; wherein each of the Q-tag peptides is linked to the C-terminus of one of the antibody heavy chains; and wherein one of the Q-tag peptides is linked to an immunomodulating oligonucleotide (P) via an amide bond with the glutamine residue of the Q-tag peptide and linker (L).

In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains is linked to a C-terminal Q-tag peptide (Q) and comprises the amino acid sequence, with Q, of SEQ ID NO:68; wherein the immunomodulating oligonucleotide comprises the sequence of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO:34; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 75; wherein each of the antibody heavy chains comprises the amino acid sequence of SEQ ID NO:89; wherein the immunomodulating oligonucleotide comprises the sequence of SEQ ID NO: 35; wherein each of the Q-tag peptides (Q) comprises the amino acid sequence of SEQ ID NO: 49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:88; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:87; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:89; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:88; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:87; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:88; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:89; wherein the immunomodulating oligonucleotide and the linker comprise the structure of SEQ ID NO:35; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:89; wherein the immunomodulating oligonucleotide and the linker comprise the structure of SEQ ID NO:34; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:88; wherein the immunomodulating oligonucleotide and the linker comprise the structure of SEQ ID NO:34; wherein each of the Q-tag peptides (Q) comprises the amino acid sequence of SEQ ID NO: 49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:87; wherein the immunomodulating oligonucleotide and the linker comprise the structure of SEQ ID NO:34; wherein each of the Q-tag peptides (Q) comprises the amino acid sequence of SEQ ID NO: 49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:89; wherein the immunomodulating oligonucleotide and the linker comprise the structure of SEQ ID NO:34; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:88; wherein the immunomodulating oligonucleotide and the linker comprise the structure of SEQ ID NO:34; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:87; wherein the immunomodulating oligonucleotide and the linker comprise the structure of SEQ ID NO:34; wherein each of the Q-tag peptides (Q) comprises the amino acid sequence of SEQ ID NO: 49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:89; wherein the immunomodulating oligonucleotide and the linker comprise the structure of SEQ ID NO:163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:88; wherein the immunomodulating oligonucleotide and the linker comprise the structure of SEQ ID NO: 163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:75; wherein each of the antibody heavy chains comprises the amino acid sequence of SEQ ID NO:87; wherein the immunomodulating oligonucleotide and the linker comprise the structure of SEQ ID NO:163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:89; wherein the immunomodulating oligonucleotide and the linker comprise the structure of SEQ ID NO:163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:88; wherein the immunomodulating oligonucleotide and the linker comprise the structure of SEQ ID NO:163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:73; wherein each of the antibody heavy chains comprises the amino acid sequence of SEQ ID NO:87; wherein the immunomodulating oligonucleotide and the linker comprise the structure of SEQ ID NO:163; wherein each of the Q-tag peptides (Q) comprises the amino acid sequence of SEQ ID NO:49; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:79; wherein each of the antibody heavy chains is linked to a C-terminal Q-tag peptide (Q) and comprises the amino acid sequence, with Q, of SEQ ID NO:66; wherein the immunomodulating oligonucleotide comprises the sequence of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO:79; wherein each of the antibody heavy chains is linked to a C-terminal Q-tag peptide (Q) and comprises the amino acid sequence, with Q, of SEQ ID NO:67; wherein the immunomodulating oligonucleotide comprises the sequence of SEQ ID NO:35; and wherein at least one of the two Q-tag peptides is linked to the immunomodulating oligonucleotide via an amide bond with the glutamine residue of the Q-tag peptide and a linker (L). In another aspect, provided herein is a conjugate comprising an antibody (Ab) and an immunomodulating oligonucleotide (P), wherein the antibody comprises two antibody light chains, two antibody heavy chains, and two Q-tag peptides; wherein each of the antibody light chains comprises the amino acid sequence of SEQ ID NO: 79; wherein each of the antibody heavy chains is linked to a C-terminal Q-tag peptide (Q) and comprises the amino acid sequence, with Q, of SEQ ID NO:68; wherein the immunomodulating oligonucleotide comprises the sequence of SEQ ID NO:35; and wherein at least one of the two Q-tag peptides is linked to the immunomodulating oligonucleotide via an amide bond with the glutamine residue of the Q-tag peptide and a linker (L). In some embodiments, the conjugate has a DAR of 1 or 2.

In another aspect, provided herein is a method for preparing a conjugate that comprises (i) an antibody or antigen-binding fragment thereof (Ab) that specifically binds human an extracellular domain of a human SIRP-α polypeptide and (ii) one or more immunomodulating oligonucleotides (P), comprising: contacting the Ab with the oligonucleotide P in the presence of a transglutaminase; wherein the antibody or antigen-binding fragment is linked to one or more Q-tag peptides (Q) comprising the amino acid sequence RPQGF (SEQ ID NO:47); wherein each P independently comprises the following formula:

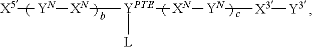

wherein X^{5′} is a 5′ terminal nucleoside; X^{3′} is a 3′ terminal nucleoside; Y^{PTE} is an internucleoside phosphotriester; Y^{3′} is a terminal phosphotriester; each X^{N} is independently a nucleoside; each Y^{N} is independently an internucleoside linker; b and c are each independently an integer from 1 to 25; with the proviso that the sum of b and c is at least 5; and L is a linker moiety comprising a terminal amine; and wherein the antibody or antigen-binding fragment comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO: 59), a CDR-L2 comprising the amino acid sequence of SDDKRPS (SEQ ID NO:60), and a CDR-L3 comprising the amino acid sequence of GGYDQSSYTNP (SEQ ID NO:61). In another aspect, provided herein is a method for preparing a conjugate that comprises (i) an antibody or antigen-binding fragment thereof (Ab) that specifically binds human an extracellular domain of a human SIRP-α polypeptide and (ii) one or more immunomodulating oligonucleotides (P), wherein the antibody or antigen-binding fragment is linked to one or more Q-tag peptides (Q) comprising the amino acid sequence RPQGF (SEQ ID NO:47); wherein the antibody or antigen-binding fragment comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61); and wherein each immunomodulating oligonucleotide is linked to a Q-tag peptide via an amide bond with the glutamine residue of the Q-tag peptide and a linker (L) as shown in formula (A),

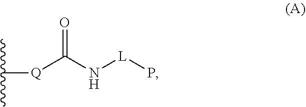

wherein:
   [symbol] indicates the point of attachment of each Q to the antibody or antigen-binding fragment thereof (Ab);
   each Q independently comprises a Q-tag peptide sequence RPQGF (SEQ ID NO: 47);
   each L is independently a bond or a linker moiety connected to Q via an amide bond with the glutamine residue; and
   each P is independently an immunomodulating oligonucleotide;
comprising contacting a compound of formula (B)

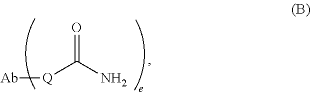

wherein Ab and Q are as defined for formula (A) above, and e is an integer from 1 to 20, with one or more immunomodulating oligonucleotides P, wherein each P independently comprises the following formula:

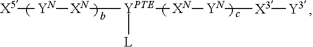

wherein
   X^{5′} is a 5′ terminal nucleoside;
   X^{3′} is a 3′ terminal nucleoside;
   Y^{PTE} is an internucleoside phosphotriester;
   Y^{3′} is a terminal phosphotriester;
   each X^{N} is independently a nucleoside;
   each Y^{N} is independently an internucleoside linker;
   b and c are each independently an integer from 1 to 25; with the proviso that the sum of b and c is at least 5; and
   L is a linker moiety comprising a terminal amine,
in the presence of a transglutaminase. In some embodiments, each immunomodulating oligonucleotide is independently an oligonucleotide of formula (C) or formula (D) is selected from the group consisting of the oligonucleotides of Table 15 and Table 17.

In some embodiments, each Q-tag peptide sequence comprises the peptide sequence RPQGFGPP (SEQ ID NO:49). In some embodiments, the Ab comprises two antibody light chains, two antibody heavy chains, and two Q-tag peptides (Q) having at least one glutamine residue; wherein one Q-tag peptide is linked to the C-terminus of each of the two antibody heavy chains. In some embodiments, the conjugate has a DAR of 1 or 2. In some embodiments, the conjugate has a DAR of 1, and the method further comprises separating the conjugate having a DAR of 1 from free oligonucleotide, unconjugated antibody, and conjugates having a DAR of 2.

In another aspect, provided herein is a pharmaceutical composition comprising the conjugate of any one of the above embodiments and a pharmaceutically acceptable carrier.

In another aspect, provided herein is a method for treating cancer, comprising administering to an individual an effective amount of the conjugate according to any one of the above embodiments or the pharmaceutical composition according to any one of the above embodiments. In another aspect, provided herein is the conjugate according to any one of the above embodiments or the pharmaceutical composition according to any one of the above embodiments for use in a method for treating cancer. In another aspect, provided herein are uses of the conjugate according to any one of the above embodiments or the pharmaceutical composition according to any one of the above embodiments in the manufacture of a medicament, e.g., for use in treating cancer. In another aspect, provided herein is a method for activating myeloid cells, comprising administering to an individual in need thereof an effective amount of the conjugate according to any one of the above embodiments or the pharmaceutical composition according to any one of the above embodiments. In another aspect, provided herein is a method for inducing TLR9 signaling in myeloid cells, comprising administering to an individual in need thereof an effective amount of the conjugate according to any one of the above embodiments or the pharmaceutical composition according to any one of the above embodiments. In some embodiments, the individual has cancer. In some embodiments, the cancer is a liquid tumor. In other embodiments, the cancer is a solid tumor. In some embodiments, the cancer is lung cancer, squamous cell cancer, cholangiocarcinoma (e.g., intrahepatic cholangiocarcinoma), brain tumors, glioblastoma, head and neck cancer, hepatocellular cancer, colorectal cancer, skin cancer, lung cancer, endometrial cancer, liver cancer, bladder cancer, gastric or stomach cancer, pancreatic cancer, cervical cancer, ovarian cancer, cancer of the urinary tract, urothelial cancer, breast cancer, peritoneal cancer, uterine cancer, salivary gland cancer, kidney or renal cancer, prostate cancer, vulval cancer, thyroid cancer, anal carcinoma, penile carcinoma, testis or testicular cancer, melanoma, multiple myeloma and B-cell lymphoma, non-Hodgkin's lymphoma (NHL), acute lymphoblastic leukemia (ALL), chronic lymphocytic leukemia (CLL), acute myeloid leukemia (AML), Merkel cell carcinoma, hairy cell leukemia, or chronic myeloblastic leukemia (CML). In some embodiments, the cancer is known or predicted to be non-responsive to an inhibitor of PD-L1 or PD-1 (e.g., when administered as a monotherapy, or when administered in the absence of an anti-SIRP-α antibody). In some embodiments, the individual does not achieve a significant therapeutic response to an inhibitor of PD-L1 or PD-1 (e.g., an antibody that binds PD-L1 or PD-1). In some embodiments, prior to administration of the conjugate or composition, the individual has been treated with an inhibitor of PD-L1 or PD-1 (e.g., an antibody that binds PD-L1 or PD-1). In some embodiments, the inhibitor of PD-L1 or PD-1 is pembrolizumab, nivolumab, cemiplimab-rwlc, atezolizumab, dostarlimab-gxly, durvalumab, or avelumab. In some embodiments, prior to administration of the conjugate or composition, the individual has been treated with an inhibitor of PD-L1 or PD-1 (e.g., an antibody that binds PD-L1 or PD-1) and did not respond to said treatment with the inhibitor of PD-L1 or PD-1 (e.g., when administered as a monotherapy, or when administered in the absence of an anti-SIRP-α antibody). In some embodiments, the cancer is melanoma or renal cancer, e.g., that expresses or overexpresses SIRP-α. In some embodiments, cells of the cancer express human SIRP-α. In some embodiments, cells of the cancer do not express human SIRP-α. In some embodiments, the methods further comprise administering to the individual an additional therapeutic agent. In some embodiments, the additional therapeutic agent comprises an immunotherapy, chemotherapy, radiation therapy, cell-based therapy, anti-cancer vaccine, or anti-cancer agent. In some embodiments, the methods further comprise administering to the individual an inhibitor of PD-L1 or PD-1 (e.g., an antibody that binds PD-L1 or PD-1). In some embodiments, the methods comprise administering to the individual an inhibitor of PD-L1 or PD-1, wherein: (a) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO:35; and wherein at least one of the two Q-tag peptides is linked to the immunomodulating oligonucleotide via an amide bond with the glutamine residue of the Q-tag peptide and a linker (L); (b) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO: 35; and wherein at least one of the two Q-tag peptides is linked to the immunomodulating oligonucleotide via an amide bond with the glutamine residue of the Q-tag peptide and a linker (L); (c) the antibody of the conjugate comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO: 35; and wherein at least one of the two Q-tag peptides is linked to the immunomodulating oligonucleotide via an amide bond with the glutamine residue of the Q-tag peptide and a linker (L); (d) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L); (e) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L); (f) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO: 163; and wherein at least one of the two Q-tag peptides is linked to the immunomodulating oligonucleotide via an amide bond with the glutamine residue of the Q-tag peptide and a linker (L); (g) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:68; wherein the immunomodulating oligonucleotide and the linker comprise the structure of SEQ ID NO: 34; and wherein at least one of the two Q-tag peptides is linked to the immunomodulating oligonucleotide via an amide bond with the glutamine residue of the Q-tag peptide and a linker (L); (h) the antibody of the conjugate comprises two antibody light chains, two antibody heavy chains, and two Q-tag peptides; wherein each of the antibody light chains comprises the amino acid sequence of SEQ ID NO: 73; wherein each of the antibody heavy chains is linked to a C-terminal Q-tag peptide (Q) and comprises the amino acid sequence, with Q, of SEQ ID NO:67; wherein the immunomodulating oligonucleotide and the linker comprise the structure of SEQ ID NO: 34; and wherein at least one of the two Q-tag peptides is linked to the immunomodulating oligonucleotide via an amide bond with the glutamine residue of the Q-tag peptide and a linker (L); or (i) the antibody of the conjugate comprises two antibody light chains, two antibody heavy chains, and two Q-tag peptides; wherein each of the antibody light chains comprises the amino acid sequence of SEQ ID NO:73; wherein each of the antibody heavy chains is linked to a C-terminal Q-tag peptide (Q) and comprises the amino acid sequence, with Q, of SEQ ID NO:66; wherein the immunomodulating oligonucleotide and the linker comprise the structure of SEQ ID NO: 34; and wherein at least one of the two Q-tag peptides is linked to the immunomodulating oligonucleotide via an amide bond with the glutamine residue of the Q-tag peptide and a linker (L).

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE FIGURES

The present application can be understood by reference to the following description taken in conjunction with the accompanying figures.

FIGS. 1A and 1B depict the activity of immunomodulating oligonucleotides alone in human PBMCs based upon observed increased expression of HLADR (FIG. 1A) and CD40 (FIG. 1B).

FIGS. 2A-2C show the effect of immunomodulating oligonucleotides on increasing B cell numbers and activation. FIG. 2A depicts the observed effect on B cell numbers by the various immunomodulating polypeptides alone. FIGS. 2B-2C depict the observed activation of B cells (via detection of CD40 expression) produced by the immunomodulating oligonucleotides alone.

FIG. 3 shows the percentage yields of transglutaminase-mediated conjugations and deconjugations with a polyethylene glycol linker (—NH—C(═O)-PEG₂₃-NH₂) and various Q-tags. Shown are SEQ ID Nos: 39-47 and 50-52.

FIGS. 4A and 4B show the percentage change of conjugation and decongjuation over time in transglutaminase conjugation of two Q-tag peptides (LSLSPGLLQGG, SEQ ID NO:39; and RPQGF, SEQ ID NO:47).

FIG. 5 shows activity of indicated free CpG oligonucleotides on human PBMCs, as assayed by CD40 expression on CD19+ B cells.

FIG. 6 shows activity of indicated free CpG oligonucleotides on human PBMCs, as assayed by Ramos NFkb Reporter Assay.

FIGS. 7A-7C show activity of indicated free CpG oligonucleotides on human PBMCs from three different donor lines (D559, D804 and D643), as observed by CD40 expression.

FIG. 8 shows activity of indicated free CpG oligonucleotides on human PBMCs, as assayed by CD40 expression on CD19+ B cells.

FIGS. 9A-9D show a schematic diagram of exemplary conjugates, in accordance with some embodiments. Exemplary antibody:CpG conjugates with an engineered Q-tag (RPQGFGPP; SEQ ID NO:49) fused to the C-terminus of the heavy chain are shown in FIG. 9A (with a DAR 1) and in FIG. 9B (with DAR 2). Exemplary antibody:CpG conjugates with a naturally-occurring Q-tag (Q295) exposed for conjugation by an N297A mutation are shown in FIG. 9C (with a DAR 1) and in FIG. 9D (with DAR 2).

FIGS. 10A-10D show activation of human myeloid cells (e.g., monocytes and dendritic cells) by anti-SIRP-α antibody-CpG oligonucleotide conjugates. Activation was assessed by CD86 expression using flow cytometry.

FIGS. 11A-11D show stimulation of interferon regulatory factor (IRF7; FIGS. 11A & 11B) and interleukin-6 (IL-6; FIGS. 11C & 11D) production in human myeloid cells (e.g., monocytes and dendritic cells) by anti-SIRP-α antibody-CpG oligonucleotide conjugates.

FIGS. 12A-12D show stimulation of cytokine secretion in human PBMCs by anti-SIRP-α antibody-CpG oligonucleotide conjugates. Shown are secretion of IFN-α2 (FIG. 12A), IFN-γ (FIG. 12B), IL-6 (FIG. 12C), and IL-10 (FIG. 12D).

FIG. 13 shows the ability of CpG oligonucleotides to activate CD40 expression in myeloid cells. Shown are the effects on CD14+ monocytes (left) and dendritic cells (right).

FIG. 14 shows the stimulation of tumor phagocytosis by monocyte-derived M2 macrophages upon treatment with anti-SIRP-α antibody-CpG oligonucleotide conjugate, as compared to unconjugated antibody, or media control.

FIGS. 15A & 15B show anti-tumor effects of anti-SIRP-α antibody-CpG oligonucleotide conjugates using either mouse IgG2a or mouse IgG1 Fc region in a RENCA (SIRP-α positive) syngeneic tumor model.

FIGS. 16A & 16B show activation of monocytes when PBMCs are co-cultured in the presence of SIRP-α-positive or SIRP-α-negative tumor cell lines by three anti-SIRP-α antibody-CpG oligonucleotide conjugates, each conjugated to a different Fc domain (human IgG4, human IgG1, and human IgG1-AAA), as compared to the unconjugated antibodies. DLD-1 cells in FIG. 16A were transduced to overexpress SIRP-α (SIRP-α-positive), whereas the parental DLD-1 cells in FIG. 16B did not express SIRP-α (SIRP-α-negative).

FIG. 17A shows a diagram of anti-SIRP-α antibody-CpG oligonucleotide conjugate.

FIGS. 17B-17D show that the anti-SIRP-α antibody-CpG oligonucleotide conjugate is a potent TLR9 agonist with targeted activation across species. Human PBMCs (FIG. 17B), cynomolgus PBMCs (FIG. 17C), or mouse splenocytes (FIG. 17D) were stimulated with anti-hSIRP-αantibody, anti-hSIRP-α antibody-CpG oligonucleotide conjugate, CpG 7-7 oligo, or anti-mSIRP-α antibody-CpG oligonucleotide conjugate with murine reactive mT-CpG for 24 hr or 48 hrs and surface marker expression was assayed by flow cytometry.

FIGS. 18A & 18B show that anti-SIRP-α antibody-CpG oligonucleotide conjugate specifically targets and activates SIRP-α-positive immune cells in human PBMC cultures. Human PBMCs were stimulated with anti-SIRP-α antibody-CpG oligonucleotide conjugate or anti-CD22 antibody-CpG oligonucleotide conjugate for 24 hr and surface marker expression on monocytes (FIG. 18A) and B cells (FIG. 18B) was assayed by flow cytometry.

FIGS. 19 & 20 show activation of myeloid cells co-cultured with SIRP-α-positive DLD-1 tumor cells (FIG. 19) vs. SIRP-α-negative DLD-1 tumor cells (FIG. 20).

FIGS. 21A & 21B show phagocytosis of SIRP-α-positive DLD-1 tumor cells (FIG. 21A) or SIRP-α-negative DLD-1 tumor cells (FIG. 21B) in the presence of human monocyte-derived macrophages and anti-SIRP-α antibody-CpG oligonucleotide conjugate or anti-SIRP-α antibody. % Phagocytosis was determined by flow cytometry.

FIGS. 22A & 22B show anti-tumor activity of anti-SIRP-α antibody-CpG oligonucleotide conjugate in mouse syngeneic model. Mice bearing MC38 overexpressing SIRP-α (FIG. 22A) or parental MC38 cells (FIG. 22B) were dosed intraperitoneally (i.p.) twice, three days apart with anti-mSIRP-α antibody conjugated with murine reactive mT-CpG at 1 mg/kg (squares) or PBS control (triangles). Arrows indicate doses administered.

FIGS. 23A & 23B show single-agent anti-tumor activity of anti-SIRP-α antibody-CpG oligonucleotide conjugate in mouse RENCA model. In FIG. 23A, mice bearing RENCA tumor cells were dosed intraperitoneally (i.p.) three times, three days apart with anti-mSIRP-α antibody conjugated with murine reactive mT-CpG at 10 mg/kg (squares) or PBS (triangles). In FIG. 23B, a separate cohort was dosed with anti-PD-1 at 10 mg/kg (closed triangles) three times, three days apart or PBS (empty triangles). Arrows indicate doses administered.

FIG. 24A shows the effect of anti-SIRP-α antibody-CpG oligonucleotide conjugate in a mouse CT26 syngeneic tumor model. Mice bearing CT26 tumor cells were intraperitoneally (i.p.) treated with anti-mSIRP-α antibody conjugated with murine reactive mT-CpG at 1 mg/kg, or PBS control. Arrows indicate doses administered.

FIG. 24B shows the results of a follow-up study in the mouse CT26 syngeneic tumor model. Mice bearing CT26 tumor cells were treated with anti-SIRP-α antibody-CpG oligonucleotide conjugate at a suboptimal dose at 0.3 mg/kg, anti-PD-1 antibody at 10 mg/kg, both in combination, or PBS control (FIG. 24B). The anti-SIRP-α antibody-CpG oligonucleotide conjugate had a heavy chain comprising the sequence of SEQ ID NO:91 and a light chain comprising the sequence of SEQ ID NO:111, conjugated to mouse CpG oligo 4523 (SEQ ID NO: 121). Arrows indicate doses administered. P-values comparing combination group vs. anti-PD-1 were calculated using unpaired t-test.

FIG. 25 shows anti-tumor activity of anti-SIRP-α antibody conjugates in combination with anti-PD-L1 antibody in a mouse syngeneic tumor model. Mice bearing B16F10 tumor cells were intraperitoneally (i.p.) treated with either anti-SIRP-α antibody conjugated with murine reactive mT-CpG 4523 at 30 mg/kg, anti-PD-L1 antibody at 10 mg/kg, both in combination, or PBS control.

FIG. 26 shows anti-tumor activity of anti-SIRP-α antibody conjugate in mice that were unresponsive to prior anti-PD-1 treatment. Mice bearing CT26 colon carcinoma cells were treated with PBS or anti-PD-1 (10 mg/kg). Anti-PD-1-treated mice were considered non-responders if tumor measurement exceeded initial size and was greater than 250 mm³. Mice unresponsive to anti-PD-1 were re-randomized by tumor volume with an average size of 338 mm³ on day 11 into 3 new treatment cohorts: 1 mg/kg anti-SIRP-α antibody conjugate alone (cohort 1); 1 mg/kg anti-SIRP-α antibody conjugate in combination with 10 mg/kg anti-PD-1 (cohort 2); or 10 mg/kg anti-PD-1 monotherapy (cohort 3). Treatments were administered intraperitoneally with a total of 2 doses every 3 days. Mean tumor volume (mm³)±SEM was plotted over time for each cohort. Mpk=mg/kg.

FIGS. 27A & 27B show activation of TLR9 pathway signaling by anti-SIRP-α antibody or anti-SIRP-α antibody:CpG oligonucleotide conjugate, as compared to CpG oligo alone. TLR9 activation was assessed in 2 ways simultaneously: interferon regulatory factor (IRF) pathway by monitoring the activity of an inducible secreted Lucia luciferase (FIG. 27A), and NF-kB pathway by monitoring the activity of an inducible secreted embryonic alkaline phosphatase (SEAP) (FIG. 27B). Results are expressed as fold induction over media control vs. concentration.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

As discussed supra, CpG ODNs are often susceptible to degradation in serum and/or exhibit uneven tissue distribution, off-target activity, and local toxicity in vivo; thus, their pharmacokinetic properties may limit their development as potential therapeutics.

One solution is to conjugate the immunomodulating oligonucleotides (e.g., CpG ODNs) with a targeting moiety for specifically targeted tissues or cells to overcome the uneven distribution of the oligonucleotide. See US 2018/0312536. Particularly, transglutaminase-mediated reaction can be used to conjugate a polypeptide targeting moiety containing a glutamine residue with a CpG ODN containing a primary amine group. Microbial transglutaminase (mTG) is from the species Streptomyces mobaraensis. The mTG catalyzes under pH-controlled aqueous conditions (including physiological conditions) a transamidation reaction between a ‘reactive’ glutamine of a protein and a ‘reactive’ lysine residue whereas the latter can also be a simple, low molecular weight primary amine such as a 5-aminopentyl group. For an endogenous glutamine on a protein to be recognized as an mTG-substrate two criteria seem important: 1) the presence of hydrophobic amino acids in the peptide sequence adjacent to the glutamine residue and 2) the positioning of the glutamine on a loop with local chain flexibility enhancing reactivity toward mTG.

The present disclosure is based, at least in part, on the discovery that anti-SIRP-α antibody-CpG oligonucleotide conjugates are effective in activating human myeloid cells (e.g., monocytes and dendritic cells), stimulating cytokine production, and stimulating tumor cell phagocytosis. Means of preparing these conjugates are also described. Particularly, the conjugation can be performed by a transglutaminase (TG)-mediated reaction. Also provided are intermediate compounds which can be used to prepare these conjugates.

The present disclosure demonstrates that anti-SIRP-α antibody-CpG oligonucleotide conjugates promote myeloid cell (e.g., monocyte and dendritic cell) activation, activation of both IRF7 and NFkB pathways, stimulate cytokine production (e.g., IL-6, IFN-α2, IFN-γ, and IL-10), and induce tumor cell phagocytosis by monocyte-derived macrophages. In addition, anti-SIRP-α antibody-CpG oligonucleotide conjugates were found to mediate anti-tumor activity. Such conjugates were found to integrate TLR9 activation with blockade of CD47-SIRP-α interaction of myeloid cells, leading to anti-tumor immune responses from both the innate and adaptive immune systems. Anti-SIRP-α antibody-CpG oligonucleotide conjugates had the additional benefit of preferential tumor cell targeting in SIRP-α-expressing (SIRP-α-positive) tumor models.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Throughout this application, unless the context indicates otherwise, references to a compound of Formula (A)-(D) include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (A)-(D) include polymorphs, solvates, co-crystals, isomers, tautomers and/or oxides thereof. In some embodiments, references to a compound of Formula (A)-(D) include polymorphs, solvates, and/or co-crystals thereof. In some embodiments, references to a compound of Formula (A)-(D) include isomers, tautomers and/or oxides thereof. In some embodiments, references to a compound of Formula (A)-(D) include solvates thereof.

“Alkyl” encompasses straight and branched carbon chains having the indicated number of carbon atoms, for example, from 1 to 20 carbon atoms, or 1 to 8 carbon atoms, or 1 to 6 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “amino,” as used herein, represents-N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —COOR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, —SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In certain embodiments, an amino group is —NHR^{N1}, in which R^{N1} is optionally substituted alkyl. Non-limiting examples of —NHR^{N1}, in which R^{N1} is optionally substituted alkyl, include: optionally substituted alkylamino, a proteinogenic amino acid, a non-proteinogenic amino acid, a C₁₋₆ alkyl ester of a proteinogenic amino acid, and a C₁₋₆ alkyl ester of a non-proteinogenic amino acid. The amino acid employed is optionally in the L-form.

The term “immunomodulating oligonucleotide” as used herein, represents a oligonucleotide construct containing a total of from 6 to 50 contiguous nucleosides covalently bound together by internucleoside bridging groups independently selected from the group consisting of internucleoside phosphoesters and optionally internucleoside abasic spacers. The immunomodulating oligonucleotides are capped at 5′- and 3′-termini with 5′- and 3′-capping groups, respectively. The immunomodulating oligonucleotides are capable of modulating an innate immune response, as determined by, e.g., a change in the activation of intracellular signaling pathway(s) including but not limited to NFκB, a change in the expression of an activation marker or a change in the secretion of at least one inflammatory cytokine or at least one type I interferon in an immune cell (e.g., antigen-presenting cell) to which an immunomodulating oligonucleotide was delivered (e.g., in comparison to another immune cell (e.g., antigen-presenting cell) to which an immunomodulating oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered). The immunomodulating oligonucleotide may contain a conjugating group or, if the immunomodulating oligonucleotide is part of a conjugate, a linker bonded to a targeting moiety and optionally to one or more (e.g., 1 to 6) auxiliary moieties (e.g., polyethylene glycols). The conjugating group or the linker may be part of the phosphotriester or the terminal capping group.

The term “immunostimulating oligonucleotide” as used herein, represents an immunomodulating oligonucleotide capable of activating an immune response, as determined by, e.g., an increase in the activation of intracellular signaling pathway(s) such as NFκB or an increase in levels of cell surface marker(s) of activation or function or an increase in the secretion of at least one inflammatory cytokine or at least one type I interferon in an immune cell (e.g., antigen-presenting cell) to which an immunostimulating oligonucleotide was delivered (e.g., in comparison to another immune cell (e.g., antigen-presenting cell) to which an immunostimulating oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered). In some embodiments, the immunostimulating oligonucleotide contains at least one cytidine-p-guanosine (CpG) sequence, in which p is an internucleoside phosphodiester (e.g., phosphate or phosphorothioate) or an internucleoside phosphotriester or phosphothiotriester. As used herein, the CpG-containing immunostimulating oligonucleotide can be naturally existing, such as CpG ODNs of bacterial or viral origins, or synthetic. For example, in some embodiments, the CpG sequence in the immunostimulating oligonucleotide contains 2′-deoxyribose.

The term “immunosuppressive oligonucleotide” as used herein, represents an immunomodulating oligonucleotide capable of antagonizing an immune response, as determined by e.g., a reduction in the activation or lack of activation of NFκB or lack on increase in the levels of cell surface marker(s) of activation of function or a reduction or lack of increase in the secretion of at least one inflammatory cytokine or at least one type I interferon in an immune cell (e.g., antigen-presenting cell) to which an immunosuppressive oligonucleotide was delivered (e.g., in comparison to another immune cell (e.g., antigen-presenting cell) to which an immunosuppressive oligonucleotide was not delivered) or in an immune cell that interacts with an immune cell (e.g., antigen-presenting cell) to which an immunomodulating oligonucleotide was delivered (including direct cell-to-cell interactions as well as indirect stimulation, e.g., from one or more cytokines secreted by the cell to which an immunomodulating oligonucleotide was delivered).

It is to be understood that the terms “oligonucleotide” and “oligonucleotide” may be used interchangeably herein. It is further to be understood that the terms “immunomodulating oligonucleotide,” “immunostimulating oligonucleotide,” “immunosuppressive oligonucleotide,” and “conjugate” encompass salts of the immunomodulating oligonucleotide, immunostimulating oligonucleotide, immunosuppressive oligonucleotide and conjugate, respectively. For example, the terms “immunomodulating oligonucleotide,” “immunostimulating oligonucleotide,” “immunosuppressive oligonucleotide,” and “conjugate” encompasses both the protonated, neutral form (P—XH moiety, where X is O or S) of a phosphate, phosphorothioate, or phosphorodithioate and the deprotonated, ionic form (P—X⁻ moiety, where X is O or S) of a phosphate, phosphorothioate, or phosphorodithioate. Accordingly, it is to be understood that the phosphoesters and phosphodiesters described as having one or more of R^{E1}, R^{E2}, and R^{E3} as hydrogen encompass salts, in which the phosphate, phosphorothioate, or phosphorodithioate is present in a deprotonated, ionic form. In addition, the terms “free,” “naked,” and “unconjugated” referring to immunomodulating oligonucleotides, immunostimulating oligonucleotides, immunosuppressive oligonucleotides, and/or oligonucleotides (e.g., CpG oligonucleotides) may be used interchangeably herein.

The term “phosphotriester,” as used herein, refers to a phosphoester, in which all three valences are substituted with non-hydrogen substituents. The phosphotriester consists of phosphate, phosphorothioate, or phosphorodithioate; one or two bonds to nucleoside(s), or abasic spacer(s), and/or phosphoryl group(s); and one or two groups independently selected from the group consisting of a bioreversible group; a non-bioreversible group; an auxiliary moiety; a conjugating group; and a linker bonded to a targeting moiety and optionally to one or more (e.g., 1 to 6) auxiliary moieties. A terminal phosphotriester includes one bond to a group containing a nucleoside and two groups independently selected from the group consisting of a bioreversible group; a non-bioreversible group; an auxiliary moiety; a conjugating group; a phosphoryl group; and a linker bonded to a targeting moiety and optionally to one or more (e.g., 1 to 6) auxiliary moieties. In some embodiments, a terminal phosphotriester contains 1 or 0 linkers bonded to a targeting moiety and optionally to one or more (e.g., 1 to 6) auxiliary moieties. An internucleoside phosphotriester includes two bonds to nucleoside-containing groups. A phosphotriester may be a group of the following structure:

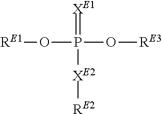

wherein:
   each of X^{E1} and X^{E2} is independently O or S;
   each or R^{E1} and R^{E3} is independently a bond to a nucleoside; a sugar analogue of an abasic spacer; a bioreversible group; a non-bioreversible group; an auxiliary moiety; a conjugating group; a linker bonded to a targeting moiety; a linker bonded to a targeting moiety and one or more (e.g., 1 to 6) auxiliary moieties; or the phosphorus atom in a group of formula —P(═X^{E1}) (—X^{E2}-R^{E2}A)-O—,
      where R^{E2}A is hydrogen; a bioreversible group; a non-bioreversible group; an auxiliary moiety; a conjugating group; a linker bonded to a targeting moiety; or a linker bonded to a targeting moiety and one or more (e.g., 1 to 6) auxiliary moieties; and
   R^{E2} is a bioreversible group; a non-bioreversible group; an auxiliary moiety; a conjugating group; a linker bonded to a targeting moiety; or a linker bonded to a targeting moiety and one or more (e.g., 1 to 6) auxiliary moieties;
   provided that at least one of R^{E1} and R^{E3} is a bond to a group containing at least one nucleoside.

If both R^{E1} and R^{E3} are bonds to groups containing at least one nucleoside, the phosphotriester is an internucleoside phosphotriester. If one and only one of R^{E1} and R^{E3} is a bond to a group containing a nucleoside, the phosphotriester is a terminal phosphotriester.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, omithine, hypusine, 2-aminoisobutyric acid, dehydroalanine, gamma-aminobutyric acid, citrulline, beta-alanine, alpha-ethyl-glycine, alpha-propyl-glycine and norleucine.

The terms “antibody,” “immunoglobulin,” and “Ig” are used interchangeably herein, and are used in the broadest sense and specifically cover, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies. An antibody can be human, humanized, chimeric and/or affinity matured as well as an antibody from other species, for example, mouse and rabbit.

The term “antibody” is intended to include a polypeptide product of B cells within the immunoglobulin class of polypeptides that is able to bind to a specific antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxyl-terminal portion of each chain includes a constant region. See Borrebaeck (ed.) (1995) Antibody Engineering, Second Ed., Oxford University Press.; Kuby (1997) Immunology, Third Ed., W.H. Freeman and Company, New York. Antibodies also include, but are not limited to, synthetic antibodies, monoclonal antibodies, recombinant antibodies, multispecific antibodies (including bispecific antibodies), human antibodies, humanized antibodies, camelized antibodies, chimeric antibodies, intrabodies, anti-idiotypic (anti-Id) antibodies, and functional fragments thereof, which refers a portion of an antibody heavy or light chain polypeptide that retains some or all of the binding activity of the antibody from which the fragment is derived. Non-limiting examples of functional fragments of an antibody include single-chain Fvs (scFv) (e.g., including monospecific or bispecific), Fab fragments, F(ab′) fragments, F(ab)₂ fragments, F(ab′)₂ fragments, disulfide-linked Fvs (sdFv), Fd fragments, Fv fragments, scRv-Fc, nanobody, diabody, triabody, tetrabody, and minibody. In some embodiments, the antibody comprises an Fc variant that has reduced or ablated effector function. In particular, antibodies provided herein include immunoglobulin molecules and immunologically active portions of immunoglobulin molecules, for example, antigen-binding domains or molecules that contain an antigen-binding site that binds to the antigen (e.g., one or more complementarity determining regions (CDRs) of an anti-CD56 antibody or an anti-SIRPα antibody). Such antibody fragments are described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics 1993, 22, 189-224; Plückthun and Skerra, Meth. Enzymol. 1989, 178, 497-515; and Day, Advanced Immunochemistry, Second Ed., Wiley-Liss, Inc., New York, NY (1990). The antibodies provided herein can be of any type (e.g., IgG, IgE, IgM, IgD, IgA, and IgY), any class (e.g., IgG1, IgG2, IgG3, IgG4, IgA1, and IgA2), or any subclass (e.g., IgG2a and IgG2b) of an immunoglobulin molecule.

The term “antigen” refers to a predetermined target to which an antibody can selectively bind. A target antigen can be a polypeptide, carbohydrate, nucleic acid, lipid, hapten, or fragment thereof, or other naturally occurring or synthetic compound. In one embodiment, the target antigen is a polypeptide.

The terms “antigen-binding fragment,” “antigen-binding domain,” and “antigen-binding region” refer to a portion of an antibody that comprises the amino acid residues that interact with an antigen (e.g., a polypeptide, carbohydrate, nucleic acid, lipid, hapten, or fragment thereof, or other naturally occurring or synthetic compound) and confer on the binding agent its specificity and affinity for the antigen (e.g., complementarity determining regions (CDRs)).

The term “specific binding,” “specifically binds to,” or “specific for” a particular polypeptide or an epitope on a particular polypeptide target can be exhibited, for example, by a molecule (e.g., an antibody) having a dissociation constant (K_{d}) for the target of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, or at least about 10⁻¹² M. In one embodiment, the term “specific binding” refers to binding where a molecule binds to a particular polypeptide or epitope on a particular polypeptide without substantially binding to any other polypeptide or polypeptide epitope.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Stites et al. (eds.), Appleton & Lange, Norwalk, CT, 1994, page 71 and Chapter 6.

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “variable region residue numbering as in Kabat” or “amino acid position numbering as in Kabat”, and variations thereof, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, including, for example, by AbM, Chothia, Contact, IMGT and AHon.

An “intact” antibody is one comprising an antigen-binding site as well as a CL and at least heavy chain constant regions, CH1, CH2 and CH3. The constant regions may include human constant regions or amino acid sequence variants thereof. Preferably, an intact antibody has one or more effector functions.

The term “antibody fragment” refers to a portion of an intact antibody, preferably the antigen-binding or variable region of the intact antibody. Examples of antibody fragments include, without limitation, Fab, Fab′, F(ab′)2, and Fv fragments; diabodies and di-diabodies (see, e.g., Holliger et al., Proc. Natl. Acad. Sci. U.S.A. 1993, 90, 6444-8; Lu et al., J. Biol. Chem. 2005, 280, 19665-72; Hudson et al., Nat. Med. 2003, 9, 129-134; WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 1999, 50, 98-101 Streltsov et al., Proc. Natl. Acad. Sci. U.S.A. 2004, 101, 12444-12449); and multispecific antibodies formed from antibody fragments.

The term “functional fragment,” “binding fragment,” or “antigen-binding fragment” of an antibody refers to a molecule that exhibits at least one of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids and a carboxyl-terminal portion that includes a constant region. The constant region can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxyl-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a beta klotho epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., Nature 1975, 256, 495; or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature 1991, 352, 624-628 and Marks et al., J. Mol. Biol. 1991, 222, 581-597, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Exemplary methods of producing monoclonal antibodies are provided in the Examples herein.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 1986, 321, 522-525; Riechmann et al., Nature 1988, 332, 323-329; Presta, Curr. Opin. Biotechnol. 1992, 3, 394-398; Carter et al., Proc. Natl. Acad. Sci. U.S.A. 1992, 89, 4285-4289; and U.S. Pat. Nos. 6,800,738, 6,719,971, 6,639,055, 6,407,213, and 6,054,297.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 1991, 227, 381; Marks et al., J. Mol. Biol. 1991, 222, 581) and yeast display libraries (Chao et al., Nature Protocols 2006, 1, 755-768). Also available for the preparation of human monoclonal antibodies are methods described in Cole et al., Monoclonal Antibodies and Cancer Therapy, Alan R. Liss, p. 77 (1985); Boerner et al., J. Immunol. 1991, 147, 86-95. See also van Dijk and van de Winkel, Curr. Opin. Pharmacol. 2001, 5, 368-374. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 1995, 6, 561-566; Brüggemann and Taussing, Curr. Opin. Biotechnol. 1997, 8, 455-458; and U.S. Pat. Nos. 6,075,181 and 6,150,584 regarding XENOMOUSE™ technology). See also, for example, Li et al., Proc. Natl. Acad. Sci. U.S.A. 2006, 103, 3557-3562 regarding human antibodies generated via a human B-cell hybridoma technology.

A “CDR” refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32, 1-75. CDR region sequences also have been defined structurally by Chothia as those residues that are not part of the conserved β-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods, 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

“Cycloalkynyl” refers to an unsaturated hydrocarbon group within a cycloalkyl having at least one site of acetylenic unsaturation (i.e., having at least one moiety of the formula C≡C). Cycloalkynyl can consist of one ring, such as cyclooctyne, or multiple rings. One cycloalkynyl moiety is an unsaturated cyclic hydrocarbon having from 5 to 10 annular carbon atoms (a “C₅-C₁₀ cycloalkynyl”). Examples include cyclopentyne, cyclohexyne, cycloheptyne, cyclooctyne, cyclononyne, and the like.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl”, as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

The term “DAR” refers to a drug-antibody ratio of an oligonucleotide-antibody conjugate, more specifically an immunomodulating oligonucleotide-antibody ratio. In some instances, for example, an oligonucleotide-antibody conjugate may be described herein as having a DAR of 1 or as a DAR1 conjugate, wherein the oligonucleotide-antibody ratio is 1-to-1. In other instances, an an oligonucleotide-antibody conjugate may be described herein as having a DAR of 2 or as a DAR2 conjugate, wherein the oligonucleotide-antibody ratio is 2-to-1.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyridine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyridine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “effective amount” or “therapeutically effective amount” of a substance is at least the minimum concentration required to bring about a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

The terms “protein,” “polypeptide” and “peptide” are used herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Typically, a protein for use herein will have a molecular weight of at least about 5-20 kDa, alternatively at least about 20-100 kDa, or at least about 100 kDa. Also included within the definition are, for example, proteins containing one or more analogs of an amino acid (including, for example, unnatural amino acids, etc.), as well as other modifications known in the art.

A “pharmaceutically acceptable salt” is a salt form that is non-toxic, biologically tolerable, or otherwise biologically suitable for administration to the subject. See generally Berge et al. (1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

The term “cancer” or “tumor” refers to the presence of cells possessing characteristics typical of cancer-causing cells, such as uncontrolled proliferation, immortality, metastatic potential, rapid growth and proliferation rate, and certain characteristic morphological features. Cancer cells are often in the form of a solid tumor, which is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. In some embodiments, a solid tumor does not need to have measurable dimensions. Cancer cells may also in the form of a liquid tumor, which cancer cells may exist alone or disseminated within an animal. As used herein, the terms “disseminated tumor” and “liquid tumor” are used interchangeably, and include, without limitation, leukemia and lymphoma and other blood cell cancers.

The term “leukemia” refers to a type of cancer of the blood or bone marrow characterized by an abnormal increase of immature white blood cells called “blasts.” Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

As used herein, the term “cancer” includes premalignant as well as malignant cancers, and also includes primary tumors (e.g., those whose cells have not migrated to sites in the subject's body other than the site of the original tumor) and secondary tumors (e.g., those arising from metastasis, the migration of tumor cells to secondary sites that are different from the site of the original tumor), recurrent cancer and refractory cancer.

The terms “cancer recurrence” and “cancer relapse” are used interchangeably and refer to the return of a sign, symptom or disease after a remission. The recurrent cancer cells may re-appear in the same site of the primary tumor or in another location, such as in secondary cancer. The cancer cells may re-appear in the same diseased form as the primary cancer or a different diseased form. For example, in some embodiments, a primary cancer is a solid tumor, and the recurrent cancer is a liquid tumor. In other embodiments, a primary cancer is a liquid tumor, and the recurrent cancer is a solid tumor. In yet other embodiments, the primary cancer and the recurrent cancer are both solid tumors, or both liquid tumors. In some embodiments, the recurrent tumor expresses at least one tumor-associated antigen that is also expressed by the primary tumor.

The term “refractory cancer” as used herein refers to a cancer that does not respond to a treatment, for example, a cancer that is resistant at the beginning of treatment (e.g., treatment with an immunotherapy) or a cancer that may become resistant during treatment. The terms “respond,” “response” or “responsiveness” refer to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

As used herein, cancers include, but are not limited to, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, B cell cancer, e.g., multiple myeloma, Waldenström's macroglobulinemia, the heavy chain diseases, such as, for example, alpha chain disease, gamma chain disease, and mu chain disease, benign monoclonal qammopathy, and immunocytic amyloidosis, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, cholangiocarcinoma (e.g., intrahepatic cholangiocarcinoma), squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “cancer therapy” or “cancer therapeutic agent” as used herein, refers to those therapies or agents that can exert anti-tumor effect or have an anti-tumor activity. Such anti-tumor effect or anti-tumor activity can be exhibited as a reduction in the rate of tumor cell proliferation, viability, or metastatic activity. A possible way of showing anti-tumor activity is to show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The terms “treat,” “treating,” and “treatment” are meant to include alleviating or abrogating a condition, disorder, or disease, or one or more of the symptoms associated with the condition, disorder, or disease; or alleviating or eradicating the cause(s) of the condition, disorder, or disease itself.

The terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject's risk of acquiring a condition, disorder, or disease.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocyclyl, aralkyl, aminosulfonyl, sulfonylamino, sulfonyl, oxo, and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “Q-tag,” as used herein, refers to a portion of a polypeptide containing glutamine residue that, upon transglutaminase-mediated reaction with a compound containing —NH₂ amine, provides a conjugate containing the portion of polypeptide, in which the glutamine residue includes a side chain modified to include the amide bonded to the compound. Q-tags are known in the art. In some embodiments, the Q tag is attached to the C terminal of the heavy chain of the antibody. In some embodiments, the Q tag is attached to the light chain of the antibody. In some embodiments, the Q tag is naturally occurring. For example, mutation of N297 to N297A exposes Q295 of the antibody, where the conjugation could occur (numbering according to EU index, e.g., as listed in Edelman, G. M. et al., Proc. Natl. Acad. USA, 63, 78-85 (1969) and Kabat, E. A. et al., Sequences of proteins of immunological interest. 5th Edition—US Department of Health and Human Services, NIH publication no 91-3242, pp 662,680,689 (1991)). In some embodiments, the Q tag is within the Fc domain of the antibody.

### II. Conjugates

Immunostimulating oligonucleotides have been used in a variety of therapeutic applications. To improve targeting specificity and in vivo distribution, the immunomodulating oligonucleotides (e.g., CpG ODNs) can be conjugated to a targeting moiety (e.g., polypeptides, such as SIRP-α antibodies). Particularly, transglutaminase-mediated reaction can be used to conduct such a conjugation reaction due to its high reaction rates and suitable site specificity. The present disclosure provides polypeptide-oligonucleotide conjugates (such as SIRP-α antibody-oligonucleotide conjugates) exhibiting favorable activity.

The present disclosure is based, at least in part, on the discovery that anti-SIRP-α antibody-CpG oligonucleotide conjugates are effective in activating human myeloid cells (e.g., monocytes and dendritic cells), stimulating cytokine production, and mediating anti-tumor activity against SIRP-α-positive and SIRP-α-negative tumor cells. These conjugates integrate TLR9 activation with blockade of CD47-SIRP-α interaction of myeloid cells, leading to anti-tumor immune responses from both the innate and adaptive immune systems. As such, the present disclosure provides oligonucleotide-SIRP-α-antibody conjugates with robust manufacturability and strong activity in various preclinical models.

Provided herein are oligonucleotide-SIRP-α-antibody conjugates (i.e., SIRP-α antibodies conjugates to oligonucleotides; SIRP-α antibody-conjugates; anti-SIRP-α antibody-CpG oligonucleotide conjugates) wherein the oligonucleotide and SIRP-α antibody are attached together via a linking moiety. In some embodiments, one SIRP-α antibody can be conjugated to one or more oligonucleotides. In some embodiments, the oligonucleotide-antibody conjugate is a conjugate comprising a SIRP-α antibody or antigen-binding fragment thereof and one or more immunomodulating oligonucleotides (P), wherein the antibody or antigen-binding fragment is linked to one or more Q-tag peptides (Q) comprising at least one glutamine residue, wherein each immunomodulating oligonucleotide is linked to a Q-tag peptide via an amide bond with the glutamine residue of the Q-tag peptide and a linker (L) as shown in Formula (A):

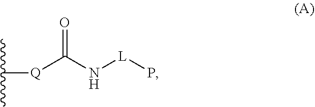

or a stereoisomer, a mixture of two or more diastereomers, a tautomer, or a mixture of two or more tautomers thereof; or a pharmaceutically acceptable salt, solvate, or hydrate thereof;
wherein:
   [symbol] indicates the point of attachment of each Q to the antibody or antigen-binding fragment thereof (Ab);
   each Q is independently a Q-tag peptide sequence comprising at least one glutamine residue;
   each L is independently a bond or a linker moiety connected to Q via an amide bond with the glutamine residue; and
   each P is independently an immunomodulating oligonucleotide.

In some embodiments, the conjugate is a conjugate comprising a SIRP-α antibody or antigen-binding fragment thereof and one or more immunomodulating oligonucleotides (P), wherein the SIRP-α antibody or antigen-binding fragment is linked to one or more Q-tag peptides (Q) that comprise the amino acid sequence RPQGF (SEQ ID NO:47), wherein each immunomodulating oligonucleotide is linked to a Q-tag peptide via an amide bond with the glutamine residue of the Q-tag peptide and a linker (L) as shown in Formula (A),

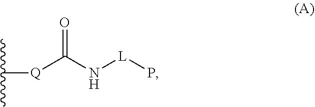

or a stereoisomer, a mixture of two or more diastereomers, a tautomer, or a mixture of two or more tautomers thereof; or a pharmaceutically acceptable salt, solvate, or hydrate thereof;
wherein:
   [symbol] indicates the point of attachment of each Q to the antibody or antigen-binding fragment thereof (Ab);
   each Q independently comprises a Q-tag peptide comprising a peptide sequence RPQGF (SEQ ID NO:47);
   each L is independently a bond or a linker moiety connected to Q via an amide bond with the glutamine residue; and
   each P is independently an immunomodulating oligonucleotide.

In other embodiments, the conjugate is a conjugate comprising a SIRP-α antibody or antigen-binding fragment thereof and one or more immunomodulating oligonucleotides (P), wherein the antibody or antigen-binding fragment is linked to one or more Q-tag peptides (Q) comprising at least one glutamine residue, wherein each immunomodulating oligonucleotide is linked to a Q-tag peptide via an amide bond with the glutamine residue of the Q-tag peptide and a linker (L) as shown in formula (A),

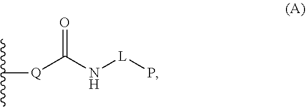

or a stereoisomer, a mixture of two or more diastereomers, a tautomer, or a mixture of two or more tautomers thereof; or a pharmaceutically acceptable salt, solvate, or hydrate thereof;
wherein:
   [symbol] indicates the point of attachment of each Q to the antibody or antigen-binding fragment thereof (Ab);
   each Q is independently a Q-tag peptide comprising at least one glutamine residue;
   each L is independently a bond or a linker moiety connected to Q via an amide bond with the glutamine residue; and
   each P is independently an immunomodulating oligonucleotide selected from the group consisting of the oligonucleotides of Table 15.

In one embodiment, the oligonucleotide-SIRP-α antibody conjugate has a DAR ranging from about 1 to about 20, from about 1 to about 10, from about 1 to about 8, from about 1 to about 4, or from about 1 to about 2. In another embodiment, the oligonucleotide-SIRP-α antibody conjugate has a DAR of about 1, about 2, about 3, about 4, about 5, about 6, about 7, or about 8.

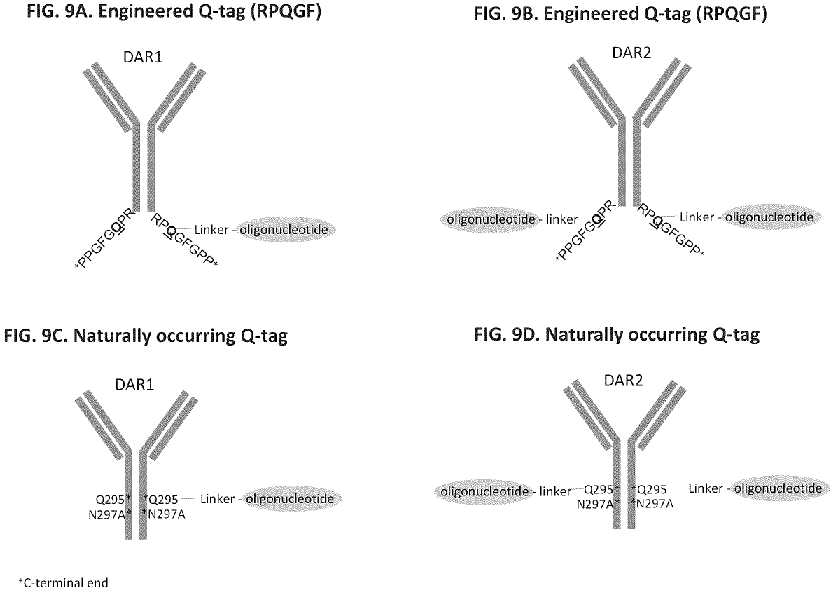

In some embodiments, the conjugate comprises one or more, two or more, three or more, four or more, five or more, six or more, seven or more, eight or more, nine or more, ten or more, or twenty or more Q-tag peptides. In some embodiments, the conjugate comprises one, two, three, four, five, six, seven, eight, nine, ten, or twenty Q-tag peptides. In some embodiments, the conjugate has 2 Q-tag peptides. In some embodiments, the conjugate comprises one or more, two or more, three or more, four or more, five or more, six or more, seven or more, eight or more, nine or more, ten or more, or twenty or more immunomodulating oligonucleotides. In some embodiments, the conjugate comprises one, two, three, four, five, six, seven, eight, nine, ten, or twenty immunomodulating oligonucleotides. In some embodiments, the conjugate has one immunomodulating oligonucleotide. An exemplary conjugate is shown in FIGS. 9A-9D.

### Immunomodulating Oligonucleotides

In one aspect, the oligonucleotide in the oligonucleotide-SIRP-α antibody conjugate is an immunomodulating (e.g., immunostimulating) oligonucleotide. In certain embodiments, the immunomodulating oligonucleotide. comprises a 5-modified uridine or 5-modified cytidine. In certain embodiments, the inclusion of 5-modified uridine (e.g., 5-ethynyl-uridine) at the 5′-terminus of the immunomodulating oligonucleotide (e.g., among the two 5′-terminal nucleosides) enhances the immunomodulating properties of the oligonucleotide. In certain embodiments, the immunomodulating oligonucleotide is shorter (e.g., comprising a total of from about 6 to about 16 nucleotides or from about 12 to about 14 nucleotides) than a typical CpG ODN, which is from 18 to 28 nucleotides in length. In certain embodiments, the shorter immunomodulating oligonucleotide (e.g., those comprising a total of from about 6 to about 16 nucleotides or from about 12 to about 14 nucleotides) retains the immunomodulating activity of a longer, typical CpG ODN; or exhibits higher immunomodulating activity (e.g., as measured by NFκB activation or by the changes in the expression levels of cell surface markers of activation or function such as CD40, HLADR, CD69 or CD80 or by the changes in the levels of at least one cytokine (e.g., IL-6 or IL-10), as compared to the longer CpG ODN. In certain embodiments, the immunomodulating oligonucleotide comprises an abasic spacer. In certain embodiments, the immunomodulating oligonucleotide comprises an internucleoside phosphotriester.

In certain embodiments, the immunomodulating oligonucleotide provided herein exhibits stability (e.g., stability against nucleases) that is superior to that of a CpG ODN containing mostly internucleoside phosphate (e.g., more than 50% of internucleoside phosphates) without substantially sacrificing its immunostimulating activity. This effect can be achieved, e.g., by incorporating at least 50% (e.g., at least 70%) internucleoside phosphorothioates or phosphorodithioates or through the inclusion of internucleoside phosphotriesters and/or internucleoside abasic spacers. Phosphotriesters and abasic spacers are also convenient for conjugation to a targeting moiety. Phosphate-based phosphotriesters and abasic spacers can also be used for reduction of off-target activity, relative to oligonucleotides with fully phosphorothioate backbones. Without wishing to be bound by theory, this effect may be achieved by reducing self-delivery without disrupting targeting moiety-mediated delivery to target cells. Accordingly, a oligonucleotide provided herein can include about 15 or fewer, about 14 or fewer, about 13 or fewer, about 12 or fewer, about 11 or fewer, or about 10 or fewer contiguous internucleoside phosphorothioates. For example, an immunostimulating oligonucleotide comprising a total of from about 12 to about 16 nucleosides can contain about 10 or fewer contiguous internucleoside phosphorothioates.

The immunostimulating oligonucleotide provided herein can contain a total of about 50 or fewer, about 30 or fewer, about 28 or fewer, or about 16 or fewer nucleosides. The immunostimulating oligonucleotide can contain a total of at least 6, about 10 or more, or about 12 or more nucleosides. For example, the immunostimulating oligonucleotide can contain a total of from about 6 to about 30, from about 6 to about 28, from about 6 to about 20, from about 6 to about 16, from about 10 to about 20, from about 10 to about 16, from about 12 to about 28, from about 12 to about 20, or from about 12 to about 16 nucleosides.

In certain embodiments, the immunostimulating oligonucleotide comprises one or more phosphotriesters (e.g., internucleoside phosphotriesters) and/or phosphorothioates (e.g., from about 1 to about 6 or from about 1 to about 4), e.g., at one or both termini (e.g., within the six 5′-terminal nucleosides or the six 3′-terminal nucleosides). The inclusion of one or more internucleoside phosphotriesters and/or phosphorothioates can enhance the stability of the oligonucleotide by reducing the rate of exonuclease-mediated degradation.

In certain embodiments, the immunostimulating oligonucleotide comprises a phosphotriester or a terminal phosphodiester, where the phosphotriester or the terminal phosphodiester comprises a linker bonded to a targeting moiety or a conjugating group and optionally to one or more (e.g., from about 1 to about 6) auxiliary moieties. In certain embodiments, the immunostimulating oligonucleotide comprises only one linker. In certain embodiments, the immunostimulating oligonucleotide comprises only one conjugating group.

The oligonucleotide provided herein can be a hybridized oligonucleotide including a strand and its partial or whole complement. The hybridized oligonucleotides can have at least 6 complementary base pairings (e.g., about 6, about 7, about 8, about 9, about 10, about 11, about 12, about 13, about 14, about 15, about 16, about 17, about 18, about 19, about 20, about 21, about 22, or about 23), up to the total number of the nucleotides present in the included shorter strand. For example, the hybridized portion of the hybridized oligonucleotide can contain about 6, about 7, about 8, about 9, about 10, about 11, about 12, about 13, about 14, about 15, about 16, about 17, about 18, about 19, about 20, about 21, about 22, or about 23 base pairs.

In one aspect, the oligonucleotide in the oligonucleotide-SIRP-α antibody conjugate comprises one or more CpG sites. In some embodiments, the oligonucleotide comprises at least 1, at least 2, or at least 3 CpG sites. In some embodiments, the oligonucleotide is an antisense oligonucleotide. As used herein, a “modified nucleotide” is a nucleotide other than a ribonucleotide (2′-hydroxyl nucleotide). In some embodiments, at least 50%, at least 55%, at least 60%, at least 65%, at least 70%, at least 75%, at least 80%, at least 85%, at least 90%, at least 95%, at least 97%, at least 98%, at least 99%, or 100% of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides (cPrpN). The 2′-modified nucleotides (i.e. a nucleotide with a group other than a hydroxyl group at the 2′ position of the five-membered sugar ring) include, but are not limited to, 2′-O-alkyl nucleotides, 2′-deoxy-2′-halo nucleotides, 2′-deoxy nucleotides, 2′-methoxyethyl (2′-O-2-methoxylethyl) nucleotides, 2′-amino nucleotides, 2′aminoalkyl nucleotides, and 2′-alkyl nucleotides. In some embodiments, modified nucleotide is selected from the group consisting of 5-bromo-2′-O-methyluridine, 5-bromo-2′-deoxyuridine, 2′-O-methyluridine, 2′-deoxyuridine, 2′-O-methylthymidine, 2′-O-methylcytidine, 2′-O-(2-methoxyethyl) thymidine and 8-oxo-7,8-dihydro-2′-deoxyguanosine. It is not necessary for all positions in a given compound to be uniformly modified. Conversely, more than one modification may be incorporated in a single oligonucleotide or even in a single nucleotide thereof. The oligonucleotides may be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-Me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil (e.g., 5-bromouracil and 5-iodouracil), cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl, 8-oxo and other 8-substituted adenines and guanines, 5-halo (e.g., 5-bromo and 5-iodo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, one or more nucleotides of the oligonucleotide are linked by non-standard linkages or backbones (e.g., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorothioate linkages. In some embodiments, the oligonucleotide comprises at least 2, at least 3, at least 4, at least 5, at least 6, at least 7, at least 8, at least 9, at least 10, at least 11, at least 12, at least 13, at least 14, or at least 15 phosphorodithioate linkages. In some embodiments, the oligonucleotide comprises 1, 2, 3, 4, 5, 6, 7, 8, 9, 10, 11, 12, 13, 14, or 15 phosphorothioate linkages. In some embodiments, the oligonucleotide comprises 1, 2, 3, 4, 5, 6, 7, 8, 9, 10, 11, 12, 13, 14, or 15 phosphorodithioate linkages. In some embodiments, the phosphorothioate internucleoside linkages or phosphorodithioate internucleoside linkages are between the nucleotides at positions 1-3, 2-4, 3-5, 4-6, 4-5, 6-8, 7-9, 8-10, 9-11, 10-12, 11-13, 12-14, 13-15, 14-16, 15-17, 16-18, 17-19, 18-20 or 19-21 from the 5′ end of the oligonucleotide. In some embodiments, the oligonucleotide comprises one or more modified nucleotides and one or more modified internucleoside linkages.

In some embodiments, the oligonucleotide comprises a terminal cap. In some embodiments, the terminal cap is at the 3′ end of the oligonucleotide. In some embodiments, the terminal cap is at the 5′ end of the oligonucleotide. In some embodiments, the terminal cap is at the 5′ end and 3′ end of the oligonucleotide. The term “terminal cap” can also be referred to as “cap,” and has meaning generally accepted in the art. For example, the term refers to a moiety, which can be a chemically modified nucleotide or non-nucleotide that can be incorporated at one or more termini of one or more nucleic acid molecules of the invention. These terminal modifications can protect the nucleic acid molecule from exonuclease degradation, and can help in delivery and/or localization within a cell. In non-limiting examples, the cap includes, but is not limited to a polymer; a ligand; locked nucleic acid (LNA); glyceryl; an abasic ribose residue; inverted deoxy abasic residue; an inverted nucleotide; 4′,5′-methylene nucleotide; 1-(beta-D-erythrofuranosyl) nucleotide; 5′-mercapto moieties; 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; 5′-5′-inverted nucleotide moiety; 5′-5′-inverted abasic moiety; 5′-phosphoramidate; 5′-phosphorothioate; 1,4-butanediol phosphate; 5′-amino; bridging and/or nonbridging 5′-phosphoramidate; phosphorothioate and/or phosphorodithioate; or bridging or nonbridging methylphosphonate moiety. In some embodiments, the oligonucleotide comprises one or more terminal cap molecules. In some embodiments, [N] is a 3′ terminal cap. In some embodiments, the 3′ terminal cap is O-(3-hydroxypropyl)phosphorothioate.

In some embodiments, the oligonucleotide is about 10-30, about 10-15, about 15-20, about 20-25, about 25-30, about 15-25 nucleotides in length. In some embodiments, the oligonucleotide is about 18, 19, 20, 21, 22, 23, 24 or 25 nucleotides in length.

In another aspect, the oligonucleotide of the conjugate comprises:

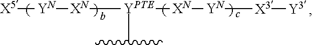

wherein
   b and c are each independently an integer from 1 to 25; with the proviso that the sum of b and c is at least 5;
[symbol] * indicates the point of attachment of the immunomodulating oligonucleotide P to the rest of the conjugate;
   X^{5′} is a 5′ terminal nucleoside having the structure

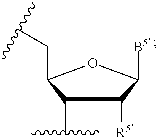

X^{3′} is a 3′ terminal nucleoside having the structure

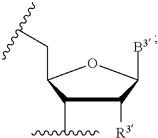

Y^{PTE} is an internucleoside phosphotriester having the structure

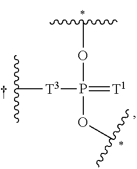

wherein * indicates the points of attachment to the rest of the oligonucleotide and [symbol] † indicates the point of attachment to the linker L, or, if L is absent, [symbol] † indicates the point of attachment to the Q tag peptide Q at the glutamine residue via an amide bond;
Y³ is a terminal phosphotriester having the structure

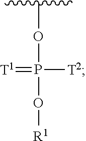

each X^{N} is independently a nucleoside having the structure

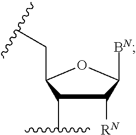

each Y^{N} is independently an internucleoside linker having the structure

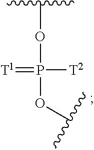

wherein each B^{N} is independently a modified or unmodified nucleobase;
each R^{N} is independently —H or —O—C₁₋₄-alkyl, wherein the C₁₋₄-alkyl of the —O—C₁₋₄-alkyl is optionally further substituted by —O—C₁₋₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄-alkyl is optionally further substituted by —O—C₁₋₄-alkyl;
each T₁ is independently O or S;
each T² is independently O⁻ or S⁻; and
T³ is a group comprising an oligoethylene glycol moiety; and
R¹ is C₁₋₄-alkylene-hydroxy.

In certain embodiments, the oligonucleotide comprises a nucleotide with a modified nucleobase. In some embodiments, B^{5′} is a modified nucleobase. In other embodiments, B^{3′} is a modified nucleobase. In some embodiments, B^{5′} is an unmodified nucleobase. In other embodiments, B³ is an unmodified nucleobase. In still other embodiments, at least one B^{N} is a modified nucleobase.

In certain embodiments, b is an integer ranging from about 1 to about 15. In certain embodiments, b is an integer of about 1, about 2, about 3, about 4, about 5, about 6, about 7, about 8, about 9, about 10, about 11, about 12, about 13, about 14, or about 15. In certain embodiments, b is an integer of about 3, about 4, about 11, or about 14. In certain embodiments, b is an integer of about 3. In certain embodiments, b is an integer of about 4. In certain embodiments, b is an integer of about 11. In certain embodiments, b is an integer of about 14.

In certain embodiments, c is an integer ranging from about 0 to about 10. In certain embodiments, c is an integer of about 0, about 1, about 2, about 3, about 4, about 5, about 6, about 7, about 8, about 9, or about 10. In certain embodiments, c is an integer of about 0 or about 8. In certain embodiments, c is an integer of about 0. In certain embodiments, c is an integer of about 8.

In certain embodiments, b is an integer of about 3 and c is an integer of about 8. In certain embodiments, b is an integer of about 4 and c is an integer of about 8. In certain embodiments, b is an integer of about 11 and c is an integer of about 0. In certain embodiments, b is an integer of about 14 and c is an integer of about 0.

In certain embodiments, b and c together in total are ranging from about 5 to about 20. In certain embodiments, b and c together in total are ranging from about 5 to about 15. In certain embodiments, b and c together in total are about 5, about 6, about 7, about 8, about 9, about 10, about 11, about 12, about 13, about 14, or about 15. In certain embodiments, b and c together in total are about 8, about 9, about 10, about 11, about 12, about 13, or about 14. In certain embodiments, b and c together in total are about 11. In certain embodiments, b and c together in total are about 12. In certain embodiments, b and c together in total are about 14.

In certain embodiments, each X^{N} is independently a 2′-deoxyribonucleoside or a 2′-modified ribonucleoside. In certain embodiments, each X^{N} is independently 2′-deoxyadenosine (A), 2′-deoxyguanosine (G), 2′-deoxycytidine (C), a 5-halo-2′-deoxycytidine, 2′-deoxythymidine (T), 2′-deoxyuridine (U), a 5-halo-2′-deoxyuridine, a 2′-fluororibonucleoside, a 2′-methoxyribonucleoside, or a 2′-(2-methoxyethoxy) ribonucleoside. In certain embodiments, each X^{N} is independently a 2′-deoxyribonucleoside. In certain embodiments, each X^{N} is independently 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, each X^{N} is independently 2′-deoxyadenosine, 2′-deoxyguanosine, 2′-deoxycytidine, 2′-deoxythymidine, 5-bromo-2′-deoxyuridine, or 5-iodo-2′-deoxyuridine.

In certain embodiments, X^{3′} is a 2′-deoxyribonucleoside or a 2′-modified ribonucleoside. In certain embodiments, X^{3′} is a 2′-deoxyribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy) ribonucleoside. In certain embodiments, X^{3′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{3′} is 2′-deoxythymidine. In certain embodiments, X^{3′} is a 2′-deoxyribonucleoside with a substituted pyrimidine base. In certain embodiments, X^{3′} is a 2′-deoxyribonucleoside with a 5-substituted pyrimidine base. In certain embodiments, X^{3′} is 2′-deoxythymidine, a 5-halo-2′-deoxycytidine, or a 5-halo-2′-deoxyuridine. In certain embodiments, X^{3′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{3′} is 2′-deoxythymidine, 5-bromo-2′-deoxyuridine, or 5-iodo-2′-deoxyuridine. In certain embodiments, X^{3′} is a terminal nucleotide comprising a 3′ capping group. In certain embodiments, the 3′ capping group is a terminal phosphoester. In certain embodiments, the 3′ capping group is 3-hydroxyl-propylphosphoryl (i.e., —P(O₂)—OCH₂CH₂CH₂OH).

In certain embodiments, X^{5′} is a 2′-deoxyribonucleoside or a 2′-modified ribonucleoside. In certain embodiments, X^{5′} is a 2′-deoxyribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, a 5-halo-2′-deoxyuridine, a 2′-fluororibonucleoside, a 2′-methoxyribonucleoside, or a 2′-(2-methoxyethoxy) ribonucleoside. In certain embodiments, X^{5′} is 2′-deoxyadenosine, 2′-deoxyguanosine, 2′-deoxycytidine, a 5-halo-2′-deoxycytidine, 2′-deoxythymidine, 2′-deoxyuridine, or a 5-halo-2′-deoxyuridine. In certain embodiments, X^{5′} is a 2′-deoxyribonucleoside with a substituted pyrimidine base. In certain embodiments, X^{5′} is a 2′-deoxyribonucleoside with a 5-substituted pyrimidine base. In certain embodiments, X^{5′} is 2′-deoxythymidine, a 5-halo-2′-deoxycytidine, or a 5-halo-2′-deoxyuridine. In certain embodiments, X^{5′} is a 5-halo-2′-deoxycytidine. In some embodiments, X^{5′} is a 2′-deoxyuridine, a 5-halo-2′-deoxyuridine, 2′-methoxyuridine, or a 5-halo-2′-methoxyuridine. In certain embodiments, X^{5′} is a 5-halo-2′-deoxyuridine. In certain other embodiments, X^{5′} is a 2′-deoxyuridine. In certain embodiments, X^{5′} is a 5-halo-2′-methoxyuridine. In certain other embodiments, X^{5′} is a 2′-methoxyuridine. In certain embodiments, X^{5′} is 2′-deoxythymidine, 5-bromo-2′-deoxycytidine, 5-iodo-2′-deoxycytidine, 5-bromo-2′-deoxyuridine, or 5-iodo-2′-deoxyuridine. In certain embodiments, X^{5′} is 2′-deoxythymidine, 5-bromo-2′-deoxyuridine, or 5-iodo-2′-deoxyuridine. In certain embodiments, X^{5′} is 5-bromo-2′-deoxyuridine. In certain embodiments, X^{5′} is 5-iodo-2′-deoxyuridine. In certain embodiments, X^{5′} has a 3′-phosphorothioate group. In certain embodiments, X^{5′} has a 3′-phosphorothioate group with a chirality of Rp. In certain embodiments, X^{5′} has a 3′-phosphorothioate group with a chirality of Sp.

In certain embodiments, Y^{PTE} is an internucleoside phosphothiotriester.

In some embodiments, Y^{PTE} is

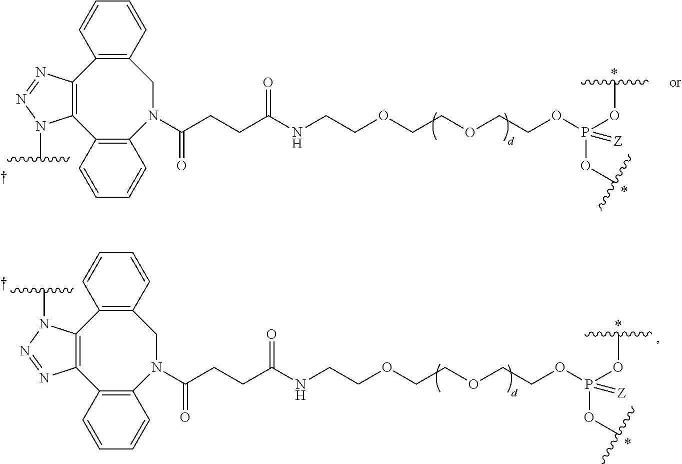

wherein Z is O or S; d is an integer ranging from about 0 to about 50; the two [symbol] * on the right side of the structure indicate the points of attachment to the oligonucleotide P; and the [symbol] † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In some embodiments, Y^{PTE} is

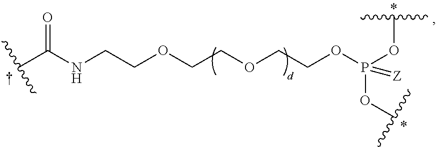

wherein Z is O or S; d is an integer ranging from about 0 to about 50; the two [symbol] * on the right side of the structure indicate the points of attachment to the oligonucleotide P; and the [symbol] † on the left side of the structure indicates the point of attachment to the rest of the conjugate. In certain embodiments, Z is O. In certain embodiments, Z is S. In certain embodiments, d is an integer ranging from about 0 to about 10. In certain embodiments, d is an integer ranging from about 0 to about 5. In certain embodiments, d is an integer of about 0, about 1, about 2, about 3, about 4, or about 5. In certain embodiments, d is an integer of about 0, about 1, or about 3.

In certain embodiments, the oligonucleotide comprises one additional internucleoside phosphotriester. In one embodiment, the additional internucleoside phosphotriester is a C₁₋₆ alkylphosphotriester. In another embodiment, the additional internucleoside phosphotriester is ethylphosphotriester.

In certain embodiments, the oligonucleotide comprises one 5-halo-2′-deoxyuridine. In one embodiment, the 5-halo-2′-deoxyuridine is 5-fluoro-2′-deoxyuridine, 5-bromo-2′-deoxyuridine, or 5-iodo-2′-deoxyuridine. In another embodiment, the 5-halo-2′-deoxyuridine is 5-bromo-2′-deoxyuridine or 5-iodo-2′-deoxyuridine. In yet another embodiment, the 5-halo-2′-deoxyuridine is 5-fluoro-2′-deoxyuridine. In yet another embodiment, the 5-halo-2′-deoxyuridine is 5-bromo-2′-deoxyuridine. In still another embodiment, the 5-halo-2′-deoxyuridine is 5-iodo-2′-deoxyuridine.

In certain embodiments, the oligonucleotide comprises three or more 2′-deoxycytidines. In certain embodiments, the oligonucleotide comprises three 2′-deoxycytidines.

In certain embodiments, the oligonucleotide comprises four or more 2′-deoxyguanosines. In certain embodiments, the oligonucleotide comprises four 2′-deoxyguanosines.

In certain embodiments, the oligonucleotide comprises three 2′-deoxycytidines and four 2′-deoxyguanosines. In certain embodiments, the oligonucleotide comprises one, two, or three CG dinucleotides. In certain embodiments, the oligonucleotide comprises three CG dinucleotides.

In certain embodiments, the oligonucleotide comprises three or more 2′-deoxythymidines. In certain embodiments, the oligonucleotide comprises three, four, five, six, seven, or eight 2′-deoxythymidines. In certain embodiments, the oligonucleotide comprises three, four, five, or eight 2′-deoxythymidines.

In certain embodiments, the oligonucleotide does not comprise a 2′-deoxyadenosine. In certain embodiments, the oligonucleotide comprises one or two 2′-deoxyadenosines.

In certain embodiments, the oligonucleotide has a length ranging from about 5 to about 20 or from about 6 to about 15. In certain embodiments, the oligonucleotide has a length of about 6, about 7, about 8, about 9, about 10, about 11, about 12, about 13, about 14, or about 15. In certain embodiments, the oligonucleotide has a length of about 10, about 11, about 12, about 13, about 14, or about 15.

In certain embodiments, the oligonucleotide comprises one or more internucleoside phosphorothioates. In certain embodiments, all the internucleoside phosphoesters in the oligonucleotide are internucleoside phosphorothioates. In certain embodiments, the oligonucleotide comprises one or more chiral internucleoside phosphorothioates.

In certain embodiments, the oligonucleotides comprising a sequence of N¹N²CGN³CG(T)ₓGN⁴CGN⁵T (SEQ ID NO:174), or a stereoisomer, a mixture of two or more diastereomers, a tautomer, or a mixture of two or more tautomers thereof; or a pharmaceutically acceptable salt, solvate, or hydrate thereof are as described in, for example, WO2018/189382 A1.

In one embodiment, the oligonucleotide comprises a sequence of N¹N²CGN³CG(T)ₓGN⁴CGN⁵T (SEQ ID NO:174), or a stereoisomer, a mixture of two or more diastereomers, a tautomer, or a mixture of two or more tautomers thereof; or a pharmaceutically acceptable salt, solvate, or hydrate thereof; wherein:
x is an integer ranging from about 1 to about 4;
N¹ is absent or 2′-deoxythymidine;
N² is a 2′-deoxyribonucleotide with a modified nucleobase;
N³ is 2′-deoxyadenosine or 2′-deoxythymidine, each optionally comprising a 3′-phosphotriester;
N⁴ is 2′-deoxyadenosine or 2′-deoxythymidine;
N⁵ is 2′-deoxythymidine optionally comprising a 3′-phosphotriester; and
C is 2′-deoxycytidine and G is 2′-deoxyguanosine.

In certain embodiments, in N¹N²CGN³CG(T)ₓGN⁴CGN⁵T (SEQ ID NO:174), x is an integer of about 1, about 2, about 3, or about 4. In certain embodiments, in N¹N²CGN³CG(T)ₓGN⁴CGN⁵T (SEQ ID NO:174), x is an integer of about 1. In certain embodiments, in N¹N²CGN³CG(T)ₓGN⁴CGN⁵T (SEQ ID NO:174), x is an integer of about 4.

In certain embodiments, in N¹N²CGN³CG(T)ₓGN⁴CGN⁵T (SEQ ID NO:174), N¹ is absent. In certain embodiments, in N¹N²CGN³CG(T)ₓGN⁴CGN⁵T (SEQ ID NO:174), N¹ is 2′-deoxythymidine.

In certain embodiments, in N¹N²CGN³CG(T)ₓGN⁴CGN⁵T (SEQ ID NO:174), N² is a 2′-deoxyribonucleotide with a substituted pyrimidine base. In certain embodiments, in N¹N²CGN³CG(T)ₓGN⁴CGN⁵T (SEQ ID NO:174), N² is a 2′-deoxyribonucleotide with a 5-substituted pyrimidine base. In certain embodiments, in N¹N²CGN³CG(T)ₓGN⁴CGN⁵T (SEQ ID NO: 174), N² is a 5-halo-2′-deoxycytidine or a 5-halo-2′-deoxyuridine. In certain embodiments, in N¹N²CGN³CG(T)ₓGN⁴CGN⁵T (SEQ ID NO:174), N² is 5-bromo-2′-deoxyuridine or 5-iodo-2′-deoxyuridine.

In certain embodiments, in N¹N²CGN³CG(T)ₓGN⁴CGN⁵T (SEQ ID NO:174), N³ is 2′-deoxyadenosine comprising a 3′-phosphotriester. In certain embodiments, in N¹N²CGN³CG(T)ₓGN⁴CGN⁵T (SEQ ID NO:174), N³ is 2′-deoxythymidine. In certain embodiments, in N¹N²CGN³CG(T)ₓGN⁴CGN⁵T (SEQ ID NO:174), N³ is 2′-deoxythymidine comprising a 3′-phosphotriester.

In certain embodiments, in N¹N²CGN³CG(T)ₓGN⁴CGN⁵T (SEQ ID NO:174), N⁴ is 2′-deoxyadenosine. In certain embodiments, in N¹N²CGN³CG(T)ₓGN⁴CGN⁵T (SEQ ID NO:174), N⁴ is 2′-deoxythymidine.

In certain embodiments, in N¹N²CGN³CG(T)ₓGN⁴CGN⁵T (SEQ ID NO:174), N⁵ is 2′-deoxythymidine. In certain embodiments, in N¹N²CGN³CG(T)ₓGN⁴CGN⁵T (SEQ ID NO:174), N⁵ is 2′-deoxythymidine comprising a 3′-phosphotriester.

In certain embodiments, the oligonucleotide of N¹N²CGN³CG(T)ₓGN⁴CGN⁵T (SEQ ID NO: 174) comprises one or more internucleoside phosphorothioates or phosphorotdithioates. In certain embodiments, the oligonucleotide of N¹N²CGN³CG(T)ₓGN⁴CGN⁵T (SEQ ID NO:174) comprises at least one chiral internucleoside phosphorothioate or phosphorotdithioates. In certain embodiments, the oligonucleotide of N¹N²CGN³CG(T)ₓGN⁴CGN⁵T (SEQ ID NO:174) comprises at least one chiral phosphorotdithioates. In certain embodiments, the oligonucleotide of N¹N²CGN³CG(T)ₓGN⁴CGN⁵T (SEQ ID NO:174) is an oligonucleotide sequence as described in, for example, WO2018/189382 A1.

With regard to use of the character ′ (e.g., with respect to 5′ and/or 3′ designations) herein, it should be noted that the character ′ may appear herein in different font, e.g., using a Times font when in text (′) and a different font (′) when used as part of a formula, but the meaning of the character ′ is intended to be the same regardless of font.

In certain embodiments, the oligonucleotide is an immunostimulating oligonucleotide. In certain embodiments, the oligonucleotide provided herein functions as a PAMS. In certain embodiments, the oligonucleotide provided herein activates innate immune response or stimulates the adaptive immune response by triggering TLR9 signaling. In certain embodiments, the oligonucleotide provided herein is a TLR9 agonist.

In certain embodiments, the oligonucleotide is a CpG oligonucleotide, comprising a modification including 5-halouridine or 5-alkynyluridine, or a truncated version thereof (e.g., those comprising a total of about 6 to about 16 nucleosides). In certain embodiments, the truncated oligonucleotide provided herein comprises a truncated oligonucleotide sequence, from which one or more 3′-terminal nucleotides are eliminated or one or more of the intra-sequence nucleotides excised).

In certain embodiments, the oligonucleotide comprises at least one immunostimulating sequence (ISS). In certain embodiments, the oligonucleotide provided herein comprises about 1, about 2, about 3, or about 4 ISS. The ISS in immunostimulating oligonucleotides is dependent on the targeted organism. The common feature of the ISS used in the oligonucleotide provided herein is the cytidine-p-guanosine sequence, in which p is an internucleoside phosphodiester (e.g., phosphate or phosphorothioate) or an internucleoside phosphotriester. In certain embodiments, cytidine and guanosine in the ISS each independently comprises 2′-deoxyribose. In certain embodiments, the oligonucleotide provided herein comprises about 1, about 2, or about 3 human ISSs. In certain embodiments, the human ISS is CG or NCG, where N is uridine, cytidine, or thymidine, or a modified uridine or cytidine; and G is guanosine or a modified guanosine. In certain embodiments, the modified uridine or cytidine is a 5-halouridine (e.g., 5-iodouridine or 5-bromouridine), a 5-alkynyluridine (e.g., 5-ethynyluridine or 5-propynyluridine), 5-heteroaryluridine, or 5-halocytidine. In certain embodiments, the modified guanosine is 7-deazaguanosine. In certain embodiments, the human ISS is NCG, in one embodiment, N is 5-halouridine. In certain embodiments, the human ISS is UCG, in one embodiment, U is 5-alkynyluridine, and in another embodiment, U is 5-ethynyluridine. In certain embodiments, the oligonucleotide provided herein targeting humans comprises an ISS within four contiguous nucleotides that include a 5′-terminal nucleotide. In certain embodiments, the oligonucleotide provided herein targeting humans comprises a 5′-terminal ISS. In certain embodiments, the oligonucleotide provided herein comprises a murine ISS. In certain embodiments, the murine ISS is a hexameric nucleotide sequence: Pu-Pu-CG-Py-Py, where each Pu is independently a purine nucleotide, and each Py is independently a pyrimidine nucleotide.

In certain embodiments, the 5′-flanking nucleotides relative to CpG in the oligonucleotide provided herein does not contain 2′-alkoxyriboses. In certain embodiments, the 5′-flanking nucleotides relative to CpG in the oligonucleotide provided herein comprises only 2′-deoxyriboses as sugars.

In certain embodiments, the oligonucleotide has (1) a high content of phosphorothioates or phosphorodithioates (e.g., at least 50%, at least 60%, at least 70%, or at least 80% of nucleosides may be linked by phosphorothioates or phosphorodithioates); (2) absence of poly-G tails; (3) nucleosides in the oligonucleotide comprises 2′-deoxyriboses or 2′-modified riboses (e.g., 2′-halo (e.g., 2′-fluoro, 2′-bromo, or 2′-iodo) or optionally substituted 2′-alkoxy (e.g., 2′-methoxy)); and/or (4) the inclusion of 5′-terminal ISS that is NCG, in which N is uridine, cytidine, or thymidine, or a modified uridine or cytidine, and G is guanosine or a modified guanosine.

In certain embodiments, the oligonucleotide suppresses the adaptive immune response by reducing activation of TLR9 signaling (e.g., through TLR9 antagonism). In certain embodiments, the immunosuppressive oligonucleotide provided herein comprises at least two 2′-alkoxynucleotides that are 5′-flanking relative to CpG as described by the formula of N¹-N²-CG, where N¹ and N² are each independently a nucleotide containing 2′-alkoxyribose (e.g., 2′-methoxyribose).

In some embodiments, the oligonucleotide has the structure

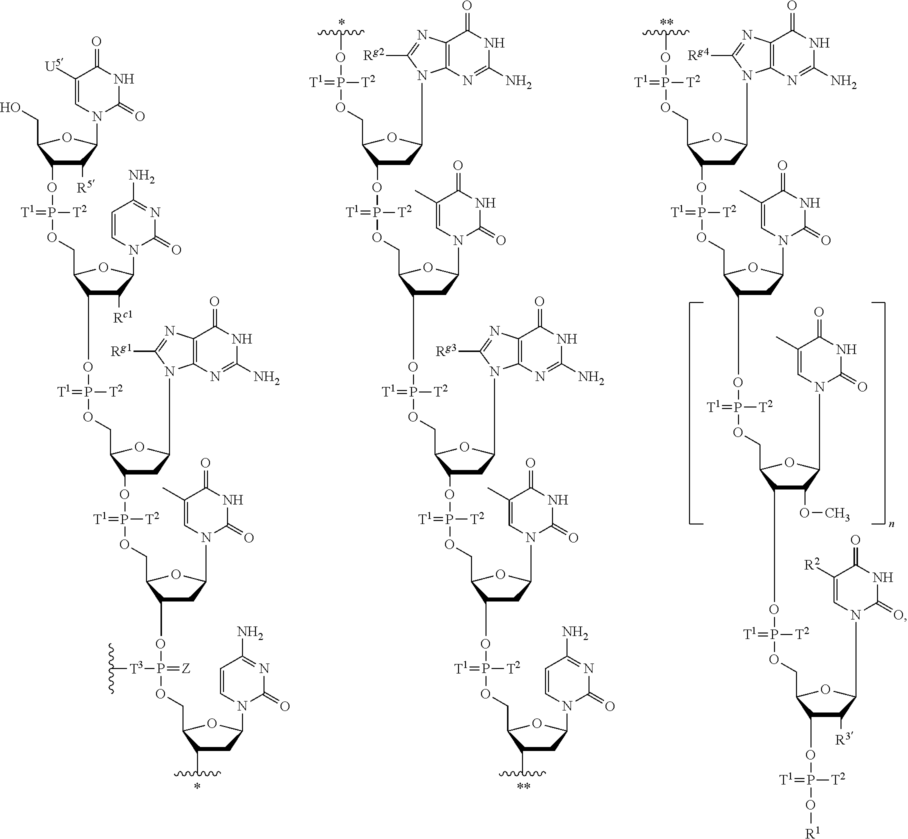

wherein
   [symbol] * and [symbol] ** indicate the points of attachment within the oligonucleotide;
   each T¹ is independently O or S;
   each T² is O⁻ or S⁻;
   T³ is a group

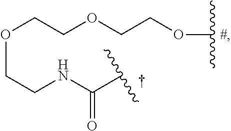 [SMILES: *OCCOCCOCCNC(*)=O]

wherein [symbol] † indicates the point of attachment to L and wherein [symbol] # indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R^{g4} are H or oxo, wherein if one R^{g1}, R^{g2}, R^{g3}, and R^{g4} is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In other embodiments, the oligonucleotide has the structure

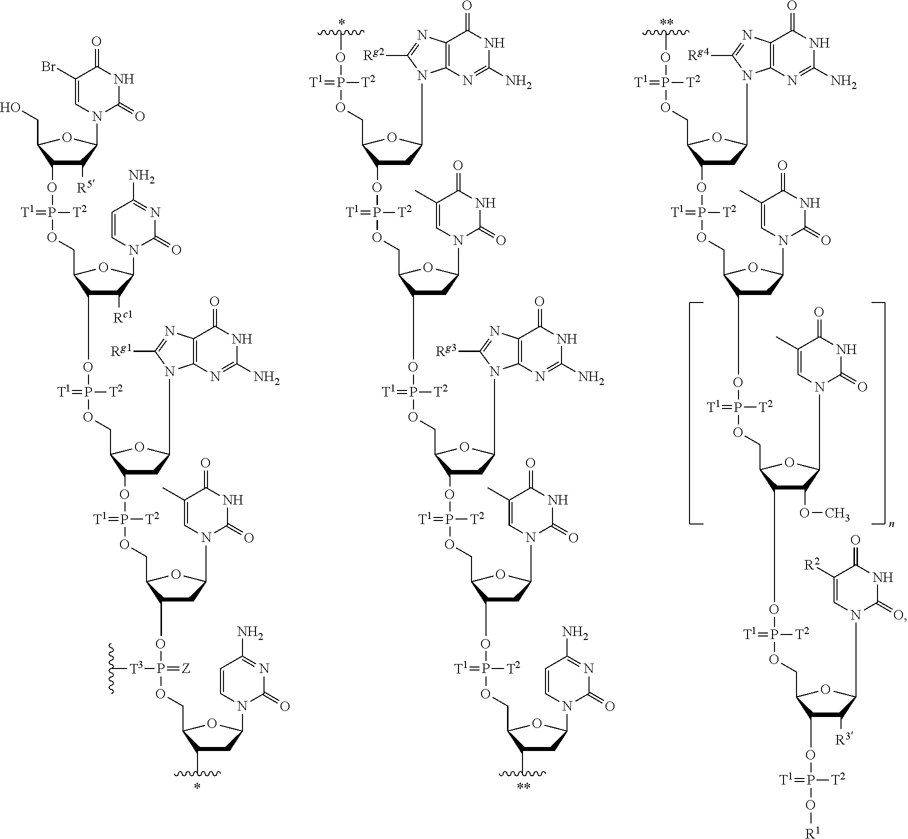

wherein
   [symbol] * and [symbol] ** indicate the points of attachment within the oligonucleotide;
   each T¹ is independently O or S;
   each T² is O⁻ or S⁻;
   T³ is a group

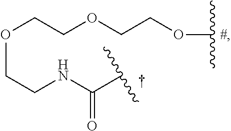 [SMILES: *OCCOCCOCCNC(*)=O]

wherein [symbol] † indicates the point of attachment to L and wherein [symbol] # indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H or oxo, wherein if one R^{g1}, R^{g2}, R^{g3}, and R^{g4} is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In still other embodiments, the oligonucleotide has the structure

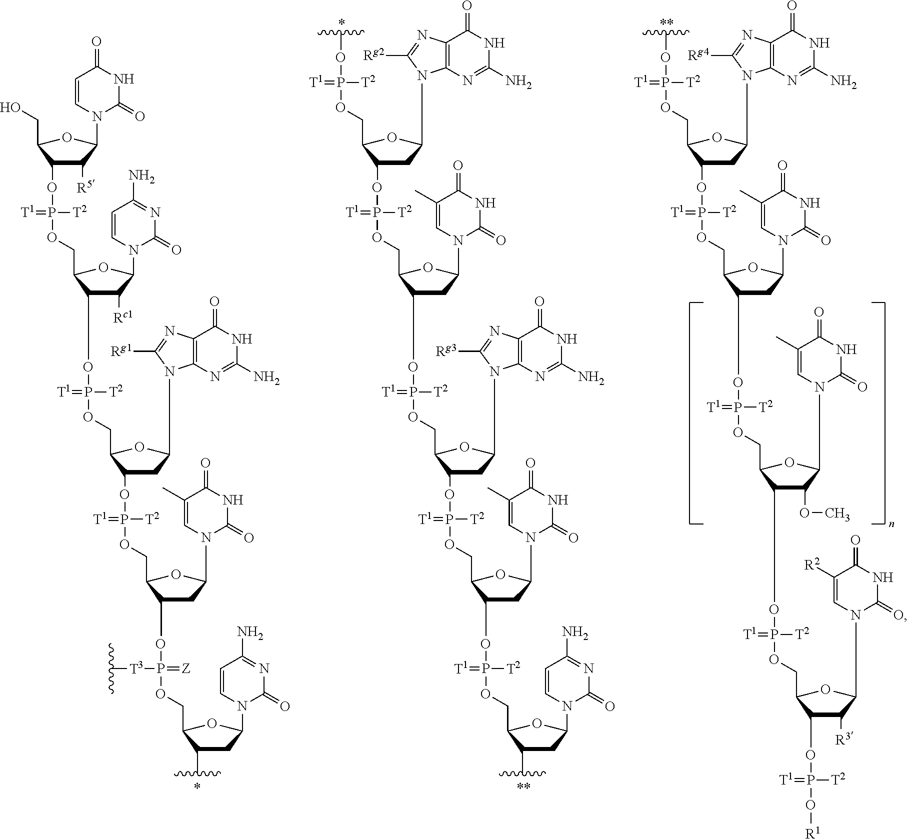

wherein
   [symbol] * and [symbol] ** indicate the points of attachment within the oligonucleotide;
   each T¹ is independently O or S;
   each T² is O⁻ or S⁻;
   T³ is a group

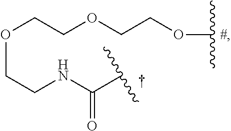 [SMILES: *OCCOCCOCCNC(*)=O]

wherein [symbol] † indicates the point of attachment to L and wherein [symbol] # indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H or oxo, wherein if one R^{g1}, R^{g2}, R^{g3}, and R^{g4} is oxo, then the carbon to which the oxo is attached has a single bond to the ring nitrogen at the 7-position;
R^{3′} is methoxy;
R¹ is C₁₋₄-alkylene-hydroxy;
R² is —H or methyl; and
n is an integer from 0 to 2.

In some embodiments, the oligonucleotide comprises one or more of unmodified sequences differing by 0, 1, 2 or 3 nucleobases from the sequences shown in Table 1. In some embodiments, the oligonucleotide comprises one or more of modified sequences differing by 0, 1, 2 or 3 nucleobases from the sequences shown in Table 2.

TABLE 1
Unmodified Oligonucleotides

| SEQ | Unmodified Oligonucleotide Sequence |
|---|---|
| ID NO. | (5′→3′) |
| 1 | tucgtcgtgacgtt |
| 2 | ucgtcgtgtcgtt |
| 129 | tcgtcgttttgtcgttttgtcgtt |

TABLE 2
Modified Oligonucleotides

| SEQ ID | Modified Oligonucleotide Sequence | |
|---|---|---|
| NO. | (5'->3') | Cmpd # |
| 3 | uscsgstscsgstsgstscsgstsT-c3 | 1.1b |
| 4 | uscsgstscsgstsgstscsgstst-c3 | 2.1b |
| 5 | uscsgstscsgstsgstscsgstst-c3 | 2.2b |
| 6 | uscsgstscsgstsgstscsgstst-c3 | 2.3b |
| 7 | uscsgstscsgstsgstscsgstst-c3 | 2.4b |
| 8 | uscsgstscsgstsgstscsgstst-c3 | 3.1b |
| 9 | uscsgstscsgstsgstscsgststst-c3 | 3.2b |
| 10 | uscsgstscsgstsgstscsgstststst-c3 | 3.3b |
| 11 | uscsgstscsgstsgstscsgstst-c3 | 4.1b |
| 12 | uscsgstscsgstsgstscsgststst-c3 | 4.2b |
| 13 | uscsgstscsgstsgstscsgststst-c3 | 4.3b |
| 14 | uscs2gstscsgstsgstscsgstst-c3 | 5.1a |
| 15 | uscsgs2tscsgstsgstscsgstst-c3 | 5.2a |
| 16 | uscsgstscs2gstsgstscsgstst-c3 | 5.3a |
| 17 | uscsgstscsgs2tsgstscsgstst-c3 | 5.4a |
| 18 | uscsgstscsgsts2gstscsgstst-c3 | 5.5a |
| 19 | uscsgstscsgstsgs2tscsgstst-c3 | 5.6a |
| 20 | uscsgstscsgstsgsts2csgstst-c3 | 5.7a |
| 21 | uscsgstscsgstsgstscs2gstst-c3 | 5.8a |
| 22 | uscsgstscsgstsgstscsgs2tst-c3 | 5.9a |
| 23 | uscsgstscsgstsgstscsgsts2t-c3 | 5.10a |
| 24 | uscsgstscsgstsgstscsgstsus2-c3 | 5.11a |
| 25 | uscs2gstscsgstsgstscsgstsus2-c3 | 5.12a |
| 26 | uscsgs2tscsgstsgstscsgststst-c3 | 6.1b |
| 27 | uscsgstscsgstsgsts2csgststst-c3 | 6.2b |
| 28 | uscsgs2tscsgstsgsts2csgststst-c3 | 6.3b |
| 29 | uscs2gstscsgstsgstscsgststst-c3 | 7.1b |
| 30 | uscsgstscsgstsgs2tscsgststst-c3 | 7.2b |
| 31 | uscs2gstscsgstsgs2tscsgststst-c3 | 7.3b |
| 32 | uscsgstscs2gstsgstscsgststst-c3 | 7.4b |
| 33 | uscsgstscsgs2tsgstscsgststst-c3 | 7.5b |
| 34 | uscsgstscsgsts2gstscsgststst-c3 | 7.6b |
| 35 | uscsgstscsgstsgsts2csgststst-c3 | 7.7b |
| 36 | uscs2gstscsgstsgsts2csgststst-c3 | 7.8b |
| 37 | uscsgs2tscsgstsgstscsgststst-c3 | 7.9b |
| 38 | uscsgstscsgstsgstscs2gststst-c3 | 7.10b |
| 130 | uscsgstscsgstsgstscsgstsT-c3 | 8.1b |
| 131 | uscsgstscsgstsgstscsgstst-c3 | 9.1b |
| 132 | uscsgstscsgstsgstscsgstst-c3 | 9.2b |
| 133 | uscsgstscsgstsgstscsgstst-c3 | 9.3b |
| 134 | uscsgstscsgstsgstscsgstst-c3 | 9.4b |
| 135 | uscsgstscsgstsgstscsgstst-c3 | 10.1b |
| 136 | uscsgstscsgstsgstscsgststst-c3 | 10.2b |
| 137 | uscsgstscsgstsgstscsgstststst-c3 | 10.3b |
| 138 | uscsgstscsgstsgstscsgstst-c3 | 12.1b |
| 139 | uscsgstscsgstsgstscsgststst-c3 | 12.2b |
| 140 | uscsgstscsgstsgstscsgststst-c3 | 12.3b |
| 141 | uscs2gstscsgstsgstscsgstst-c3 | 13.1a |
| 142 | uscsgs2tscsgstsgstscsgstst-c3 | 13.2a |
| 143 | uscsgstscs2gstsgstscsgstst-c3 | 13.3a |
| 144 | uscsgstscsgs2tsgstscsgstst-c3 | 13.4a |
| 145 | uscsgstscsgsts2gstscsgstst-c3 | 13.5a |
| 146 | uscsgstscsgstsgs2tscsgstst-c3 | 13.6a |
| 147 | uscsgstscsgstsgsts2csgstst-c3 | 13.7a |
| 148 | uscsgstscsgstsgstscs2gstst-c3 | 13.8a |
| 149 | uscsgstscsgstsgstscsgs2tst-c3 | 13.9a |
| 150 | uscsgstscsgstsgstscsgsts2t-c3 | 13.10a |
| 151 | uscsgstscsgstsgstscsgstsus2-c3 | 13.11a |
| 152 | uscs2gstscsgstsgstscsgstsus2-c3 | 13.12a |
| 153 | uscsgs2tscsgstsgstscsgststst-c3 | 14.1b |
| 154 | uscsgstscsgstsgsts2csgststst-c3 | 14.2b |
| 155 | uscsgs2tscsgstsgsts2csgststst-c3 | 14.3b |
| 156 | uscs2gstscsgstsgstscsgststst-c3 | 15.1b |
| 157 | uscsgstscsgstsgs2tscsgststst-c3 | 15.2b |
| 158 | uscs2gstscsgstsgs2tscsgststst-c3 | 15.3b |
| 159 | uscsgstscs2gstsgstscsgststst-c3 | 15.4b |
| 160 | uscsgstscsgs2tsgstscsgststst-c3 | 15.5b |
| 161 | uscsgstscsgsts2gstscsgststst-c3 | 15.6b |
| 162 | uscsgstscsgstsgsts2csgststst-c3 | 15.7a |
| 163 | uscsgstscsgstsgsts2csgststst-c3 | 15.7b |
| 164 | uscs2gstscsgstsgsts2csgststst-c3 | 15.8b |
| 165 | uscsgs2tscsgstsgstscsgststst-c3 | 15.9b |
| 166 | uscsgstscsgstsgstscs2gststst-c3 | 15.10b |

*u: 5-Bromo-2′-deoxyuridine
g: 8-oxo-7,8-dihydro-2′-deoxyguanosine
u: 5-Bromo 2′-OMe uridine
c: 2′-OMe-Cytidine
t: 2′-OMe-Thymidine
u: 2′-OMe-Uridine
u: 2′-deoxyuridine
T: 2′-OMOE thymidine
ts: phosphotriester liner-PEG₂₄-NH₂ 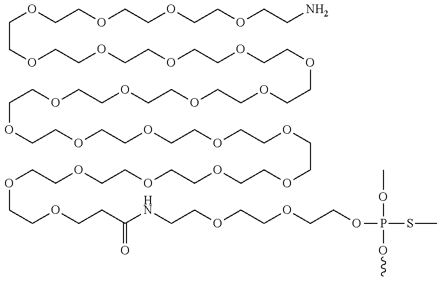
following thymidine;
ts: 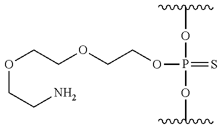 [SMILES: *OP(=S)(O*)OCCOCCOCCN] phosphotriester liner following thymidine;
Lower case: 2′-deoxy nucleotide
s: phosphorothioate linkage
s2: phosphorodithioate linkage
c3: 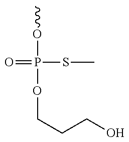 s2-c3:

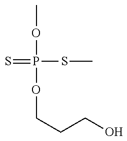

In some embodiments, the oligonucleotide is functionalized with a chemical tag for attachment to the linking moiety. In some embodiments, the chemical tag is attached to an inter-nucleoside linkage of the oligonucleotide. In some embodiments, the chemical tag is attached to a 5′ inter-nucleoside linkage. In some embodiments, the chemical tag is attached to a 3′ inter-nucleoside linkage. In some embodiments, the inter-nucleoside linkage is a phosphorothioate linkage. In some embodiments, the inter-nucleoside linkage is a phosphorodithioate linkage. In some embodiments, the chemical tag is closer to the 5′ end than the 3′ end of the oligonucleotide. In some embodiments, the chemical tag is attached to a nucleobase.

### Linking Moieties

In another aspect, the oligonucleotide is conjugated to the SIRP-α antibody via a linking moiety. The length, rigidity and chemical composition of the linking moiety impact the conjugation reaction rates and the stability of the resulting conjugates. In some embodiments, the linking moiety comprises polyethylene glycol (PEG). In some embodiments, the PEG contains about 10-50 ethylene glycol units. In some embodiments, the linking moiety is an aliphatic chain.

For Formula (A), the linking moiety is represented by L. In some embodiments, the linker L comprises an oligoethylene glycol or polyethylene glycol moiety. In certain embodiments, the linker L is a group having the structure

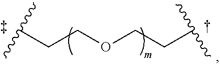

wherein [symbol] † indicates the point of attachment to Y^{PTE}, and [symbol] ‡ indicates the point of attachment to the rest of the conjugate.

In other embodiments, the linker L is a group having the structure

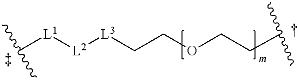

wherein [symbol] † indicates the point of attachment to Y^{PTE}, and [symbol] ‡ indicates the point of attachment to the rest of the conjugate. In some embodiments, L¹ is absent. In some embodiments, L¹ is unsubstituted alkyl. In some embodiments, L¹ is independently an unsubstituted C₁₋₆ alkyl. In some embodiments, each L¹ is methyl or ethyl. In some embodiments, L¹ is independently a substituted alkyl. In some embodiments, L¹ is independently a substituted C₁₋₆ alkyl. In some embodiments, L¹ is C₁₋₆ alkyl substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, cyano, azido, halo, hydroxyl, nitro, carboxyl, thiol, thioalkyl, alkyl, alkenyl, alkynyl, heterocyclyl, aminosulfonyl, sulfonylamino, sulfonyl and oxo.

In some embodiments, L² is absent. In some embodiments, L² is unsubstituted or substituted alkyl.

In some embodiments, L³ is absent. In some embodiments, L³ is a linker moiety. In some embodiments, the linker moiety is an unsubstituted or substituted alkyl. In some embodiments, the linker moiety is independently an unsubstituted C₁₋₆ alkyl. In some embodiments, the linker moiety is methyl or ethyl. In some embodiments, the linker moiety is independently a substituted alkyl. In some embodiments, the linker moiety is independently a substituted C₁₋₆ alkyl. In some embodiments, the linker moiety is C₁₋₆ alkyl substituted with one or more substituents selected from the group consisting of alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, cyano, azido, halo, hydroxyl, nitro, carboxyl, thiol, thioalkyl, alkyl, alkenyl, alkynyl, heterocyclyl, aminosulfonyl, sulfonylamino, sulfonyl and oxo. In some embodiments, the linker moiety is an amino acid residue. In some embodiments, the amino acid is selected from the group consisting of glycine, alanine, glutamic acid and proline. In some embodiments, the linker is methyl. In some embodiments, the linker moiety is —R⁵C(O)R⁶NHR⁷—, wherein R⁵, and R⁷ are independently absent or unsubstituted or substituted alkyl and R⁶ is an amino acid residue. In some embodiments, the amino acid is selected from the group consisting of glycine, alanine, glutamic acid and proline. In some embodiments, the linker moiety is —R³C(O)NHR⁴—, wherein R³ and R⁴ are independently absent or unsubstituted or substituted alkyl. In some embodiments, R³ is methylene and R⁴ is —(CH₂)₄—. In some embodiments, R³ is methylene and R⁴ is absent. When more than one oligonucleotide (i.e., p=2), the two L¹ can be different or same, the two L² can be different or same and the two L³ can be different or same.

In some embodiments, m is about 3-10, about 10-15, about 15-20, about 20-25, about 25-30, about 5-16, about 15-30, about 15-25 or about 20-30. In some embodiments, m is 20, 21, 22, 23, 24 or 25.

### Anti-SIRP-Alpha Antibodies and Q-Tag Peptides

Described herein, in various embodiments, are antibodies which specifically bind SIRP-α (i.e., anti-SIRP-α antibodies, SIRP-α antibodies, antibodies targeting SIRP-α), particularly antibodies which specifically bind human SIRP-α, and conjugates thereof. In some embodiments, the antibodies bind human SIRP-α polypeptide (e.g., an extracellular domain of a human SIRP-α polypeptide, such as a D1 domain). In some embodiments, SIRP-α refers to human SIRP-α, and the antibodies specifically bind human SIRP-α. SIRP-α gene and polypeptide sequences (e.g., human gene and polypeptide sequences) are known in the art (see exemplary sequences infra). In some embodiments, the SIRP-α conjugate (i.e., anti-SIRP-α conjugate) specifically binds to a cell (e.g., a myeloid cell or a tumor cell) that expresses a SIRP-α polypeptide, e.g., a human SIRP-α polypeptide, on its cell surface. Human SIRP-α is also known as BIT, MFR, P84, SIRP, MYD-1, SHPS1, CD172A, and PTPNS1. In some embodiments, a human SIRP-α polypeptide refers to a polypeptide encoded by a human SIRPA gene, e.g., as described by NCBI Ref Seq ID No. 140885.

In some embodiments, the SIRP-α antibody is selected from a group consisting of a polyclonal antibody, a monoclonal antibody, a humanized antibody, a human antibody, a chimeric antibody, and an antibody fragment. In some embodiments, the SIRP-α antibody is a full-length antibody, e.g., comprising an Fc region (including but not limited to the exemplary Fc regions described herein). In some embodiments, the SIRP-α antibody fragment is selected from the group consisting of Fab, Fab′, Fab′-SH, F(ab′)₂, Fv fragments, scFv, single domain antibody, single heavy chain antibody and single light chain antibody. In some embodiments, the SIRP-α antibody fragment is selected from the group consisting of Fab, Fab′, Fab′-SH, F(ab′)₂, Fv fragments, and scFv fragments.

Any of the anti-SIRP-α antibodies described herein may find use in any of the conjugates of the present disclosure. In some embodiments, a SIRP-α antibody of the present disclosure comprises a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58). In some embodiments, a SIRP-α antibody of the present disclosure comprises a light chain variable (VL) domain that comprises a CDR-L1 comprising the amino acid sequence of SGGSYSSYYYA (SEQ ID NO:59), a CDR-L² comprising the amino acid sequence of SDDKRPS (SEQ ID NO:60), and a CDR-L³ comprising the amino acid sequence of GGYDQSSYTNP (SEQ ID NO:61). In some embodiments, a SIRP-α antibody of the present disclosure comprises a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58); and a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61).

In some embodiments, a SIRP-α antibody of the present disclosure comprises a VH domain comprising the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62). In some embodiments, a SIRP-α antibody of the present disclosure comprises a VL domain comprising an amino acid sequence selected from the group consisting of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVL (SEQ ID NO:63), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTKLTVL (SEQ ID NO:64), and SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTELTVL (SEQ ID NO:65). In some embodiments, a SIRP-α antibody of the present disclosure comprises a VH domain comprising the amino acid sequence of SEQ ID NO:62 and a VL domain comprising the amino acid sequence of SEQ ID NO:63. In some embodiments, a SIRP-α antibody of the present disclosure comprises a VH domain comprising the amino acid sequence of SEQ ID NO:62 and a VL domain comprising the amino acid sequence of SEQ ID NO:64. In some embodiments, a SIRP-α antibody of the present disclosure comprises a VH domain comprising the amino acid sequence of SEQ ID NO:62 and a VL domain comprising the amino acid sequence of SEQ ID NO: 65.

In some embodiments, a SIRP-α antibody of the present disclosure comprises an antibody heavy chain comprising an antibody VH domain (e.g., comprising the amino acid sequence of SEQ ID NO:62) and an Fc region. In some embodiments, a SIRP-α antibody of the present disclosure (including C-terminal Q-tag peptide) comprises an antibody heavy chain comprising an amino acid sequence selected from the group consisting of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLGRPQGFGPP (SEQ ID NO:66), EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEA AGAPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGRPQGFGPP (SEQ ID NO:67), and EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTQKSLSLSPGRPQGFGPP (SEQ ID NO:68). In some embodiments, a SIRP-α antibody of the present disclosure comprises an antibody heavy chain comprising an amino acid sequence selected from the group consisting of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLG (SEQ ID NO:87), EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEA AGAPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:88), and EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:89). In some embodiments, a SIRP-α antibody of the present disclosure comprises an antibody heavy chain comprising an amino acid sequence selected from the group consisting of SEQ ID Nos: 87-89 and a Q-tag peptide or sequence of the present disclosure.

In some embodiments, a SIRP-α antibody of the present disclosure comprises an antibody heavy chain comprising the amino acid sequence of SEQ ID NO:66. In some embodiments, a SIRP-α antibody of the present disclosure comprises an antibody heavy chain comprising the amino acid sequence of SEQ ID NO:66 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:66 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:66 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:66 and an antibody light chain comprising a VL domain that comprises the amino acid sequence of SEQ ID NO:65.

In some embodiments, a SIRP-α antibody of the present disclosure comprises an antibody heavy chain comprising the amino acid sequence of SEQ ID NO:87. In some embodiments, a SIRP-α antibody of the present disclosure comprises an antibody heavy chain comprising the amino acid sequence of SEQ ID NO:87 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:87 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:87 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:87 and an antibody light chain comprising a VL domain that comprises the amino acid sequence of SEQ ID NO:65.

In some embodiments, a SIRP-α antibody of the present disclosure comprises an antibody heavy chain comprising the amino acid sequence of SEQ ID NO:67. In some embodiments, a SIRP-α antibody of the present disclosure comprises an antibody heavy chain comprising the amino acid sequence of SEQ ID NO:67 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:67 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:67 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:67 and an antibody light chain comprising a VL domain that comprises the amino acid sequence of SEQ ID NO:65.

In some embodiments, a SIRP-α antibody of the present disclosure comprises an antibody heavy chain comprising the amino acid sequence of SEQ ID NO:88. In some embodiments, a SIRP-α antibody of the present disclosure comprises an antibody heavy chain comprising the amino acid sequence of SEQ ID NO:88 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:88 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:88 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:88 and an antibody light chain comprising a VL domain that comprises the amino acid sequence of SEQ ID NO:65.

In some embodiments, a SIRP-α antibody of the present disclosure comprises an antibody heavy chain comprising the amino acid sequence of SEQ ID NO:68. In some embodiments, a SIRP-α antibody of the present disclosure comprises an antibody heavy chain comprising the amino acid sequence of SEQ ID NO:68 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:68 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:68 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:68 and an antibody light chain comprising a VL domain that comprises the amino acid sequence of SEQ ID NO:65.

In some embodiments, a SIRP-α antibody of the present disclosure comprises an antibody heavy chain comprising the amino acid sequence of SEQ ID NO:89. In some embodiments, a SIRP-α antibody of the present disclosure comprises an antibody heavy chain comprising the amino acid sequence of SEQ ID NO:89 and an antibody light chain comprising a VL domain that comprises the amino acid sequence of SEQ ID NO:63, 64, or 65. In some embodiments, a SIRP-α antibody of the present disclosure comprises an antibody heavy chain comprising the amino acid sequence of SEQ ID NO:89 and an antibody light chain comprising a VL domain that comprises the amino acid sequence of SEQ ID NO:63. In some embodiments, a SIRP-α antibody of the present disclosure comprises an antibody heavy chain comprising the amino acid sequence of SEQ ID NO:89 and an antibody light chain comprising a VL domain that comprises the amino acid sequence of SEQ ID NO:64. In some embodiments, a SIRP-α antibody of the present disclosure comprises an antibody heavy chain comprising the amino acid sequence of SEQ ID NO:89 and an antibody light chain comprising a VL domain that comprises the amino acid sequence of SEQ ID NO:65.

In some embodiments, a SIRP-α antibody of the present disclosure comprises an antibody light chain comprising an antibody VL domain (e.g., comprising the amino acid sequence of SEQ ID NO:63, 64, or 65) and an antibody constant light chain (CL) domain. In some embodiments, the CL domain is a kappa CL domain (e.g., a human kappa CL domain). In some embodiments, the CL domain is a lambda CL domain (e.g., a human lambda CL domain). In some embodiments, the CL domain is a human IGLC1 or IGLC2 domain. In some embodiments, a SIRP-α antibody of the present disclosure comprises an antibody light chain comprising the amino acid sequence of GQPKANPTVTLFPPSSEELQANKATLVCLISDFYPGAVTVAWKADGSPVKAGVETTKPS KQSNNKYAASSYLSLTPEQWKSHRSYSCQVTHEGSTVEKTVAPTECS (SEQ ID NO:69), GQPKANPTVTLFPPSSEELQANKATLVCLISDFYPGAVTVAWKADGSPVKAGVETTKPS KQSSDKYAASSYLSLTPEQWKSHRSYSCQVTHEGSTVEKTVAPTECS (SEQ ID NO:70), or GQPKAAPSVTLFPPSSEELQANKATLVCLISDFYPGAVTVAWKADSSPVKAGVETTTPS KQSNNKYAASSYLSLTPEQWKSHRSYSCQVTHEGSTVEKTVAPTECS (SEQ ID NO:71).

In some embodiments, a SIRP-α antibody of the present disclosure comprises an antibody light chain comprising an antibody VL domain that comprises the amino acid sequence of SEQ ID NO:63, 64, or 65 and an antibody CL domain that comprises the amino acid sequence of SEQ ID NO:69, 70, or 71. In some embodiments, a SIRP-α antibody of the present disclosure comprises an antibody light chain comprising an antibody VL domain that comprises the amino acid sequence of SEQ ID NO:63 and an antibody CL domain that comprises the amino acid sequence of SEQ ID NO:69. In some embodiments, a SIRP-α antibody of the present disclosure comprises an antibody light chain comprising an antibody VL domain that comprises the amino acid sequence of SEQ ID NO:63 and an antibody CL domain that comprises the amino acid sequence of SEQ ID NO:70. In some embodiments, a SIRP-α antibody of the present disclosure comprises an antibody light chain comprising an antibody VL domain that comprises the amino acid sequence of SEQ ID NO:63 and an antibody CL domain that comprises the amino acid sequence of SEQ ID NO:71. In some embodiments, a SIRP-α antibody of the present disclosure comprises an antibody light chain comprising an antibody VL domain that comprises the amino acid sequence of SEQ ID NO:64 and an antibody CL domain that comprises the amino acid sequence of SEQ ID NO:69. In some embodiments, a SIRP-α antibody of the present disclosure comprises an antibody light chain comprising an antibody VL domain that comprises the amino acid sequence of SEQ ID NO:64 and an antibody CL domain that comprises the amino acid sequence of SEQ ID NO:70. In some embodiments, a SIRP-α antibody of the present disclosure comprises an antibody light chain comprising an antibody VL domain that comprises the amino acid sequence of SEQ ID NO:64 and an antibody CL domain that comprises the amino acid sequence of SEQ ID NO:71. In some embodiments, a SIRP-α antibody of the present disclosure comprises an antibody light chain comprising an antibody VL domain that comprises the amino acid sequence of SEQ ID NO:65 and an antibody CL domain that comprises the amino acid sequence of SEQ ID NO:69. In some embodiments, a SIRP-α antibody of the present disclosure comprises an antibody light chain comprising an antibody VL domain that comprises the amino acid sequence of SEQ ID NO:65 and an antibody CL domain that comprises the amino acid sequence of SEQ ID NO:70. In some embodiments, a SIRP-α antibody of the present disclosure comprises an antibody light chain comprising an antibody VL domain that comprises the amino acid sequence of SEQ ID NO:65 and an antibody CL domain that comprises the amino acid sequence of SEQ ID NO:71.

In some embodiments, a SIRP-α antibody of the present disclosure comprises an antibody light chain comprising an amino acid sequence selected from the group consisting of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVLGQPKANPTVTL FPPSSEELQANKATLVCLISDFYPGAVTVAWKADGSPVKAGVETTKPSKQSNNKYAASS YLSLTPEQWKSHRSYSCQVTHEGSTVEKTVAPTECS (SEQ ID NO:72), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTKLTVLGQPKANPTVTL FPPSSEELQANKATLVCLISDFYPGAVTVAWKADGSPVKAGVETTKPSKQSNNKYAASS YLSLTPEQWKSHRSYSCQVTHEGSTVEKTVAPTECS (SEQ ID NO:73), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTELTVLGQPKANPTVTLF PPSSEELQANKATLVCLISDFYPGAVTVAWKADGSPVKAGVETTKPSKQSNNKYAASSY LSLTPEQWKSHRSYSCQVTHEGSTVEKTVAPTECS (SEQ ID NO:74), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVLGQPKANPTVTL FPPSSEELQANKATLVCLISDFYPGAVTVAWKADGSPVKAGVETTKPSKQSSDKYAASS YLSLTPEQWKSHRSYSCQVTHEGSTVEKTVAPTECS (SEQ ID NO:75), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTKLTVLGQPKANPTVTL FPPSSEELQANKATLVCLISDFYPGAVTVAWKADGSPVKAGVETTKPSKQSSDKYAASS YLSLTPEQWKSHRSYSCQVTHEGSTVEKTVAPTECS (SEQ ID NO:76), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTELTVLGQPKANPTVTLF PPSSEELQANKATLVCLISDFYPGAVTVAWKADGSPVKAGVETTKPSKQSSDKYAASSY LSLTPEQWKSHRSYSCQVTHEGSTVEKTVAPTECS (SEQ ID NO:77), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVLGQPKAAPSVTL FPPSSEELQANKATLVCLISDFYPGAVTVAWKADSSPVKAGVETTTPSKQSNNKYAASS YLSLTPEQWKSHRSYSCQVTHEGSTVEKTVAPTECS (SEQ ID NO:78), SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTKLTVLGQPKAAPSVTL FPPSSEELQANKATLVCLISDFYPGAVTVAWKADSSPVKAGVETTTPSKQSNNKYAASS YLSLTPEQWKSHRSYSCQVTHEGSTVEKTVAPTECS (SEQ ID NO:79), and SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTELTVLGQPKAAPSVTLF PPSSEELQANKATLVCLISDFYPGAVTVAWKADSSPVKAGVETTTPSKQSNNKYAASSY LSLTPEQWKSHRSYSCQVTHEGSTVEKTVAPTECS (SEQ ID NO:80).

In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:72. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:73. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:74. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:75. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:76. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:77. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:78. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:79. In some embodiments, a SIRP-α antibody of the present disclosure comprises an antibody heavy chain comprising the amino acid sequence of SEQ ID NO:66 or 87 and an antibody light chain comprising the amino acid sequence of SEQ ID NO:80.

In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:72. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:73. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:74. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:75. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:76. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:77. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:78. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:79. In some embodiments, a SIRP-α antibody of the present disclosure comprises an antibody heavy chain comprising the amino acid sequence of SEQ ID NO:67 or 88 and an antibody light chain comprising the amino acid sequence of SEQ ID NO:80.

In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:72. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:73. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:74. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:75. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:76. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:77. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:78. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:79. In some embodiments, a SIRP-α antibody of the present disclosure comprises an antibody heavy chain comprising the amino acid sequence of SEQ ID NO:68 or 89 and an antibody light chain comprising the amino acid sequence of SEQ ID NO:80.

In some embodiments, the SIRP-α antibody further comprises at least one Q-tag peptide sequence. In some embodiments, at least one Q-tag is attached to the heavy chain of the SIRP-α antibody. In certain embodiments, at least one Q-tag is fused to the C-terminus of the heavy chain of the SIRP-α antibody. In other embodiments, at least one Q-tag is attached to the light chain of the SIRP-α antibody. In still further embodiments, at least one Q-tag is within the Fc domain or region. Exemplary and non-limiting Q-tag peptide sequences are disclosed herein and may find use in any of the antibodies or conjugates of the present disclosure.

In some embodiments, an antibody or conjugate of the present disclosure binds, or is capable of binding, an extracellular domain (e.g., the D1 domain) of a human SIRP-α v1 polypeptide and/or a human SIRP-α v2 polypeptide. In some embodiments, the human SIRP-α v1 polypeptide comprises the amino acid sequence of EEELQVIQPDKSVLVAAGETATLRCTATSLIPVGPIQWFRGAGPGRELIYNQKEGHFPRV TTVSDLTKRNNMDFSIRIGNITPADAGTYYCVKFRKGSPDDVEFKSGAGTELSVRAKPS (SEQ ID NO:81). In some embodiments, the human SIRP-α v2 polypeptide comprises the amino acid sequence of EEELQVIQPDKSVSVAAGESAILHCTVTSLIPVGPIQWFRGAGPARELIYNQKEGHFPRVT TVSESTKRENMDFSISISNITPADAGTYYCVKFRKGSPDTEFKSGAGTELSVRAKPS (SEQ ID NO: 82). In some embodiments, an antibody or conjugate of the present disclosure binds, or is capable of binding, an extracellular domain (e.g., the D1 domain) of a human SIRP-α v1 polypeptide and a human SIRP-α v2 polypeptide. In some embodiments, an antibody or conjugate of the present disclosure binds, or is capable of binding, an extracellular domain (e.g., the D1 domain) of a human SIRP-α v1 polypeptide and/or a human SIRP-α v2 polypeptide with a dissociation constant (K_{D}) of less than 100 nM, less than 10 nM, less than 1 nM, or 1 pM or less.

In some embodiments, an antibody or conjugate of the present disclosure binds an extracellular domain (e.g., the D1 domain) of a monkey SIRP-α polypeptide (e.g., the D1 domain of a monkey SIRP-α polypeptide). In some embodiments, an antibody or conjugate of the present disclosure binds, or is capable of binding, an extracellular domain (e.g., the D1 domain) of a cynomolgus SIRP-α polypeptide (e.g., found in the organism Macaca fascicularis). In some embodiments, the cynomolgus SIRP-α polypeptide comprises the amino acid sequence of EEELQVIQPEKSVSVAAGESATLNCTATSLIPVGPIQWFRGVGPGRELIYHQKEGHFPRV TPVSDPTKRNNMDFSIRISNITPADAGTYYCVKFRKGSPDVELKSGAGTELSVRAKPS (SEQ ID NO:84). In some embodiments, an antibody or conjugate of the present disclosure binds, or is capable of binding, an extracellular domain (e.g., the D1 domain) of a mouse SIRP-α polypeptide (e.g., the D1 domain of a mouse SIRP-α polypeptide). In some embodiments, the mouse SIRP-α polypeptide comprises the amino acid sequence of KELKVTQPEKSVSVAAGDSTVLNCTLTSLLPVGPIKWYRGVGQSRLLIYSFTGEHFPRVT NVSDATKRNNMDFSIRISNVTPEDAGTYYCVKFQKGPSEPDTEIQSGGGTEVYVLAKPS (SEQ ID NO:83).

In some embodiments, an antibody or conjugate of the present disclosure binds, or is capable of binding, an extracellular domain (e.g., the D1 domain) of a human SIRP-β polypeptide and/or a human SIRP-γ polypeptide. In some embodiments, a human SIRP-β polypeptide refers to a polypeptide encoded by a human SIRPB1 gene, e.g., as described by NCBI Ref Seq ID No. 10326. In some embodiments, a human SIRP-γ polypeptide refers to a polypeptide encoded by a human SIRPG gene, e.g., as described by NCBI Ref Seq ID No. 55423. In some embodiments, the human SIRP-β polypeptide comprises the amino acid sequence of EDELQVIQPEKSVSVAAGESATLRCAMTSLIPVGPIMWFRGAGAGRELIYNQKEGHFPR VTTVSELTKRNNLDFSISISNITPADAGTYYCVKFRKGSPDDVEFKSGAGTELSVRAKPS (SEQ ID NO:85). In some embodiments, the human SIRP-γ polypeptide comprises the amino acid sequence of EEELQMIQPEKLLLVTVGKTATLHCTVTSLLPVGPVLWFRGVGPGRELIYNQKEGHFPR VTTVSDLTKRNNMDFSIRISSITPADVGTYYCVKFRKGSPENVEFKSGPGTEMALGAKPS (SEQ ID NO:86).

In some embodiments, an antibody or conjugate of the present disclosure binds, or is capable of binding, an extracellular domain (e.g., the D1 domain) of a human SIRP-α v1 polypeptide, a human SIRP-α v2 polypeptide, a cynomolgus SIRP-α polypeptide, a mouse SIRP-α polypeptide, a human SIRP-β polypeptide, and/or a human SIRP-γ polypeptide. In some embodiments, an antibody or conjugate of the present disclosure binds, or is capable of binding, an extracellular domain (e.g., the D1 domain) of a human SIRP-α v1 polypeptide, a human SIRP-α v2 polypeptide, a cynomolgus SIRP-α polypeptide, a mouse SIRP-α polypeptide, a human SIRP-β polypeptide, and a human SIRP-γ polypeptide. Cross-reactivity across mammalian species such as human, monkeys, and mice may be advantageous, e.g., for preclinical testing of the antibodies or conjugates of the present disclosure.

In some embodiments, an antibody or conjugate of the present disclosure modulates SIRP-α signaling in a cell expressing a human SIRP-α polypeptide. In some embodiments, an antibody or conjugate of the present disclosure antagonizes SIRP-α signaling in a cell expressing a human SIRP-α polypeptide. In some embodiments, an antibody or conjugate of the present disclosure interferes with SIRP-α signaling in a cell expressing a human SIRP-α polypeptide. In some embodiments, an antibody or conjugate of the present disclosure agonizes SIRP-α signaling in a cell expressing a human SIRP-α polypeptide. In some embodiments, SIRP-α signaling includes one or more intracellular signaling events mediated by activation of a SIRP-α polypeptide, including without limitation tyrosine phosphorylation of the intracellular region of SIRP-α, phosphatase (e.g., SHP1) binding, adaptor protein binding (e.g., SCAP2, FYB, and/or GRB2), and nitric oxide production. Various assays for measuring SIRP-α signaling in a cell include without limitation SIRP-α phosphorylation, SHP1 and SHP2 co-immunoprecipitation, PI3-kinase signaling, cytokine production (both inflammatory IL-12, IL-23, TNF, IFN and suppressive cytokines IL-10, IL-4, IL-13, cell surface markers levels for M1 and M2 macrophage markers) or dendritic cell activation and function; Kharitonenkov, A. et al. (1997) Nature 386:181-6; Ochi, F. et al. (1997) Biochem. Biophys. Res. Commun. 239:483-7; Kim, E. J. et al. (2013) Inflammation Research 62:377-86; Yi, T. et al. (2015) Immunity 43:764-75.

In some embodiments, the cell expressing a human SIRP-α polypeptide is a leukocyte. In some embodiments, the cell is a macrophage, dendritic cell, neutrophil, eosinophil, or myeloid-derived suppressor cell (MDSC). In some embodiments, an antibody or conjugate of the present disclosure decreases or antagonizes SIRP-α signaling in a cell expressing a human SIRP-α polypeptide by at least 20%, at least 30%, at least 40%, at least 50%, at least 60%, at least 70%, at least 80%, or at least 90%, e.g., using one or more of the SIRP-α signaling assays described herein or otherwise known in the art. In some embodiments, an antibody or conjugate of the present disclosure increases or agonizes SIRP-α signaling in a cell expressing a human SIRP-α polypeptide by at least 20%, at least 30%, at least 40%, at least 50%, at least 60%, at least 70%, at least 80%, or at least 90%, e.g., using one or more of the SIRP-α signaling assays described herein or otherwise known in the art.

In some embodiments, an antibody or conjugate of the present disclosure modulates an intercellular phenotype mediated by SIRP-α. In some embodiments, an antibody or conjugate of the present disclosure enhances phagocytosis by a macrophage expressing a human SIRP-α polypeptide. For example, phagocytic activity of a macrophage treated or contacted with an antibody or conjugate of the present disclosure can be compared with phagocytic activity of a macrophage not treated or contacted with the antibody or conjugate, or phagocytic activity of a macrophage that expresses a human SIRP-α polypeptide and is treated or contacted with an antibody or conjugate of the present disclosure can be compared with phagocytic activity of a macrophage that does not express a human SIRP-α polypeptide and is treated or contacted with the antibody or conjugate. Exemplary phagocytosis assays may be found, e.g., in Wieskopf, K. et al (2013) Science 341:88 and Willingham, S. B. et al. (2012) Proc. Natl. Acad. Sci. 109:6662-7. In some embodiments, an antibody or conjugate of the present disclosure increases phagocytosis by a macrophage expressing a human SIRP-α polypeptide by at least 20%, at least 30%, at least 40%, at least 50%, at least 60%, at least 70%, at least 80%, or at least 90%, e.g., using one or more of the phagocytosis assays described herein or otherwise known in the art.

In some embodiments, an antibody or conjugate of the present disclosure enhances activation of dendritic cell(s) expressing a human SIRP-α polypeptide (e.g., an increased level of activation of individual dendritic cells, or an increased proportion of dendritic cells that are activated within a sample population). For example, activation of dendritic cell(s) treated or contacted with an antibody or conjugate of the present disclosure can be compared with activation of dendritic cell(s) not treated or contacted with the antibody or conjugate, or activation of dendritic cell(s) that express a human SIRP-α polypeptide and are treated or contacted with an antibody or conjugate of the present disclosure can be compared with activation of dendritic cell(s) that do not express a human SIRP-α polypeptide and are treated or contacted with the antibody or conjugate. Exemplary dendritic cell activation assays are described herein. In some embodiments, an antibody or conjugate of the present disclosure increases dendritic cell (e.g., expressing a human SIRP-α polypeptide) activation by at least 20%, at least 30%, at least 40%, at least 50%, at least 60%, at least 70%, at least 80%, or at least 90%, e.g., using one or more of the dendritic cell activation assays described herein or otherwise known in the art.

In some embodiments, an antibody or conjugate of the present disclosure inhibits in vivo growth of a tumor that expresses CD47 (e.g., human CD47). For example, in vivo growth of a tumor that expresses CD47 and is treated with an antibody or conjugate of the present disclosure can be compared against in vivo growth of a tumor that expresses CD47 and is not treated with an antibody or conjugate of the present disclosure. Exemplary in vivo tumor growth assays are described herein. In some embodiments, an antibody or conjugate of the present disclosure inhibits in vivo growth of a tumor that expresses CD47 by at least 20%, at least 30%, at least 40%, at least 50%, at least 60%, at least 70%, at least 80%, or at least 90%, e.g., using one or more of the in vivo tumor growth assays described herein or otherwise known in the art.

In some embodiments, an antibody or conjugate of the present disclosure inhibits in vivo growth of a tumor that expresses SIRP-α (e.g., human SIRP-α). For example, in vivo growth of a tumor that expresses SIRP-α and is treated with an antibody or conjugate of the present disclosure can be compared against in vivo growth of a tumor that expresses SIRP-α and is not treated with an antibody or conjugate of the present disclosure. Exemplary in vivo tumor growth assays are described herein. In some embodiments, an antibody or conjugate of the present disclosure inhibits in vivo growth of a tumor that expresses SIRP-α by at least 20%, at least 30%, at least 40%, at least 50%, at least 60%, at least 70%, at least 80%, or at least 90%, e.g., using one or more of the in vivo tumor growth assays described herein or otherwise known in the art.

In some embodiments, an antibody or conjugate of the present disclosure blocks binding between an extracellular domain (e.g., the D1 domain) of a human SIRP-α polypeptide and a human CD47 polypeptide, e.g., an IgSF domain of a human CD47 polypeptide. For example, the antibody or conjugate and the CD47 polypeptide may “compete” for the same SIRP-α epitope, and/or antibody or conjugate binding to SIRP-α may be mutually exclusive with CD47 binding to SIRP-α. The binding interface between SIRP-α and CD47, as well as residues of both proteins that participate in binding, are known; see Hatherley, D. et al. (2007) J. Biol. Chem. 282:14567-75 and Nakaishi, A. et al. (2008) J. Mol. Biol. 375:650-60. Exemplary assays for determining whether an antibody or conjugate blocks CD47 binding to SIRP-α are known in the art. In some embodiments, an antibody or conjugate of the present disclosure blocks binding between an extracellular domain (e.g., the D1 domain) of a human SIRP-α polypeptide and an IgSF domain of a human CD47 polypeptide in an in vitro assay, e.g., using purified SIRP-α and/or CD47 polypeptides. In some embodiments, a “blocking” anti-SIRP-α antibody or conjugate of the present disclosure binds to the extracellular domain of a SIRP-α polypeptide (e.g., the D1 domain) at one or more amino acid positions that are also bound by CD47 in the CD47: SIRP-α complex. In some embodiments, an antibody or conjugate of the present disclosure blocks binding between an extracellular domain (e.g., the D1 domain) of a human SIRP-α polypeptide expressed on the surface of a first cell and an IgSF domain of a human CD47 polypeptide expressed on the surface of a second cell, e.g., an in vivo binding assay between polypeptides expressed on the surface of cells. In some embodiments, the in vivo assay may assess binding between an extracellular domain (e.g., the D1 domain) of a human SIRP-α polypeptide expressed on the surface of a first cell and an IgSF domain of a human CD47 polypeptide expressed on the surface of a second cell by assaying one or more aspects of SIRP-α signaling, e.g., one or more intracellular signaling events mediated by activation of a SIRP-α polypeptide, including without limitation tyrosine phosphorylation of the intracellular region of SIRP-α, phosphatase (e.g., SHP1) binding, adaptor protein binding (e.g., SCAP2, FYB, and/or GRB2), cytokine production (e.g. IL-10, IL-1β, IFN or TNF), and nitric oxide production; and/or one or more intercellular phenotypes, including without limitation macrophage phagocytosis and other activating or suppressive phenotypes of macrophages, neutrophils, dendritic cells, eosinophils, and myeloid-derived suppressor cells (MDSCs).

In some embodiments, which may be combined with any of the foregoing embodiments, the SIRP-α antibody or conjugate comprises an Fc region. In certain embodiments, the Fc region is a human Fc region selected from the group consisting of an IgG1 Fc region, an IgG2 Fc region, and an IgG4 Fc region. In some embodiments, the Fc region is a wild-type human IgG1, IgG2, or IgG4 Fc region. In some embodiments, the Fc region is a human Fc region comprising one or more amino acid substitutions that reduce or eliminate one or more effector functions, as compared with the effector function(s) of a human Fc region that lacks the amino acid substitution(s). In some embodiments, the Fc region is a human IgG4 Fc region comprising an S228P substitution, amino acid position numbering according to EU index. In some embodiments, the Fc region is a human IgG1 Fc region comprising L234A, L235A, and G237A substitutions, amino acid position numbering according to EU index. In still yet further embodiments, the Fc region is: (a) a human IgG1 Fc region comprising L234A, L235A, and/or G237A substitutions, amino acid position numbering according to EU index; (b) a human IgG2 Fc region comprising A330S and/or P331S substitutions, amino acid position numbering according to EU index; (c) a human IgG4 Fc region comprising S228P and/or L235E substitutions, amino acid position numbering according to EU index; (d) a human IgG1 Fc region comprising an N297A substitution, amino acid position numbering according to EU index; (e) a human IgG1 Fc region comprising a D265A substitution, amino acid position numbering according to EU index; or (f) a human IgG2 Fc region comprising an N297A substitution, amino acid position numbering according to EU index. In some embodiments, the Fc region is a human Fc region comprising one or more amino acid substitutions that reduce or eliminate binding to human C1q, as compared with the binding of a human Fc region that lacks the amino acid substitution(s). In some embodiments, the Fc region is a human Fc region comprising one or more amino acid substitutions that reduce or eliminate antibody-dependent cellular cytotoxicity (ADCC), as compared with the ADCC of a human Fc region that lacks the amino acid substitution(s). Exemplary Fc regions are provided in the antibody heavy chain sequences disclosed herein.

Antibodies that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and, release of inflammatory mediators, placental transfer, and control of immunoglobulin production. Additionally, binding of the C1 component of complement to antibodies can activate the complement system. Activation of complement can be important for the lysis of cellular pathogens. However, the activation of complement can also stimulate the inflammatory response and can also be involved in autoimmune hypersensitivity or other immunological disorders. Variant Fc regions with reduced or ablated ability to bind certain Fc receptors are useful for developing therapeutic antibodies and Fc-fusion polypeptide constructs which act by targeting, activating, or neutralizing ligand functions while not damaging or destroying local cells or tissues. In some embodiments, the Fc region has wild-type or native CDC activity.

In some embodiments, an Fc domain can refer to a dimer of two Fc domain monomers. In a wild-type Fc domain, two Fc domain monomers dimerize by the interaction between the two CH3 antibody constant domains, as well as one or more disulfide bonds that form between the hinge domains of the two dimerized Fc domain monomers. In some embodiments, an Fc domain is mutated to lack effector functions, for example a “dead Fc domain.” In some embodiments, each of the Fc domain monomers in an Fc domain includes amino acid substitutions in the CH2 antibody constant domain to reduce the interaction or binding between the Fc domain and an Fc receptor, such as an Fcγ receptor (FcγR), an Fcα receptor (FcαR), or an Fcε (FcεR).

The Fc domain is not involved directly in binding an antibody to its target, but can be involved in various effector functions, such as participation of the antibody in antibody-dependent cellular toxicity. In some embodiments, the Fc domain in an antibody or conjugate of the disclosure comprises one or more amino acid substitutions, additions or insertions, deletions, or any combinations thereof that lead to decreased effector function such as decreased antibody-dependent cell-mediated cytotoxicity (ADCC), decreased complement-dependent cytolysis (CDC), decreased antibody-dependent cell-mediated phagocytosis (ADCP), or any combinations thereof. In some embodiments, the antibodies or conjugates of the disclosure are characterized by decreased binding (e.g., minimal binding or absence of binding) to a human Fc receptor and decreased binding (e.g., minimal binding or absence of binding) to complement protein C1q. In some embodiments, the antibodies or conjugates of the disclosure are characterized by decreased binding (e.g., minimal binding or absence of binding) to human FcγRI, FcγRIIA, FcγRIIB, FcγRIIIB, FcγRIIIB, or any combinations thereof, and C1q. To alter or reduce an antibody-dependent effector function, such as ADCC, CDC, ADCP, or any combinations thereof, in some embodiments, the Fc domains in antibodies or conjugates of the disclosure are of the IgG class and comprise one or more amino acid substitutions at E233, L234, L235, G236, G237, D265, D270, N297, E318, K320, K322, A327, A330, P331, or P329 (numbering according to the EU index of Kabat (Sequences of Proteins of Immunological Interest, 5th Ed. Public Health Service, National Institutes of Health, Bethesda, MD. (1991))).

In some embodiments, SIRP-α antibodies or conjugates comprising a non-native Fc region described herein exhibit reduced or ablated binding to at least one of Fcγ receptors CD16a, CD32a, CD32b, CD32c, and CD64 as compared to a polypeptide construct comprising a native Fc region. In some cases, the SIRP-α antibodies or conjugates described herein exhibit reduced or ablated binding to CD16a, CD32a, CD32b, CD32c, and CD64 Fcγ receptors.

CDC refers to a form of cytotoxicity in which the complement cascade is activated by the complement component C1q binding to antibody Fc. In some embodiments, SIRP-α antibodies or conjugates comprising a non-native Fc region described herein exhibit at least a 5%, 10%, 15%, 20%, 30%, 40%, 50%, 60%, 70%, 80%, 90% or greater reduction in C1q binding compared to the antibody or conjugate comprising a wild-type Fc region. In some cases, antibodies or conjugates comprising a non-native Fc region as described herein exhibit reduced CDC as compared to antibodies or conjugates comprising a wild-type Fc region. In some embodiments, antibodies or conjugates comprising a non-native Fc region as described herein exhibit at least a 5%, 10%, 15%, 20%, 30%, 40%, 50%, 60%, 70%, 80%, 90% or greater reduction in CDC compared to antibodies or conjugates comprising a wild-type Fc region. In some cases, antibodies or conjugates comprising a non-natural Fc variant as described herein exhibit negligible CDC as compared to antibodies or conjugates comprising a wild-type Fc region.

In some embodiments, the Fc variants herein are minimally glycosylated or have reduced glycosylation relative to a wild-type sequence. In some embodiments, deglycosylation is accomplished with a mutation of N297A, or by mutating N297 to any amino acid which is not N. In some embodiments, deglycosylation is accomplished by disrupting the motif N-Xaa1-Xaa2-Xaa3 (SEQ ID NO:175), wherein N=asparagine; Xaa1=any amino acid except P (proline); Xaa2=T (threonine), S (serine) or C (cysteine); and Xaa3=any amino acid except P (proline). In one embodiment, the N-Xaa1-Xaa2-Xaa3 (SEQ ID NO:175) motif refers to residues 297-300 as designated according to Kabat et al., 1991. In some embodiments, a mutation to any one or more of N, Xaa1, Xaa2, or Xaa3 results in deglycosylation of the Fc variant.

In some embodiments, variants of antibody IgG constant regions (e.g., Fc variants) possess a reduced capacity to specifically bind Fcγ receptors or have a reduced capacity to induce phagocytosis. In some embodiments, variants of antibody IgG constant regions (e.g., Fc variants) possess a reduced capacity to specifically bind Fcγ receptors and have a reduced capacity to induce phagocytosis. For example, in some embodiments, an Fc domain is mutated to lack effector functions, typical of a “dead” Fc domain. For example, in some embodiments, an Fc domain includes specific amino acid substitutions that are known to minimize the interaction between the Fc domain and an Fcγ receptor. In some embodiments, an Fc domain monomer is from an IgG1 antibody and includes one or more of amino acid substitutions L234A, L235A, G237A, and N297A (as designated according to the EU numbering system per Kabat et al., 1991). In some embodiments, one or more additional mutations are included in such IgG1 Fc variant. Non-limiting examples of such additional mutations for human IgG1 Fc variants include E318A and K322A. In some instances, a human IgG1 Fc variant has up to 12, 11, 10, 9, 8, 7, 6, 5 or 4 or fewer mutations in total as compared to wild-type human IgG1 sequence. In some embodiments, one or more additional deletions are included in such IgG1 Fc variant. For example, in some embodiments, the C-terminal lysine of the Fc IgG1 heavy chain constant region is deleted, for example to increase the homogeneity of the polypeptide when the polypeptide is produced in bacterial or mammalian cells. In some instances, a human IgG1 Fc variant has up to 12, 11, 10, 9, 8, 7, 6, 5 or 4 or fewer deletions in total as compared to wild-type human IgG1 sequence.

In some embodiments, an Fc domain monomer is from an IgG2 or IgG4 antibody and includes amino acid substitutions A330S, P331S, or both A330S and P331S. The aforementioned amino acid positions are defined according to Kabat, et al. (1991). The Kabat numbering of amino acid residues can be determined for a given antibody by alignment at regions of homology of the sequence of the antibody with a “standard” Kabat numbered sequence. In some embodiments, the Fc variant comprises a human IgG2 Fc sequence comprising one or more of A330S, P331S and N297A amino acid substitutions (as designated according to the EU numbering system per Kabat, et al. (1991). In some embodiments, one or more additional mutations are included in such IgG2 Fc variants. Non-limiting examples of such additional mutations for human IgG2 Fc variant include V234A, G237A, P238S, V309L and H268A (as designated according to the EU numbering system per Kabat et al. (1991)). In some instances, a human IgG2 Fc variant has up to 12, 11, 10, 9, 8, 7, 6, 5, 4, 3 or fewer mutations in total as compared to wild-type human IgG2 sequence. In some embodiments, one or more additional deletions are included in such IgG2 Fc variant. For example, in some embodiments, the C-terminal lysine of the Fc IgG2 heavy chain constant region is deleted, for example to increase the homogeneity of the polypeptide when the polypeptide is produced in bacterial or mammalian cells. In some instances, a human IgG2 Fc variant has up to 12, 11, 10, 9, 8, 7, 6, 5 or 4 or fewer deletions in total as compared to wild-type human IgG2 sequence.

When the Fc variant is an IgG4 Fc variant, in some embodiments, such Fc variant comprises a S228P mutation (as designated according to Kabat, et al. (1991)), e.g., as represented in SEQ ID NO: 104 in Table 7. In some instances, a human IgG4 Fc variant has up to 12, 11, 10, 9, 8, 7, 6, 5, 4, 3, 2 or 1 mutation(s) in total as compared to wild-type human IgG4 sequence.

In some embodiments, the Fc variant includes at least one of the mutations L234A, L235A, G237A or N297A of an IgG1 Fc region or at least one of the mutations A330S, P331S or N297A of an IgG2 Fc region. In some embodiments, the Fc variant includes at least two of the mutations L234A, L235A, G237A or N297A of an IgG1 Fc region or at least two of the mutations A330S, P331S or N297A of an IgG2 Fc region. In some embodiments, the Fc variant includes at least three of the mutations L234A, L235A, G237A or N297A of an IgG1 Fc region or consists of the mutations A330S, P331S and N297A of an IgG2 Fc region. In some embodiments, the Fc variant consists of the mutations L234A, L235A, G237A and N297A. In some embodiments, the Fc variant includes the mutations L234A, L235A, and G237A of an IgG1 Fc region (IgG1 AAA).

In some embodiments, the Fc variant exhibits reduced binding to an Fc receptor of the subject compared to the wild-type human IgG Fc region. In some embodiments, the Fc variant exhibits ablated binding to an Fc receptor of the subject compared to the wild-type human IgG Fc region. In some embodiments, the Fc variant exhibits a reduction of phagocytosis compared to the wild-type human IgG Fc region. In some embodiments, the Fc variant exhibits ablated phagocytosis compared to the wild-type human IgG Fc region.

Antibody-dependent cell-mediated cytotoxicity, which is also referred to herein as ADCC, refers to a form of cytotoxicity in which secreted Ig bound onto Fc receptors (FcRs) present on certain cytotoxic cells (e.g., Natural Killer (NK) cells and neutrophils) enabling these cytotoxic effector cells to bind specifically to an antigen-bearing target cell and subsequently kill the target cell. Antibody-dependent cell-mediated phagocytosis, which is also referred to herein as ADCP, refers to a form of cytotoxicity in which secreted Ig bound onto Fc receptors (FcRs) present on certain phagocytic cells (e.g., macrophages) enabling these phagocytic effector cells to bind specifically to an antigen-bearing target cell and subsequently engulf and digest the target cell. Ligand-specific high-affinity IgG antibodies directed to the surface of target cells can stimulate the cytotoxic or phagocytic cells and can be used for such killing. In some embodiments, antibodies or conjugates comprising an Fc variant as described herein exhibit reduced ADCC or ADCP as compared to antibodies or conjugates comprising a wild-type Fc region. In some embodiments, antibodies or conjugates comprising an Fc variant as described herein exhibit at least a 5%, 10%, 15%, 20%, 30%, 40%, 50%, 60%, 70%, 80%, 90% or greater reduction in ADCC or ADCP compared to antibodies or conjugates comprising a wild-type Fc region. In some embodiments, antibodies or conjugates comprising an Fc variant as described herein exhibit ablated ADCC or ADCP as compared to antibodies or conjugates comprising a wild-type Fc region.

Complement-directed cytotoxicity, which is also referred to herein as CDC, refers to a form of cytotoxicity in which the complement cascade is activated by the complement component C1q binding to antibody Fc. In some embodiments, antibodies or conjugates comprising an Fc variant as described herein exhibit at least a 5%, 10%, 15%, 20%, 30%, 40%, 50%, 60%, 70%, 80%, 90% or greater reduction in C1q binding compared to antibodies or conjugates comprising a wild-type Fc region. In some cases, antibodies or conjugates comprising an Fc variant as described herein exhibit reduced CDC as compared to antibodies or conjugates comprising a wild-type Fc region. In some embodiments, antibodies or conjugates comprising an Fc variant as described herein exhibit at least a 5%, 10%, 15%, 20%, 30%, 40%, 50%, 60%, 70%, 80%, 90% or greater reduction in CDC compared to antibodies or conjugates comprising a wild-type Fc region. In some cases, antibodies or conjugates comprising an Fc variant as described herein exhibit negligible CDC as compared to antibodies or conjugates comprising a wild-type Fc region.

Fc variants herein include those that exhibit reduced binding to an Fcγ receptor compared to the wild-type human IgG Fc region. For example, in some embodiments, an Fc variant exhibits binding to an Fcγ receptor that is less than the binding exhibited by a wild-type human IgG Fc region to an Fcγ receptor, as described in the Examples. In some instances, an Fc variant has reduced binding to an Fcγ receptor by a factor of 10%, 20% 30%, 40%, 50%, 60%, 70%, 80%, 90%, 95%, 96%, 97%, 98%, 99% or 100% (fully ablated effector function). In some embodiments, the reduced binding is for any one or more Fcγ receptor, e.g., CD16a, CD32a, CD32b, CD32c, or CD64.

In some instances, the Fc variants disclosed herein exhibit a reduction of phagocytosis compared to its wild-type human IgG Fc region. Such Fc variants exhibit a reduction in phagocytosis compared to its wild-type human IgG Fc region, wherein the reduction of phagocytosis activity is e.g., by a factor of 10%, 20%, 30%, 40%, 50%, 60%, 70%, 80%, 90%, 95%, 96%, 97%, 98%, 99% or 100%. In some instances, an Fc variant exhibits ablated phagocytosis compared to its wild-type human IgG Fc region.

In some embodiments, a Q-tag of the present disclosure is attached to the SIRP-α antibody. In some embodiments, an oligonucleotide of the present disclosure is conjugated to a SIRP-α antibody via one or more Q tags. In some embodiments, the Q tag comprises a glutamine residue which is linked to the rest of the conjugate. In still further embodiments of the present aspect, which may be combined with any of the embodiments herein, each Q tag independently comprises or is a peptide sequence selected from the group consisting of SEQ ID NOs: 39-55. In some embodiments, each Q tag independently comprises or is a peptide sequence selected from the group consisting of the peptide sequences of Table 12. In other embodiments of the present aspect, each Q tag independently comprises or is a peptide sequence selected from the group consisting of SEQ ID NOs: 40-55. In yet other embodiments, each Q tag independently comprises or is a peptide sequence selected from the group consisting of SEQ ID NOs: 47-49. In some embodiments, the Q-tag comprises LLQGG (SEQ ID NO:172), GGGLLQGG (SEQ ID NO:173), RPQGF (SEQ ID NO:47), or RPQGFGPP (SEQ ID NO:49). In some embodiments, the Q-tag comprises a peptide sequence RPQGF (SEQ ID NO:47). In certain embodiments, the Q-tag is selected from the group consisting of RPQGF (SEQ ID NO:47), RPQGFPP (SEQ ID NO:48), and RPQGFGPP (SEQ ID NO:49). In some embodiments, the Q-tag comprises a peptide sequence RPQGFGPP (SEQ ID NO:49).

In some embodiments, the Q-tag is attached to the heavy chain of the SIRP-α antibody. In some embodiments, the Q-tag is attached to the heavy chain of the SIRP-α antibody via a linker (e.g., an amino acid or other chemical linker). In some embodiments, the Q-tag is attached to the heavy chain of the SIRP-α antibody (e.g., fused in frame with the heavy chain). In some embodiments, the Q-tag is attached at the C-terminus of the heavy chain of the SIRP-α antibody. In some embodiments, the Q-tag is fused to the C-terminus of the heavy chain of the SIRP-α antibody (e.g., in frame and contiguous with the amino acid sequence of the C-terminus). In some embodiments, the Q-tag is within the Fc domain of the SIRP-α antibody. In some embodiments, the Q tag is naturally occurring, e.g., present in the antibody (such as in the Fc domain/region) without addition of a further peptide sequence onto the antibody. For example, mutation of N297 to N297A exposes Q295 of the antibody, where the conjugation could occur. In certain embodiments wherein the Fc region comprises an N297A substitution, the conjugate further comprises an immunomodulating oligonucleotide P attached to the Q295 residue as shown in the following formula

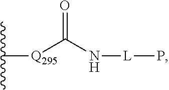

wherein L is a linker moiety connected to Q295 via an amide bond.

In some embodiments, the Q-tag comprises one or more sequences shown in Table 12.

TABLE 12
Q-tag Peptide Sequences

| SEQ ID NO. | Peptide Sequences |
|---|---|
| 39 | LSLSPGLLQGG-OH |
| 40 | WPAQGPT |
| 41 | WPQGPT |
| 42 | WAPQGPT |
| 43 | WAQGPT |
| 44 | TPGQAPW |
| 45 | PNPQLPF |
| 46 | RPQQF |
| 47 | RPQGF |
| 48 | RPQGFPP |
| 49 | RPQGFGPP |
| 50 | RPRPQQF |
| 51 | LSQSKVLG |
| 52 | WGGQLL |
| 53 | WALQRPHYSYPD |
| 54 | WALQRPYTLTES |
| 55 | WALQGPYTLTES |

In some embodiments, the conjugate provided herein is to a target specific cell and tissue in a body for targeted delivery of a conjugated payload oligonucleotide. In certain embodiments, the cell targeted by the conjugate provided herein is myeloid cell. In certain embodiments, the cell targeted by the conjugate provided herein is a T cell. In certain embodiments, the cell targeted by the conjugate provided herein is a neutrophil. In certain embodiments, the cell targeted by the conjugate provided herein is a monocyte. In certain embodiments, the cell targeted by the conjugate provided herein is a macrophage. In certain embodiments, the cell targeted by the conjugate provided herein is a dendritic cell (DC). In certain embodiments, the cell targeted by the conjugate provided herein is a mast cell. In certain embodiments, the cell targeted by the conjugate provided herein is a tumor-associated macrophage (TAM). In certain embodiments, the cell targeted by the conjugate provided herein is a myeloid-derived suppressor cell (MDSC).

In some embodiments, an antibody or conjugate of the present disclosure can be delivered as a naked protein-drug conjugate, or as a protein-drug conjugate formulated with a carrier and delivered, e.g., as encapsulated or as part of a nanocarrier, nanoparticle, liposome, polymer vesicle, or viral envelope. In some embodiments, an antibody or conjugate of the present disclosure can be delivered intracellularly, e.g., by conjugation to a protein-transduction domain or mimic. In some embodiments, an antibody or conjugate of the present disclosure can be delivered by electroporation or microinjection.

In some embodiments, a conjugate of the present disclosure targets more than one population or type of cell, e.g., from those described supra. In some embodiments, a conjugate of the present disclosure targets monocytes and/or DCs. In some embodiments, a conjugate of the present disclosure targets both monocytes, neutrophils, and DCs. In some embodiments, a conjugate of the present disclosure targets monocytes, macrophages, neutrophils, and DCs.

In some embodiments, the SIRP-α antibody or conjugate has one or more effector functions, including without limitation ADCC and/or ADCP. In some embodiments, the SIRP-α antibody or conjugate comprises a human Fc region, e.g., a human IgG Fc region.

In some embodiments, the SIRP-α antibody or conjugate of the present disclosure comprises an antibody constant domain. In some embodiments, the SIRP-α antibody or conjugate of the present disclosure comprises an antibody heavy chain constant domain and/or antibody light chain constant domain listed in Table 13. In some embodiments, the SIRP-α antibody or conjugate of the present disclosure comprises an antibody heavy chain constant domain selected from the group consisting of SEQ ID Nos: 92-107 and 178. In some embodiments, the SIRP-α antibody of the present disclosure comprises an antibody heavy chain constant domain with a Q-tag at the C-terminus of the Fc region, e.g., as shown in SEQ ID No: 95 or 178. In some embodiments, the SIRP-α antibody or conjugate of the present disclosure comprises two antibody heavy chains, each with a constant domain, wherein each of the two antibody heavy chains comprises a Q-tag at the C-terminus of the Fc region, e.g., as shown in SEQ ID No: 95 or 178. In some embodiments, the SIRP-α antibody or conjugate of the present disclosure comprises two antibody heavy chains, each with a constant domain, wherein only one of the two antibody heavy chains comprises a Q-tag at the C-terminus of the Fc region, e.g., as shown in SEQ ID No: 95 or 178. In some embodiments, the SIRP-α antibody or conjugate of the present disclosure comprises an antibody light chain constant domain selected from the group consisting of SEQ ID Nos: 108-110.

TABLE 13
Antibody constant domain sequences

| | SEQ ID | |
|---|---|---|
| Name | NO: | Sequence |
| IgG1 wildtype | 92 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_AAA_N297A | 93 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_AAA | 94 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPG |
| IgG1_AAA + Q-tag | 95 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGAPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREEMTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGRPQGFGPP |
| IgG1_N297A | 96 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_D265A | 97 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVAVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG1_N297A/D265A | 98 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVAVSHEDPEVKFNWYVD |
| | | GVEVHNAKTKPREEQYASTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPG |
| IgG2 | 99 | ASTKGPSVFPLAPCSRSTSESTAALGCLVKDYFPEPVTVSW |
| | | NSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDGV |
| | | EVHNAKTKPREEQFNSTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSDG |
| | | SFFLYSKLTVDKSRWQQGNV |
| | | FSCSVMHEALHNHYTQKSLSLSPG |
| IgG2Da | 100 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG2Da_N297A | 101 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFASTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG2_N297A | 102 | ASTKGPSVFPLAPCSRSTSESTAALGCLVKDYFPEPVTVSW |
| | | NSGALTSGVHTFPAVLQSSG |
| | | LYSLSSVVTVPSSNFGTQTYTCNVDHKPSNTKVDKTVERK |
| | | CCVECPPCPAPPVAGPSVFL |
| | | FPPKPKDTLMISRTPEVTCVVVDVSHEDPEVQFNWYVDGV |
| | | EVHNAKTKPREEQFASTFRV |
| | | VSVLTVVHQDWLNGKEYKCKVSNKGLPAPIEKTISKTKGQ |
| | | PREPQVYTLPPSREEMTKNQ |
| | | VSLTCLVKGFYPSDIAVEWESNGQPENNYKTTPPMLDSDG |
| | | SFFLYSKLTVDKSRWQQGNV |
| | | FSCSVMHEALHNHYTQKSLSLSPG |
| IgG2Da_D265A | 103 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFNSTFRVVSVLTVVHQDWLNGKEYKCKV |
| | | SNKGLPSSIEKTISKTKGQPREPQVYTLPPSREEMTKNQVSL |
| | | TCLVKGFYPSDIAVEWESNGQPENNYKTTPPMLDSDGSFFL |
| | | YSKLTVDKSRWQQGNVFSCSVMHEALHNHYTQKSLSLSP |
| | | G |
| IgG4_S228P | 104 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P_D265A | 105 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVAVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P, | 106 | ASTKGPSVFPLAPCSRSTSESTAALGCLVKDYFPEPVTVSW |
| L235E | | NSGALTSGVHTFPAVLQSSGLYSLSSVVTVPSSSLGTKTYT |
| | | CNVDHKPSNTKVDKRVESKYGPPCPPCPAPEFEGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG4_S228P, | 107 | ASTKGPSVFPLAPCSRSTSESTAALGCLVKDYFPEPVTVSW |
| N297A | | NSGALTSGVHTFPAVLQSSGLYSLSSVVTVPSSSLGTKTYT |
| | | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFASTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LG |
| IgG1_wt + Q-tag | 178 | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRW |
| | | QQGNVFSCSVMHEALHNHYTQKSLSLSPGRPQGFGP |
| | | P |
| Human Kappa | 108 | RTVAAPSVFIFPPSDEQLKSGTASVVCLLNNFYPREAKVQW |
| | | KVDNALQSGNSQESVTEQDSKDSTYSLSSTLTLSKADYEK |
| | | HKVYACEVTHQGLSSPVTKSFNRGEC |
| Human Lambda | 109 | GQPKANPTVTLFPPSSEELQANKATLVCLISDFYPGAVTVA |
| IGLC1 | | WKADGSPVKAGVETTKPSKQSNNKYAASSYLSLTPEQWK |
| | | SHRSYSCQVTHEGSTVEKTVAPTECS |
| Human Lambda | 110 | GQPKAAPSVTLFPPSSEELQANKATLVCLISDFYPGAVTVA |
| IGLC2 | | WKADSSPVKAGVETTTPSKQSNNKYAASSYLSLTPEQWKS |
| | | HRSYSCQVTHEGSTVEKTVAPTECS |

In another aspect of the present disclosure, provided herein is a conjugate comprising a SIRP-α antibody or antigen-binding fragment thereof and one or more immunomodulating oligonucleotides (P), wherein the SIRP-α antibody or antigen-binding fragment is linked to one or more Q-tag peptides (Q) comprising the amino acid sequence RPQGF (SEQ ID NO:47), wherein each immunomodulating oligonucleotide is linked to a Q-tag peptide via an amide bond with the glutamine residue of the Q-tag peptide and a linker (L) as shown in formula (A),

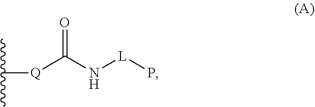

wherein:
   each Q independently comprises a Q-tag peptide sequence RPQGF (SEQ ID NO: 47);
   each L is independently a bond or a linker moiety

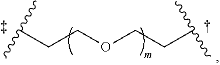

wherein m is an integer ranging from about 0 to about 50, and wherein [symbol] † indicates the point of attachment to P, and [symbol] ‡ indicates the point of attachment to the rest of the conjugate connected to Q via an amide bond with the glutamine residue;
and each P is independently an immunomodulating oligonucleotide having the structure

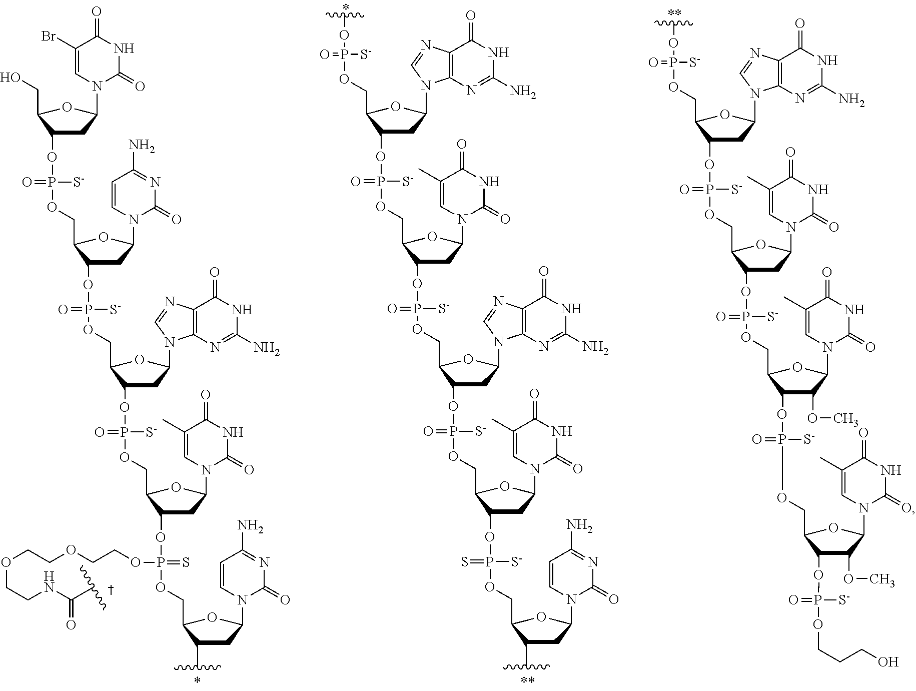

wherein [symbol] * and [symbol] ** indicate the points of attachment within the oligonucleotide, and wherein [symbol] † indicates the point of attachment to L;
wherein Ab comprises a heavy chain variable (VH) domain and a light chain variable (VL) domain, wherein the VH domain comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H3 comprising the amino acid sequence of ETWNHLFDY (SEQ ID NO:58);
wherein the VL domain comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61).

In still other aspects, provided herein is a conjugate comprising a protein, at least one Q tag peptide sequence comprising a glutamine residue, and at least one immunomodulatory oligonucleotide, wherein the Q-tag peptide sequence is naturally occurring or synthetic, and wherein the immunomodulatory oligonucleotide is linked to the Q-tag via an amide bond with the glutamine residue, wherein at least one Q-tag peptide sequence is selected from the group consisting of SEQ ID NOs: 39-55.

In some embodiments, the immunomodulatory oligonucleotide has a sequence selected from the group consisting of the oligonucleotides of Table 15 and Table 17.

In one aspect, provided herein is a SIRP-α antibody comprising at least one Q tag peptide sequence comprising a glutamine residue. In some embodiments, the Q tag peptide sequence is naturally occurring or synthetic. In certain embodiments, the Q tag peptide sequence is an internal reactive glutamine exposed by an amino acid substitution. In further embodiments, the Q tag is fused to the C-terminus of the heavy chain of the protein. In still further embodiments, at least one of the at least one Q tag peptide sequences is elected from the group consisting of SEQ ID NOs: 39-55.

In another aspect of the present disclosure, provided herein are SIRP-α antibodies of formula (B)

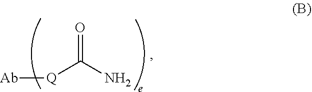

wherein:
   each Q is independently a Q-tag comprising a peptide sequence with at least one glutamine residue;
   Ab is the SIRP-α antibody or antigen-binding fragment thereof; and
   e is an integer from 1 to 20.

The SIRP-α antibodies of formula (B) may be precursors to the antibody-oligonucleotide conjugates of formula (A) as described above. Accordingly, the properties and embodiments of the antibodies as described in the previous aspect of formula (A) may be the same or different from the properties and/or embodiments of the antibodies of formula (B).

In some embodiments of the present aspect, the SIRP-α antibody or fragment thereof is a monoclonal antibody or fragment thereof. In certain embodiments, the SIRP-α antibody or fragment thereof is a Fab, F(ab′)2, Fab′-SH, Fv, scFv, single domain, single heavy chain, or single light chain antibody or antibody fragment. In other embodiments, the SIRP-α antibody or fragment thereof is a humanized, human, or chimeric antibody or fragment thereof.

In some embodiments, the SIRP-α antibody comprises an Fc region. In certain embodiments wherein the SIRP-α antibody comprises an Fc region, the Fc region is a human Fc region selected from the group consisting of an IgG1 Fc region, an IgG2 Fc region, and an IgG4 Fc region.

In certain embodiments of the present aspect, the Fc region is:
(a) a human IgG1 Fc region comprising L234A, L235A, and/or G237A substitutions, amino acid position numbering according to EU index;
(b) a human IgG2 Fc region comprising A330S and/or P331S substitutions, amino acid position numbering according to EU index; or
(c) a human IgG4 Fc region comprising S228P and/or L235E substitutions, amino acid position numbering according to EU index.

In some embodiments, the Fc region further comprises an N297A substitution, amino acid position numbering according to EU index. In other embodiments, the Fc region further comprises a D265A substitution, amino acid position numbering according to EU index. In yet further embodiments, the SIRP-α antibody comprises a human lambda light chain. In other embodiments, the SIRP-α antibody comprises a human kappa light chain.

In some embodiments, at least one Q-tag is attached to the heavy chain of the SIRP-α antibody. In certain embodiments, at least one Q-tag is fused to the C-terminus of the heavy chain of the SIRP-α antibody. In other embodiments, at least one Q-tag is attached to the light chain of the SIRP-α antibody. In still further embodiments, at least one Q-tag is within the Fc domain.

In some embodiments of the present aspect, the SIRP-α antibody is linked to from 1 to 20 Q-tags Q. In certain embodiments, the number of Q-tags linked to the SIRP-α antibody conjugate is an integer of about 1, about 2, about 3, about 4, about 5, about 6, about 7 about 8, about 9, about 10, about 11 about 12, about 13, about 14, about 15, about 16, about 17, about 18, about 19, or about 20. In certain other embodiments, 1 or 2 Q-tags is/are linked to the SIRP-α antibody or antigen-binding fragment. In yet other embodiments, the number of Q-tags linked to the SIRP-α antibody conjugate is an integer from 1 to 10, from 10 to 20, from 5 to 10, from 10 to 15, from 15 to 20, or from 1 to 5.

In still further embodiments of the present aspect, which may be combined with any of the preceding embodiments, each Q tag independently comprises or is a peptide sequence selected from the group consisting of SEQ ID NOs: 39-55. In some embodiments, each Q tag independently comprises or is a peptide sequence selected from the group consisting of the peptide sequences of Table 12. In other embodiments of the present aspect, each Q tag independently comprises or is a peptide sequence selected from the group consisting of SEQ ID NOs: 40-55. In yet other embodiments, each Q tag independently comprises or is a peptide sequence selected from the group consisting of SEQ ID NOs: 47-49. In some embodiments, the Q-tag comprises LLQGG (SEQ ID NO:172), GGGLLQGG (SEQ ID NO:173), RPQGF (SEQ ID NO: 47), or RPQGFGPP (SEQ ID NO:49). In some embodiments of the present aspect, each Q is independently a Q-tag comprising a peptide sequence RPQGF (SEQ ID NO:47). In certain embodiments, each Q-tag comprising a peptide sequence RPQGF (SEQ ID NO:47) is selected from the group consisting of RPQGF (SEQ ID NO:47), RPQGFPP (SEQ ID NO:48), and RPQGFGPP (SEQ ID NO:49). In certain embodiments, each Q tag independently comprises or is a peptide sequence RPQGFGPP (SEQ ID NO:49). In some embodiments, the antibody or conjugate comprises a heavy chain comprising the amino acid sequence of SEQ ID NO:87, 88, or 89 and a Q tag of the present disclosure.

### IV. Immunomodulating Oligonucleotides

In yet another aspect, provided herein is an immunomodulating oligonucleotide of formula (C),

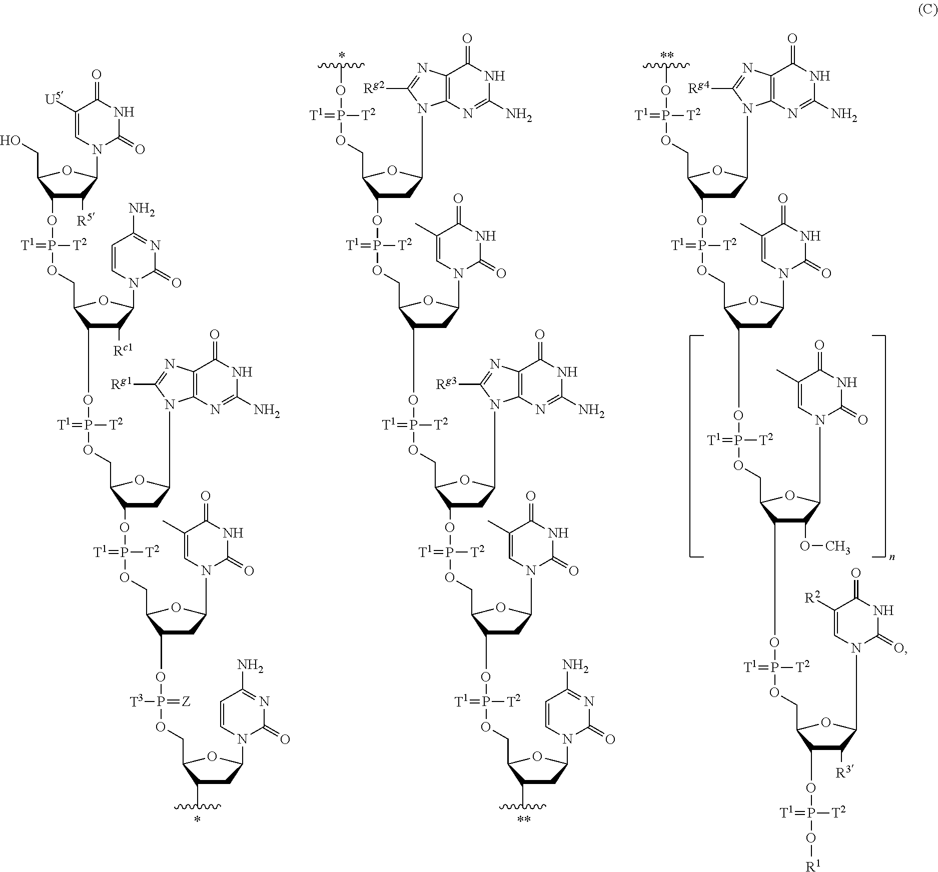

wherein
   [symbol] * and [symbol] ** indicate the points of attachment within the oligonucleotide;
   each T¹ is independently O or S;
   each T² is S⁻;
   T³ is a group

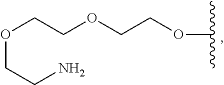 [SMILES: *OCCOCCOCCN]

wherein [symbol] indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo, and wherein if one R^{g1}, R^{g2}, R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In some embodiments of the present aspect, U^{5′} is —H In other embodiments, U^{5′} is halogen. In certain embodiments, U^{5′} is iodo or bromo. In some embodiments of the present aspect, the immunomodulatory oligonucleotide of formula (C) is an immunomodulatory oligonucleotide of formula (C′). In other embodiments of the present aspect, the immunomodulatory oligonucleotide of formula (C) is an immunomodulatory oligonucleotide of formula (C″).

In some embodiments of the present aspect, provided herein is an immunomodulatory oligonucleotide of formula (C′)

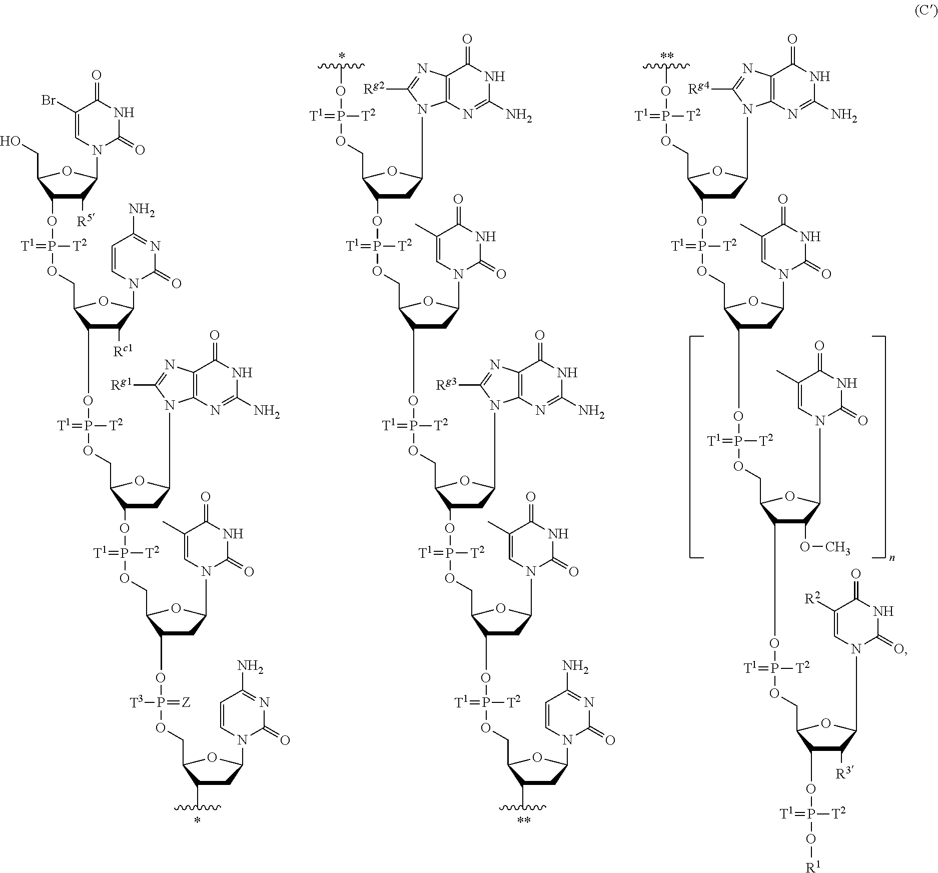

wherein
   [symbol] * and [symbol] ** indicate the points of attachment within the oligonucleotide;
   each T¹ is independently O or S;
   each T′ is S⁻;
   T³ is a group

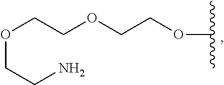 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo and wherein if one R^{g1}, R^{g2}, R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In other embodiments of the present aspect, provided herein is an immunomodulatory oligonucleotide of formula (C″)

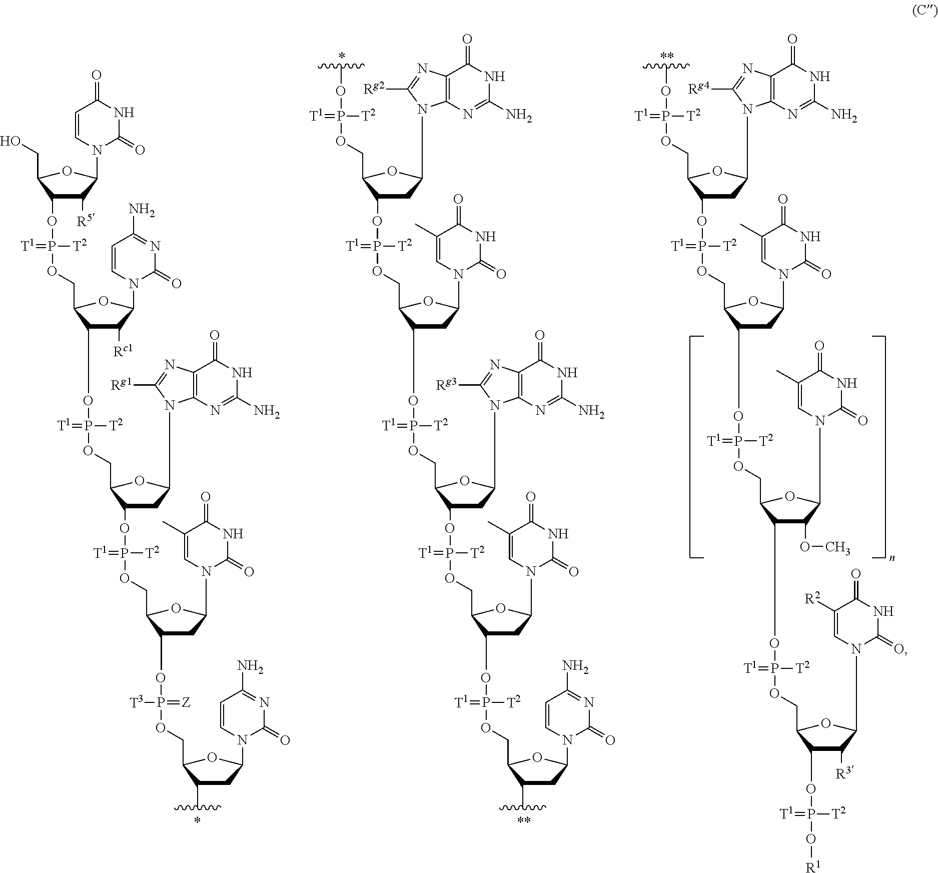

wherein
   [symbol] * and [symbol] ** indicate the points of attachment within the oligonucleotide;
   each T¹ is independently O or S;
   each T² is S⁻;
   T³ is a group

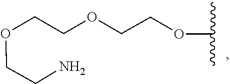 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo and wherein if one R^{g1}, R^{g2}, R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In some embodiments of the present aspect, provided herein is an immunomodulatory oligonucleotide of formula (C′)

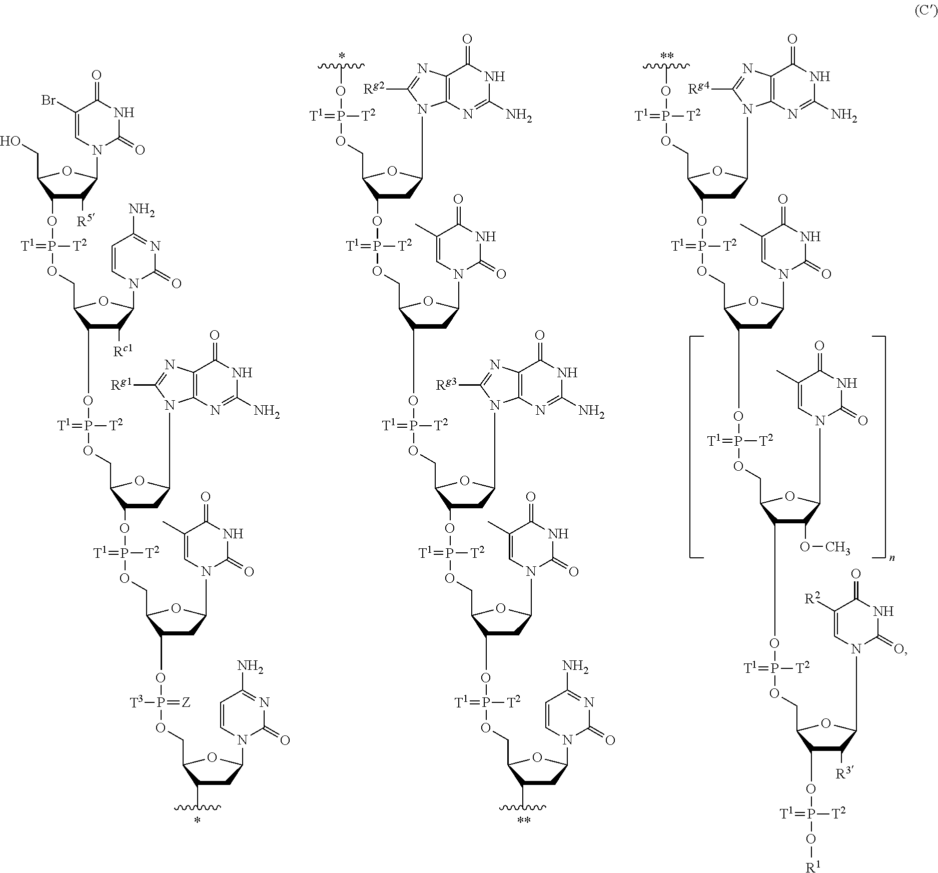

wherein:
   [symbol] * and [symbol] ** indicate the points of attachment within the oligonucleotide;
   each T¹ is independently O or S;
   each T is S⁻;
   T³ is a group

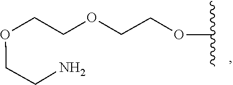 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In other embodiments of the present aspect, provided herein is an immunomodulatory oligonucleotide of formula (C″)

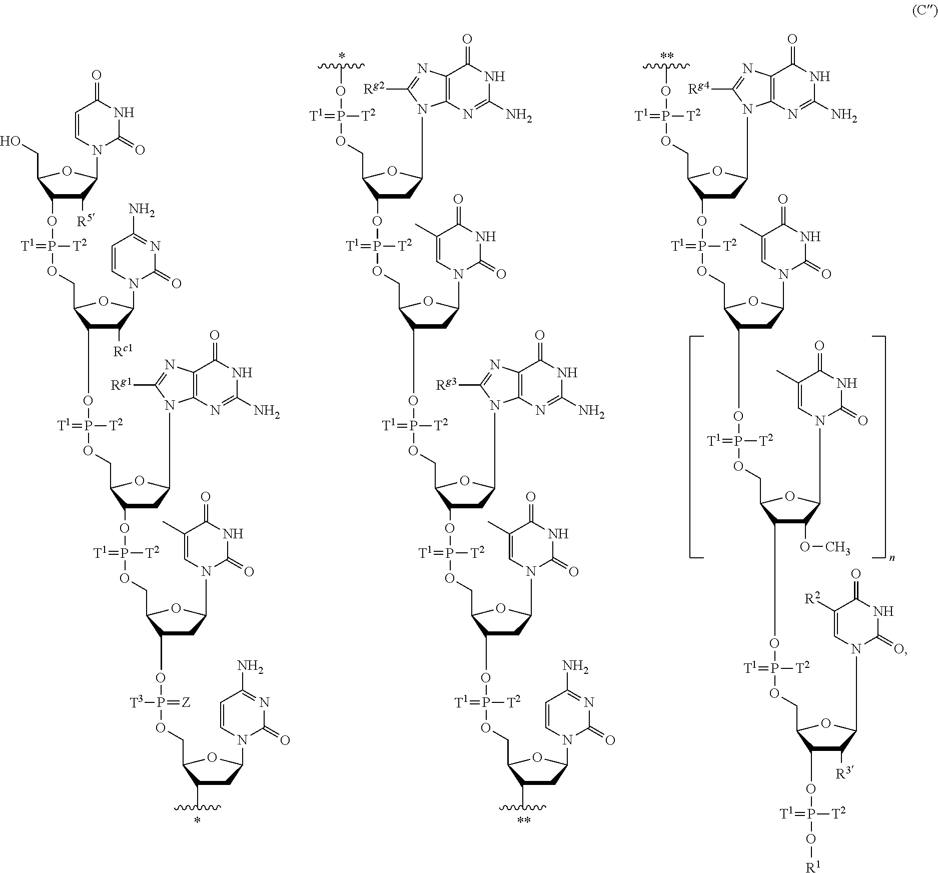

wherein:
   [symbol] * and [symbol] ** indicate the points of attachment within the oligonucleotide;
   each T¹ is independently O or S;
   each T² is S⁻;
   T³ is a group

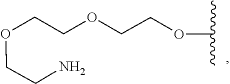 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻. The pair(s) of geminal T¹ and T² wherein T¹ is S and T² is S may also be described as phosphorodithioate linkages.

It should be recognized that in some instances wherein the oligonucleotide has at least one pair of geminal T¹ and T² wherein T¹ is S and T² is S⁻, the phosphorodithioate linkage(s) may be further described in terms of the position within the oligonucleotide at which the linkage is located. The position of the linkage may be characterized, for example, as being between two nucleoside residues, e.g., between the first and second nucleoside residues (or between nucleoside residues 1 and 2) as counted from the 5′ end of the oligonucleotide. Alternatively, the position of the linkage may be described as being located at the 3′-position of a given nucleoside residue, e.g., on the internucleoside linker immediately following the specified nucleoside residue or the 3′-position of the ′3-terminal residue.

In some embodiments wherein the oligonucleotide comprises at least one pair of geminal T¹ and T² wherein T¹ is S and T² is S⁻, and wherein n is 0, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, or between nucleoside residues 11 and 12. In some embodiments wherein the oligonucleotide comprises at least one pair of geminal T¹ and T² wherein T¹ is S and T² is S⁻, and wherein n is 0, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, or nucleoside residue 13.

In some embodiments wherein the oligonucleotide comprises at least one pair of geminal T¹ and T² wherein T¹ is S and T² is S⁻, and wherein n is 1, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, or between nucleoside residues 12 and 13. In some embodiments wherein the oligonucleotide comprises at least one pair of geminal T¹ and T² wherein T¹ is S and T² is S⁻, and wherein n is 0, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, or nucleoside residue 14.

In some embodiments wherein the oligonucleotide comprises at least one pair of geminal T¹ and T² wherein T¹ is S and T² is S⁻, and wherein n is 1, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, or between nucleoside residues 12 and 13. In some embodiments wherein the oligonucleotide comprises at least one pair of geminal T¹ and T² wherein T¹ is S and T² is S⁻, and wherein n is 1, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, or nucleoside residue 14.

In some embodiments wherein the oligonucleotide comprises at least one pair of geminal T¹ and T² wherein T¹ is S and T² is S⁻, and wherein n is 2, the at least one phosphorodithioate linkage is between nucleoside residues 1 and 2, between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, between nucleoside residues 10 and 11, between nucleoside residues 11 and 12, between nucleoside residues 12 and 13, or between residues 13 and 14. In some embodiments wherein the oligonucleotide comprises at least one pair of geminal T¹ and T² wherein T¹ is S and T² is S⁻, and wherein n is 2, the at least one phosphorodithioate linkage is located at the 3′-position of nucleoside residue 1, nucleoside residue 2, nucleoside residue 3, nucleoside residue 5, nucleoside residue 6, nucleoside residue 7, nucleoside residue 8, nucleoside residue 9, nucleoside residue 10, nucleoside residue 11, nucleoside residue 12, nucleoside residue 13, nucleoside residue 14, or residue 15.

In still other embodiments wherein the oligonucleotide has at least two phosphorodithioate linkages or comprises at least two pairs of geminal T¹ and T² wherein T¹ is S and T² is S⁻, the positions of one or both phosphorodithioate linkages or pairs of T¹ and T² may be specified. It should be recognized that the positions of one or both phosphorodithioate linkages may be independently varied.

In some embodiments of the present aspect, provided herein is an immunomodulatory oligonucleotide of formula (C′)

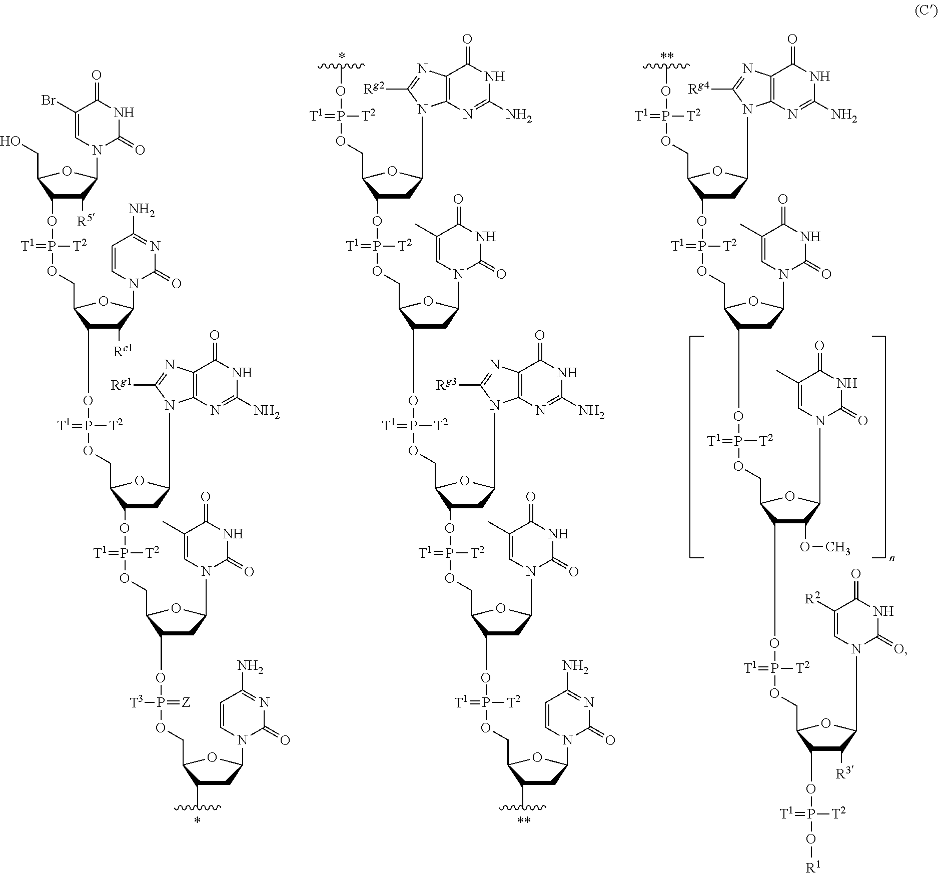

wherein:
   [symbol] * and [symbol] ** indicate the points of attachment within the oligonucleotide;
   each T¹ is independently O or S;
   each T² is S⁻;
   T³ is a group

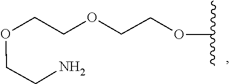 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In other embodiments of the present aspect, provided herein is an immunomodulatory oligonucleotide of formula (C″)

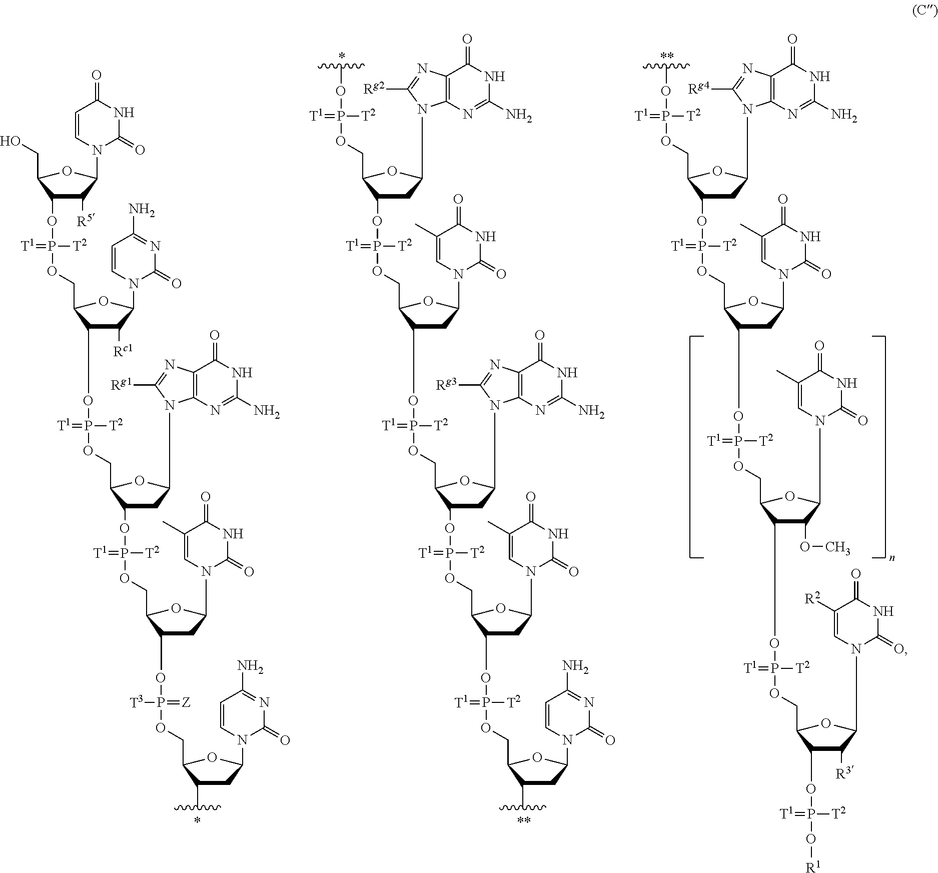

wherein:
   [symbol] * and [symbol] ** indicate the points of attachment within the oligonucleotide;
   each T¹ is independently O or S;
   each T² is S⁻;
   T³ is a group

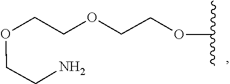 [SMILES: *OCCOCCOCCN]

wherein [symbol] indicates the point of attachment to the rest of the oligonucleotide;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻.

In still yet another embodiment of the present aspect, provided herein is an oligonucleotide of formula (C)

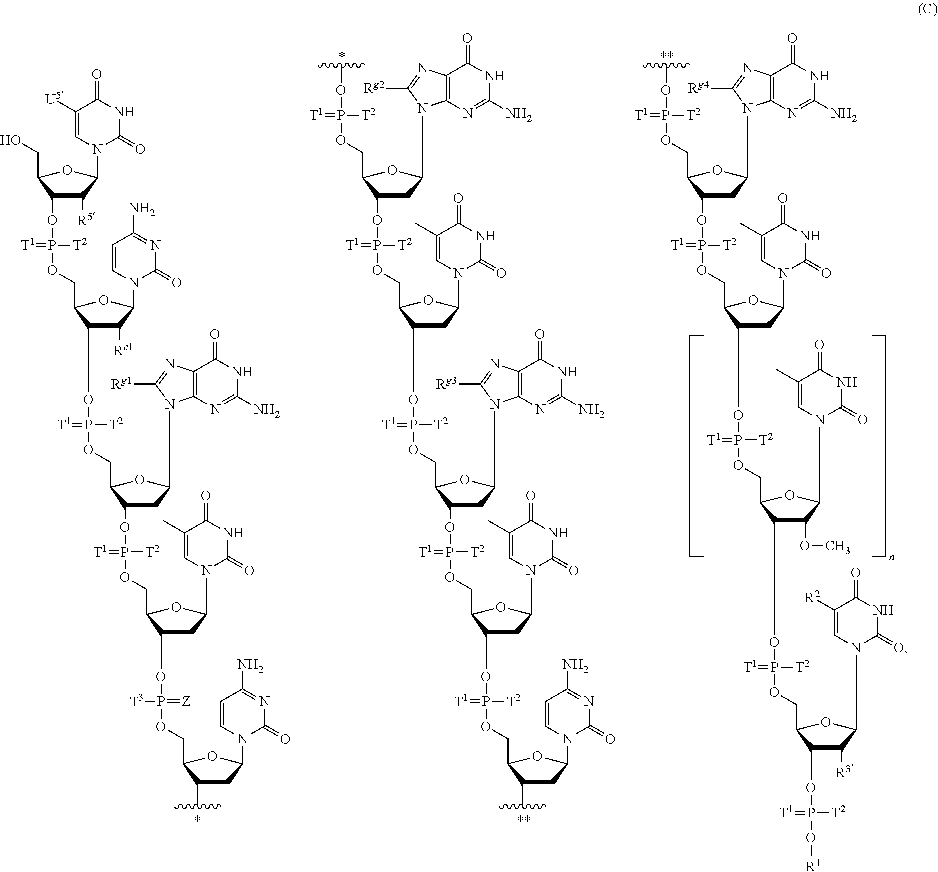

wherein [symbol] * and [symbol] ** indicate the points of attachment within the oligonucleotide;
each T¹ is independently O or S;
each T² is S⁻;
provided that the oligonucleotide comprises at least one pair of geminal T¹ and T² wherein T¹ is S and T² is S,
T³ is a group

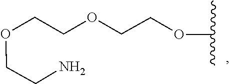 [SMILES: *OCCOCCOCCN]

wherein [symbol] indicates the point of attachment to the rest of the oligonucleotide;
Z is O or S;
U^{5′} is —H or halogen;
R^{5′} is —H;
R^{c1} is —H;
R^{g1}, R^{g2}, R^{g3}, and R^{g4} are H;
R^{3′} is methoxy;
R¹ is —(CH₂)₃—OH;
R² is -methyl; and
n is 1,
or a pharmaceutically acceptable salt thereof.

In some embodiments of any of the foregoing, the at least one pair of geminal T¹ and T² wherein T¹ is S and T² is S is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In still other embodiments of the foregoing, the oligonucleotide comprises at least two pairs of of geminal T¹ and T² wherein T¹ is S and T² is S, and wherein the at least two pairs of of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, the oligonucleotide comprises one or two pairs of geminal T¹ and T² wherein T¹ is S and T² is S, and wherein the one or two pairs of geminal T¹ and T² are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain embodiments, the oligonucleotide comprises one pair of geminal T¹ and T² wherein T¹ is S and T² is S, and wherein the pair of geminal T¹ and T² is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain other embodiments, the oligonucleotide comprises two pairs of geminal T¹ and T² wherein T¹ is S and T² is S, and wherein the two pairs of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, R⁵′ is H. In other embodiments, R⁵′ is methoxy. In some embodiments, R^{c1} is H. In yet other embodiments, R^{c1} is methoxy. In still further embodiments, R² is methyl. In still other embodiments, R² is H. In yet other additional embodiments, which may be combined with any of the preceding embodiments, T³ is

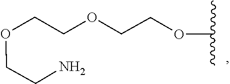 [SMILES: *OCCOCCOCCN]

In still other embodiments, T³ is

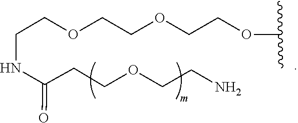

In certain embodiments, m is an integer from 20 to 25.

In another aspect, the immunomodulating oligonucleotide of formula (C) is an oligonucleotide selected from the group consisting of the oligonucleotides of Table 14 and Table 15, or a pharmaceutically acceptable salt thereof. In still other embodiments, the immunomodulating oligonucleotide of formula (C) is an oligonucleotide selected from the group consisting of the oligonucleotides of Table 15, or a pharmaceutically acceptable salt thereof.

TABLE 14
Modified Oligonucleotide Structures (with PEG₃NH₂)

| Cmpd | |
|---|---|
| # | Structure |
| 1.1a | 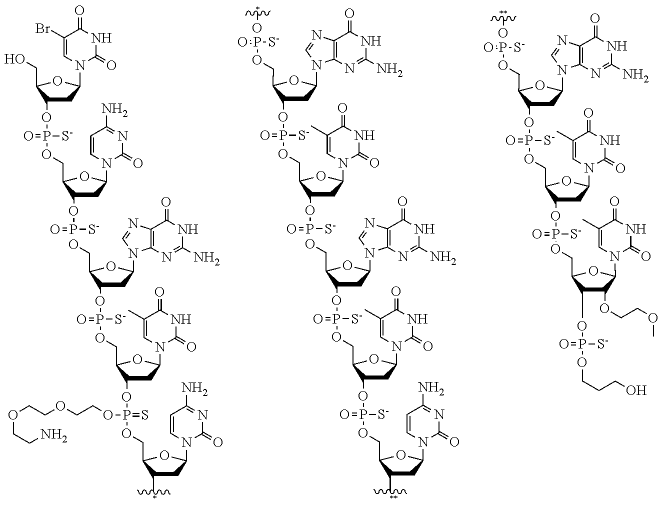 |
| 2.1a | 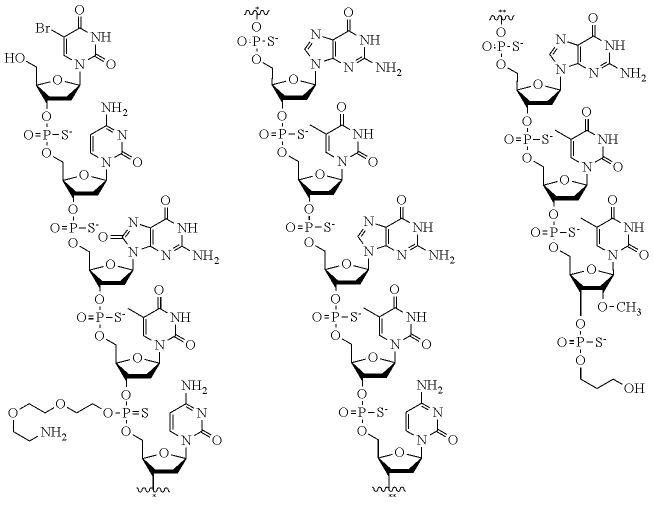 |
| 2.2a | 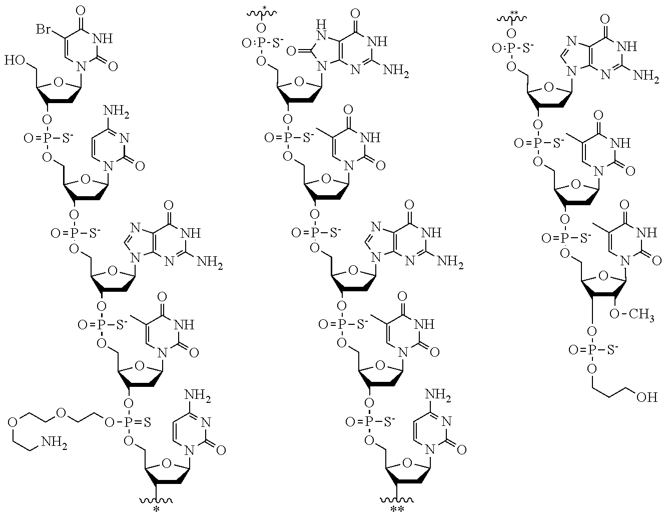 |
| 2.3a | 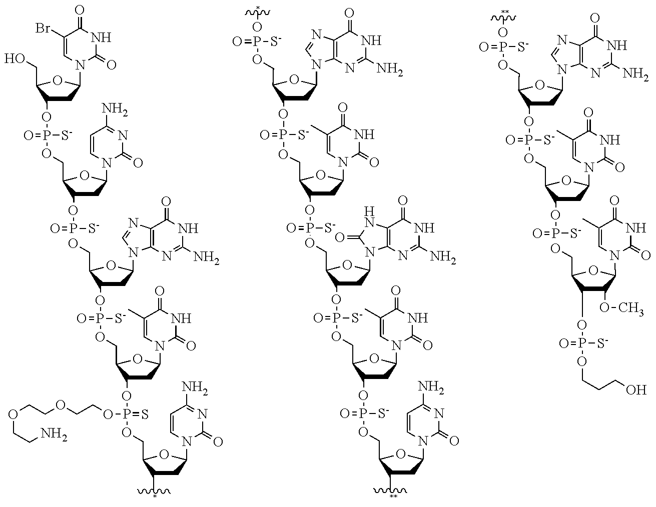 |
| 2.4a | 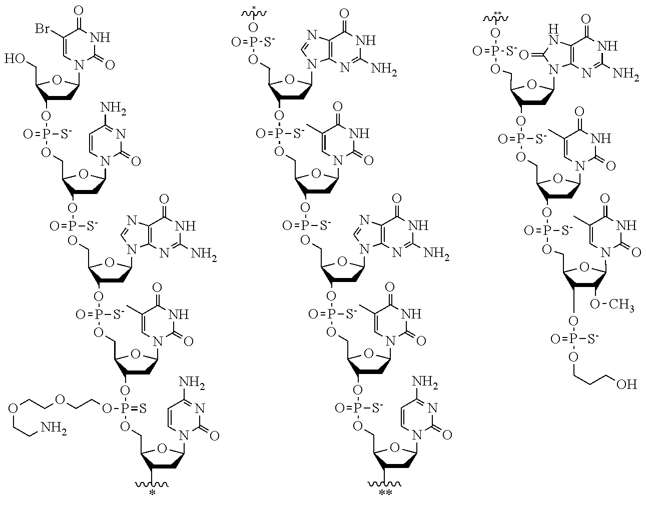 |

TABLE 15
Modified Oligonucleotide Structures (with -PEG₃NH₂)

| Cmpd | |
|---|---|
| # | Structure |
| 3.1a | 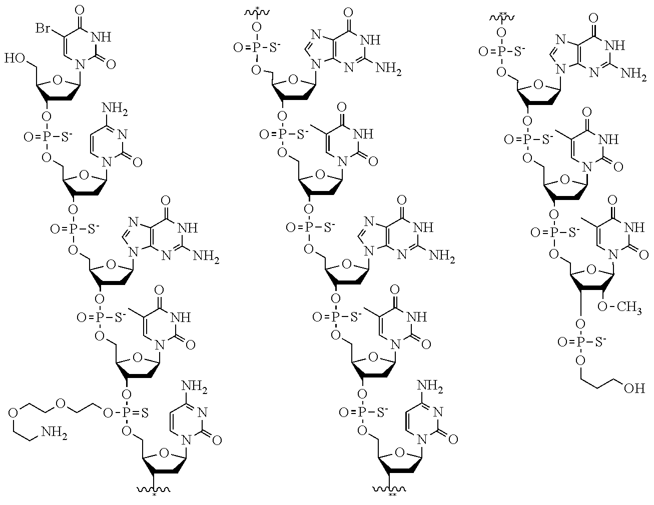 |
| 3.2a | 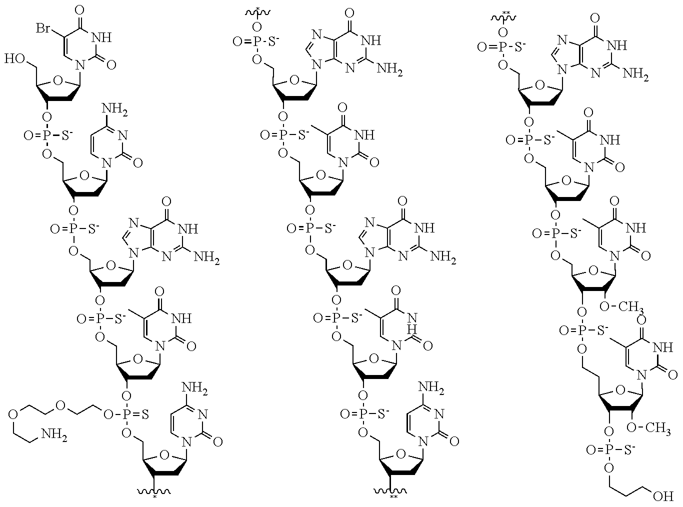 |
| 3.3a | 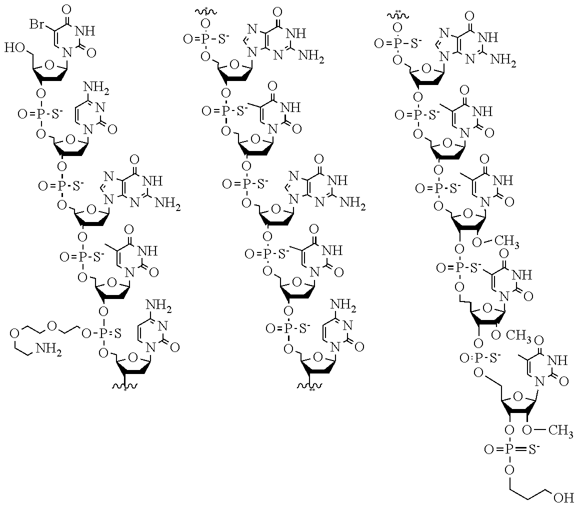 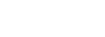 |
| 4.1a | 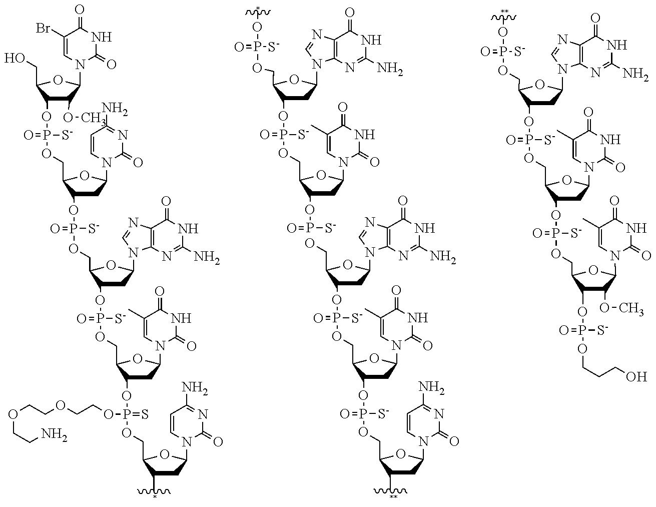 |
| 4.2a | 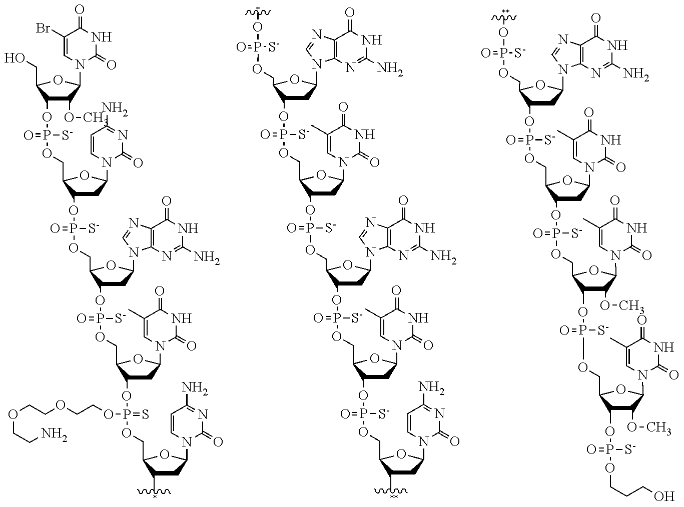 |
| 4.3a | 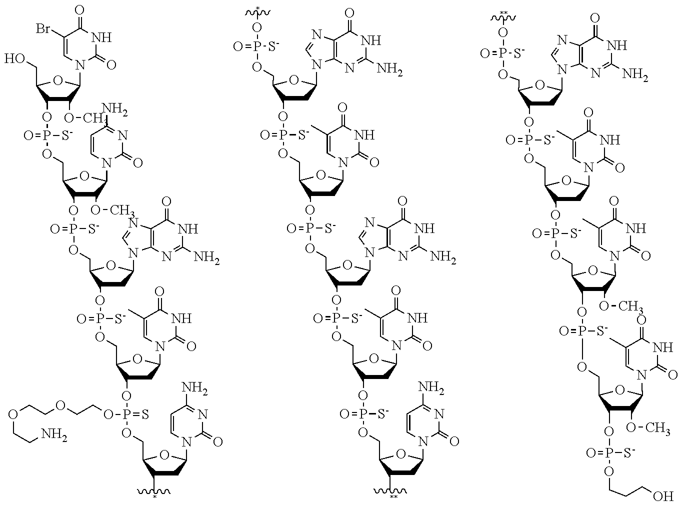 |
| 5.1a | 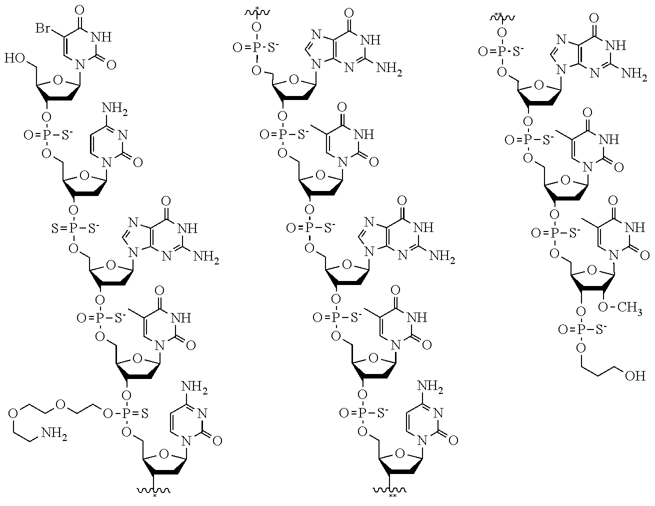 |
| 5.2a | 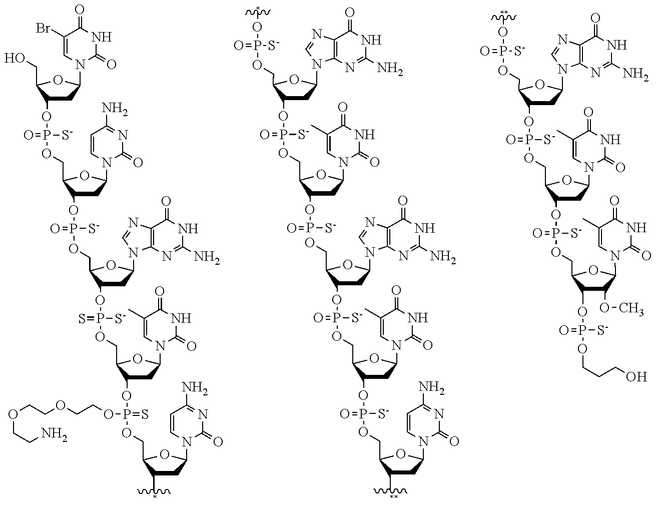 |
| 5.3a | 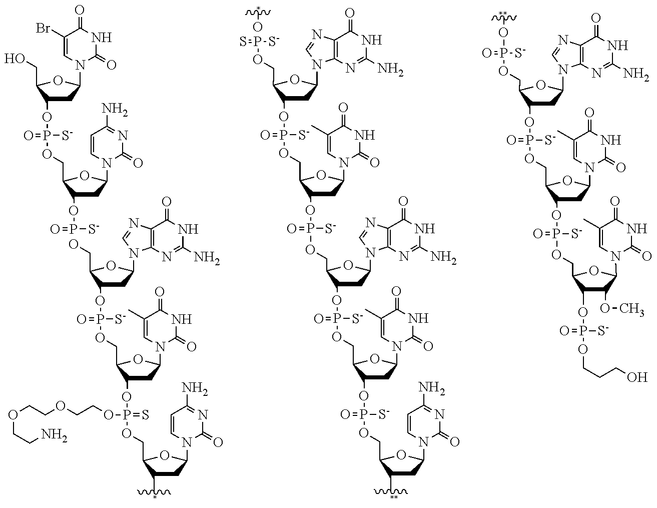 |
| 5.4a | 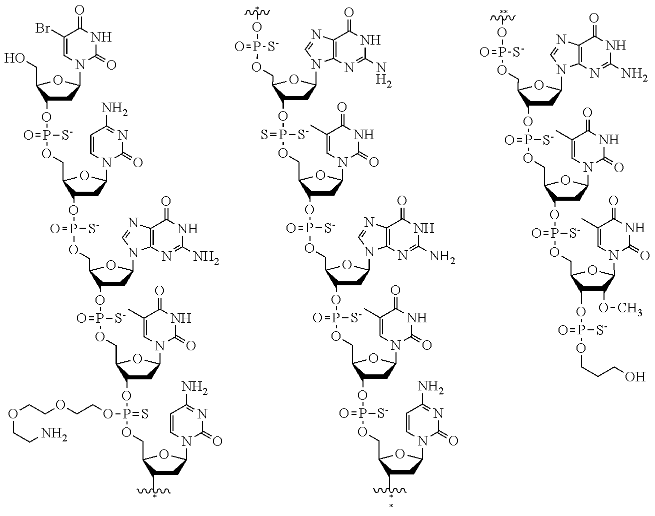 |
| 5.5a | 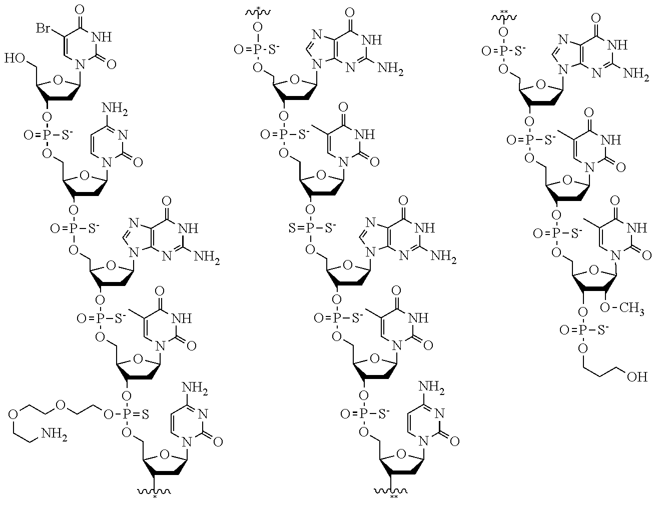 |
| 5.6a | 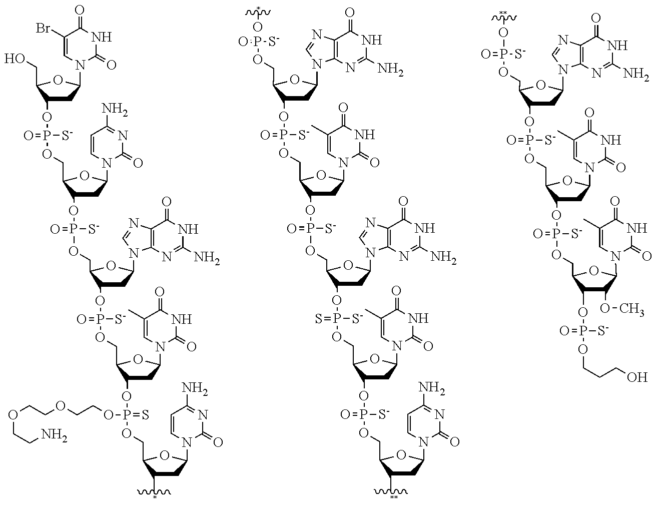 |
| 5.7a | 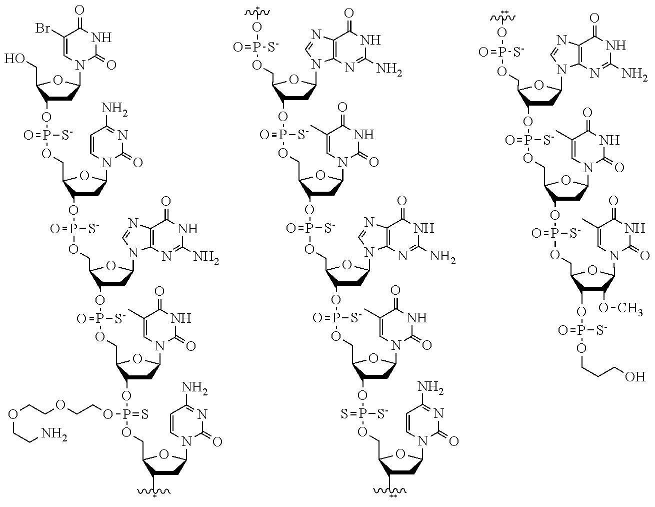 |
| 5.8a | 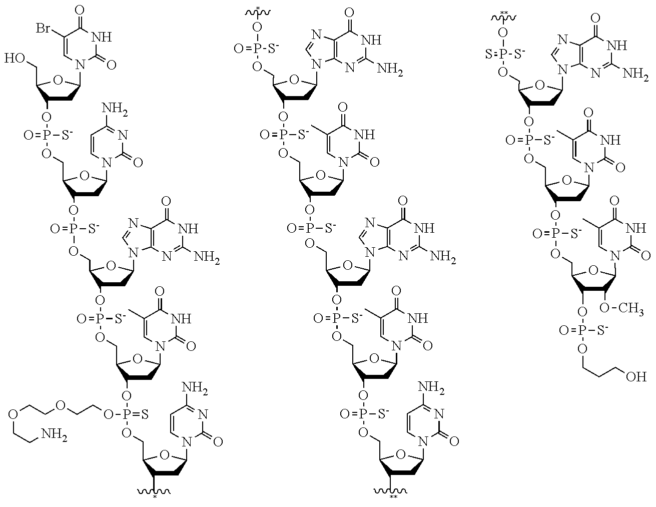 |
| 5.9a | 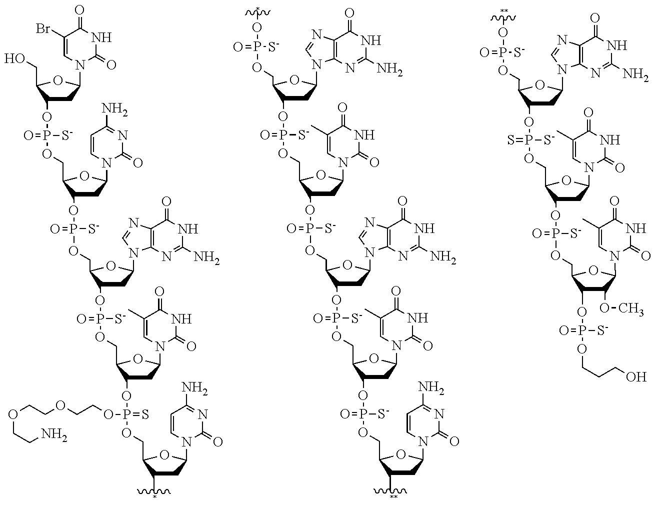 |
| 5.10a | 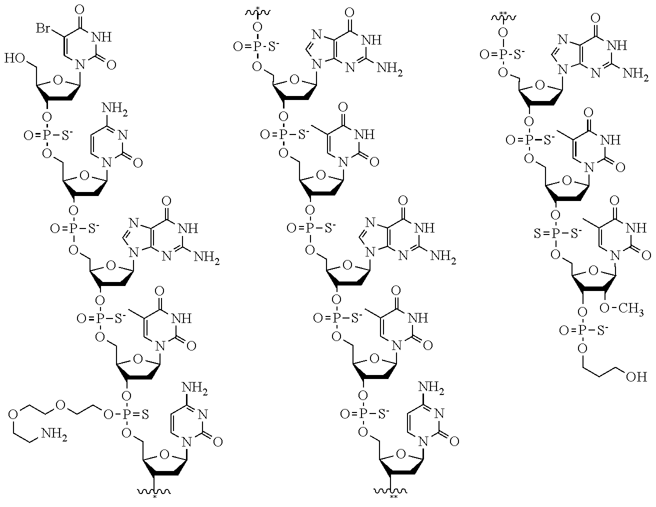 |
| 5.11a | 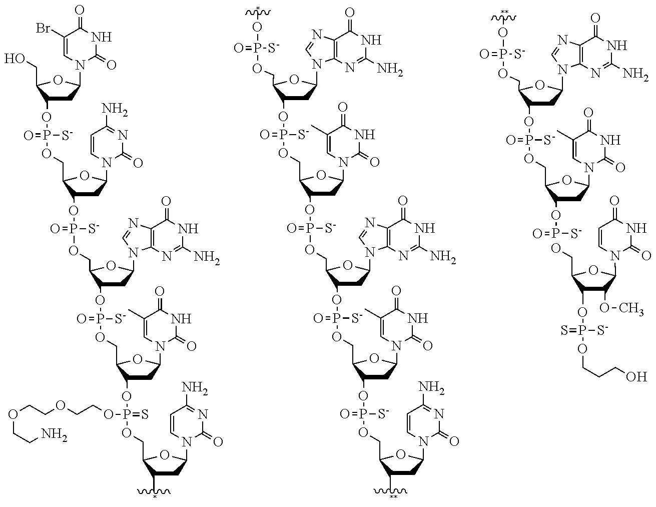 |
| 5.12 | 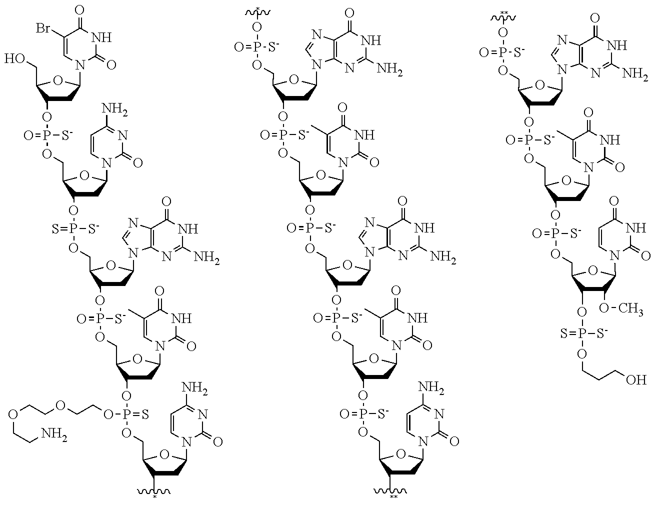 |
| 6.1a | 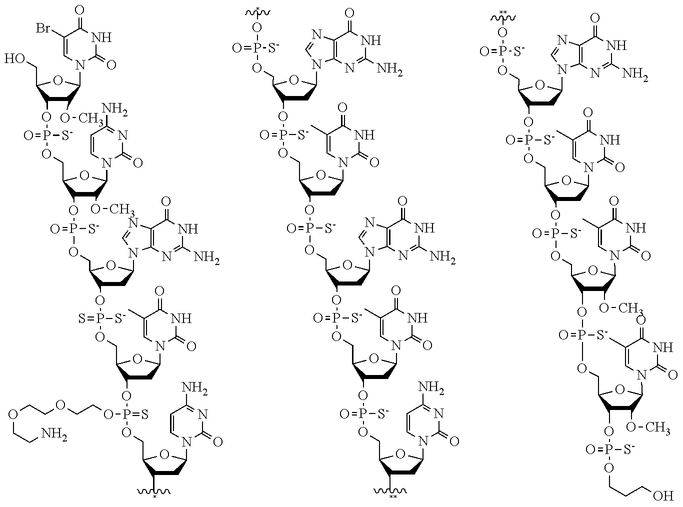 |
| 6.2a | 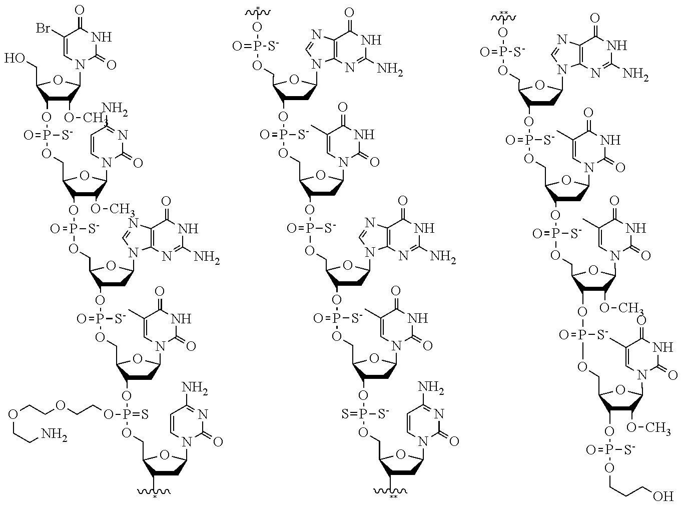 |
| 6.3a | 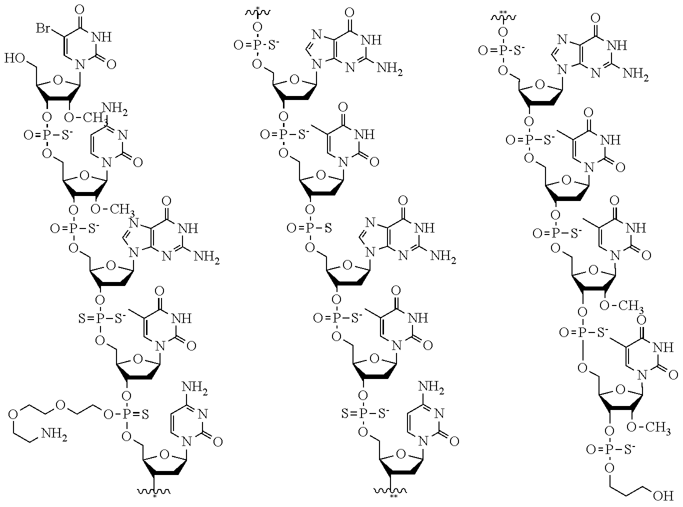 |
| 7.1a | 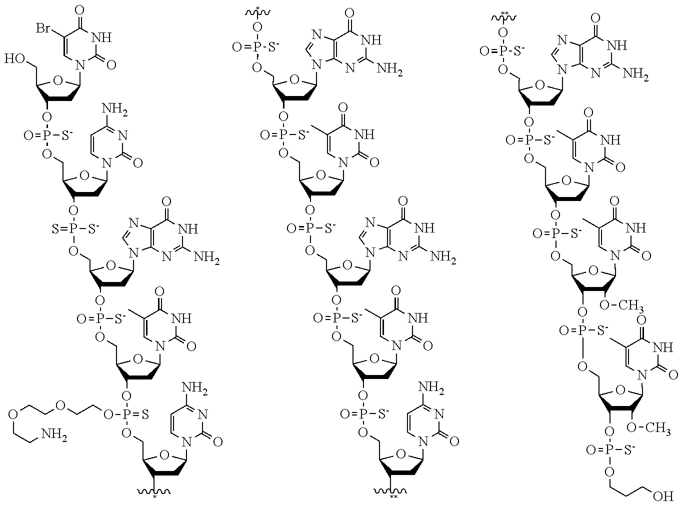 |
| 7.2a | 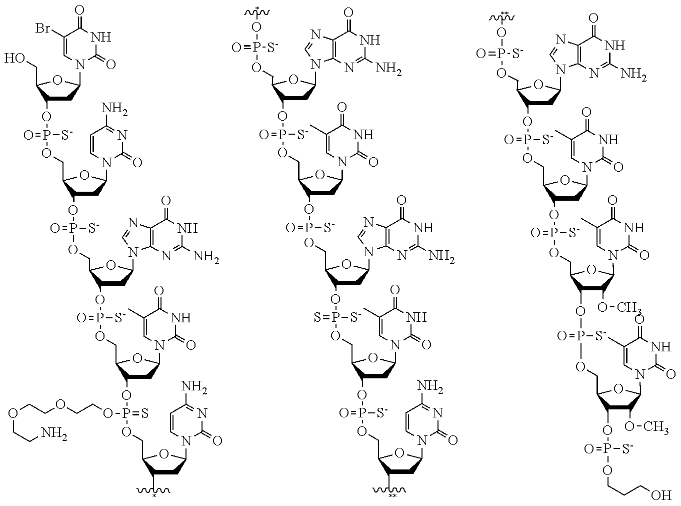 |
| 7.3a | 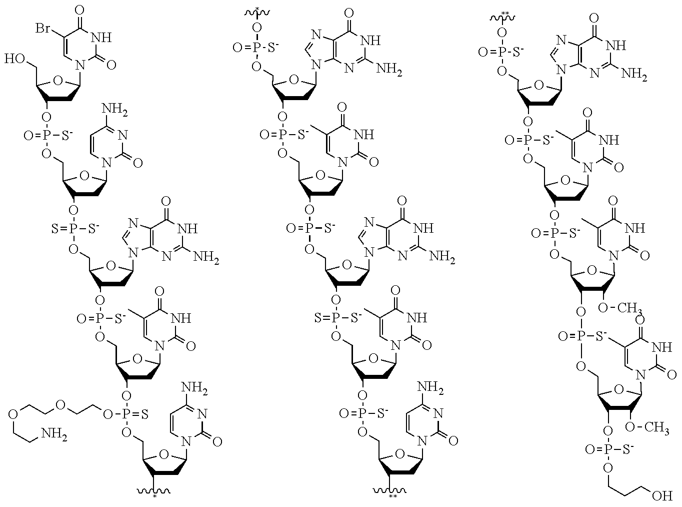 |
| 7.4a | 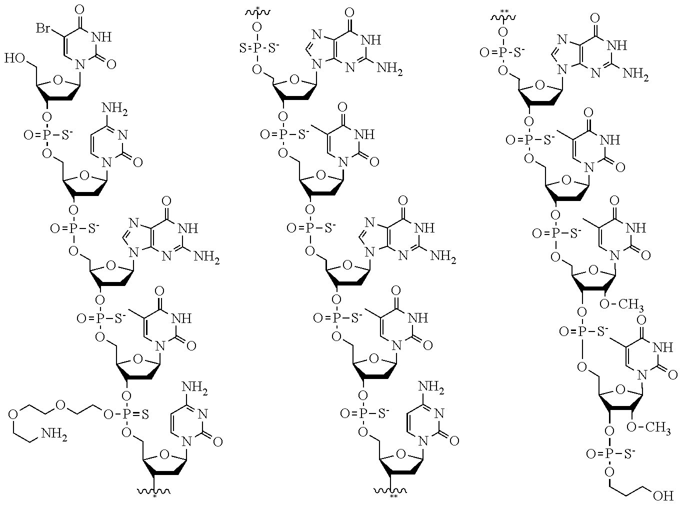 |
| 7.5a | 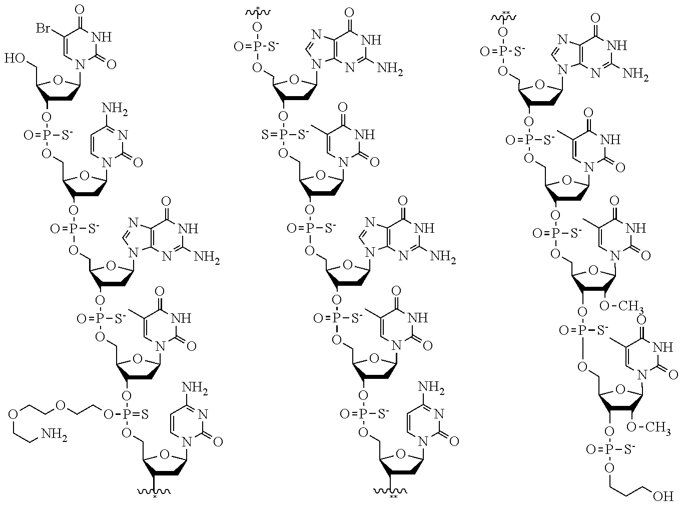 |
| 7.6a | 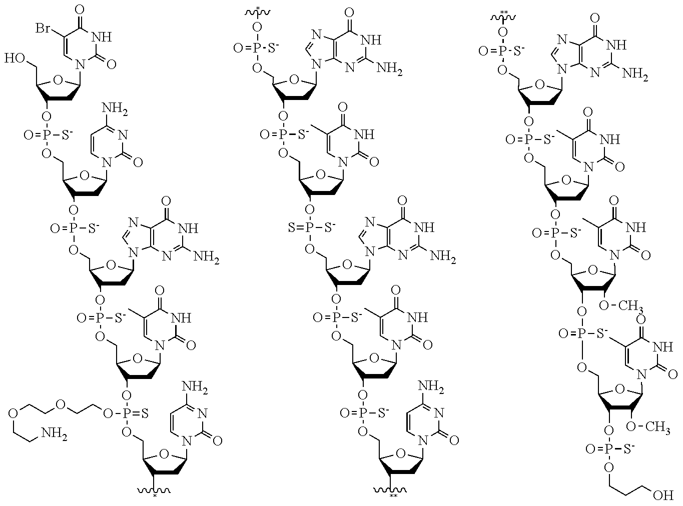 |
| 7.7a | 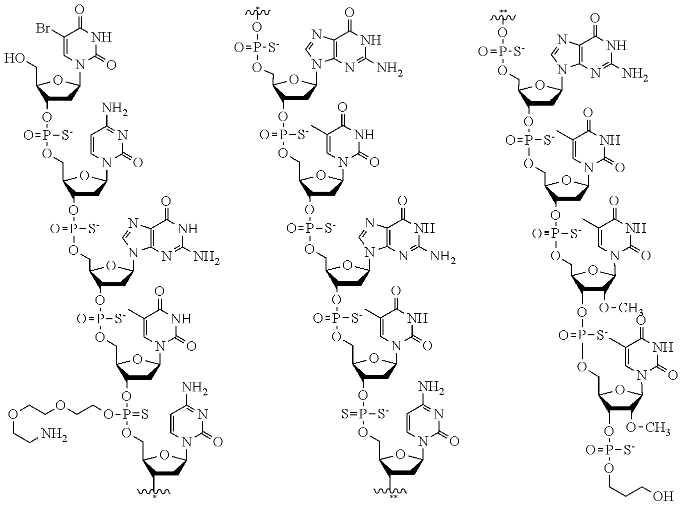 |
| 7.8a | 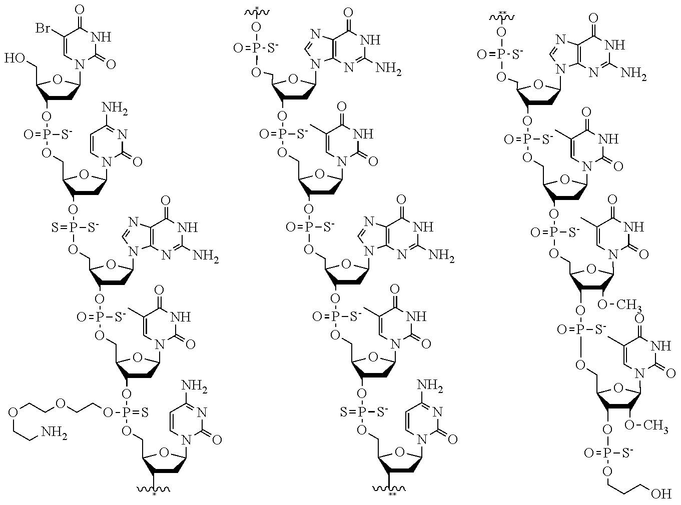 |
| 7.9a | 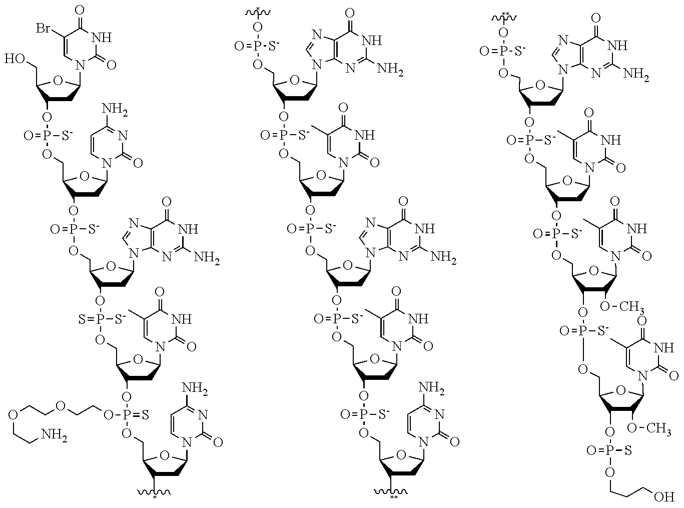 |
| 7.10a | 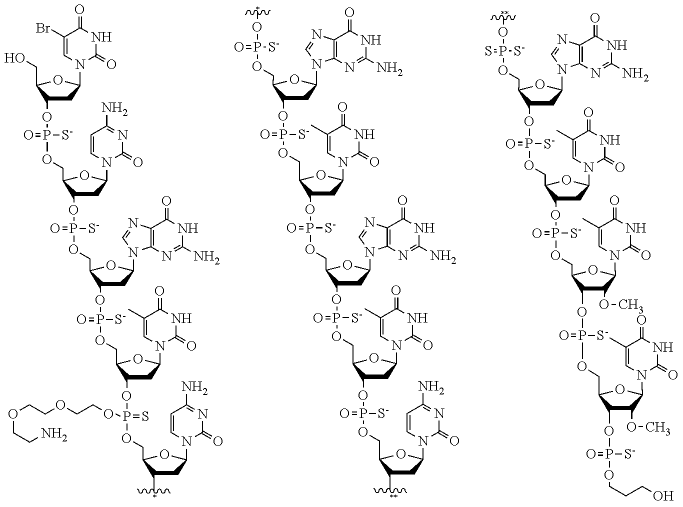 |
| 15.7a | 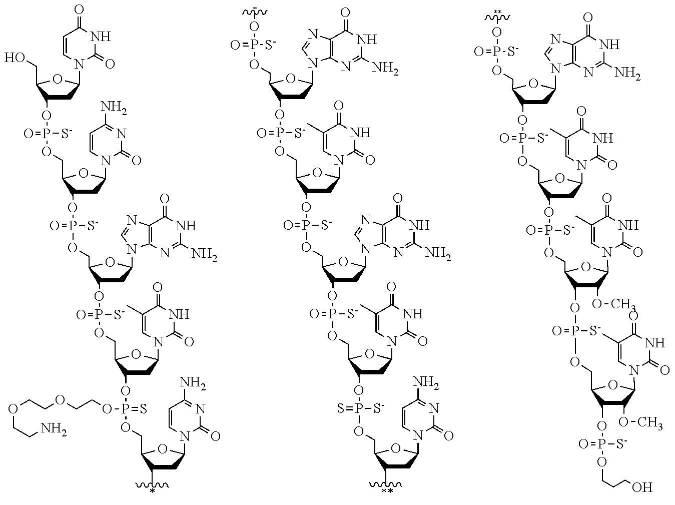 |

In some embodiments, the immunomodulating oligonucleotides of formula (C) may be used as precursors to prepare conjugates comprising a SIRP-α antibody or antigen-binding fragment thereof and one or more immunomodulating oligonucleotides of formula (C) linked via Q-tag as shown in the structures of formula (A) as described herein.

In some embodiments, the immunmodulating oligonucleotides of formula (C) may be modified to attach a linker moiety L to the terminal group T³ in formula (C) to provide immunomodulating oligonucleotides of formula (D). In still another aspect, provided herein are immunomodulating oligonucleotides of formula (D)

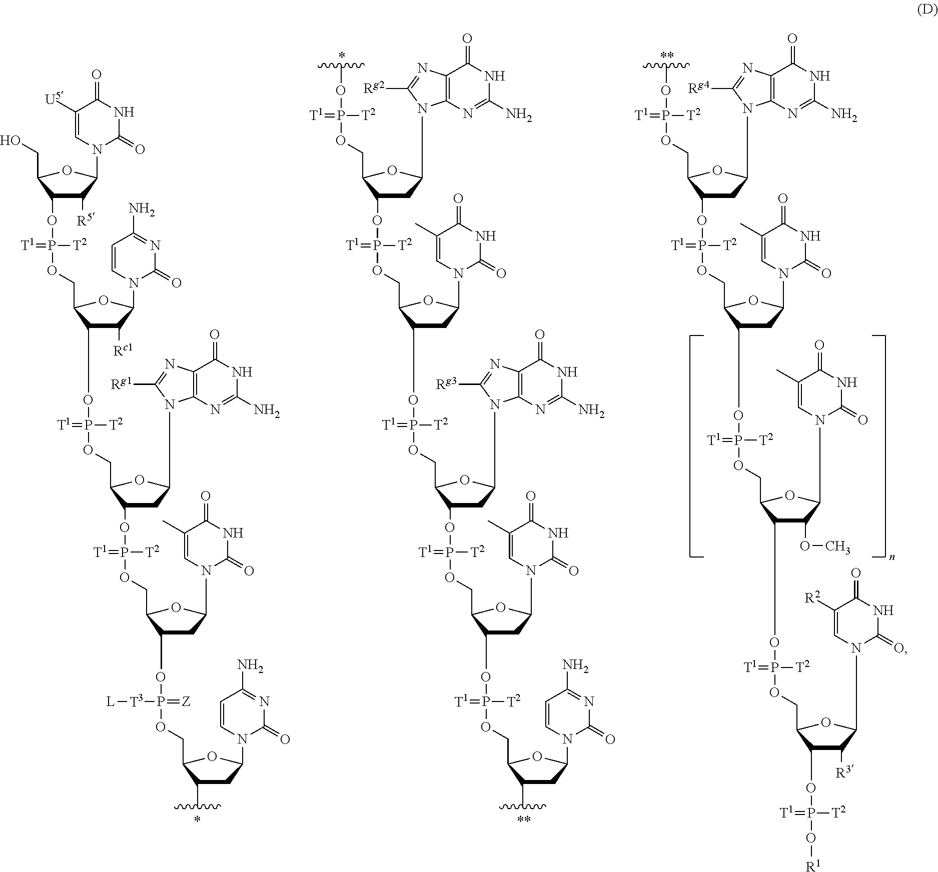

wherein
   [symbol] * and [symbol] ** indicate the points of attachment within the oligonucleotide;
   each T¹ is independently O or S;
   each T² is S;
   T³ is a group

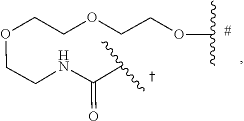 [SMILES: *OCCOCCOCCNC(*)=O]

wherein [symbol] † indicates the point of attachment to L and wherein [symbol] # indicates the point of attachment to the rest of the oligonucleotide;
L is a group

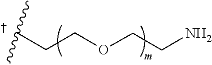

wherein m is an integer from 0 to 50 and wherein [symbol] † indicates the point of attachment to the rest of the oligonucleotide via T³;
Z is O or S;
U^{5′} is —H or halogen;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo and wherein if one of R^{g1}, R^{g2}. R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In some embodiments of the present aspect, U^{5′} is —H In other embodiments, U^{5′} is halogen. In certain embodiments, U^{5′} is iodo or bromo. In some embodiments of the present aspect, the immunomodulatory oligonucleotide of formula (D) is an immunomodulatory oligonucleotide of formula (D′). In other embodiments of the present aspect, the immunomodulatory oligonucleotide of formula (D) is an immunomodulatory oligonucleotide of formula (D″).

In some embodiments of the present aspect, provided herein is an immunomodulatory oligonucleotide of formula (D′)

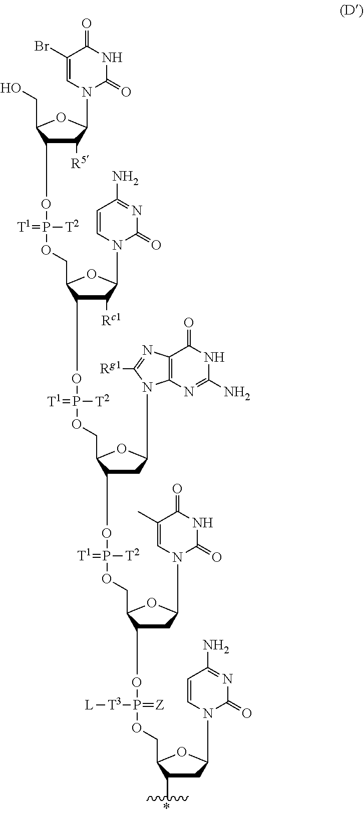

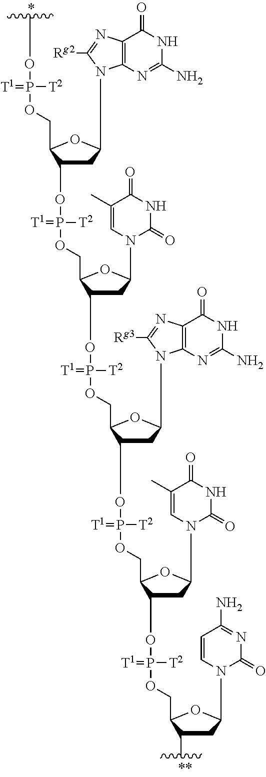

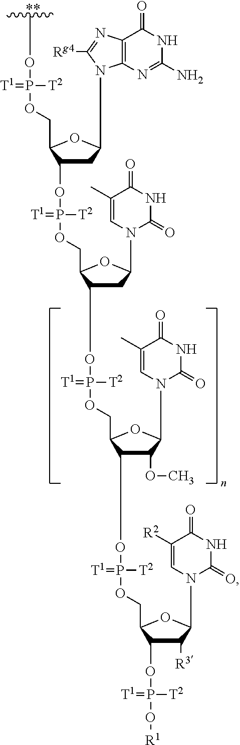

wherein
   [symbol] * and [symbol] ** indicate the points of attachment within the oligonucleotide;
   each T¹ is independently O or S;
   each T² is S⁻;
   T³ is a group

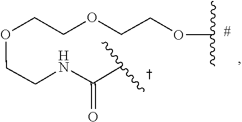 [SMILES: *OCCOCCOCCNC(*)=O]

wherein [symbol] † indicates the point of attachment to L and wherein [symbol] # indicates the point of attachment to the rest of the oligonucleotide;
L is a group

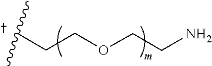

wherein m is an integer from 0 to 50 and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo and wherein if one of R^{g1}, R^{g2}, R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In other embodiments of the present aspect, provided herein is an immunomodulatory oligonucleotide of formula (D″)

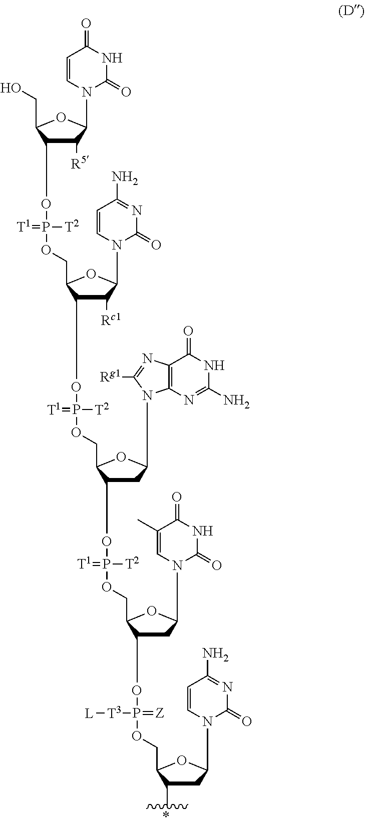

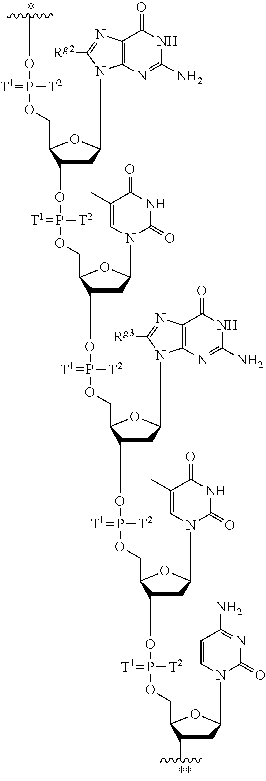

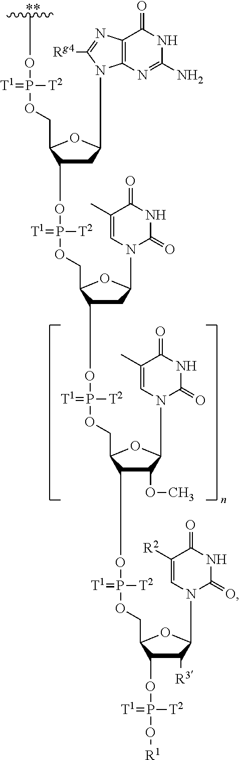

wherein
   [symbol] * and [symbol] ** indicate the points of attachment within the oligonucleotide;
   each T¹ is independently O or S;
   each T² is S⁻;
   T³ is a group

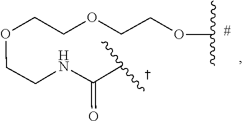 [SMILES: *OCCOCCOCCNC(*)=O]

wherein [symbol] † indicates the point of attachment to L and wherein [symbol] # indicates the point of attachment to the rest of the oligonucleotide;
L is a group

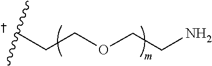

wherein m is an integer from 0 to 50 and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R^{g4} are H or oxo, optionally wherein at least one of R^{g1}, R^{g2}, R^{g3}, and R^{g4} is oxo and wherein if one of R^{g1}, R^{g2}. R^{g3}, and R^{g4} is oxo, then the carbon to which the oxo is attached has a single bond to the ring nitrogen at the 7-position;
R^{3′} is methoxy or 2-methoxyethoxy;
R¹ is —(CH₂)₃—OH;
R² is —H or methyl; and
n is an integer from 0 to 2,
or a pharmaceutically acceptable salt thereof.

In some embodiments, if one of R^{g1}, R^{g2}, R^{g3}, and R^{g4} is oxo, then the carbon to which the oxo is attached has a single bond to the ring nitrogen at the 7-position.

In some embodiments of the present aspect, the present disclosure also provides immunomodulating oligonucleotides of formula (D′)

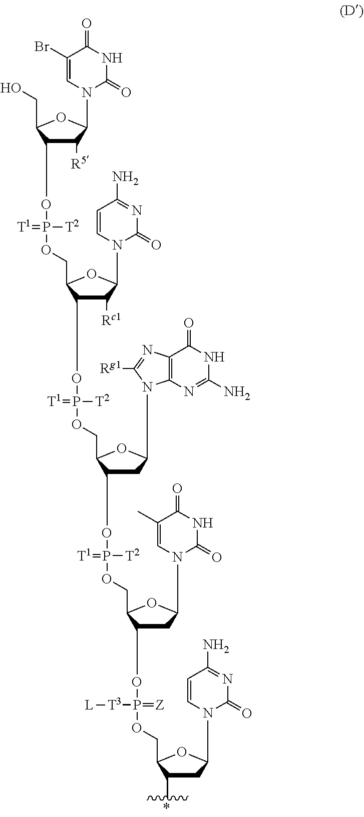

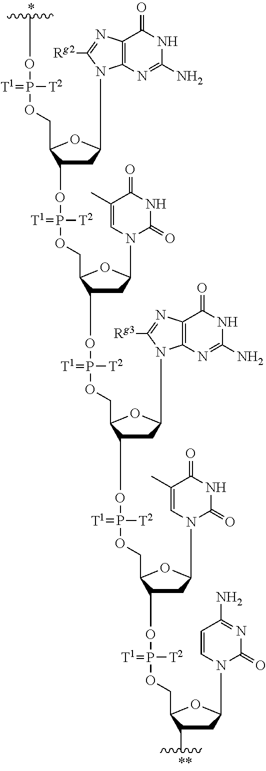

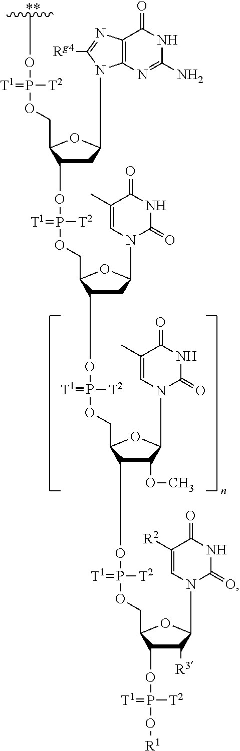

wherein
   [symbol] * and [symbol] ** indicate the points of attachment within the oligonucleotide;
   each T¹ is independently O or S;
   each T² is S—;
   T³ is a group

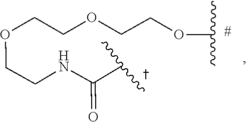 [SMILES: *OCCOCCOCCNC(*)=O]

wherein [symbol] † indicates the point of attachment to L and wherein [symbol] # indicates the point of attachment to the rest of the oligonucleotide;
L is a group

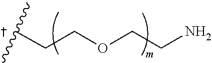

wherein m is an integer from 0 to 50 and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In still other embodiments of the present aspect, provided herein is an immunomodulatory oligonucleotide of formula (D″)

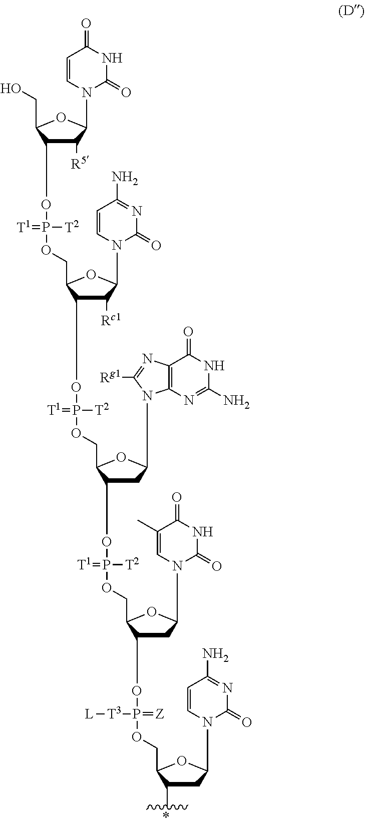

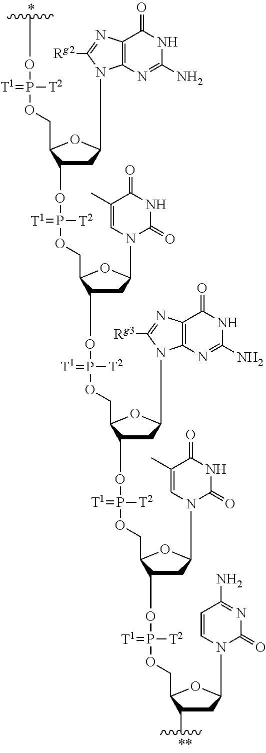

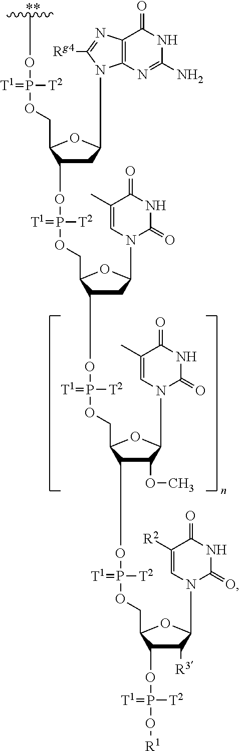

wherein
   [symbol] * and [symbol] ** indicate the points of attachment within the oligonucleotide;
   each T¹ is independently O or S;
   each T² is S⁻;
   T³ is a group

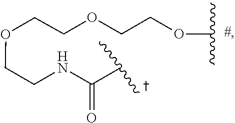 [SMILES: *OCCOCCOCCNC(*)=O]

wherein [symbol] † indicates the point of attachment to L and wherein [symbol] # indicates the point of attachment to the rest of the oligonucleotide;
L is a group

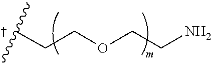

wherein m is an integer from 0 to 50 and wherein [symbol] † indicates the point of attachment to the rest of the oligonucleotide via T³;
Z is O or S;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2,
or a pharmaceutically acceptable salt thereof.

In some embodiments of the present aspect, Z is S. In additional embodiments, the oligonucleotide comprises at least one pair of geminal T¹ and T² wherein T¹ is S and T² is S⁻. In certain embodiments, the oligonucleotide comprises at least two pairs of geminal T¹ and T² wherein T¹ is S and T² is S⁻.

In still yet another embodiment of the present aspect, provided herein is an oligonucleotide of formula (D)

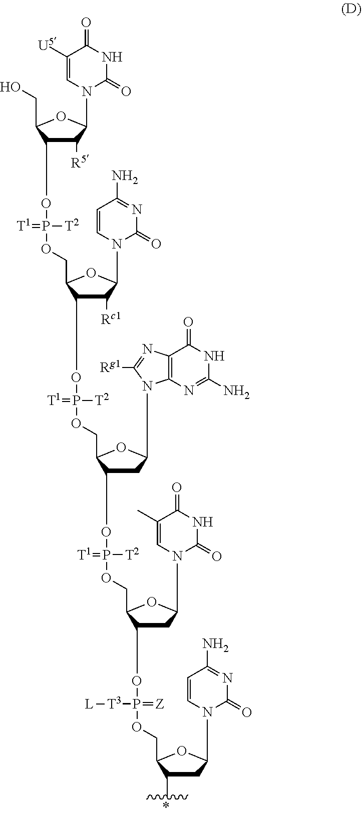

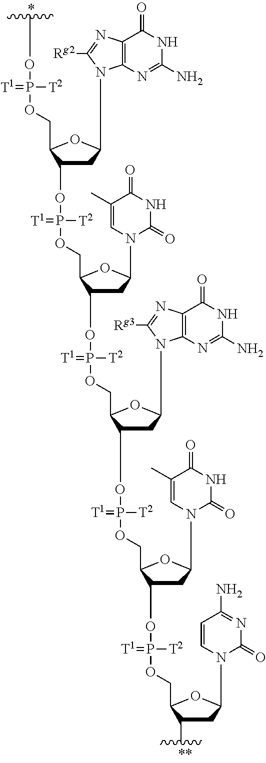

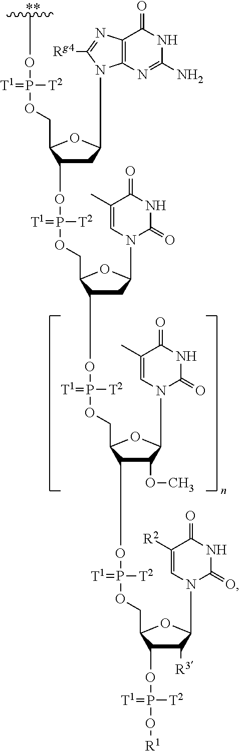

wherein [symbol] * and [symbol] ** indicate the points of attachment within the oligonucleotide;
each T¹ is independently O or S;
each T² is S⁻;
provided that the oligonucleotide comprises at least one pair of geminal T¹ and T² wherein T¹ is S and T² is S,
T³ is a group

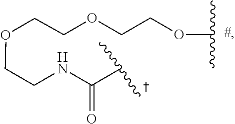 [SMILES: *OCCOCCOCCNC(*)=O]

wherein [symbol] † indicates the point of attachment to L and wherein [symbol] # indicates the point of attachment to the rest of the oligonucleotide;
L is a group

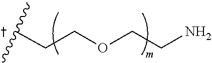

wherein m is an integer from 0 to 50 and wherein [symbol] † indicates the point of attachment to the rest of the oligonucleotide via T³;
Z is O or S;
U^{5′} is —H or halogen;
R^{5′} is —H;
R^{c1} is —H;
R^{g1}, R^{g2}, R^{g3}, and R^{g4} are H;
R^{3′} is methoxy;
R¹ is —(CH₂)₃—OH;
R² is -methyl; and
n is 1,
or a pharmaceutically acceptable salt thereof.

In some embodiments of any of the foregoing, the at least one pair of geminal T¹ and T² wherein T¹ is S and T² is S is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In still other embodiments of the foregoing, the oligonucleotide comprises at least two pairs of of geminal T¹ and T² wherein T¹ is S and T² is S, and wherein the at least two pairs of of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, the oligonucleotide comprises one or two pairs of geminal T¹ and T² wherein T¹ is S and T² is S, and wherein the one or two pairs of geminal T¹ and T² are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain embodiments, the oligonucleotide comprises one pair of geminal T¹ and T² wherein T¹ is S and T² is S, and wherein the pair of geminal T¹ and T² is between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11. In certain other embodiments, the oligonucleotide comprises two pairs of geminal T¹ and T² wherein T¹ is S and T² is S, and wherein the two pairs of geminal T¹ and T² wherein T¹ is S and T² is S are between nucleoside residues 2 and 3, between nucleoside residues 3 and 4, between nucleoside residues 5 and 6, between nucleoside residues 6 and 7, between nucleoside residues 7 and 8, between nucleoside residues 8 and 9, between nucleoside residues 9 and 10, or between nucleoside residues 10 and 11.

In some embodiments, R⁵′ is H. In other embodiments, R⁵′ is methoxy. In some embodiments, R^{c1} is H. In yet other embodiments, R^{c1} is methoxy. In still further embodiments, R² is methyl. In still other embodiments, R² is H. In yet other additional embodiments, which may be combined with any of the preceding embodiments, T³ is

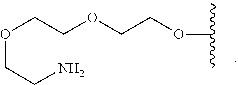 [SMILES: *OCCOCCOCCN]

In still other embodiments, T³ is

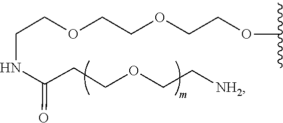

In certain embodiments, m is an integer from 20 to 25.

In another aspect, the immunomodulating oligonucleotide of formula (D) is an oligonucleotide selected from the group consisting of the oligonucleotides of Table 16 and Table 17, or a pharmaceutically acceptable salt thereof. In still further embodiments of the present aspect, the oligonucleotide of formula (D) is selected from the group consisting of the oligonucleotides of Table 17, or a pharmaceutically acceptable salt thereof.

TABLE 16
Modified Oligonucleotide Structures (with -PEG₃NHCOPEG₂₄NH₂)

| Cmpd | |
|---|---|
| # | Structure |
| 1.1b | 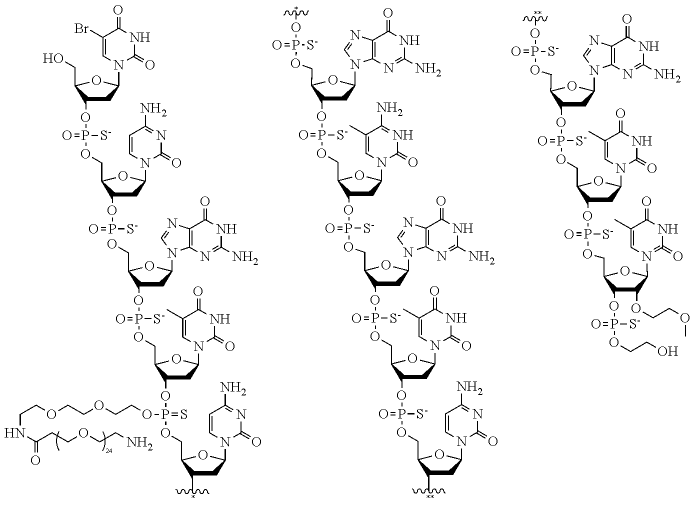 |
| 2.1b | 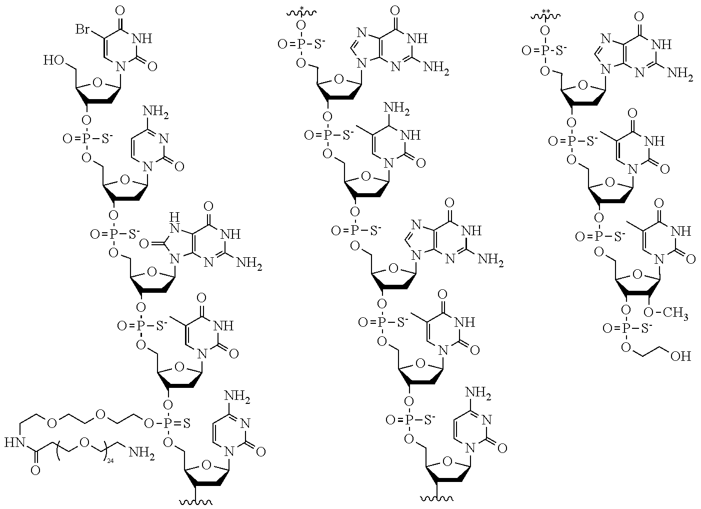 |
| 2.2b | 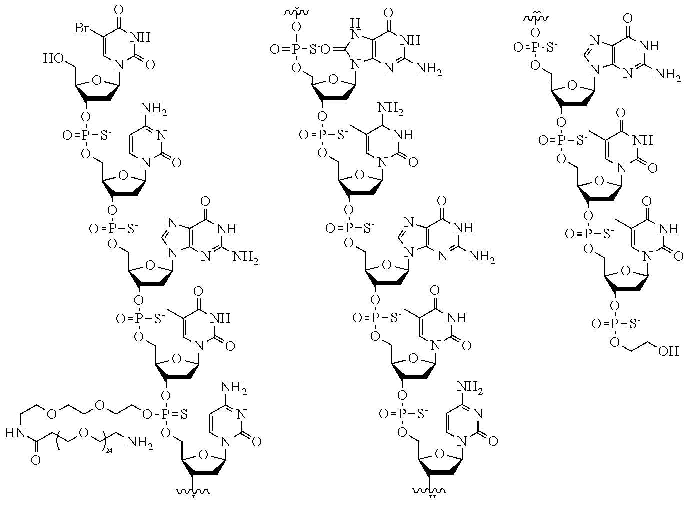 |
| 2.3b | 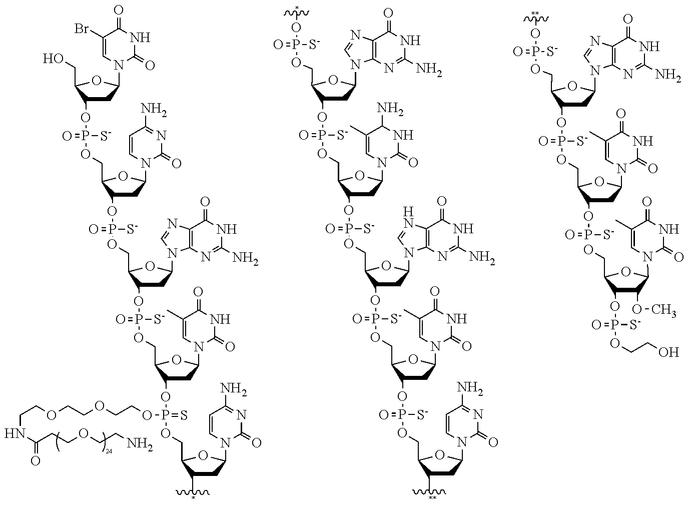 |
| 2.4b | 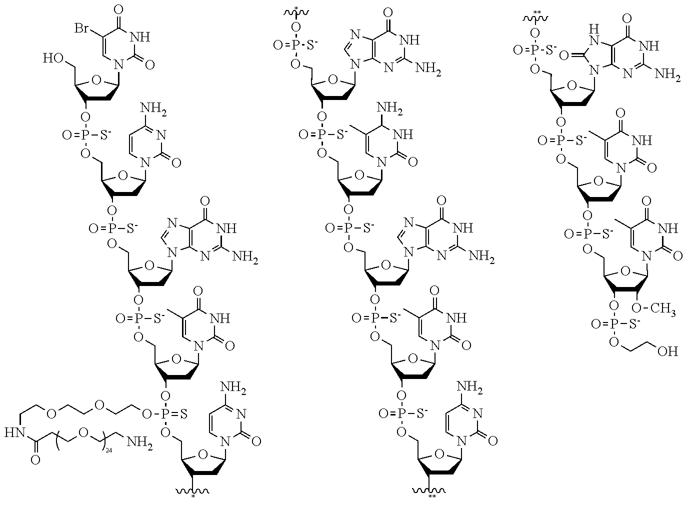 |

TABLE 17
Modified Oligonucleotide Structures (with -PEG₃NHCOPEG₂₄NH₂)

| Cmpd | |
|---|---|
| # | Structure |
| 3.1b | 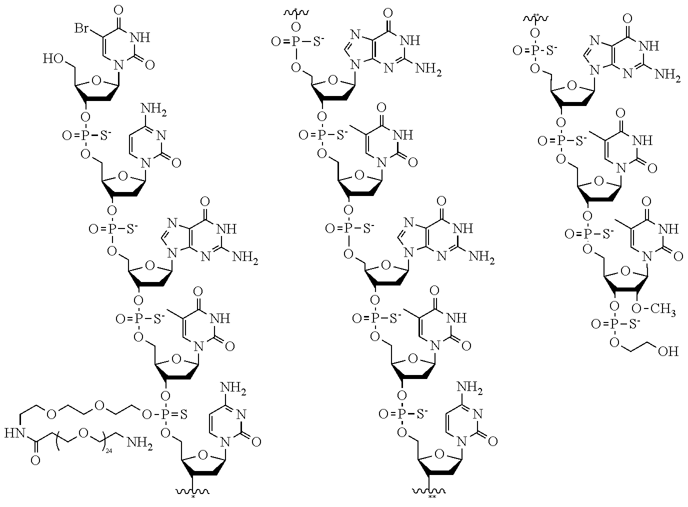 |
| 3.2b | 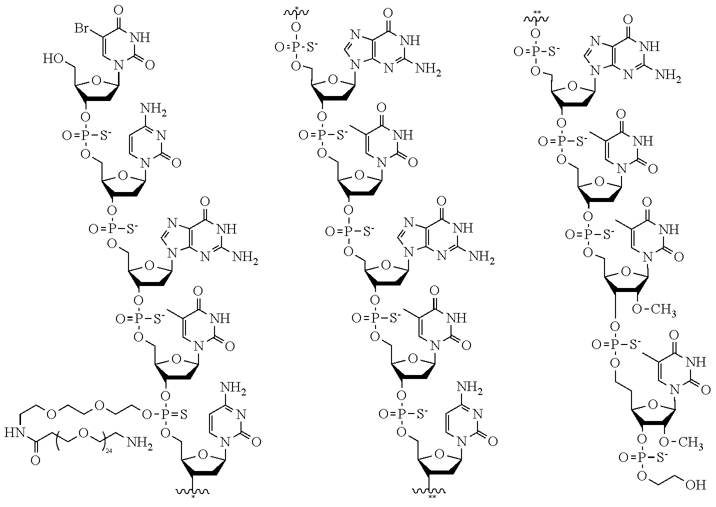 |
| 3.3b | 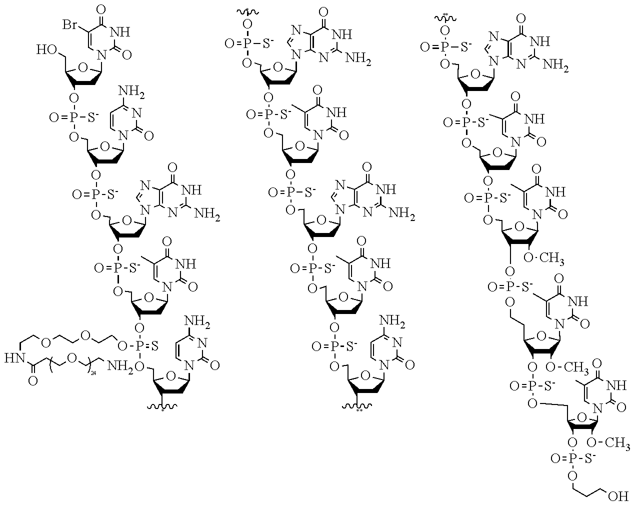 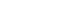 |
| 4.1b | 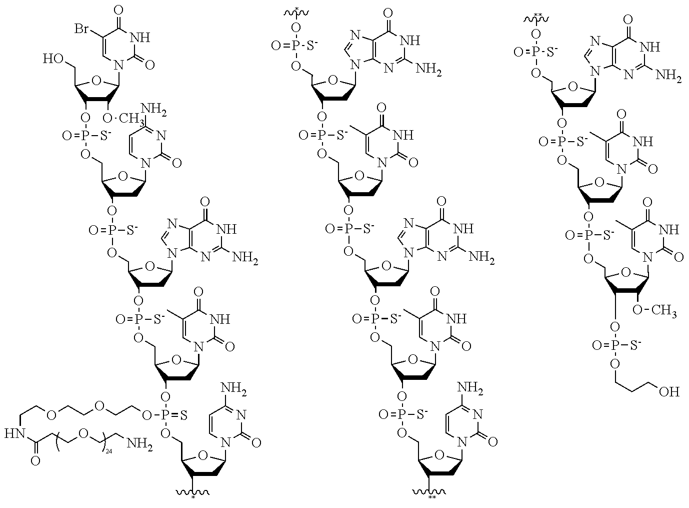 |
| 4.2b | 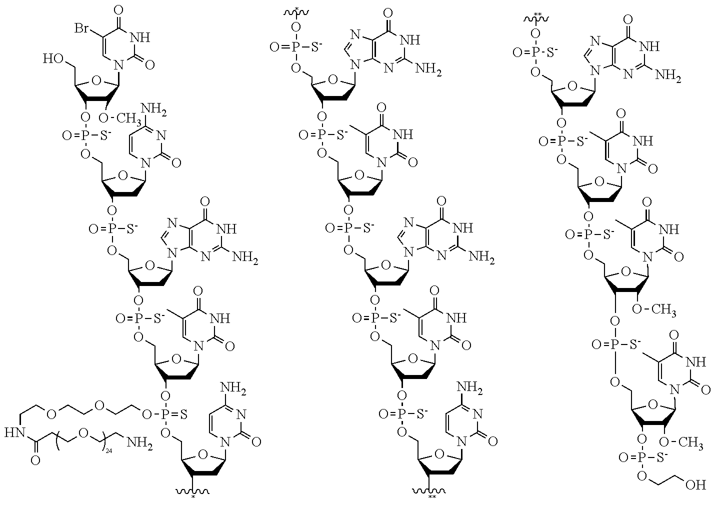 |
| 4.3b | 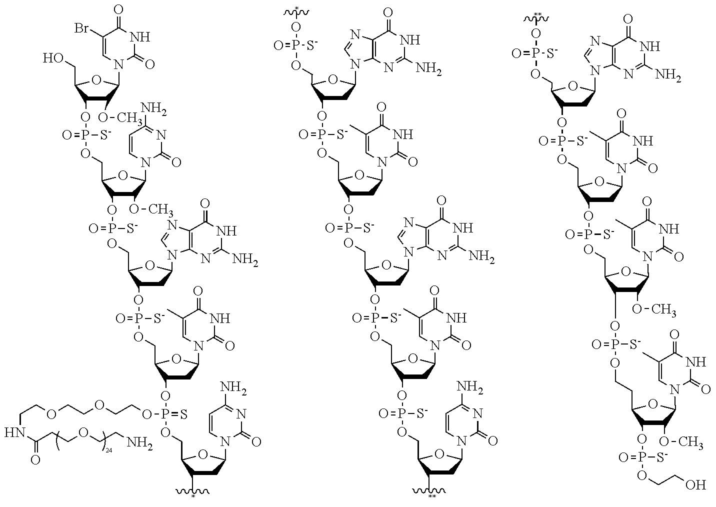 |
| 5.1b | 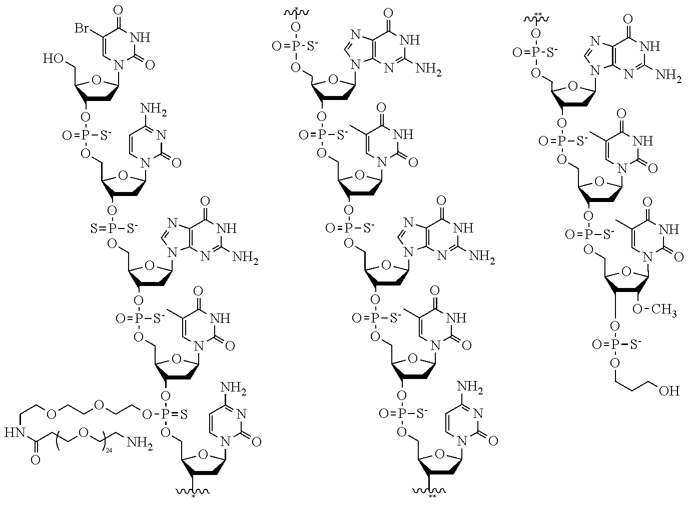 |
| 5.2b | 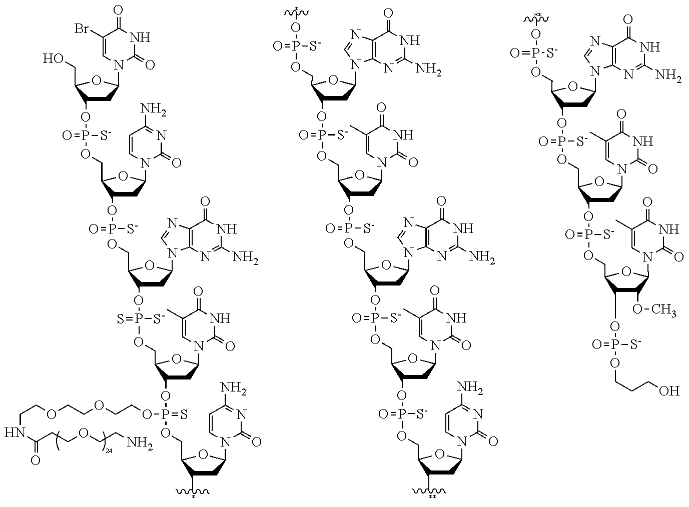 |
| 5.3b | 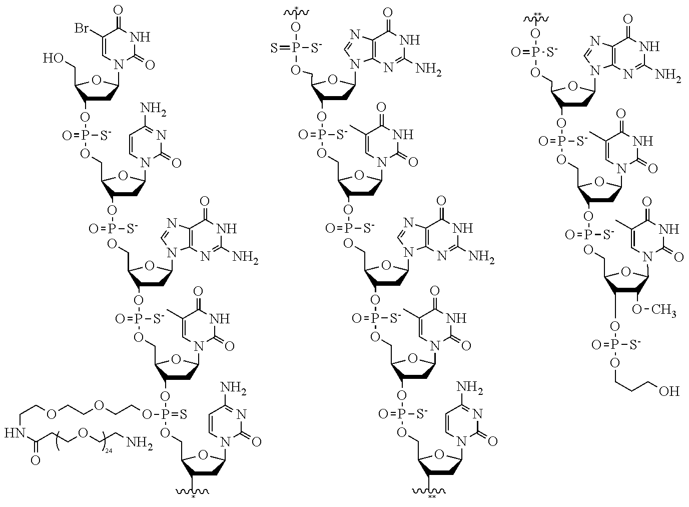 |
| 5.4b | 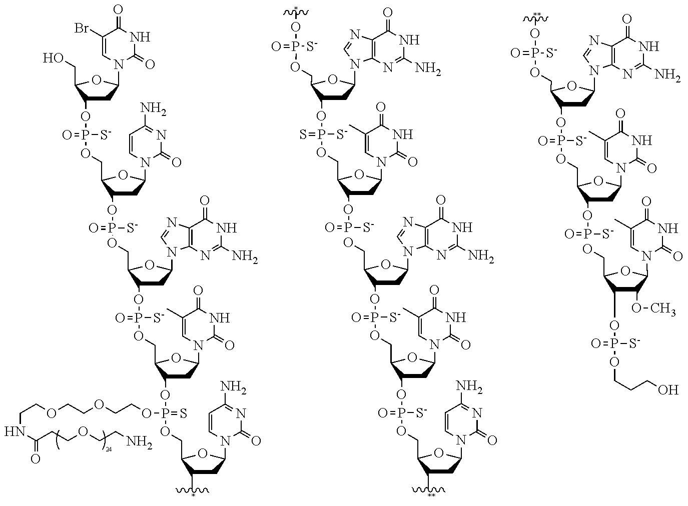 |
| 5.5b | 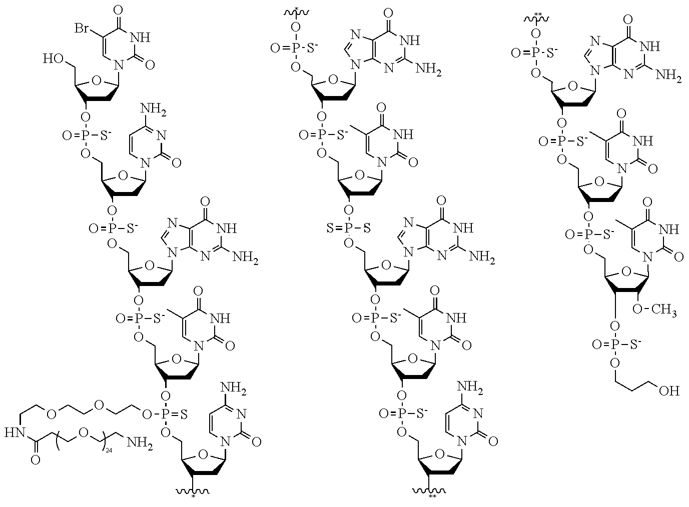 |
| 5.6b | 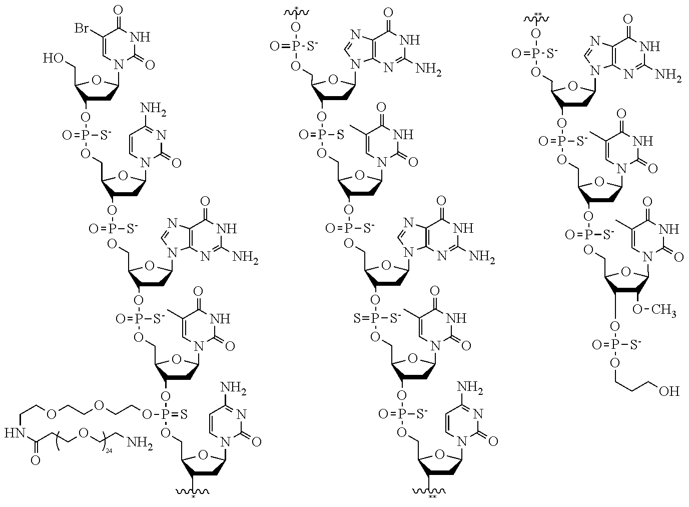 |
| 5.7b | 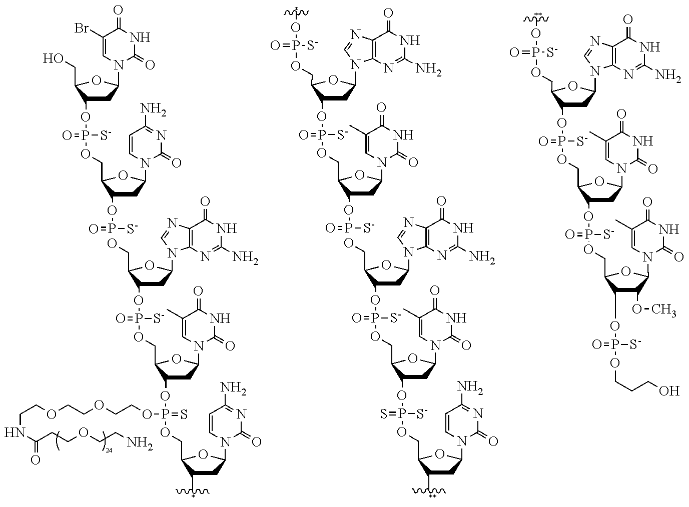 |
| 5.8b | 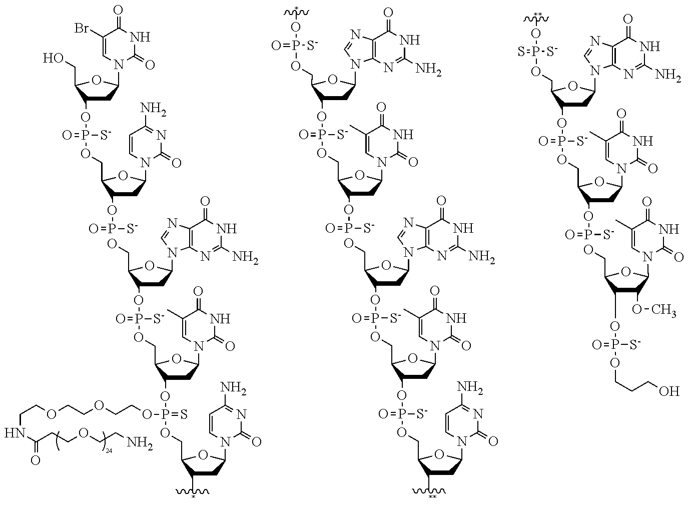 |
| 5.9b | 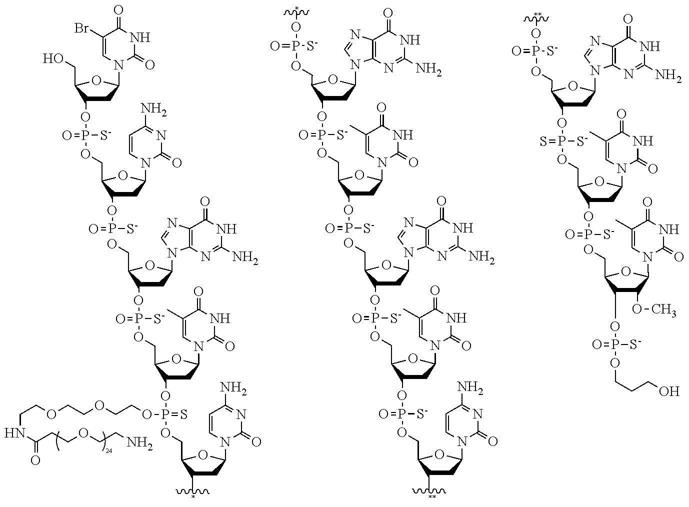 |
| 5.10b | 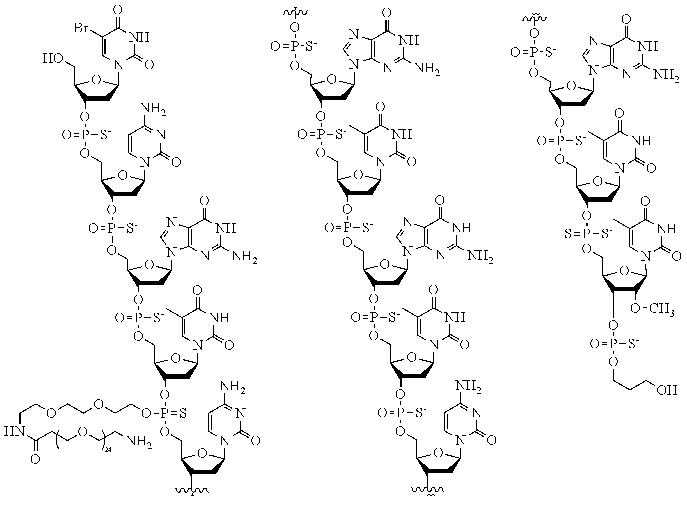 |
| 5.11b | 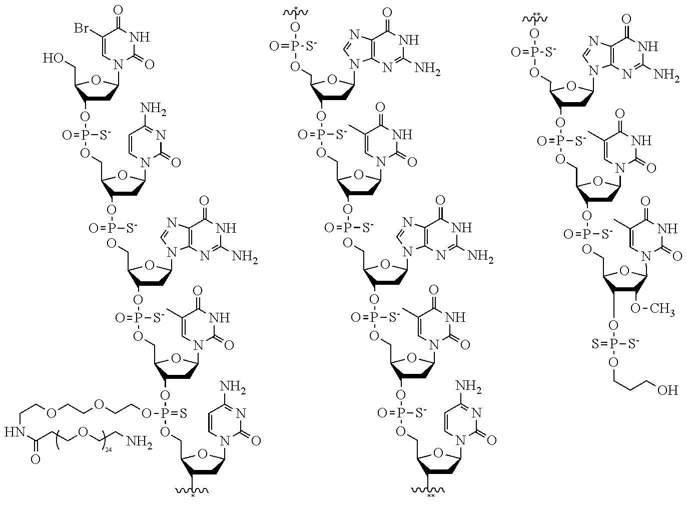 |
| 5.12b | 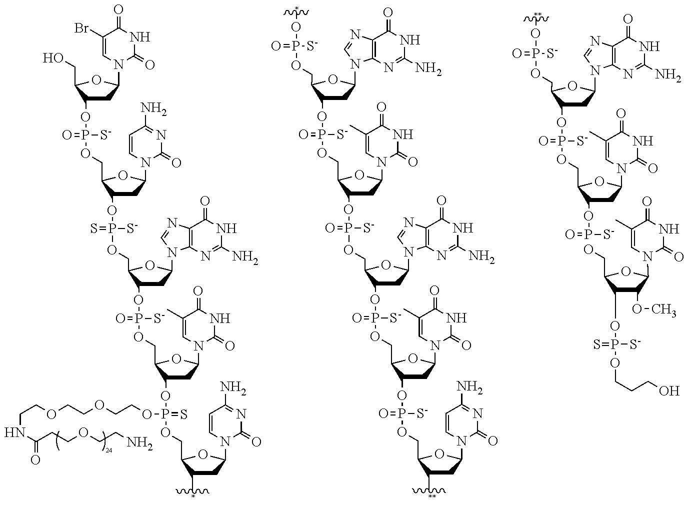 |
| 6.1b | 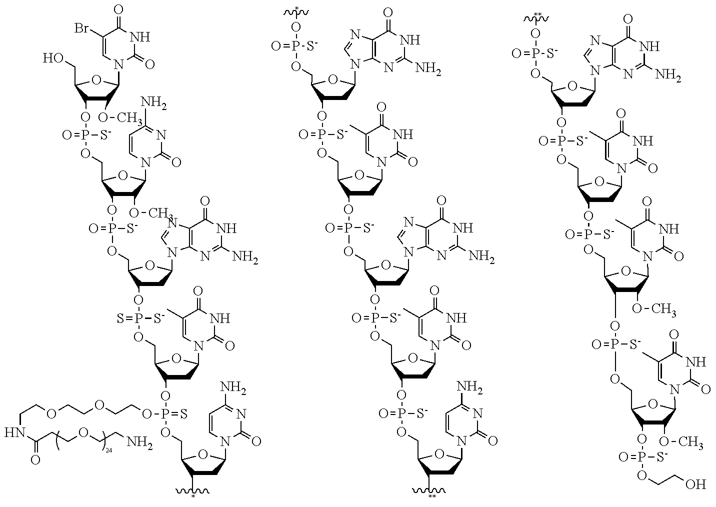 |
| 6.2b | 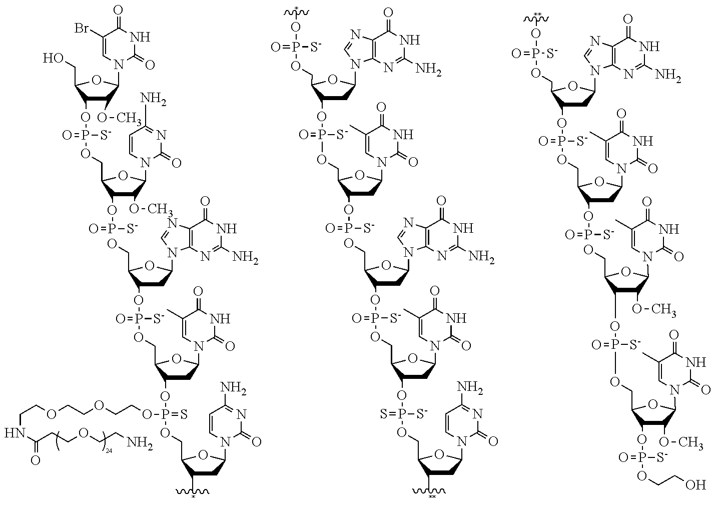 |
| 6.3b | 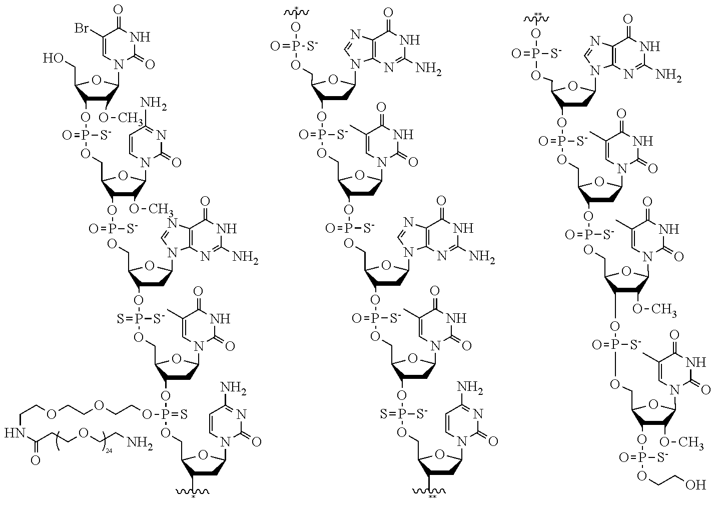 |
| 7.1b | 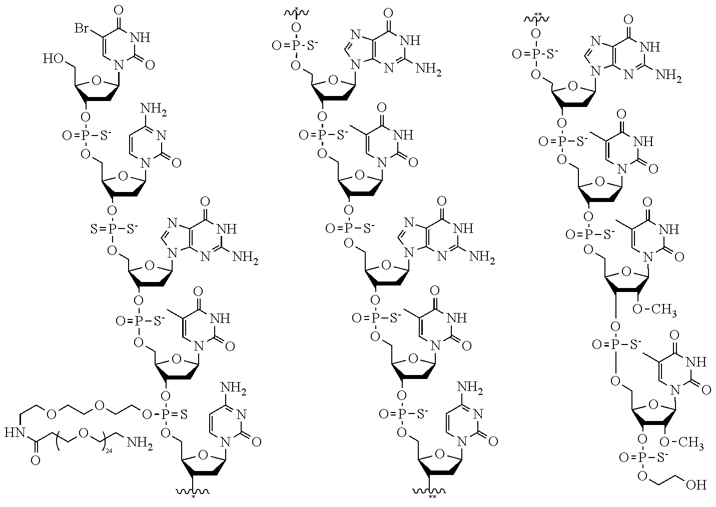 |
| 7.2b | 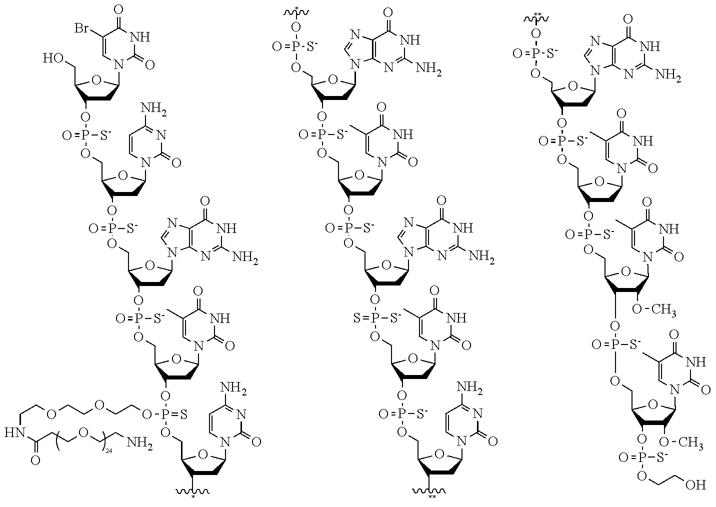 |
| 7.3b | 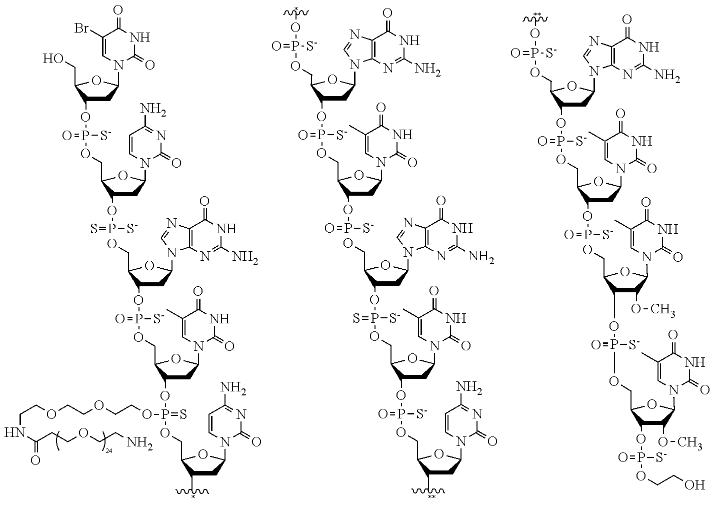 |
| 7.4b | 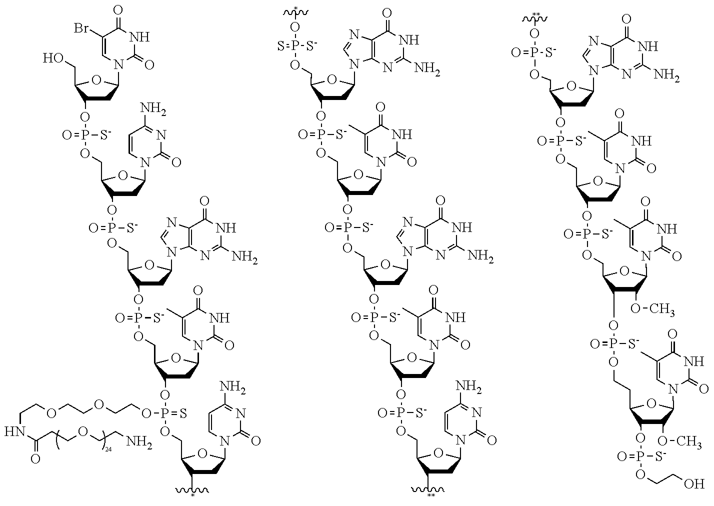 |
| 7.5b | 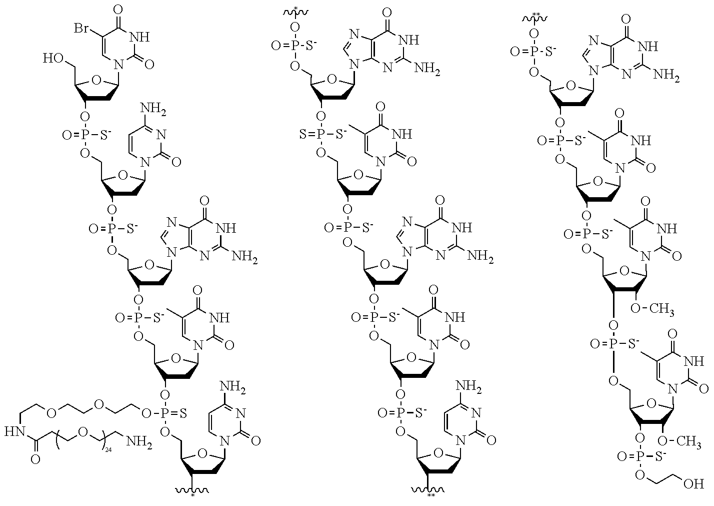 |
| 7.6b | 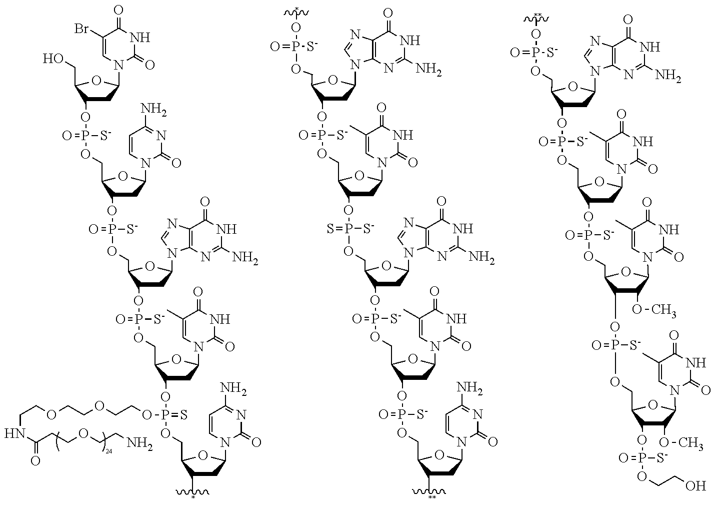 |
| 7.7b | 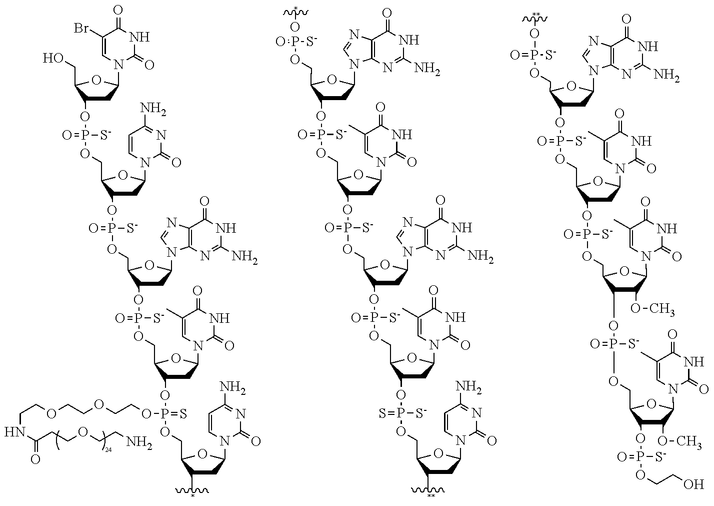 |
| 7.8b | 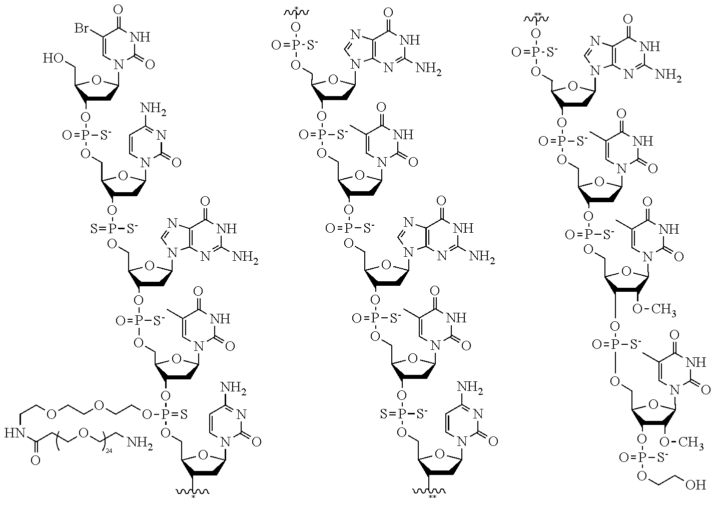 |
| 7.9b | 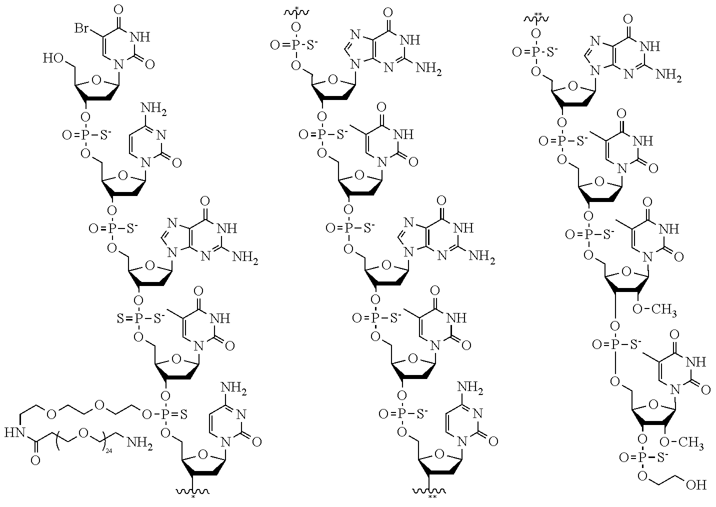 |
| 7.10b | 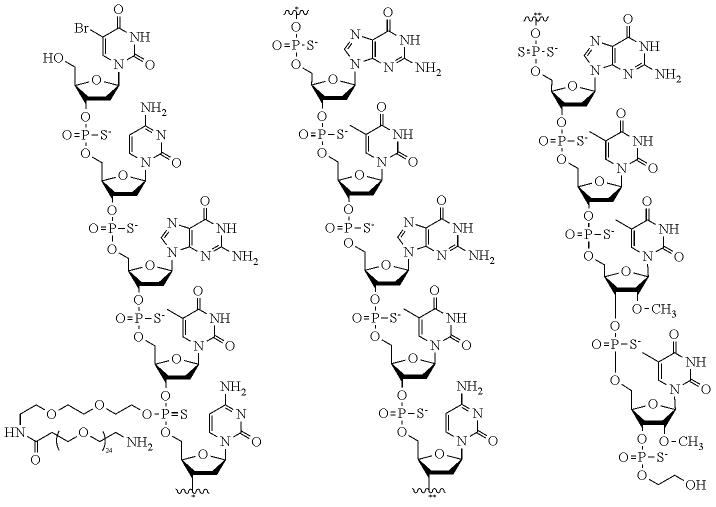 |
| 15.7b | 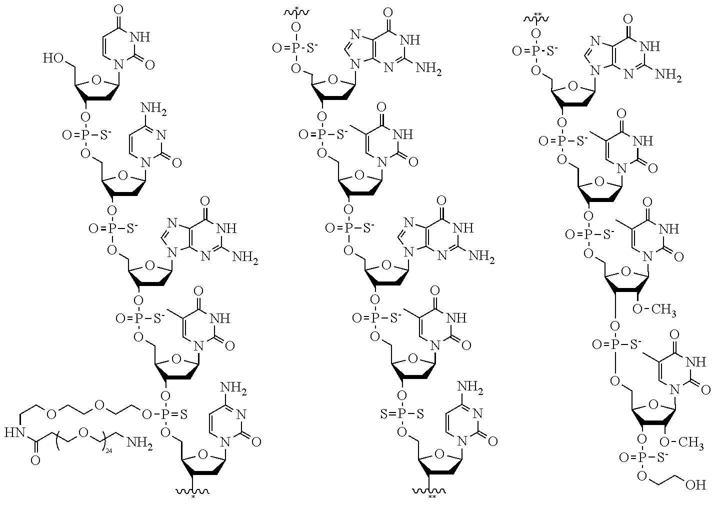 |

As with the oligonucleotides of formula (C), the immunomodulating oligonucleotides of formula (D) may be used as precursors to prepare conjugates comprising a SIRP-α antibody or antigen-binding fragment thereof and one or more immunomodulating oligonucleotides of formula (D) linked via Q-tag as shown in the structures of formula (A) as described herein.

The immunomodulating oligonucleotides of formulae (C) and (D) as described herein may be prepared according to methods known in the art. A general method for the preparation of immunomodulating oligonucleotides, including those provided in the present disclosure, is described below.

### General Oligonucleotide Synthesis:

### General Scheme:

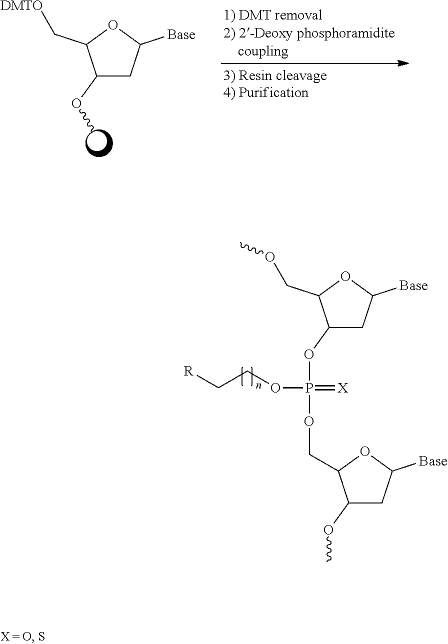

### Experimental Details:

Automated oligonucleotide synthesis (1 μmol scale) was carried out on MerMade 6 or 12 with the following reagents and solvents:
Oxidizer—0.02M I₂ in THF/pyridine/H₂O (60 s oxidation per cycle),
Sulfurizing Reagent II—dithiazole derivative/pyridine/acetonitrile (0.05 M, in 6:4 pyridine:acetonitrile) (60 s per cycle)
Deblock—3% trichloroacetic acid (2×40 s deblocks per cycle),
Cap Mix A—THF/2,6-lutidine/Ac₂O (60 s capping per cycle), and
Cap Mix B—16% methyl imidazole in THF (60 s capping per cycle)

Exceptions to standard oligonucleotide synthesis conditions were as follows:
CPG supports with a non-nucleosidic linker called Uny-linker was used.
All 2′-deoxyribose-phosphoramidites were resuspended to 100 mM in 100% anhydrous acetonitrile prior to synthesis, except some of the modified 2′-deoxy-phosphoramidites were dissolved to 100 mM in THF/acetonitrile mixture (1:4) depend on the solubility of the starting material.
Phosphoramidite activation was performed with a 2.5-fold molar excess of 5-benzylthio-1H-tetrazole (BTT). Activated 2′-deoxyribose-phosphoramidites were coupled for 2×1 minute coupling per insertion and modified phosphoramidites were coupled for 2×3 minute coupling per insertion.
Sulfurization of the backbone was performed with 0.05M Sulfurizing Reagent II in pyridine/acetonitrile (6:4) for 1 min.

### Oligonucleotide Deprotection & Purification Protocol:

Following automated oligonucleotide synthesis, solid support and base protecting groups (such as A-Bz, C-Ac, G-iBu, etc.) and methyl esters of phosphotriesters were cleaved and de-protected in 1 mL of AMA (1:1 ratio of 36% aq. ammonia and 40% methylamine in methanol) for 2 h or more at room temperature followed by centrifugal evaporation.

Crude oligonucleotide pellets were resuspended in 100 μL of 50% acetonitrile, briefly heated to 65° C. and vortexed thoroughly.

For oligonucleotide purification, 100 μL crude oligonucleotides were injected onto RP-HPLC with the following buffers/gradient:
Buffer A=50 mM TEAA in Water;
Buffer B=90% Acetontrile; and
Flow Rate=1 mL/min;
Gradient:
   0-2 min (100% Buffer A/0% Buffer B),
   2-42 min (0% to 60% Buffer B), and
   42-55 min (60% to 100% Buffer B).

### DBCO Conjugation and Purification Protocol:

DBCO NHS ester was conjugated to the crude 2′-deoxy DMT-oligonucleotide as described here. The crude oligonucleotide pellet was suspended into 45 μL DMSO, briefly heated to 65° C. and vortexed thoroughly. 5 μL of DIPEA was added followed by DBCO-NHS ester (30 eq), which was pre-dissolved in DMSO (1M). The reaction was allowed to stand for 10 minutes or until product formation was confirmed by MALDI. Total 80 μL of crude oligonucleotide samples were injected onto RP-HPLC with the following buffers/gradient:
Buffer A=50 mM TEAA in Water
Buffer B=90% Acetonitrile
Flow Rate=1 mL/min
Gradient:
   0-2 min (90% Buffer A/10% Buffer B)
   2-42 min (0% to 60% Buffer B)
   42-55 min (60% to 100% Buffer B).

Across the dominant RP-HPLC peaks, 0.5 mL fractions were collected and analyzed by MALDI-TOF mass spectrometry to confirm presence of desired mass. Mass-selected, purified fractions were frozen and lyophilized. Once dry, fractions were re-suspended, combined with corresponding fractions, frozen and lyophilized.

DMT Cleavage: lyophilized pellets were suspended in 20 μL of 50% acetonitrile and added 80 μL of acetic acid, samples were kept standing at room temperature for 1 h, frozen and lyophilized. The dried samples were re-dissolved in 20% acetonitrile and desalted through NAP 10 (Sephadex™-G25 DNA Grade) columns. Collected, pure fractions were frozen and lyophilized for final product.

### Methods for Attaching Oligonucleotides to Linking Moiety

### Cu-Catalyzed Click Reaction

### Copper-THPTA Complex Preparation

A 5 mM aqueous solution of copper sulfate pentahydrate (CuSO₄-5H₂O) and a 10 mM aqueous solution of tris(3-hydroxypropyltriazolylmethyl)amine (THPTA) were mixed 1:1 (v/v) (1:2 molar ratio) and allowed to stand at room temperature for 1 hour. This complex can be used to catalyze Huisgen cycloaddition, e.g., as shown in the general conjugation schemes below.

### General Procedure (100 nM Scale):

To a solution of 710 μL of water and 100 μL tert-butanol (10% of final volume) in a 1.7 mL Eppendorf tube was added 60 μL of the copper-THPTA complex followed by 50 μL of a 2 mM solution of the oligo, 60 μL of a 20 mM aqueous sodium ascorbate solution and 20 μL of a 10 mM solution of targeting moiety-azide. After thorough mixing the solution was allowed to stand at room temperature for 1 hour. Completion of the reaction was confirmed by gel analysis. The reaction mixture is added to a screw cap vial containing 5-10 fold molar excess of SiliaMetS® TAAcONa (resin bound EDTA sodium salt). The mixture is stirred for 1 hour. This mixture is then eluted through an Illustra™ Nap™-10 column Sephadex™. The resulting solution is then frozen and lyophilized overnight.

### Attachment Through Amide Linkage:

Conjugation through amidation may be performed under the amidation reaction conditions known in the art. See, e.g., Aaronson et al., Bioconjugate Chem. 22:1723-1728, 2011.

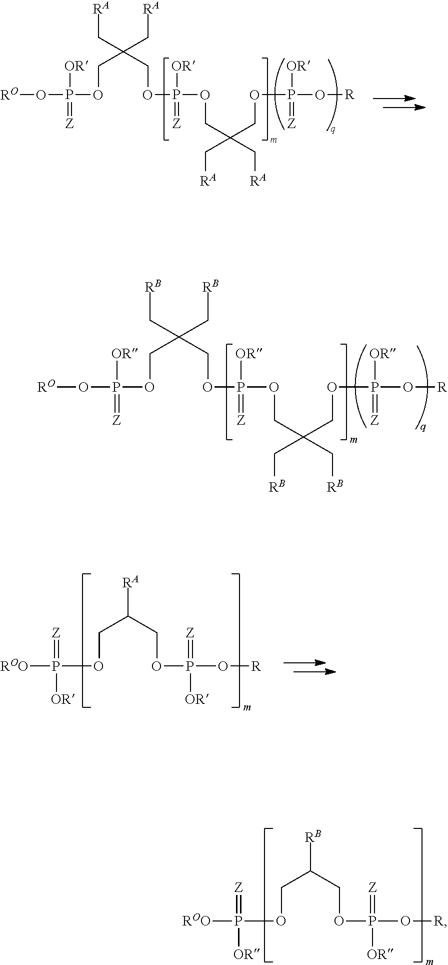

where
each q is 0 or 1;
each m is an integer from 0 to 5;
Z is O or S;
R^{O} is a bond to a nucleoside in an oligonucleotide;
R is a bond to H, a nucleoside in an oligonucleotide, to solid support, or to a capping group (e.g., —(CH₂)₃—OH);
each R′ is independently H, -Q¹-Q^{A1}, a bioreversible group, or a non-bioreversible group;
each R″ is independently H, -Q¹-Q^{A}-Q¹-T, a bioreversible group, or a non-bioreversible group;
each R^{A} is independently H or —OR^{C}, where R^{C} is -Q¹-Q^{A1}, a bioreversible group, or a non-bioreversible group;
each R^{B} is independently H or —OR^{D}, where R^{D} is -Q¹-Q^{A}-Q²-T, a bioreversible group, or a non-bioreversible group;
where
   each Q¹ is independently a divalent, trivalent, tetravalent, or pentavalent group, in which one valency is bonded to Q^{A} or Q^{A1}, the second valency is open, and each of the remaining valencies, when present, is independently bonded to an auxiliary moiety;
   each Q² is independently a divalent, trivalent, tetravalent, or pentavalent group, in which one valency is bonded to Q^{A}, the second valency is bonded to T, and each of the remaining valencies, when present, is independently bonded to an auxiliary moiety;
   Q^{A} is optionally substituted C₂₋₁₂ heteroalkylene containing —C(O)—N(H)— or —N(H)—C(O)—;
   Q^{A1} is —NHR^{N1} or —COOR¹², where R^{N1} is H, N-protecting group, or optionally substituted C₁₋₆ alkyl, and R¹² is H, optionally substituted C₁₋₆ alkyl, or O-protecting group; and
   T is a linking moiety,
   provided that the starting materials contain at least one-Q¹-Q^{A1}, and products contain-Q¹-Q^{A}-Q²-T.

### Solution Phase Attachment:

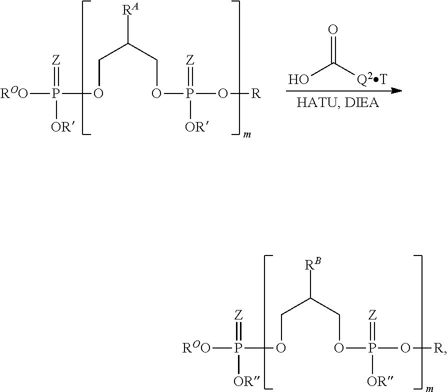 [0752] where
m is an integer from 0 to 5;
Z is O or S;
R^{O} is a bond to a nucleoside in an oligonucleotide;
R is a bond to H, a nucleoside in an oligonucleotide, or to a capping group;
each R′ is independently H, -Q¹-NH₂, a bioreversible group, or a non-bioreversible group;
each R″ is independently H, -Q¹-NH—CO-Q²-T, a bioreversible group, or a non-bioreversible group;
each R^{A} is independently H or —OR^{C}, where R^{C} is -Q¹-NH₂, a bioreversible group, or a non-bioreversible group;
each R^{B} is independently H or —OR^{D}, where R^{D} is -Q¹-NH—CO-Q²-T, a bioreversible group, or a non-bioreversible group;
where
   each Q¹ is independently a divalent, trivalent, tetravalent, or pentavalent group, in which one valency is bonded to —NH—CO— or —NH₂, the second valency is open, and each of the remaining valencies, when present, is independently bonded to an auxiliary moiety;
   each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
   T is a linking moiety,
   provided that the starting material contains-Q¹-NH₂, and the product contains-Q¹-NH—CO-Q²-T.

### On-Support Attachment:

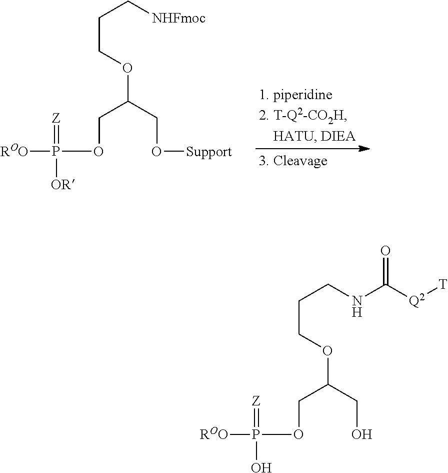 [0767] where
Z is O or S;
R^{O} is a bond to a nucleoside in an oligonucleotide;
each Q² is independently a divalent, trivalent, tetravalent, or pentavalent group, in which one valency is bonded to —NH—CO—, the second valency is a bond to T, and each of the remaining valencies, when present, is independently bonded to an auxiliary moiety; and
T is a linking moiety.

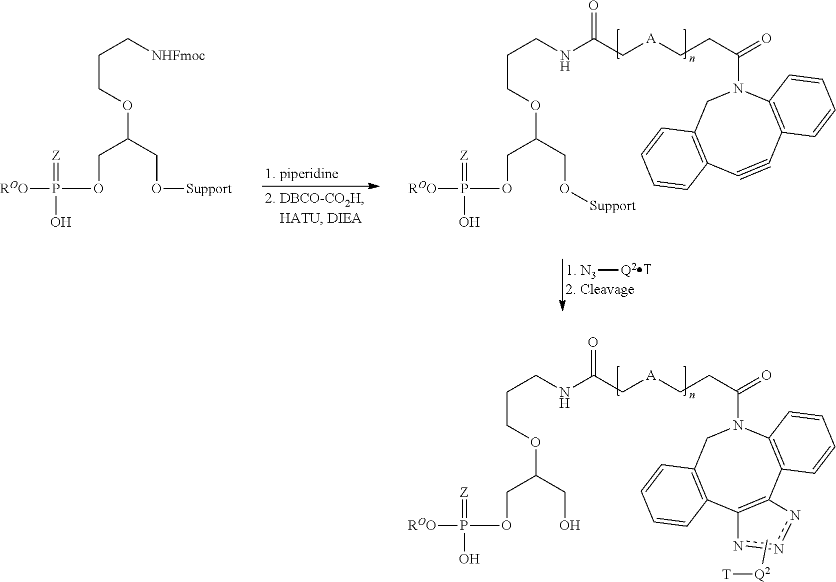

where
n is an integer from 1 to 8;
A is O or —CH₂—;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

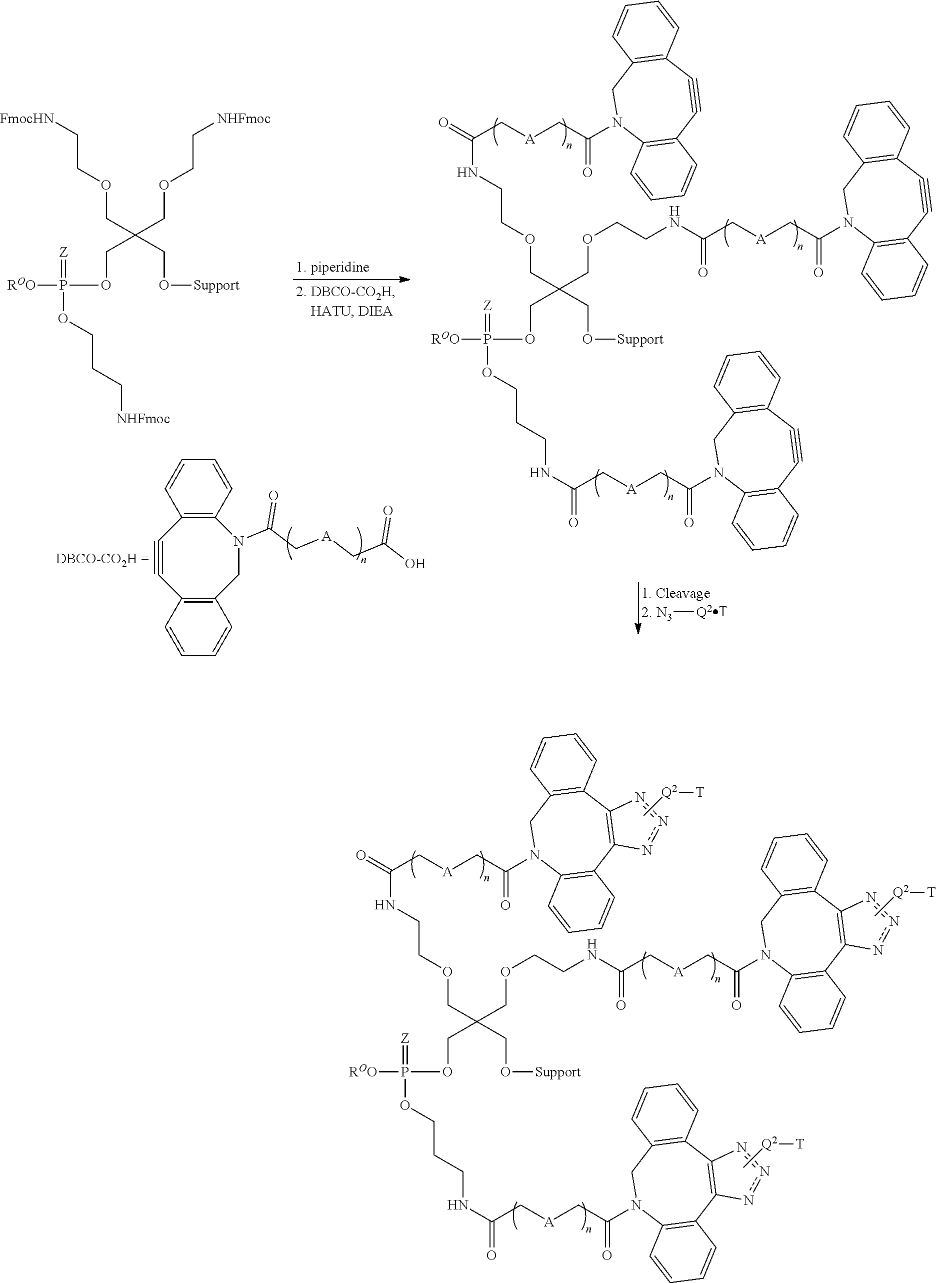

where
n is an integer from 1 to 8;
A is O or —CH₂—;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and
T is a linking moiety.

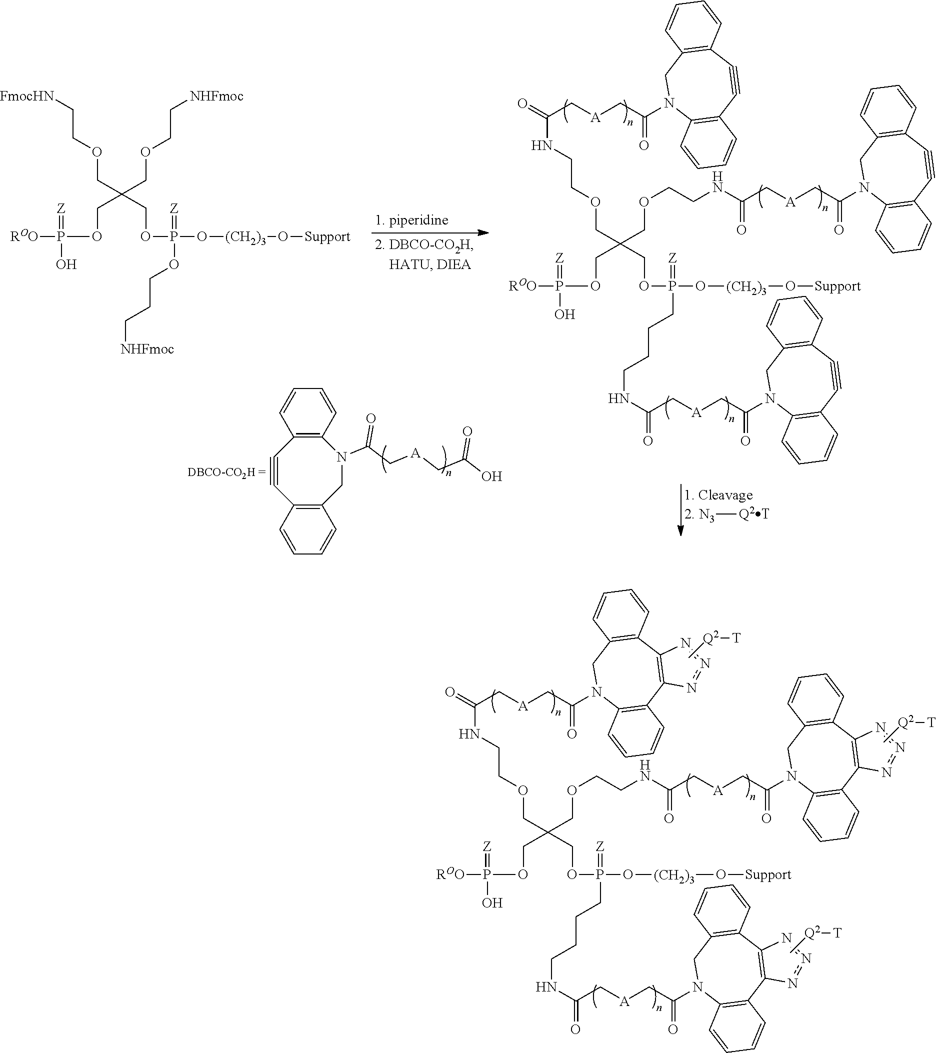

where
n is an integer from 1 to 8;
A is O or —CH₂;
Z is O or S;
R^{O} is a bond to a nucleoside in an oligonucleotide;
each Q² is independently a divalent, trivalent, tetravalent, or pentavalent group; in which one valency is bonded to the azide or triazole, a second valency is bonded to T, and each of the remaining valencies, when present, is independently bonded to an auxiliary moiety; and each T is independently a linking moiety.

### Representative Example of Fmoc Deprotection of a Phosphotriester:

An oligonucleotide including a phosphotriester with Fmoc-protected amine was subjected to deprotection conditions resulting in Fmoc deprotection without observable conversion of the phosphotriester into a phosphodiester.

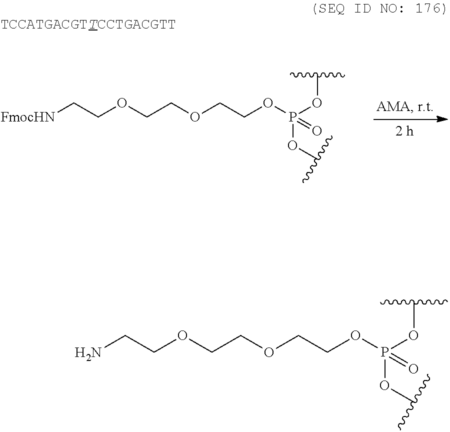

### DBCO-NHS Conjugation to TCCATGACGTTCCTGACGTT (SEQ ID NO:176)—Representative Example:

DBCO-NHS conjugation to the amino group in the phosphotriester was complete in 10 min at room temperature, as evidenced by mass spectrometric analysis.

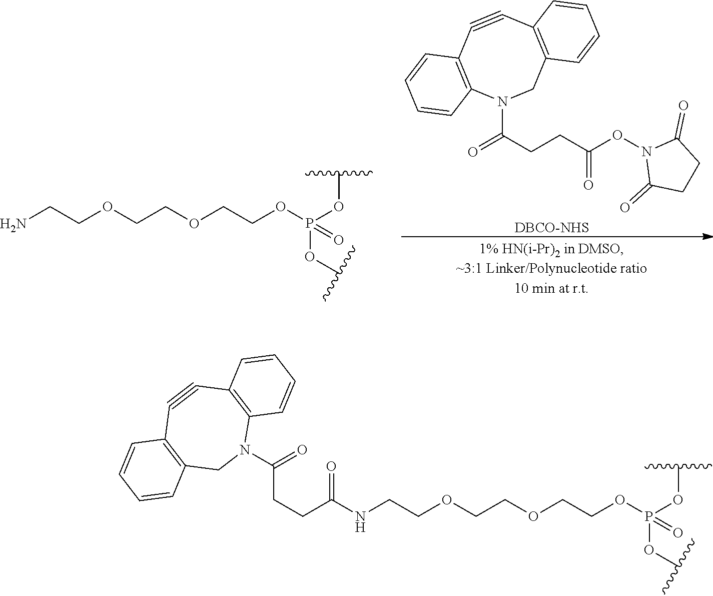

RP-HPLC purification of TCCATGACGTTCCTGACGTT (SEQ ID NO:176) containing a DBCO conjugating group was performed using the following conditions:
Buffer A=50 mM TEAA in Water;
Buffer B=90% Acetontrile; and
Flow Rate=1 mL/min;
Gradient:
   0-2 min (100% Buffer A/0% Buffer B),
   2-22 min (0% to 100% Buffer B), and
   22-25 min (100% Buffer B).

A similar procedure may be used to prepare an oligonucleotide using, e.g., 2′-modified nucleoside phosphoramidites, such as those described herein. Such a procedure is provided in International Patent application PCT/US2015/034749; the disclosure of the disulfide phosphotriester oligonucleotide synthesis in PCT/US2015/034749 is hereby incorporated by reference.

### V. Methods of Conjugation

Provided herein are methods for preparing a conjugate comprising a SIRP-α antibody or antigen-binding fragment thereof and one or more immunomodulating oligonucleotides linked via one or more Q-tag peptides as shown in the structure of Formula (A). In some embodiments, the methods comprise combining a SIRP-α antibody comprising at least one Q-tag peptide sequence with at least one exposed glutamine residue and an oligonucleotide under conditions sufficient to induce conjugation, i.e., amidation reaction between the CpG and Q tag. In other embodiments, the methods comprise reacting a SIRP-α antibody comprising at least one Q-tag peptide sequence with at least one exposed glutamine residue and an oligonucleotide under chemical conditions sufficient to induce conjugation. In still other embodiments, the methods comprise reacting a SIRP-α antibody comprising at least one Q-tag peptide sequence with at least one exposed glutamine residue and an oligonucleotide under enzymatic conditions, e.g., with transglutaminase, sufficient to induce conjugation.

### Transglutaminase-Mediated Conjugation Reaction Conditions

In one aspect, provided herein is a method of preparing a conjugate of formula (A), comprising combining one or more immunomodulating oligonucleotides (P) and a SIRP-α antibody comprising one or more glutamine residues. In one aspect, provided herein is a method of preparing a conjugate comprising a SIRP-α antibody or antigen-binding fragment (Ab) and one or more immunomodulating oligonucleotides (P), wherein the SIRP-α antibody or antigen-binding fragment is linked to one or more Q-tag peptides (Q) comprising the amino acid sequence RPQGF (SEQ ID NO:47), and wherein each immunomodulating oligonucleotide is linked to a Q-tag peptide via an amide bond with the glutamine residue of the Q-tag peptide and a linker (L) as shown in formula (A),

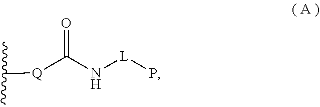

wherein:
   [symbol] indicates the point of attachment of each Q to the SIRP-α antibody or antigen-binding fragment thereof (Ab);
   each Q independently comprises a Q-tag peptide sequence RPQGF (SEQ ID NO: 47);
   each L is independently a bond or a linker moiety connected to Q via an amide bond with the glutamine residue; and
   each P is independently an immunomodulating oligonucleotide;
comprising contacting a compound of formula (B)

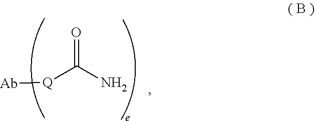

wherein Ab and Q are as defined for formula (A) above, and e is an integer from 1 to 20, with one or more immunomodulating oligonucleotides P, wherein each P independently has the following formula:

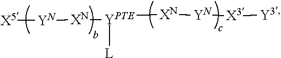

wherein
   X^{5′} is a 5′ terminal nucleoside;
   X^{3′} is a 3′ terminal nucleoside;
   Y^{PTE} is an internucleoside phosphotriester;
   Y^{3′} is a terminal phosphotriester;
   each X^{N} is independently a nucleoside;
   each Y^{N} is independently an internucleoside linker;
   b and c are each independently an integer from 1 to 25; with the proviso that the sum of b and c is at least 5; and
   L is a linker moiety having a terminal amine,
in the presence of a transglutaminase.

In another aspect, method for preparing a conjugate comprising a SIRP-α antibody or antigen-binding fragment (Ab) and one or more immunomodulating oligonucleotides (P), wherein the SIRP-α antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A),

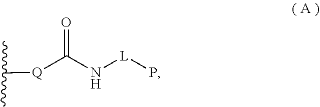

wherein:
   [symbol] indicates the point of attachment of each Q to the SIRP-α antibody or antigen-binding fragment thereof (Ab);
   each Q is independently a Q-tag peptide having at least one glutamine residue;
   each L is independently a bond or a linker moiety connected to Q via an amide bond with the glutamine residue; and
   each P is independently an immunomodulating oligonucleotide;
comprising contacting a compound of formula (B)

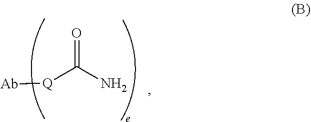

wherein Ab and Q are as defined for formula (A) above, and e is an integer from 1 to 20, with one or more immunomodulating oligonucleotides P, wherein each oligonucleotide P is independently an immunomodulating oligonucleotide of formula (C) or formula (D), in the presence of a transglutaminase.

In some embodiments, the conjugate comprises one or more, two or more, three or more, four or more, five or more, six or more, seven or more, eight or more, nine or more, ten or more, or twenty or more Q-tag peptides. In some embodiments, the conjugate comprises one, two, three, four, five, six, seven, eight, nine, ten, or twenty Q-tag peptides. In some embodiments, the conjugate has 2 Q-tag peptides. In some embodiments, the conjugate comprises one or more, two or more, three or more, four or more, five or more, six or more, seven or more, eight or more, nine or more, ten or more, or twenty or more immunomodulating oligonucleotides. In some embodiments, the conjugate comprises one, two, three, four, five, six, seven, eight, nine, ten, or twenty immunomodulating oligonucleotides. In some embodiments, the conjugate has one immunomodulating oligonucleotide.

In another aspect, the method comprises combining a compound of Formula (C) and a SIRP-α antibody of formula (B) comprising one or more glutamine residues in the presence of a transglutaminase. In some embodiments, the method comprises contacting a compound of Formula (D) and a SIRP-α antibody of formula (B) comprising one or more glutamine residues in the presence of a transglutaminase. In some embodiments, the final concentration of the compound of Formula (C) or Formula (D) is in the range of about 1-100 μM. In some embodiments, the final concentration of the Q tag comprising antibody is in the range of about 1-500 μM. In some embodiments, the final concentration of transglutaminase is in the range of about 1-500 μM. In some embodiments, the final concentration of transglutaminase is in the range of about 1-50 μM, about 50-100 μM, about 100-150 μM, about 150-200 μM, about 200-250 μM, about 250-300 μM, about 300-400 μM, about 400-500 μM, about 100-125 μM, about 125-150 μM, about 150-175 μM, about 175-200 μM, about 200-225 μM, about 225-250 μM, about 250-275 μM, about 275-300 μM, about 300-325 μM or about 325-350 μM.

In some embodiments, the ratio of the Q tag comprising antibody and the compound of Formula (C) or Formula (D) is in the range of about 1:1-250:1, about 1:1-5:1, about 5:1-10:1, about 10:1-20:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1 or about 100:1-250:1 by weight. In some embodiments, the ratio of the compound of Formula (C) or Formula (D) and the antibody is about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1 or about 20:1 by molarity.

In some embodiments, the ratio of the Q tag comprising antibody and transglutaminase is in the range of about 1:1-500:1, about 1:1-5:1, about 5:1-10:1, about 10:1-20:1, about 20:1-30:1, about 30:1-40:1, about 40:1-50:1, about 50:1-75:1, about 75:1-100:1, about 100:1-150:1, about 150:1-200:1, about 200:1-250:1, about 1:1-25:1, about 25:1-50:1, about 50:1-75:1, about 75:1-100:1, about 100:1-150:1, about 150:1-200:1, about 200:1-250:1, about 250:1-300:1, about 300:1-400:1 or about 400:1-500:1 by weight. In some embodiments, the ratio of the peptide and transglutaminase is about 15:1, about 16:1, about 17:1, about 18:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1 or about 50:1 by molarity.

In some embodiments, the ratio of Q tag:CpG:transglutaminase is about 1:1.3:10. In some embodiments, the ratio of Q tag:CpG:transglutaminase is about 1:1.5:10. In some embodiments, the ratio of Q tag:CpG:transglutaminase is about 1:1.3:15.

In some embodiments, the reaction is incubated at greater than 15° C., greater than 20° C., greater than 25° C., greater than 30° C., greater than 35° C., greater than 40° C., greater than 45° C., or greater than 50° C. In some embodiments, the reaction is incubated at about room temperature. In some embodiments, the reaction is incubated for at least 10 minute, 20 minutes, 30 minutes, 45 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours or 60 hours.

In some embodiments, the method described herein produces the compound of Formula (A) at greater than about 5%, greater than about 10%, greater than about 15%, greater than about 20%, greater than about 25%, greater than about 30%, greater than about 35%, greater than about 40%, greater than about 45%, greater than about 50%, greater than about 60%, greater than about 70%, greater than about 80%, greater than about 90%, greater than about 95%, greater than about 97% or greater than about 99% as compared to the peptide.

In some embodiments, the pH of the reaction is in the range of about 4-10. In some embodiments, the pH of the reaction is in the range of about 4-6, about 6-8 or about 8-10. In some embodiments, the pH of the reaction is in the range of about 7-8.

In another aspect, reactions useful for attaching a linking moiety to an oligonucleotide are known in the art, including, but not limited to Hüisgen cycloaddition (metal-catalyzed or metal-free) between an azido and an alkyne-based conjugating group (e.g., optionally substituted C₆₋₁₆ heterocyclylene containing an endocyclic carbon-carbon triple bond or optionally substituted C₈₋₁₆ cycloalkynyl) to form a triazole moiety; the Diels-Alder reaction between a dienophile and a diene/hetero-diene; bond formation via pericyclic reactions such as the ene reaction; amide or thioamide bond formation; sulfonamide bond formation (e.g., with azido compounds); alcohol or phenol alkylation (e.g., Williamson alkylation), condensation reactions to form oxime, hydrazone, or semicarbazide group; conjugate addition reactions by nucleophiles (e.g., amines and thiols); disulfide bond formation; and nucleophilic substitution (e.g., by an amine, thiol, or hydroxyl nucleophile) at a carbonyl (e.g., at an activated carboxylic acid ester, such as pentafluorophenyl (PFP) ester or tetrafluorophenyl (TFP) ester) or at an electrophilic arene (e.g., SNAr at an oligofluorinated arene, a fluorobenzonitrile group, or fluoronitrobenzene group).

In certain embodiments, the attachment reaction is a dipolar cycloaddition, and the conjugation moiety includes azido, optionally substituted C₆₋₁₆ heterocyclylene containing an endocyclic carbon-carbon triple bond, or optionally substituted C₈₋₁₆ cycloalkynyl. The complementary reactive group and the conjugating group are selected for their mutual complementarity. For example, an azide is used in one of the conjugating group and the complementary reactive group, while an alkyne is used in the other of the conjugating group and the complementary reactive group.

In some embodiments, each Q-tag peptide sequence comprises the peptide sequence RPQGFGPP (SEQ ID NO:49). In some embodiments, the Ab comprises two antibody light chains, two antibody heavy chains, and two Q-tag peptides (Q) having at least one glutamine residue; wherein one Q-tag peptide is linked to the C-terminus of each of the two antibody heavy chains. In some embodiments, the conjugate has a DAR of 1 or 2. In some embodiments, the conjugate has a DAR of 1, and the method further comprises separating the conjugate having a DAR of 1 from free oligonucleotide, unconjugated antibody, and conjugates having a DAR of 2.

### Attachment of Linking Moiety to the Oligonucleotide

A linking moiety can be attached to an oligonucleotide by forming a bond between a attaching group in the oligonucleotide and a complementary reactive group bonded to the linking moiety. In certain embodiments, the linking moiety, is modified to include a complementary reactive group. Methods of introducing such complementary reactive groups into a linking moiety is known in the art.

In certain embodiments, the complementary reactive group is optionally substituted C₂₋₁₂ alkynyl, optionally substituted N-protected amino, azido, N-maleimido, S-protected thiol,

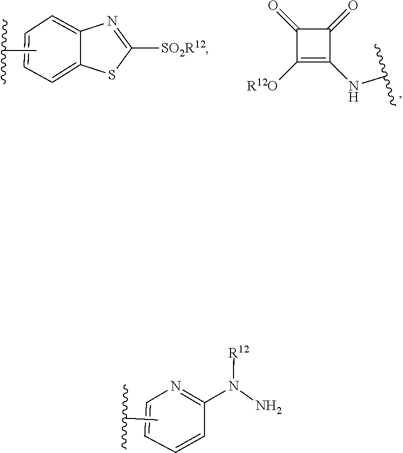

or a N-protected moiety thereof,

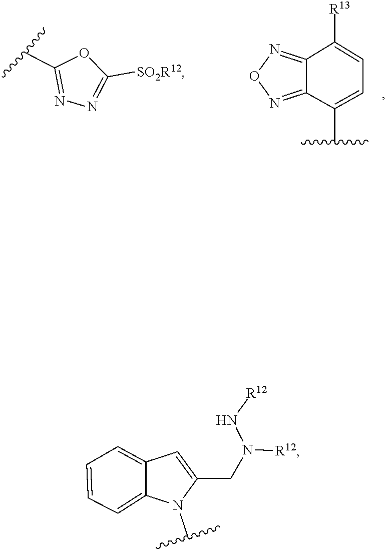

optionally substituted C₆₋₁₆ heterocyclyl containing an endocyclic carbon-carbon triple bond

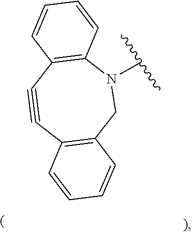 [SMILES: *N1Cc2ccccc2C#Cc2ccccc21]

1,2,4,5-tetrazine group

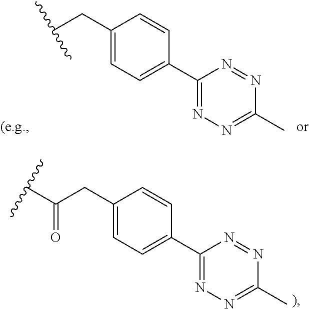 [SMILES: *Cc1ccc(-c2nnc(C)nn2)cc1 ; *C(=O)Cc1ccc(-c2nnc(C)nn2)cc1]

optionally substituted C₈₋₁₆ cycloalkynyl

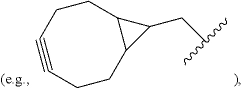 [SMILES: *CC1C2CCC#CCCC12]

—NHR^{N1}, optionally substituted C₄₋₈ strained cycloalkenyl (e.g., trans-cyclooctenyl or norbornenyl), or optionally substituted C₁₋₁₆ alkyl containing —COOR¹² or —CHO;
wherein:
   R^{N1} is H, N-protecting group, or optionally substituted C₁₋₆ alkyl;
   each R¹² is independently H, optionally substituted C₁₋₆ alkyl, or O-protecting group (e.g., a carboxyl protecting group); and
   R¹³ is halogen (e.g., F).

In certain embodiments, the complementary reactive group is protected until the conjugation reaction. For example, a complementary reactive group that is protected can include —COOR^{PGO} or —NHR^{PGN}, where R^{PGO} is an O-protecting group (e.g., a carboxyl protecting group), and R^{PGN} is an N-protecting group.

### VI. Pharmaceutical Compositions

The SIRP-α antibodies and conjugates of the present invention, such as the conjugates comprising structures of formula (A), antibodies of formula (B), and immunomodulating oligonucleotides of formulae (C), (C′), (C″), (D), (D′) and (D″), or a pharmaceutically acceptable salt of any of the foregoing, or any subgroup thereof may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets. Because of their ease in administration, tablets and capsules represent the most advantageous oral dosage unit forms, in which case solid pharmaceutical carriers are employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. The compounds of the present invention may also be administered via oral inhalation or insufflation in the form of a solution, a suspension or a dry powder using any art-known delivery system.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

The pharmaceutical compositions including a conjugate described herein can be delivered to a cell, group of cells, tumor, tissue, or subject using delivery technologies known in the art. In general, any suitable method recognized in the art for delivering a nucleic acid-protein conjugate (in vitro or in vivo) can be adapted for use with a herein described compositions. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the herein described pharmaceutical compositions may comprise one or more pharmaceutically acceptable excipients. In some embodiments, the pharmaceutical compositions described herein can be formulated for administration to a subject.

As used herein, a pharmaceutical composition or medicament includes a pharmacologically effective amount of at least one of the described therapeutic compounds or conjugates and one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The compound or conjugate can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.).

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active compound will be in the range of 25-400 mg per 1-18 weeks or 1-6 months. In some embodiments, an effective amount of an active compound will be in the range of 50-125 mg per 4 weeks or per one month. In some embodiments, an effective amount of an active ingredient will be in the range of from about 0.5 to about 3 mg/kg of body weight per dose. In some embodiments, an effective amount of an active ingredient will be in the range of from about 25-400 mg per dose. In some embodiments, an effective amount of an active ingredient will be in the range of from about 50-125 mg per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum.

For treatment of disease or for formation of a medicament or composition for treatment of a disease, the pharmaceutical compositions described herein including a SIRP-α antibody or conjugate can be combined with an excipient or with a second therapeutic agent or treatment including, but not limited to: a second or other conjugates, a small molecule drug, an antibody, an antibody fragment, and/or a vaccine.

The described SIRP-α antibodies or conjugates, when added to pharmaceutically acceptable excipients or adjuvants, can be packaged into kits, containers, packs, or dispensers. The pharmaceutical compositions described herein may be packaged in pre-filled syringes or vials.

### VII. Kits

Also provided herein is a kit comprising a conjugate as described above.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### VIII. Methods of Treatment

Also provided herein are methods for treating a disease or disorder in a subject comprising administering an effective amount of a SIRP-α antibody or conjugate described herein to the subject in need thereof. Also provided herein are uses of a SIRP-α antibody or conjugate described herein in the preparation of a medicament for treating a patient in need of treatment with the oligonucleotide in the conjugate. Also provided are SIRP-α antibodies or conjugates as described herein for treating a disease or disorder in a subject in need of the treatment with the oligonucleotide in the SIRP-α antibodies or conjugates. Also provided are SIRP-α antibodies or conjugates as described herein for treating a patient comprising administering an effective amount of the SIRP-α antibody or conjugate to the patient. In some embodiments, the subject has or at the risk of developing cancer. In some embodiments, the disease or disorder is a viral infection. In some embodiments, the disease or disorder is an immunodeficiency, e.g., in which immune activation may be favorable. In some embodiments, the disease or disorder is an autoimmune and/or inflammatory disease or disorder, e.g., in which immune suppression and/or modulation may be favorable.

In some embodiments of the methods of treating cancer as described herein, the cancer being treated with the methods disclosed herein is a solid tumor. In some embodiments, the cancer being treated with the methods disclosed herein is a liquid tumor. In some embodiments, the cancer being treated with the methods disclosed herein is a solid tumor. In particular embodiments, the cancer being treated with the methods disclosed herein is breast cancer, colorectal cancer, lung cancer, head and neck cancer, melanoma, lymphoma, bile duct cancer (cholangiocarcinoma), or leukemia. In some embodiments, cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal qammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, bile duct cancer (cholangiocarcinoma), small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, cholangiocarcinoma (e.g., intrahepatic cholangiocarcinoma), squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, bile duct cancer (cholangiocarcinoma), ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated. In particular embodiments, the cancer being treated with the methods disclosed herein is selected from the list consisting of mantle cell cymphoma (MCL), diffuse large B-cell lymphoma (DLBCL), Burkitts lymphoma, multiple melanoma (MM), chronic lymphocytic leukemia (CLL), acute myeloid leukemia (AML), small lymphocytic lymphoma (SLL), hairy cell leukemia (HCL), lymphoplasmacytic lymphoma (LPL), skeletal muscle lymphoma (SML), splenic marginal zone lymphoma (SMZL), follicle center lymphoma (FCL), colorectal cancer, non-small cell lung cancer (NSCLC), head and neck cancer, breast cancer, pancreatic cancer, glioblastoma (GBM), prostate cancer, esophageal cancer, renal cell carcinoma, hepatic carcinoma, bladder cancer and gastric carcinoma. In some embodiments, the cancer is lung cancer, cholangiocarcinoma (e.g., intrahepatic cholangiocarcinoma), squamous cell cancer, brain tumors, glioblastoma, head and neck cancer, hepatocellular cancer, colorectal cancer, skin cancer, lung cancer, endometrial cancer, liver cancer, bladder cancer, gastric or stomach cancer, pancreatic cancer, cervical cancer, ovarian cancer, cancer of the urinary tract, urothelial cancer, breast cancer, peritoneal cancer, uterine cancer, salivary gland cancer, kidney or renal cancer, prostate cancer, vulval cancer, thyroid cancer, anal carcinoma, penile carcinoma, testis or testicular cancer, melanoma, multiple myeloma and B-cell lymphoma, non-Hodgkin's lymphoma (NHL), acute lymphoblastic leukemia (ALL), chronic lymphocytic leukemia (CLL), acute myeloid leukemia (AML), Merkel cell carcinoma, hairy cell leukemia, or chronic myeloblastic leukemia (CML), including metastases thereof. In some embodiments, the cancer is melanoma or renal cancer. In some embodiments, the cancer is a melanoma or renal cancer that expresses or overexpresses SIRP-α.

In some embodiments, the cancer or tumor is known or predicted to be non-responsive to an inhibitor of PD-L1 or PD-1 (e.g., when administered as a monotherapy, or when administered in the absence of an anti-SIRP-α antibody). In some embodiments, the individual to be treated by the methods of the present disclosure is known or predicted to be non-responsive to an inhibitor of PD-L1 or PD-1 (e.g., when administered as a monotherapy, or when administered in the absence of an anti-SIRP-α antibody). In some embodiments, the individual does not achieve a significant therapeutic response to an inhibitor of PD-L1 or PD-1 (e.g., prior to administration of a conjugate or composition of the present disclosure). In some embodiments, prior to administration of a conjugate or composition of the present disclosure, the individual has been treated with an inhibitor of PD-L1 or PD-1. In some embodiments, prior to administration of the conjugate or composition of the present disclosure, the individual has been treated with an inhibitor of PD-L1 or PD-1 and did not respond to said treatment with the inhibitor of PD-L1 or PD-1 (e.g., when administered as a monotherapy, or when administered in the absence of an anti-SIRP-α antibody). In some embodiments, the inhibitor of PD-L1 or PD-1 is an antibody that binds PD-L1 or PD-1. In some embodiments, the inhibitor of PD-L1 or PD-1 is pembrolizumab (KEYTRUDA®; Merck), nivolumab (OPDIVO®; Bristol Myers Squibb), cemiplimab-rwlc (LIBTAYO®; Regeneron/Sanofi), atezolizumab (TECENTRIQ®; Genentech), dostarlimab-gxly (JEMPERLI®; GlaxoSmithKline), durvalumab (IMFINZI®; AstraZeneca), or avelumab (BAVENCIO®; EMD Serono/Pfizer).

In some embodiments, cells of the cancer or tumor express or overexpress human SIRP-α (e.g., on their cell surface), i.e., a SIRP-α-positive cancer or tumor. In some embodiments, cells of the cancer or tumor do not express or overexpress human SIRP-α (e.g., on their cell surface), i.e., a SIRP-α-negative cancer or tumor. In some embodiments, cells of the cancer or tumor express or overexpress human CD47 (e.g., on their cell surface).

In some embodiments, the methods for treating cancer disclosed herein further comprise administering an additional therapeutic agent, e.g., in combination with a conjugate of the present disclosure. In some embodiments, the additional therapeutic agent comprises an immunotherapy (including but not limited to an immune checkpoint inhibitor such as an anti-PD1, anti-PD-L1, or anti-CTLA4 antibody), chemotherapy, radiation therapy, cell-based therapy, anti-cancer vaccine, or anti-cancer agent (including but not limited to a therapeutic antibody or other biologic, or a small molecule inhibitor).

In some embodiments, the methods for treating cancer disclosed herein further comprise administering an inhibitor of PD-L1 or PD-1, e.g., in combination with a conjugate of the present disclosure. In some embodiments, the inhibitor of PD-L1 or PD-1 is an antibody, e.g., an anti-PD-L1 antibody or anti-PD-1 antibody. In some embodiments, the inhibitor of PD-L1 or PD-1 is a peptide or small molecule inhibitor. Suitable examples of PD-L1 inhibitors are known in the art and include, without limitation, atezolizumab (TECENTRIQ®; Genentech), avelumab (BAVENCIO®; EMD Serono), durvalumab (IMFINZI®; AstraZeneca), KN035, CK-301, AUNP12, CA-170, and BMS-986189. Suitable examples of PD-1 inhibitors are known in the art and include, without limitation, pembrolizumab (KEYTRUDA®; Merck), nivolumab (OPDIVO®; Bristol Myers Squibb), cemiplimab-rwlc (LIBTAYO®; Regeneron/Sanofi), dostarlimab-gxly (JEMPERLI®; GlaxoSmithKline), JTX-4014, spartalizumab (PDR001), camrelizumab (SHR1210), sintilimab (IBI308), tislelizumab (BGB-A317), toripalimab (JS 001), INCMGA00012, AMP-224, and AMP-514.

In some embodiments, provided herein methods for treating a disease or disorder in a subject comprising administering an effective amount of an immunoconjugate described herein to the subject in need thereof, wherein the immunoconjugate binds to SIRP-α, such as a CpG oligonucleotide-antibody immunoconjugate comprising a SIRP-α antibody or antigen binding fragment thereof, and where the disease or disorder treated is a cancer characterized by SIRP-α overexpression. In some embodiments, such cancers include breast cancer, ovarian cancer, lung cancer, pancreatic adenocarcinoma, colon carcinoma, hepatocellular carcinoma, bladder cancer, and gallbladder cancer. In some embodiments, the immunoconjugate comprises an oligonucleotide listed in one of Tables 2 and 14-17.

In some embodiments, the methods of treatment include administration of a CpG-Ab immunoconjugate that binds to SIRP-α present on a myeloid cell and/or monocyte and the treatment results in the killing of or impairment of tumor cell(s) such that the volume, size and/or growth of the tumor is reduced or inhibited. In some embodiments, the methods of treatment include administration of a CpG-Ab immunoconjugate that binds to SIRP-α present on a tumor cell and the treatment results in the killing of or impairment of tumor cell(s) such that the volume, size and/or growth of the tumor is reduced or inhibited. In some embodiments, provided herein are methods for treating a disease or disorder in a subject comprising administering an effective amount of a CpG-Ab immunoconjugate described herein to the subject in need thereof, wherein the CpG-Ab immunoconjugate binds to SIRP-α, such as a CpG-Ab immunoconjugate comprising a SIRP-α antibody or antigen binding fragment thereof, and where the disease or disorder treated is a cancer characterized by SIRP-α-expressing tumor cells. In some embodiments, the disease or disorder treated is a cancer where tumor cells express SIRP-α. In some embodiments, provided herein are methods for treating a disease or disorder in a subject comprising administering an effective amount of a CpG-Ab immunoconjugate described herein to the subject in need thereof, wherein the CpG-Ab immunoconjugate binds to SIRP-α, such as a CpG-Ab immunoconjugate comprising a SIRP-α antibody or antigen binding fragment thereof, and where the disease or disorder treated is a cancer characterized by tumor cells that do not express SIRP-α.

In some embodiments, the cancer being treated with the methods disclosed herein is resistant to at least one immunotherapy. In some embodiments, the cancer being treated with the methods disclosed herein is resistant to at least one cancer therapy selected from the group consisting of chemotherapy, radiation, targeted therapy, vaccine therapy, and CAR-T therapy. In some embodiments, the method of treating cancer comprises co-administering to a subject having cancer (i) a therapeutically effective amount of the CpG-containing immunostimulating oligonucleotide or the CpG-antibody immunoconjugate; and (ii) the immunotherapeutic agent which the cancer being treated has shown to resist or not to respond, when the cancer is treated with the immunotherapeutic agent alone. In some embodiments, the treatment comprises treatment with an immunoconjugate of the present disclosure in combination with a therapeutic antibody, small molecule cancer treatment, cellular therapy, CAR-T, chemotherapy, radiation therapy, etc.

In particular embodiments, the cancer being treated with the methods provided herein has been shown to not to respond to a treatment with an immune checkpoint modulator. In particular embodiments, the immune checkpoint modulator is an inhibitor of PD-1. In particular embodiments, the immune checkpoint modulator is an inhibitor of PD-L1. In some embodiments, the method of treating cancer comprises co-administering to a subject having cancer (i) a therapeutically effective amount of the CpG-containing immunostimulating oligonucleotide or the CpG-Ab immunoconjugate; and (ii) a therapeutically effective amount of the inhibitor of PD-1. In some embodiments, the method of treating cancer comprises co-administering to a subject having cancer (i) a therapeutically effective amount of the CpG-containing immunostimulating oligonucleotide or the CpG-Ab immunoconjugate; and (ii) a therapeutically effective amount of the inhibitor of PD-L1. In particular, in some embodiments, the inhibitor of PD-1 is an anti-PD-1 antibody or an antigen-binding fragment thereof. In some embodiments, the inhibitor of PD-L1 is an anti-PD-L1 antibody or an antigen-binding fragment thereof. In some embodiments, the treatment is directed to a subject that does not respond to or is resistant to a PD-1 or PD-L1 inhibitor and such subject is treated with a CpG-Ab immunoconjugate that binds SIRP-α, such as a CpG-Ab immunoconjugate comprising an anti-SIRP-α antibody or antigen binding fragment thereof.

In some embodiments, the cancer being prevented or treated using the methods provided herein is an episode of cancer recurrence in a subject who is in partial or complete remission of a prior cancer. In particular embodiments, the prior cancer is a liquid cancer and the recurrent cancer being prevented or treated is a liquid cancer. In particular embodiments, the prior cancer is a solid cancer and the recurrent cancer being prevented or treated is a solid cancer. In particular embodiments, the prior cancer is a liquid cancer and the recurrent cancer being prevented or treated is a solid cancer. In particular embodiments, the prior cancer is a solid cancer and the recurrent cancer being prevented or treated is a liquid cancer.

In some embodiments, the cancer being prevented or treated using the methods provided herein is first episode of cancer recurrence in the subject after the subject showed partial or complete remission. In some embodiments, the cancer being prevented or treated using the methods provided herein is second episode of cancer recurrence in the subject after the subject showed partial or complete remission. In some embodiments, the cancer being prevented or treated using the methods provided herein is third episode of cancer recurrence in the subject after the subject showed partial or complete remission. In some embodiments, the cancer being prevented or treated using the methods provided herein is an episode of cancer recurrence subsequent to the third episode of cancer recurrence in the subject after the subject showed partial or complete remission.

In some embodiments of the methods and uses described herein, wherein upon administration of the CpG-SIRP-α-Ab immunoconjugate, the CpG-containing immunostimulating oligonucleotide specifically binds to a TLR9 receptor of the targeted cell.

### EXAMPLES

The presently disclosed subject matter will be better understood by reference to the following Examples, which are provided as exemplary of the invention, and not by way of limitation.

### Materials

Prototype peptides were made in house, but can be purchased at custom peptide suppliers (e.g CPC Scientific). Oligonucleotides were made in-house or by LGC. Transglutaminase used in these examples were isolated from various bacterial Streptoverticillium strain (e.g., Ajinomoto). The Q-tag mAbs were produced at Sino Biologicals or internally.

### Production of Oligonucleotides

Oligonucleotides were generally prepared in accordance with the solid phase synthesis scheme shown below, beginning with an initial deprotection of the solid support for the oligonucleotide synthesis, followed by coupling of the solid support with to the first nucleotide, thiolation to give the phosphothioester and repeated deprotection and coupling to give the entire oligonucleotide sequence.

The general synthesis of oligonucleotides as provided herein is described below.

Deprotection: A dimethoxytrityl-1,3-propanediol glycolate protected controlled pore glass solid support (DMTO-C3-CPG, 1000 Å, Bulk Density 0.26-0.36 g/cc, Loading 30-40 μmol/g) was reacted with 3% dichloroacetic acid in toluene (v/v) at 25° C., to give the deprotected CPG support. UV absorption of an aliquot of the reaction mixture was measured to identify the reaction endpoint (wavelength 350 nm, target minimum absorbance 0.25 OD, using a fixed watch command setting) and to confirm removal of the dimethoxytrityl protecting group.

Activation Coupling: The deprotected CPG support was coupled with the first nucleotide phosphoramidite precursor for the 3′-end, for the respective oligonucleotide to be synthesized, by adding and mixing the desired 3′ nucleotide (3 equiv.) for 5 minutes at 25° C. to the reactor containing the deprotected CPG support in the presence of an activator 5-Ethylthio-1H-tetrazole (0.5M in ACN) at 60% of the nucleotide concentration.

Thiolation Sulfurization: Following the coupling step, the linking phosphite triester moiety of the added nucleotide precursor was thiolated (or sulfurized) by adding Polyorg Sulfa (3-phenyl 1,2,4-dithiazoline-5-one), 0.15M in dry ACN, to give the phosphothioester.

Capping: After sulfurization, the CPG support and linked nucleotide were treated with two capping compositions (Capping composition A: 20% N-methylimidazole in ACN; Capping B composition B: 20% Acetic Anhydride, 30% Pyridine, 50% ACN) to block unreacted nucleotide reactants.

Repeat Synthesis: The remaining nucleotides were added in sequence from the 3′ end to the 5′ end, employing the appropriate phosphoramidite precursors in solution, by repeating the steps of deprotection, activation/coupling, thiolation/sulfurization and capping as described above to obtain the desired oligonucleotide sequence in protected form. All phosphoramidite prescursors were mixed with the CPG support for 5 minutes during the coupling step, except for dT-Thiophosphoramidite, which was mixed for 15 minutes.

Selected phosphoramidite precursors used in the synthesis are shown below. The phosphoramidite precursors were prepared in solutions with the solvents and at the concentrations, respectively shown, to be used in the coupling steps.

| Amidite | Structure | Concentration |
|---|---|---|
| DMT-dC(Ac) Amidite | 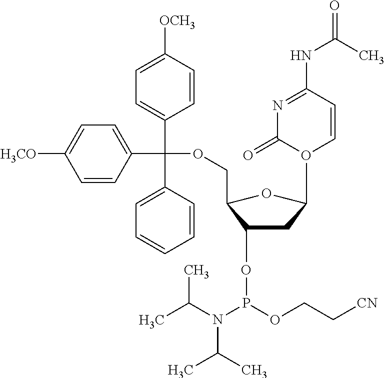 | 0.1M in dry ACN: |
| DMT-dG(dmf) Amidite | 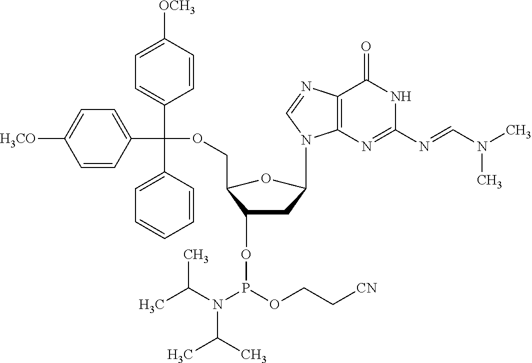 | 0.1M in dry ACN: |
| DMT-dT phosphoramidite | 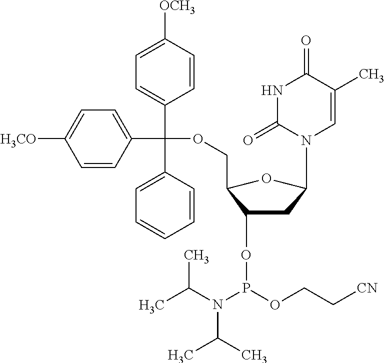 | 0.1M in dry ACN: |
| Fmoc-protected DMT-dT PEG2 NH2 Amidite | 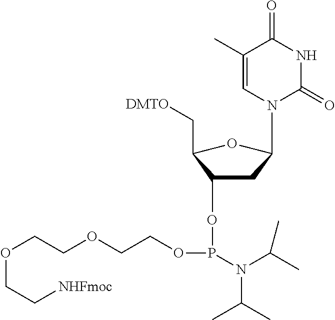 | 0.1M in dry ACN |
| 5-Br-dU-CE Phosphoramidite | 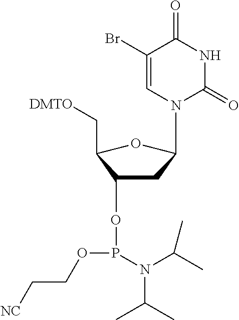 | 0.1M in dry ACN |
| dT- Thiophosphoramidite | 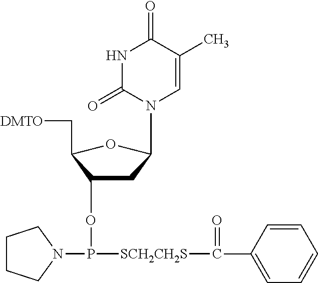 | 0.15M in dry 10% (v/v) DCM/ACN |
| 2′-O-Methyl 5- Methyl Uridine (CED phosphoramidite | 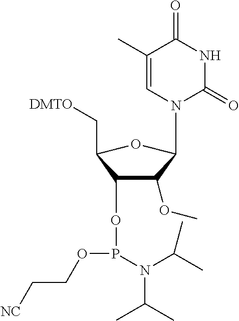 | 0.1M in dry ACN |
| dG- Thiophosphoramidite | 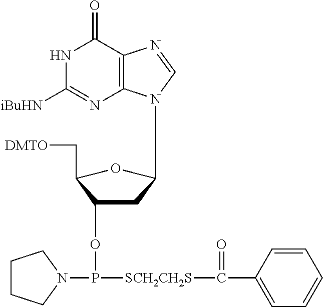 | 0.1M in dry ACN |

Exemplary Fmoc-protected oligonucleotide compounds 6.1a, 6.2a and 6.3a obtained from the synthesis steps described above are shown below. The deprotection, purification and coupling of compound 6.1a to prepare the compound 6.1b is further described below.

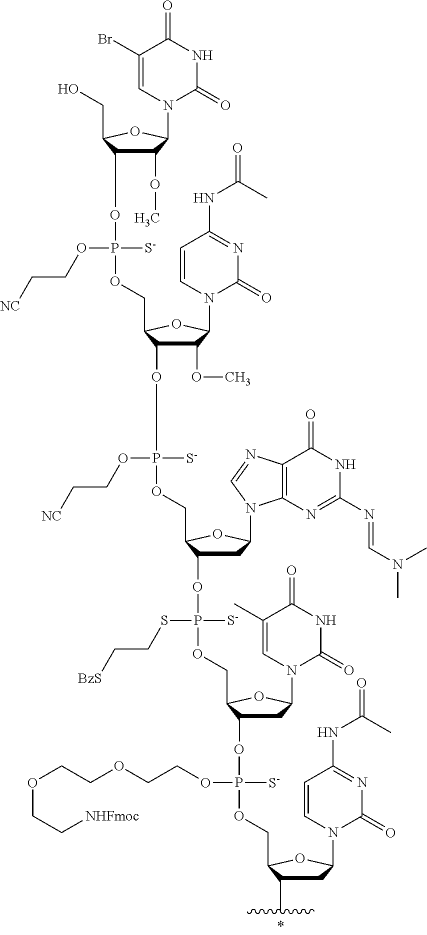

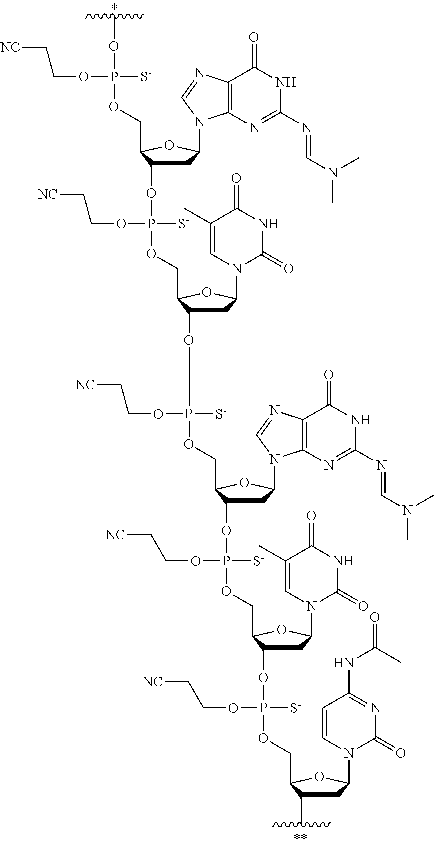

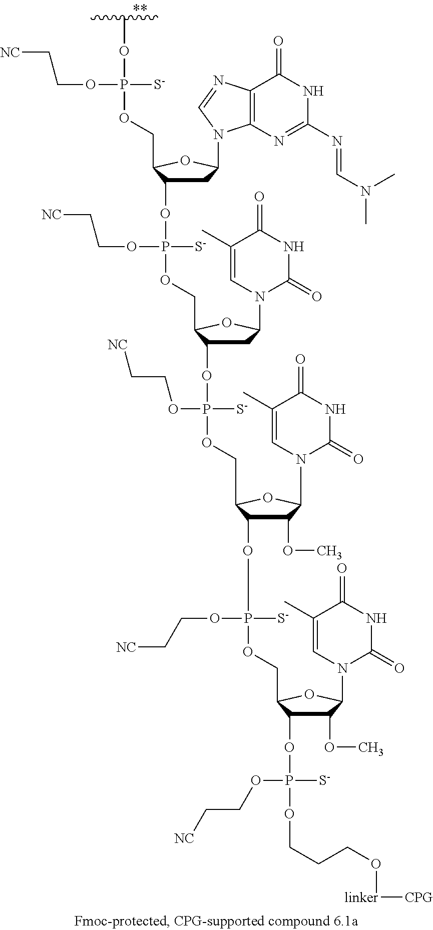

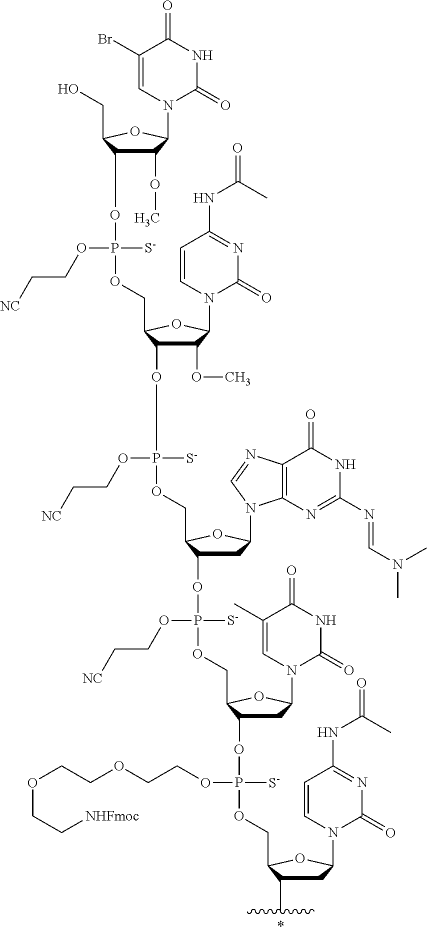

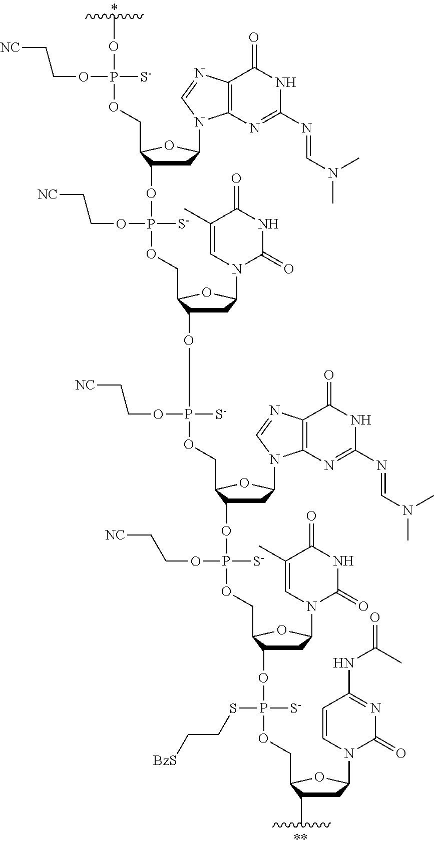

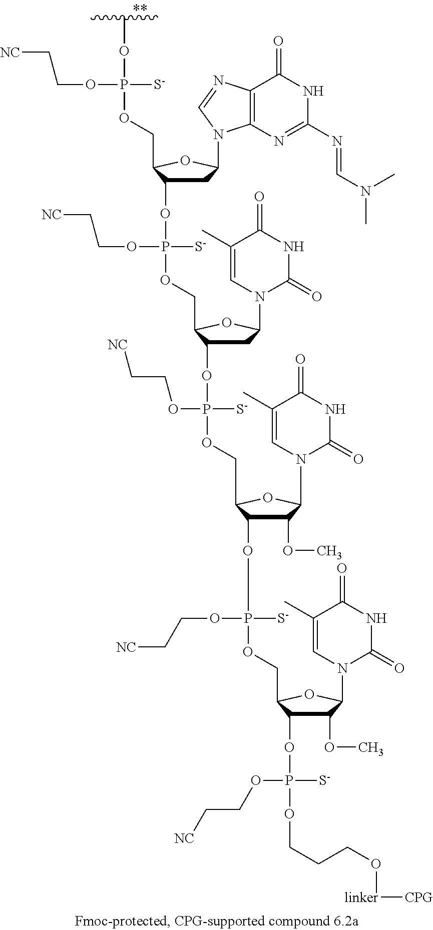

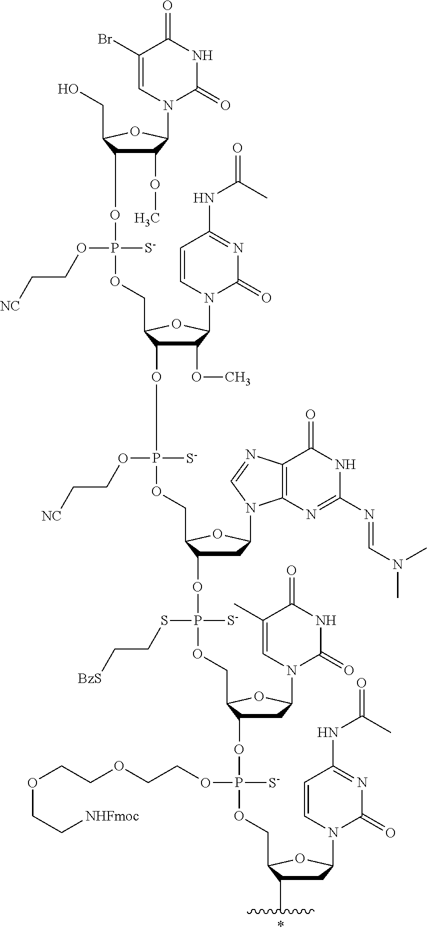

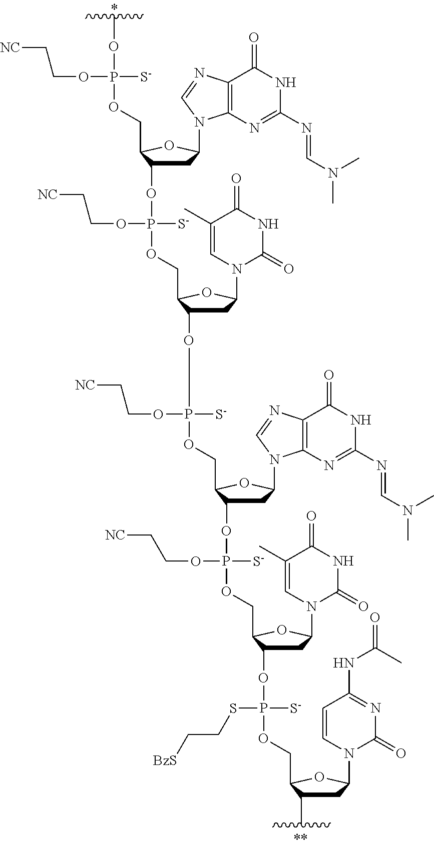

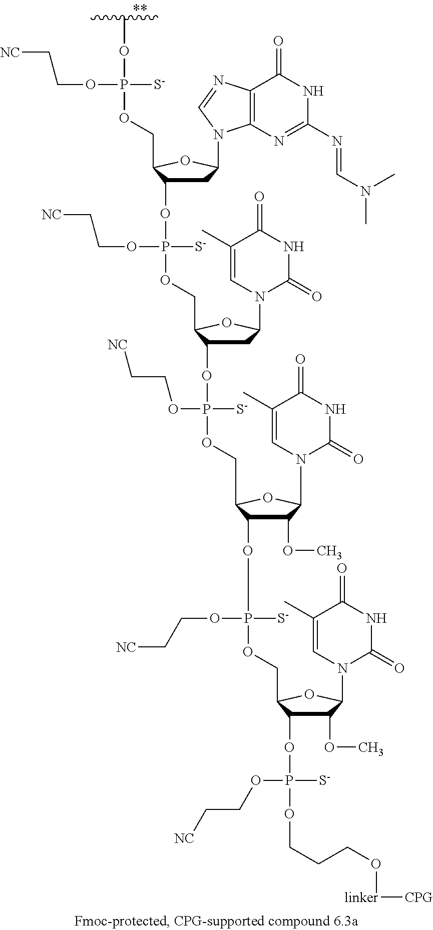

The Fmoc-protected, CPG-supported oligonucleotide compound 6.1a obtained from the synthesis above was simultaneously cleaved from the support and deprotected by reacting the CPG support with 20 mM dithiothreitol in ammonium hydroxide:methylamine, 1:1 (v/v) for 2 hours at room temperature to give crude compound 6.1a. The crude product was purified by ion-pair reversed phase HPLC (IP—RP-HPLC) and its identity confirmed by ESI-MS. Crude compound 6.1a was purified by HPLC and desalted.

Compound 6.1a was subsequently reacted with O-[2-(Fmoc-amino)-ethyl]-O′-[3-(N-succinimidyloxy)-3-oxopropyl]polyethylene glycol (Fmoc-N-amido-dPEG₂₄-NHS ester) in sodium bicarbonate buffer to give Fmoc-protected compound 6.1b. Fmoc-protected compound 6.1b was reacted with 20 mM dithiothreitol in ammonium hydroxide:methylamine, 1:1 (v/v) for 2 hours at room temperature to give crude compound 6.1b. The crude product was purified by ion-pair reversed phase HPLC (IP—RP-HPLC) and its identity confirmed by ESI-MS. Crude compound 6.1b was purified by HPLC, desalted, and lyophilized to give the purified oligonucleotide 6.1b.

### Production of Antibodies

Antibodies generated in-house are typically expressed in suspension culture of Expi293 system (ThermoFisher) according to the manufacturer's manual. The expressed antibodies are purified via Protein A capture using MabSelectLX chromatography (GE), elution with 0.1M citrate (pH 3.3) and dialyzed in final buffer composition of 1×PBS (Phosphate Buffered Saline, pH 7.4).

### One-Step Conjugation Method Via mTG (Microbial Transglutaminase)

Q-tag with the sequence RPQGFGPP (SEQ ID NO: 49) was genetically linked to the C-terminus of the heavy chain of antibody. To perform conjugation, the purified antibody (containing the engineered Q tags at the C-terminal of heavy chain) were first buffer exchanged into 25 mM Tris, 150 mM NaCl pH 8. The Ab-Q-tag containing moiety and CpG were added in molar ratio of 1:1.3 and incubated overnight with a final concentration of 1% mTG (w/v) (Ajinomoto) at room temperature. Final concentration of antibody used for conjugation is generally ˜20-25 μM. Mixture was loaded to a Q Sepharose HP (GE) equilibrated in 20% Buffer B (40 mM Tris, 2M NaCl pH8) and 80% Buffer A (40 mM Tris, pH8). Column was washed with 5 column volumes of 20% Buffer B. Separation was achieved with using a linear gradient from 20% B to 60% B in 30 column volumes. DAR1 peak fractions (Q tag conjugated with one CpG moiety) were pooled and concentrated followed by a gel filtration step using S200 (GE). Monomeric peak fractions were pooled and concentrated.

### Biological Evaluation of CpG-Nucleotides and Antibody-CpG Nucleotide Conjugates

Trima residuals were received from Vitalant and diluted 1:4 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface and resuspended in FACS buffer (PBS with 0.5% Bovine Serum Albumin (Gibco)). B cells were purified by negative selection using the B Cell Isolation Kit II, human (Miltenyi Biotec) and LS columns (Miltenyi Biotec) according to manufacturer's protocol.

PBMCs were immediately plated onto a 96-well format (500K/well) in Complete RPMI (RPMI+10% FBS). Five-fold serial dilutions were added to the cells from 100 nM to 6.4 pM of antibody and conjugated antibody and 1 uM to 64 pM of CpG oligonucleotides at 37° C. under 5% CO₂ for 48 to 96 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD19, anti-CD20, anti-CD40, anti-HLADR and anti-CD80 for B cell assays and anti-CD14, anti-CD3, anti-CD19, anti-CD14, anti-CD123, anti-CD11c and anti-CD86 for pDC assays. Cells were centrifuged and washed twice in FACS buffer and fixed in 0.5% paraformaldehyde. CountBright™ Absolute Counting Beads (Thermo Fisher) were added to each well to count the number of cells. Cells were analyzed on Attune NxT Flow Cytometer (Thermo Fisher), with subsequent data analysis by Flowjo 10.7 (Treestar). Dead cells were excluded by gating on the eFluor 780-negative population. Lineage specific cells were first excluded (CD19, CD3, CD14) prior to gating CD123⁺CD11c⁻ cells to identify pDC and gating CD19⁺, CD20⁺ or CD19⁺CD20⁺ cells to identify B cells.

### Example 1: Activities of Free Immunomodulating Oligonucleotides (CpGs) in Human PBMCs

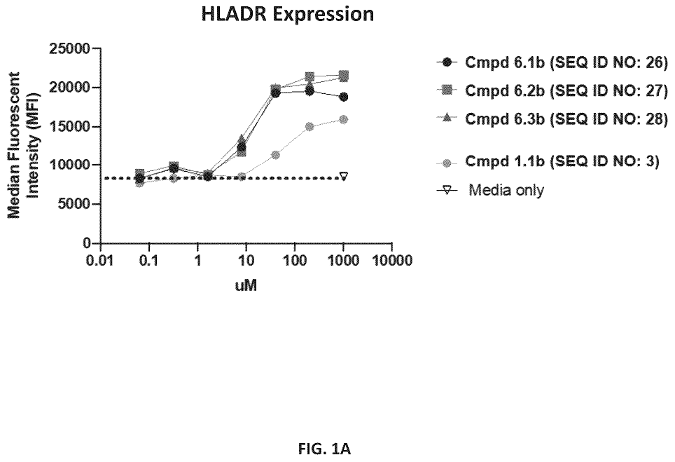

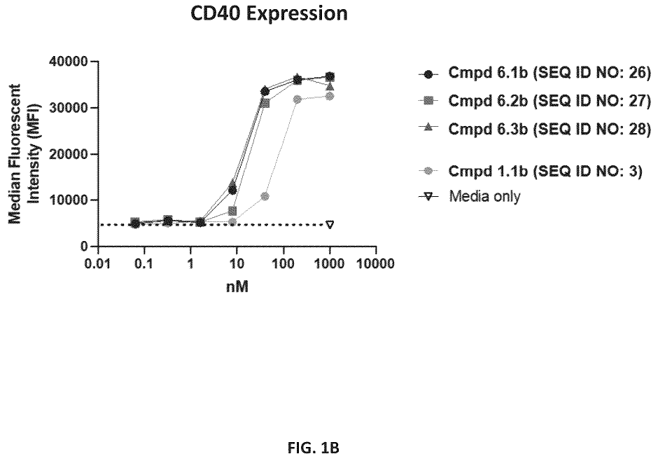

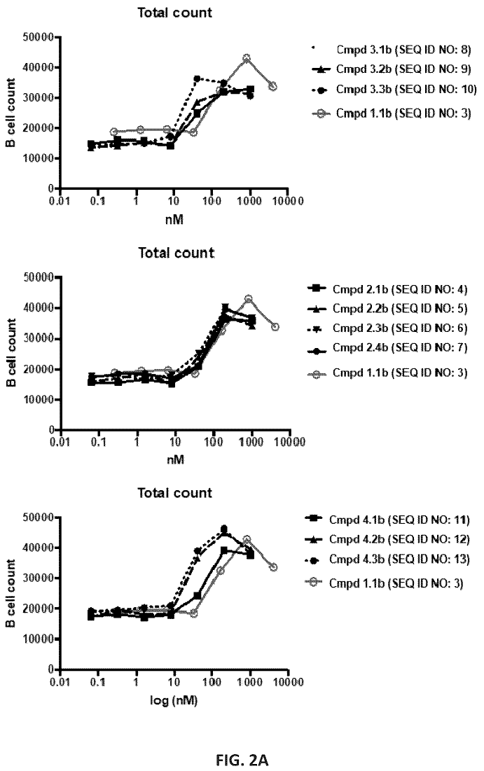

Human PBMCs were treated with free CpGs (SEQ ID NOs: 3 and 26-28) to evaluate their respective activities as observed by HLADR and CD40 expression on CD19 positive B cells (as shown in FIGS. 1A-1B). CpGs (SEQ ID NO: 26-28) all showed enhanced activities compared with 12070 CpG (SEQ ID NO: 3).

### Example 2: Activities of Immunomodulating Oligonucleotides and their Respective Antibody Conjugates

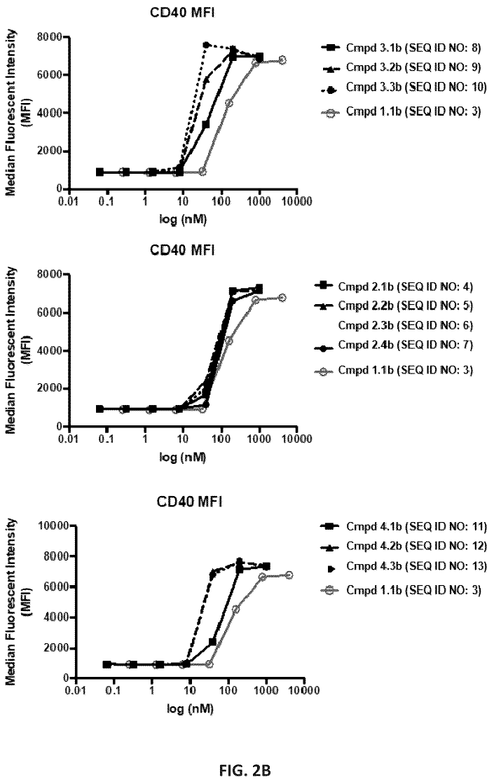

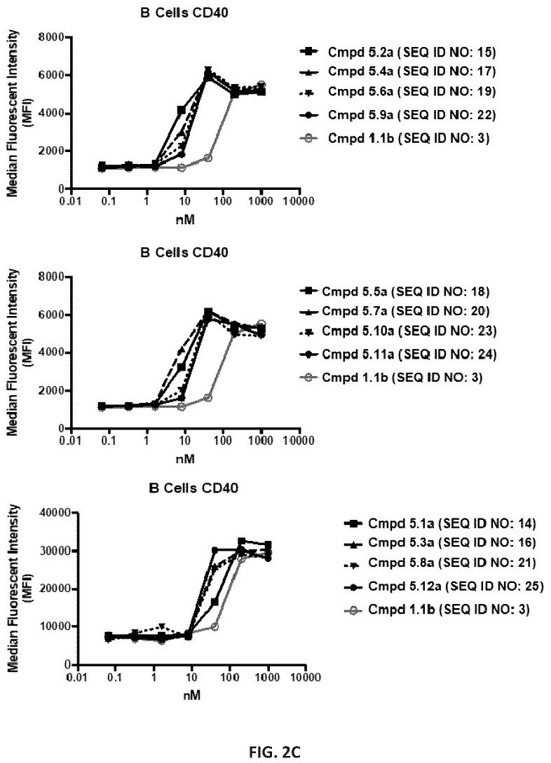

Various CpG oligonucleotides, SEQ ID NO: 3-25, were tested for their effects on proliferation and/or activation of B cells. FIGS. 2A-2C show the respective activities of select CpGs alone. All CpG oligonucleotides tested enhanced the activation of B cells after 48 hours of incubation. As determined by counting beads to calculate absolute B cell number and CD40 expression, all CpGs increased the number of B cells and CD40 expression. A select number of CpG oligonucleotides tested showed enhanced effects on B-cell proliferation and activation compared with CpG (SEQ ID NO: 3).

### Example 3: Transglutaminase-Mediated Conjugation

The transglutaminase-mediated conjugation was tested using an oligonucleotide A (with the sequence: tucgtcgtgacgtt, SEQ ID NO: 1) coordinated to a PEGylated linker (—NH—C(═O)—PEG₂₃-NH₂, structure shown below), and Q-tag peptides sequences SEQ ID NOs: 39-47 and 50-52.

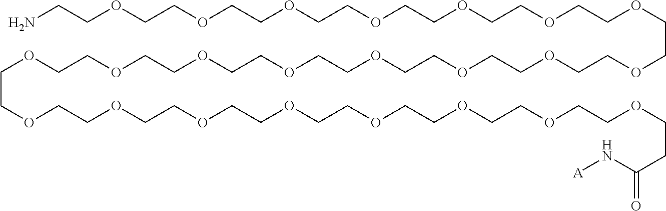

2 nmol of the Q-tag was added to 1 nmol of the linker in the present of 0.04 nmol of transglutaminase in PBS. The final concentration of linker is 50 μM. Reactions were kept at room temperature and quenched with 8 M formamide at 1 hour. The reaction solution was analyzed using reverse-phase HPLC with Xbridge C18 column (4.6×150 mm) using solvent A (50 mM TEAA in water) and solvent B (Acetonitrile) with a gradient of 20% to 60% of solvent B in 10 minutes at 60° C. Alternatively, the reaction solution was analyzed using reverse-phase HPLC with Luna 3μ C18 column (4.6×50 mm) using solvent A (0.1% TFA in water) and solvent B (0.1% TFA in Acetonitrile) with a gradient of 10% to 70% of solvent B in 10 minutes at 50° C.

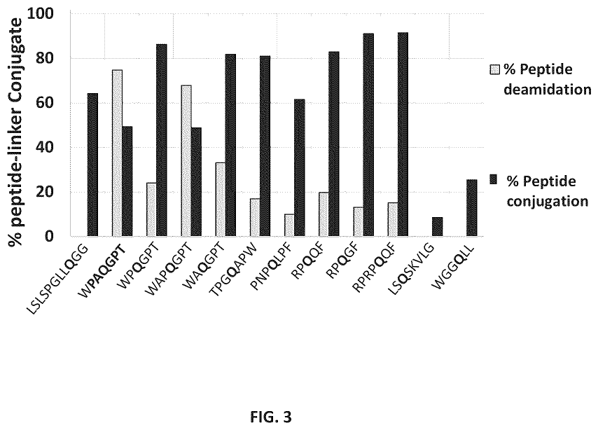

FIG. 3 shows the yields of the transglutaminase-mediated conjugation and peptide deamidation with various Q-tags. RPQGF (SEQ ID NO:47), RPQQF (SEQ ID NO:46), RPRPQQF (SEQ ID NO:50) showed high conjugate percentage and moderately low deamidation

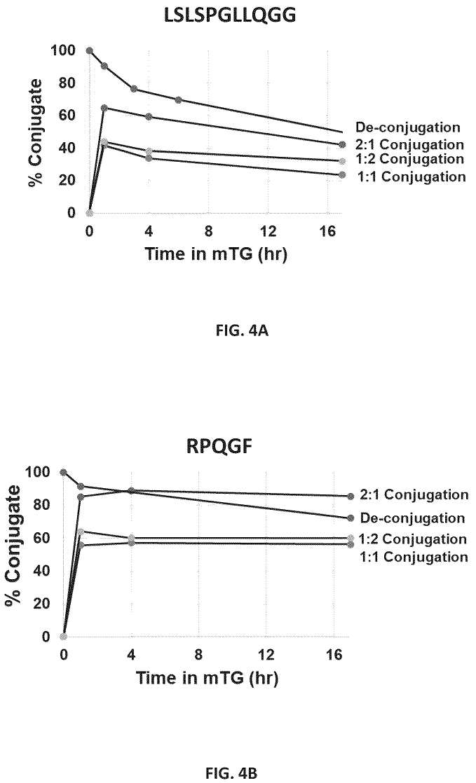

FIGS. 4A-4B show the conjugation and deconjugation of two conjugates prepared from Q-tag with SEQ ID NOs: 39 and 47 over time. RPQGF (SEQ ID NO:47) has higher percentage of conjugation with all Q-tag:linker+CpG ratio tested, over a duration of 16 hrs. Moreover, the deconjugation rate of RPQGF (SEQ ID NO:47) is also slower compared with LSLSPGLLQGG (SEQ ID NO:39).

### Example 4: Evaluation of Free CpG Activity on CD40 Expression by CD19+ B Cells

### Materials and Methods

Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin (Gibco)). After one wash, PBMCs were resuspended in Complete RPMI (RPMI+10% FBS).

PBMCs were immediately plated onto a 96-well format (500K/well) in Complete RPMI. Five-fold serial dilutions were added to the cells from 1 uM to 64 pM of CpG oligonucleotides at 37° C. under 5% CO₂ for 48 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD19, anti-CD20, anti-CD40, anti-HLADR and anti-CD80. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. B cells were identified as CD19+CD20+ cells and level of activation marker was assessed by median fluorescent intensity.

For Ramos NFkb Reporter Assay, Ramos-Blue Cells NF-kB/AP-1 Reporter B lymphocytes were purchased from Invivogen. Cells were grown and maintained in complete DMEM supplemented with 2 mM L-glutamine, 10% FBS, 100 ug/mL Normacin, Pen-Strep, 100 ug/mL Zeocin. Stimulation of the Ramos-Blue cells was performed. Briefly, cells were rinsed in growth medium without antibiotics. Cells were counted and resuspended in fresh complete DMEM without selection antibiotics at a density of 2×10⁶ cell/mL. 20 uL of 10 uM CpG 7-7, CpG 12070 and ODN2006 titrated 1:5 were added to a flat-bottom 96-well plate, 180 uL of the cell suspension were added to a final concentration of 1 uM to 64 pM of CpG. Plate was incubated at 37° C. in a 5% CO₂ incubator for 24 h. On day of assay, QB reagent and QB buffer were thawed before us. Quanti-Blue solution was prepared by adding 1 mL of QB reagent and 1 mL of 1 mL of QB buffer to 98 mL of sterile water in a sterile glass bottle. 180 μL of Quanti-Blue solution was dispensed per well into a new flat-bottom 96-well plate. 20 μL of supernatant from treated Ramos-Blue cells was then added to the 96-well plate. Plate was ten incubated for 6 h. Optical density was measured at OD655 using a plate reader (Molecular Devices), and data was tabulated in GraphPad Prism 9.0.

### Results

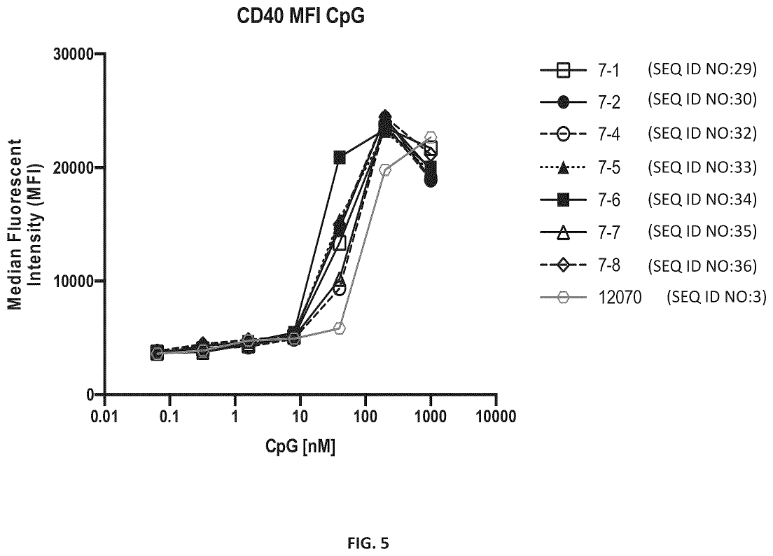

Human PBMCs were treated with free CpGs to evaluate their respective activities as observed by CD40 expression on CD19 positive B cells. As shown in FIG. 5, series 7 CpGs (SEQ ID NOS: 29, 30, and 32-36) all showed enhanced activities compared with CpG 12070 (SEQ ID NO: 3).

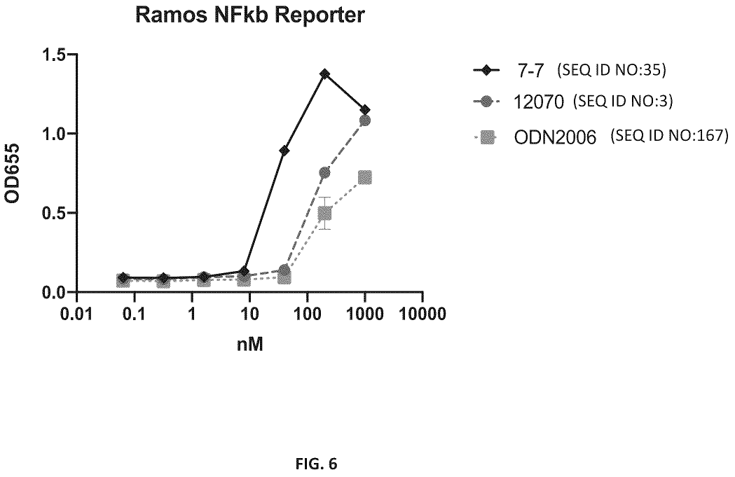

CpG oligos 7-7, 12070 and ODN2006 (5′-tcgtcgttttgtcgttttgtcgtt-3′; SEQ ID NO:167) were compared in a NFkb reporter assay. As shown in FIG. 6, CpG 7-7 showed significantly higher activity as compared to 12070 and ODN2006.

### Example 5: Evaluation of CpG Activity on PBMCs from Different Donors

The activity of CpG oligos 7-6, 7-7 and 12070 were compared for activity in PBMC cells from three different donor lines (D559, D804 and D643) as observed by CD40 expression. The evaluation of activity of the CpG oligos was performed using the same methods as Example 16 above.

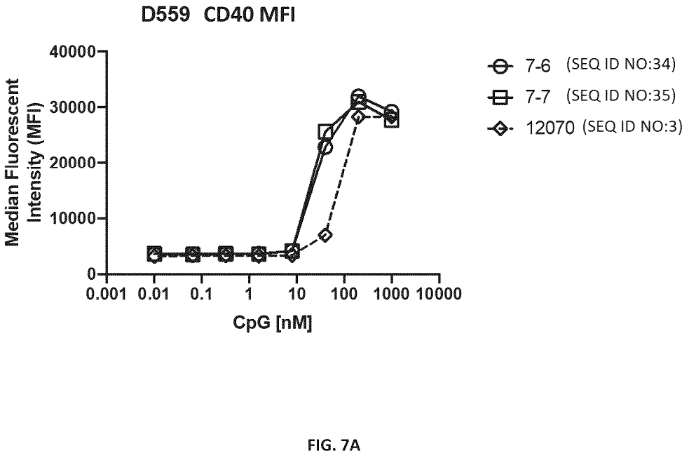

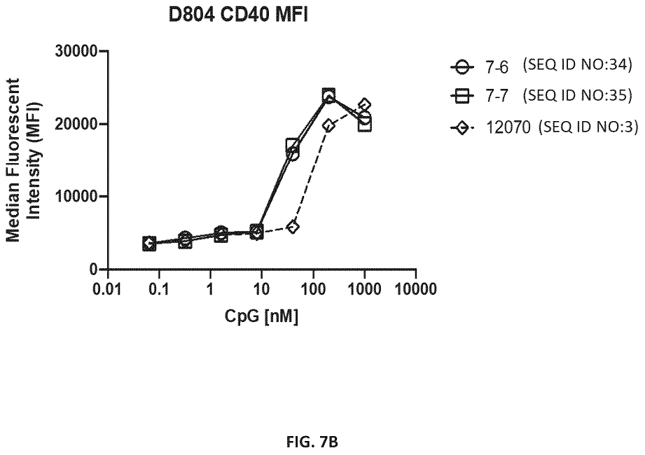

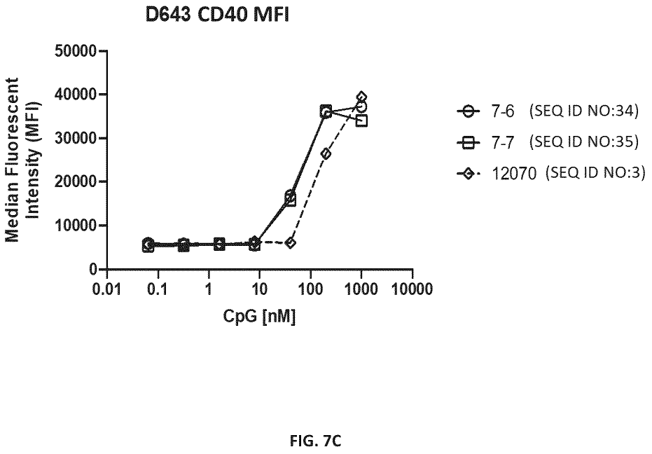

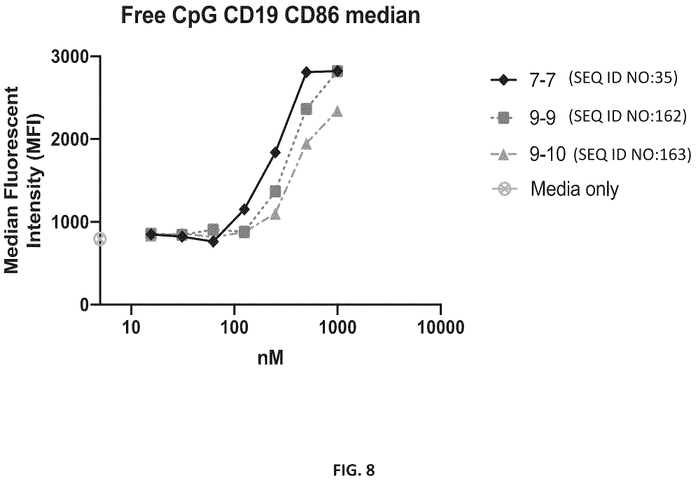

The results showed that the higher activities of 7-6 and 7-7 compared with 12070 were not dependent on the donor (FIGS. 7A-7C).

### Example 6: Contributions of 5′ Bromo 2′Deoxyuridine and PEG Linkage to CpG Activity

For evaluation of CpG oligonucleotides in human PBMCs, Trima residuals were received from Vitalant and diluted 1:4 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface and resuspended in FACS buffer (PBS with 0.5% Bovine Serum Albumin (Gibco)). PBMCs were immediately plated onto a 96-well format (500K/well) in Complete RPMI (RPMI+10% FBS). Five-fold serial dilutions were added to the cells from 1 uM to 64 pM of CpG oligonucleotides at 37° C. under 5% CO₂ for 48 to 96 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR. Blocking Reagent (Miltenyi Biotec), anti-CD19, anti-CD40, and anti-CD86. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Gating CD19+, CD20+ or CD19 CD20+ cells to identify B cells. Data was tabulated using GraphPad Prism 9.0.

As shown in FIG. 8, CpG oligonucleotides 9-9 and 9-10 without the bromo modification at the 5′ uridine activated CD86 expression. This implies that the bromo modification is not an essential component of the respective oligonucleotides.

### Example 7: Anti-SIRP-α Antibody-CpG Oligonucleotide Conjugates Activate Human Monocytes and Dendritic Cells

Anti-SIRP-α antibody-CpG oligonucleotide conjugates were assessed for activation of monocytes and dendritic cells. Human peripheral blood mononuclear cells (PBMCs) were treated with the conjugates and compared to unconjugated SIRPα antibodies.

Experiments using anti-SIRP-α antibody-CpG oligonucleotide conjugate used the 7-7 CpG oligo (SEQ ID NO:35) conjugated to anti-SIRP-α antibody comprising: (a) the heavy chain sequence of SEQ ID NO:68 and the light chain sequence of SEQ ID NO:75 (for anti-hIgG1), (b) the heavy chain sequence of SEQ ID NO:66 and the light chain sequence of SEQ ID NO:75 (for anti-hIgG4), or (c) the heavy chain sequence of SEQ ID NO:67 and the light chain sequence of SEQ ID NO:75 (for anti-hIgG1 AAA). Trima residuals were received from Vitalant and diluted 1:3 with Phosphate Buffered Saline (PBS, Gibco). Diluted blood was underplayed with 15 mL Ficoll-Paque (GE Healthcare). Tubes were centrifuged for 30 minutes at 400×g with break off. PBMCs were collected from the interface, resuspended and washed in FACS buffer (PBS with 0.5% Bovine Serum Albumin (Gibco)). After one wash, PBMCs were resuspended in Complete RPMI (RPMI+10% FBS). PBMCs were immediately plated onto a 96-well format (1e6/well) in Complete RPMI. Three-fold serial dilutions of anti-SIRP-α antibody-CpG oligonucleotide conjugates and unconjugated anti-SIRP-α antibodies were added to the cells from 300 nM to 0.41 nM at 37° C. under 5% CO₂ for 24 hours. Cells were pelleted by centrifugation for five minutes at 400×g and stained at 4° C. in Fixable Viability Dye eFluor 780 (Thermo Fisher) diluted 1:5000 in PBS. Cells were centrifuged and stained at 4° C. in FACS buffer for 60 minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86, anti-CD304, anti-CD1c, and anti-CD141 (Thermo Fisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3-CD19⁻CD14⁺ cells. CD11c high dendritic cells were identified as CD3⁻CD19⁻CD56⁻CD14⁻ HLADR⁺CD11c⁺. Conventional DCs were identified as CD3⁻CD19⁻CD56⁻CD14⁻ HLADR⁺CD11c⁺CD1c⁺ and plasmacytoid DCs were identified as CD3⁻CD19⁻CD56⁻CD14⁻ CD11c⁻HLADR⁺CD304⁺. Levels of activation marker were assessed by median fluorescent intensity.

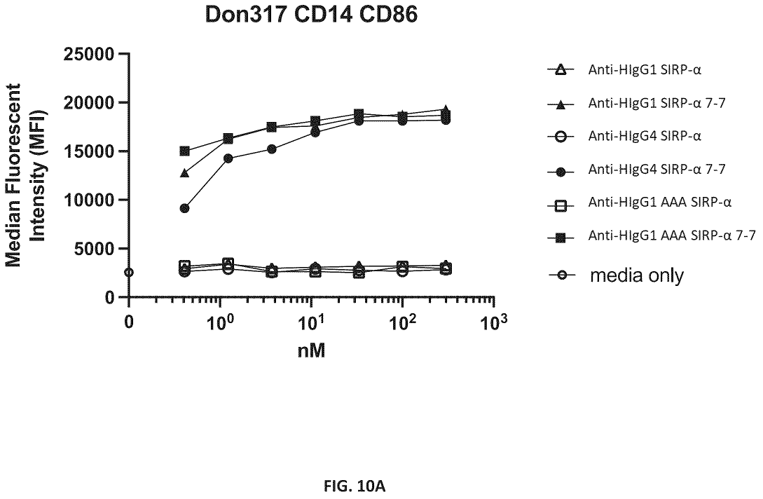

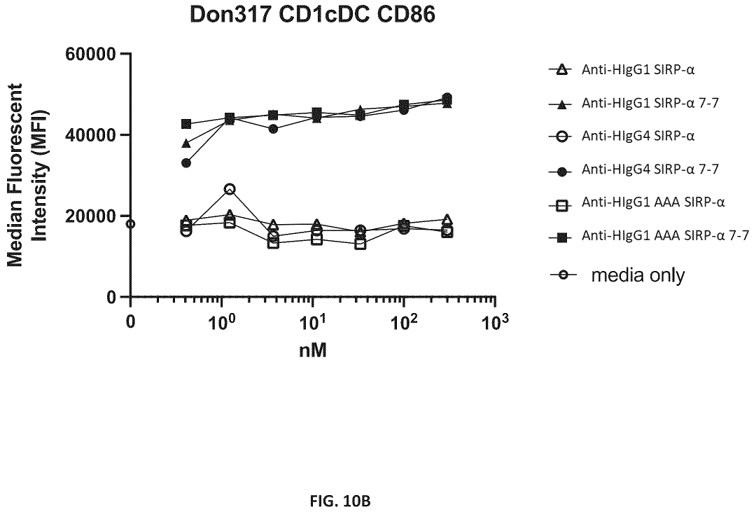

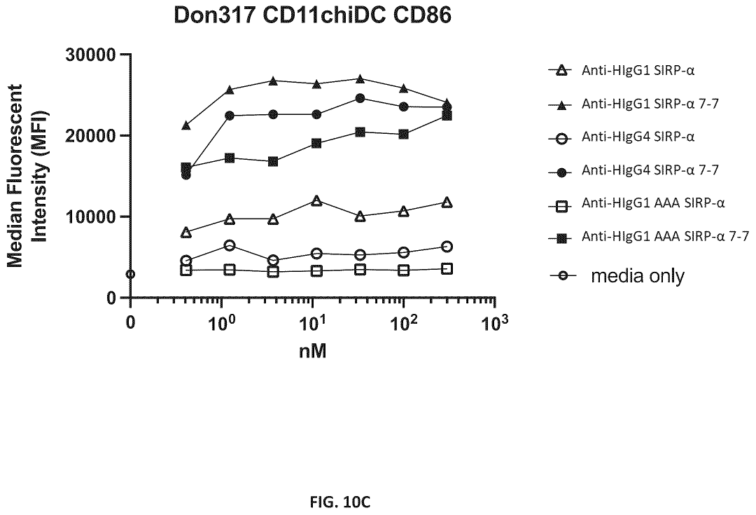

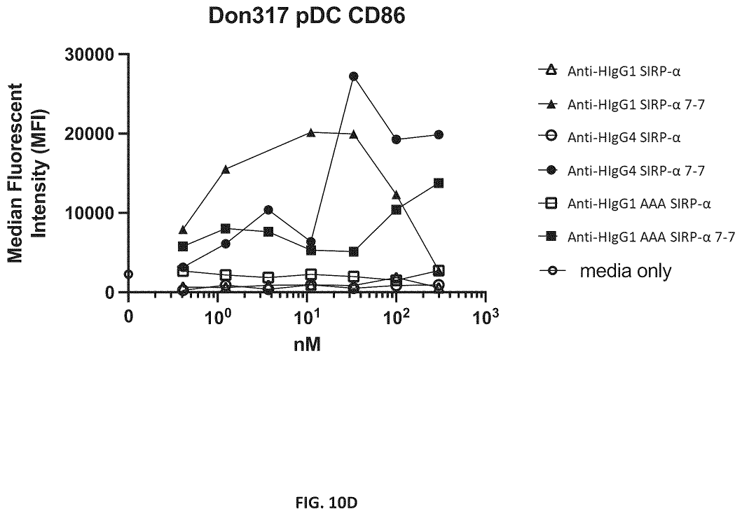

As shown in FIGS. 10A-10D, anti-SIRP-α antibody-CpG oligonucleotide conjugates exhibited robust activation of CD86 on human monocytes, conventional dendritic cells (cDC1), CD11c high dendritic cells and plasmacytoid DC (pDC) respectively. The activity of the conjugates was markedly above the unconjugated anti-SIRP-α antibodies.

### Example 8: Anti-SIRP-α Antibody-CpG Oligonucleotide Conjugates Stimulate IRF7 and IL-6 Induction

Anti-SIRP-α antibody-CpG oligonucleotide conjugates were assessed for stimulation of interferon regulatory factor 7 (IRF7) and interleukin-6 (IL-6) in myeloid cells. PBMCs were treated with the conjugates and compared to unconjugated anti-SIRP-α antibodies.

Experiments using anti-SIRP-α antibody-CpG oligonucleotide conjugate used the 7-7 CpG oligo (SEQ ID NO:35) conjugated to anti-SIRP-α antibody comprising: (a) the heavy chain sequence of SEQ ID NO:68 and the light chain sequence of SEQ ID NO:73 (for anti-hIgG1), (b) the heavy chain sequence of SEQ ID NO:66 and the light chain sequence of SEQ ID NO: 73 (for anti-hIgG4), and 12070 (SEQ ID NO: 3) conjugated to anti-SIRP-α antibody comprising the heavy chain sequence of SEQ ID NO:67 and the light chain sequence of SEQ ID NO:73 (for anti-hIgG1 AAA). Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended, and washed in FACS buffer (PBS with 0.5% Bovine Serum Albumin (Gibco)). After one wash, PBMCs were resuspended in Complete RPMI (RPMI+10% FBS). PBMCs were immediately plated onto a 96-well format (1e6/well) in Complete RPMI. Five-fold serial dilutions of anti-SIRP-α antibody-CpG oligonucleotide conjugates and unconjugated anti-SIRP-α antibodies were added to the cells from 100 nM to 6.4 pM at 37° C. under 5% CO₂ for 48 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86 (Thermo Fisher, Biolegend) Cells were centrifuged and washed twice in FACS buffer. Cells were then processed for intracellular staining using the transcription factor fixation/permeabilization concentrate and diluent (eBioscience). Briefly, cells were incubated in fresh fixation buffer by mixing 1 part of fixation/permeabilization concentrate with 3 parts of fixation permeabilization diluent. Samples were incubated for 30-60 min at 4° C. protected from light. Samples were then centrifuged at 600 g for 5 min at room temperature. Resuspended pellet with 1×permeabilization buffer followed by two rounds of washes and centrifugation at 600 g for 5 min at room temperature. Pellets were resuspended in 100 μL of permeabilization buffer and stained with anti-IL6, anti-IRF7 for 60 min at room temperature. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3⁻CD19⁻CD14⁺ cells, dendritic cells were identified as CD3⁻CD19⁻CD56⁻CD14⁻HLADR⁺CD11c⁺. Levels of activation marker were assessed by median fluorescent intensity and cytokine/chemokine expression was assessed as a % of CD14⁺ or CD11c⁺ cells.

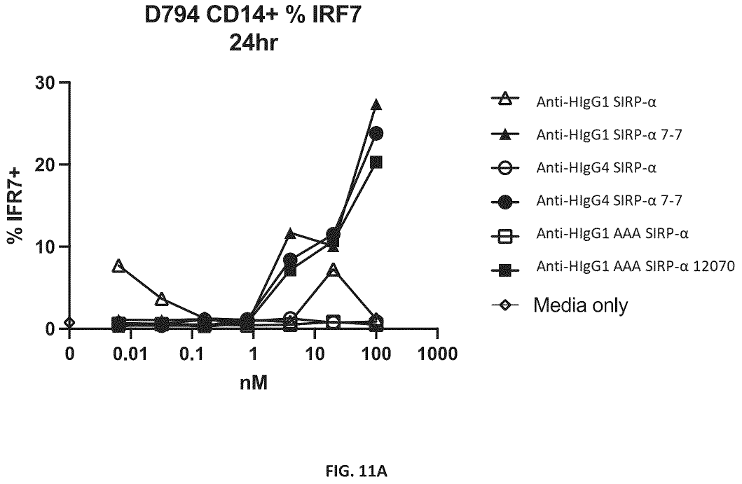

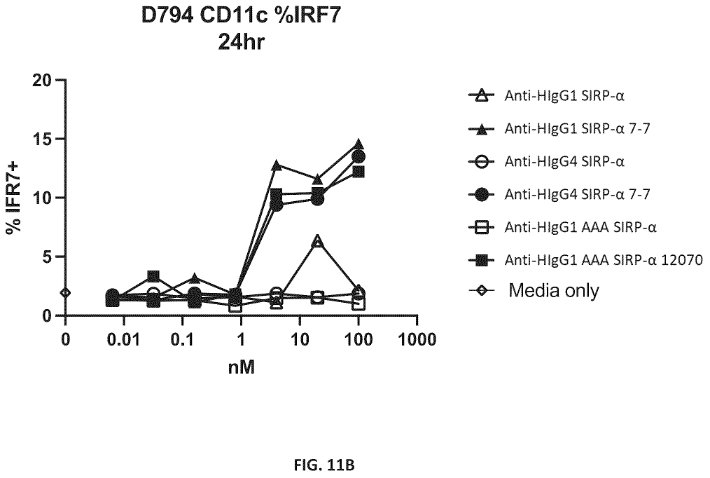

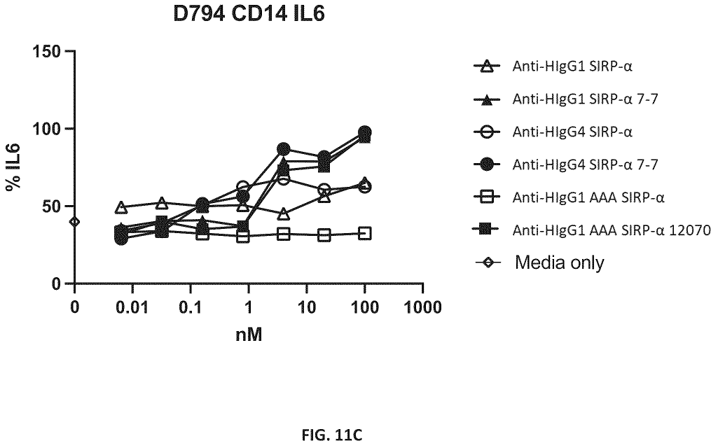

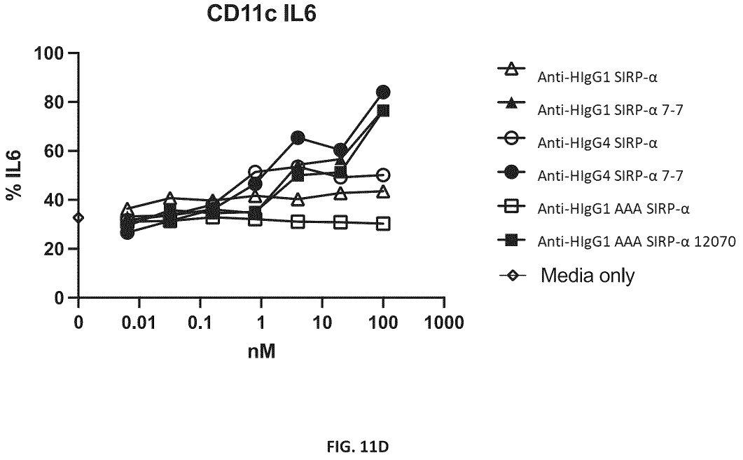

As shown in FIGS. 11A-11D, anti-SIRP-α antibody-CpG oligonucleotide conjugates exhibited robust induction of myeloid derived IRF7 and IL6 in human monocytes and dendritic cells. The activity of the conjugates was markedly above the unconjugated anti-SIRP-α antibodies.

### Example 9: Anti-SIRP-α Antibody-CpG Oligonucleotide Conjugates Enhance Cytokine Secretion

Anti-SIRP-α antibody-CpG oligonucleotide conjugates were assessed for their effect on cytokine secretion in PBMCs.

Experiments using anti-SIRP-α antibody-CpG oligonucleotide conjugate used the 7-7 CpG oligo (SEQ ID NO:35) conjugated to anti-SIRP-α antibody comprising: (a) the heavy chain sequence of SEQ ID NO:68 and the light chain sequence of SEQ ID NO:75 (for anti-hIgG1), (b) the heavy chain sequence of SEQ ID NO:66 and the light chain sequence of SEQ ID NO:75 (for anti-hIgG4), or (c) the heavy chain sequence of SEQ ID NO:67 and the light chain sequence of SEQ ID NO:75 (for anti-hIgG1 AAA). Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended, and washed in FACS buffer (PBS with 0.5% Bovine Serum Albumin (Gibco)). After one wash, PBMCs were resuspended in Complete RPMI (RPMI+10% FBS). PBMCs were immediately plated onto a 96-well format (1e6/well) in Complete RPMI. Five-fold serial dilutions of anti-SIRP-α antibody-CpG oligonucleotide conjugates and unconjugated anti-SIRP-α antibodies were added to the cells from 100 nM to 6.4 pM at at 37° C. under 5% CO₂ for 48 hours. Cells were pelleted by centrifugation for five minutes at 400×g and supernatant was collected for cytokine quantified using BioLegend's LEGENDplex™ Human Inflammation Panel I bead-based immunoassay (cat #740809) per manufacturer's recommendation. Briefly, supernatant plated in assay buffer prior to addition of cytokine specific capture beads. Plate was incubated for 2 hours at room temperature followed by two washes prior to the addition of biotinylated detection antibody for 1 hour. Without washing, PE conjugated streptavidin was added to the plate and incubate for 30 minutes at room temperature followed by two washes prior to the addition of buffer. Assay plate was then processed by flow cytometry using Attune NxT cytometer (Thermofisher). Data were analyzed with FlowJo 10.6 software (BD) and tabulated using GraphPad Prism.

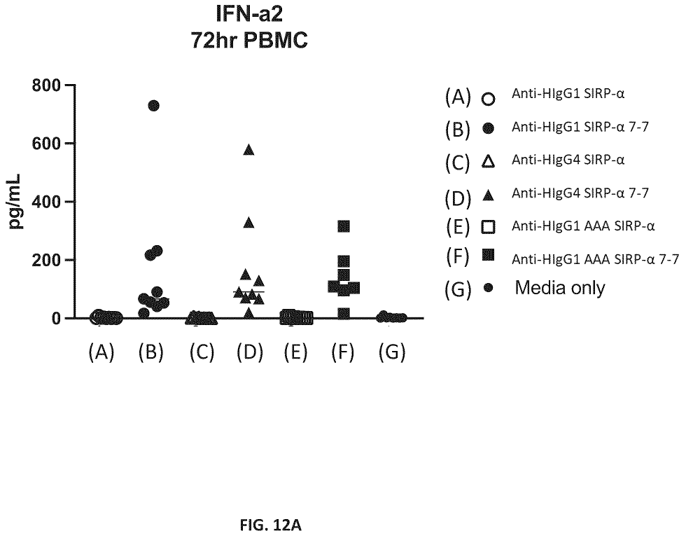

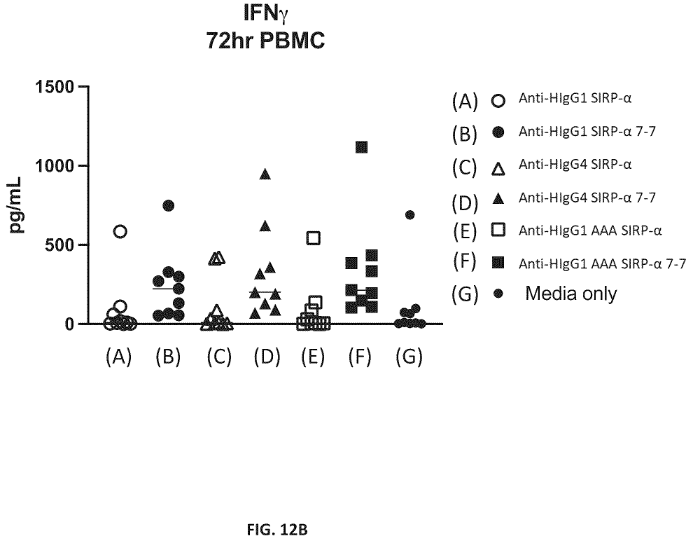

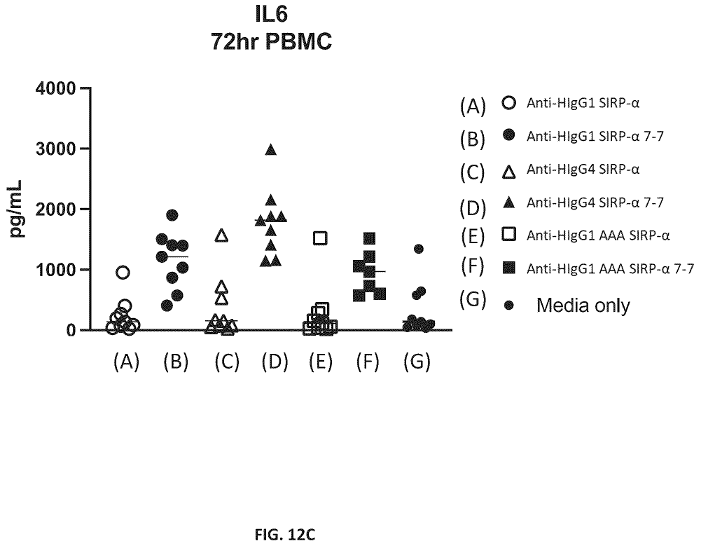

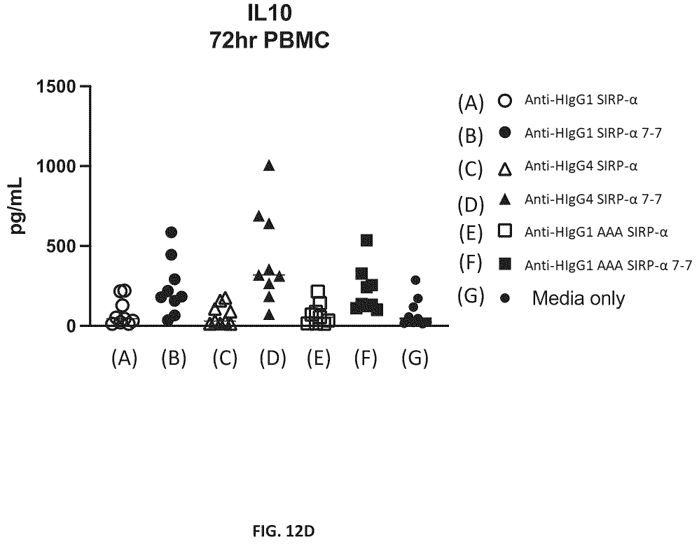

As shown in FIGS. 12A-12D, anti-SIRP-α antibody-CpG oligonucleotide conjugates stimulated cytokine secretion in human PBMCs at a level higher than the unconjugated anti-SIRP-α antibodies. These cytokines included IFN-α2, IFN-γ, IL-6, and IL-10.

### Example 10: Unconjugated CpG Oligonucleotides Activate Myeloid Cells

CpG oligonucleotides were compared for their ability to activate CD40 in myeloid cells. Two unconjugated oligonucleotides were tested, CpG 7-7 (SEQ ID NO:35) and CpG-12070 (SEQ ID NO:3).

Trima residuals were received from Vitalant and diluted 1:3 with Phosphate Buffered Saline (PBS, Gibco). Diluted blood was underplayed with 15 mL Ficoll-Paque (GE Healthcare). Tubes were centrifuged for 30 minutes at 400×g with break off. PBMCs were collected from the interface, resuspended and washed in FACS buffer (PBS with 0.5% Bovine Serum Albumin (Gibco)). After one wash, PBMCs were resuspended in Complete RPMI (RPMI+10% FBS). PBMCs were immediately plated onto a 96-well format (1e6/well) in Complete RPMI. CpG oligonucleotides were incubated with the cells at 40 nM concentration at 37° C. under 5% CO₂ for 48 hours. Cells were pelleted by centrifugation for five minutes at 400×g and stained at 4° C. in Fixable Viability Dye eFluor 780 (Thermo Fisher) diluted 1:5000 in PBS. Cells were centrifuged and stained at 4° C. in FACS buffer for 60 minutes containing FcR Blocking Reagent (Miltenyi Biotec), anti-CD14, anti-CD11c, anti-CD3, anti-CD19, anti-CD56, anti-CD16, anti-HLADR, anti-CD40, and anti-CD86 (Thermo Fisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3⁻CD19⁻CD14⁺ cells. Dendritic cells were identified as CD3⁻CD19⁻CD56⁻CD14⁻ HLADR⁺CD11c⁺. Levels of activation marker were assessed by median fluorescent intensity.

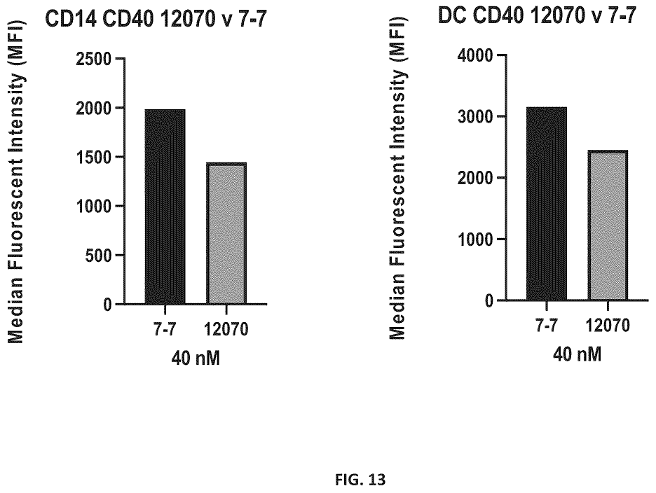

As shown in FIG. 13, the 7-7 CpG oligonucleotide showed higher activation of CD40, as compared to the 12070 CpG oligonucleotide in human myeloid cells.

### Example 11: Anti-SIRP-α Antibody-CpG Oligonucleotide Conjugate Demonstrates Tumor Phagocytosis by Monocyte-Derived M2 Macrophages Via CD47-SIRP-α Blockade

Anti-SIRP-α antibody-CpG oligonucleotide conjugate was assessed for its ability to stimulate tumor phagocytosis by monocyte-derived M2 macrophages as compared to unconjugated anti-SIRP-α antibody or media controls.

Experiments using anti-SIRP-α antibody-CpG oligonucleotide conjugate used the 7-7 CpG oligo (SEQ ID NO:35) conjugated to anti-SIRP-α antibody comprising: the heavy chain sequence of SEQ ID NO:66 and the light chain sequence of SEQ ID NO:73 (for anti-hIgG4). Human CD14 cells were purified from Trima residuals (Vitalant) with Ficoll-Paque Plus and negative selection (Monocyte Isolation Kit II, Miltenyi Biotec) according to the manufacturers' protocols. Monocyte-derived macrophages (MDM) were made by seeding 10 million CD14⁺ cells into 150 mm tissue culture dishes (Corning) in growth medium supplemented with 10% FBS and 50 ng/ml MCSF. Cells were cultured for 7-11 days. Adherent cells were detached from culture plates with TrypLE Select (Thermo Fisher Scientific). Target cells (DLD-1) were labeled with the Celltrace CFSE Cell Proliferation kit (Thermo Fisher Scientific) according to the manufacturer's instructions. 100,000 target cells and 50,000 MDMs were incubated in ultra-low attachment U-bottom 96-well plates (Corning) with 150 nM unconjugated anti-SIRPα hIgG4 and the corresponding CpG conjugate for 2 h at 37° C. For flow cytometry, cells were incubated in human FcR blocking reagent (Miltenyi Biotec) and stained with fluorochrome-labeled antibodies against CD33. To eliminate macrophage/target cell adhesion from analyses, antibody against CD326 was included. Furthermore, a pulse geometry gate of forward scatter signal area vs height was used to select for single cells. Fixable viability dye (Thermo Fisher Scientific) was used to identify live cells. Cells were acquired on a FACS Canto II flow cytometer (BD Biosciences) with subsequent analysis using FlowJo software. Percent phagocytosis indicates the percentage of viable CD33 macrophages that stain negative for CD326 and positive for CFSE.

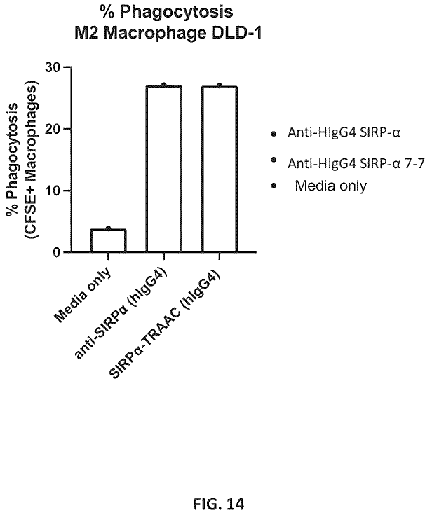

As shown in FIG. 14, the anti-SIRP-α antibody-CpG oligonucleotide conjugate showed robust stimulation of phagocytosis of DLD-1 tumor cells by M2 macrophages equivalent to the unconjugated antibody, demonstrating that the conjugate maintained the ability to work through the CD47-SIRP-α blockade.

### Example 12: Anti-Tumor Activity of Anti-SIRP-α Antibody-CpG Oligonucleotide Conjugates

Anti-SIRP-α antibody-CpG oligonucleotide conjugates were constructed with either a mouse IgG2a or a mouse IgG1 Fc domain and compared for their anti-tumor activity in RENCA, a SIRP α-positive syngeneic tumor model.

Experiments using anti-SIRP-α antibody-CpG oligonucleotide conjugate used the mouse CpG oligo 4523 (SEQ ID NO: 121) conjugated to anti-SIRP-α antibody comprising a heavy chain comprising the sequence of DVQLVESGGGVVRPGESLRLSCAASGFTFSSNAMSWVRQAPGKGLEWLAGISAGGSDT YYPASVKGRFTISRDNSKNTLYLQMNTLTAEDTAVYYCARETWNHLFDYWGLGTLVT VSSAKTTAPSVYPLAPVCGDTTGSSVTLGCLVKGYFPEPVTLTWNSGSLSSGVHTFPAVL QSDLYTLSSSVTVTSSTWPSQSITCNVAHPASSTKVDKKIEPRGPTIKPCPPCKCPAPNLL GGPSVFIFPPKIKDVLMISLSPIVTCVVVDVSEDDPDVQISWFVNNVEVHTAQTQTHRED YNSTLRVVSALPIQHQDWMSGKEFKCKVNNKDLPAPIERTISKPKGSVRAPQVYVLPPP EEEMTKKQVTLTCMVTDFMPEDIYVEWTNNGKTELNYKNTEPVLDSDGSYFMYSKLR VEKKNWVERNSYSCSVVHEGLHNHHTTKSFSRTPGRPQGFGPP (SEQ ID NO:90) for mIgG2a or DVQLVESGGGVVRPGESLRLSCAASGFTFSSNAMSWVRQAPGKGLEWLAGISAGGSDT YYPASVKGRFTISRDNSKNTLYLQMNTLTAEDTAVYYCARETWNHLFDYWGLGTLVT VSSAKTTPPSVYPLAPGSAAQTNSMVTLGCLVKGYFPEPVTVTWNSGSLSSGVHTFPAV LQSDLYTLSSSVTVPSSTWPSETVTCNVAHPASSTKVDKKIVPRDCGCKPCICTVPEVSS VFIFPPKPKDVLTITLTPKVTCVVVDISKDDPEVQFSWFVDDVEVHTAQTQPREEQFNST FRSVSELPIMHQDWLNGKEFKCRVNSAAFPAPIEKTISKTKGRPKAPQVYTIPPPKEQMA KDKVSLTCMITDFFPEDITVEWQWNGQPAENYKNTQPIMDTDGSYFIYSKLNVQKSNW EAGNTFTCSVLHEGLHNHHTEKSLSHSPGRPQGFGPP (SEQ ID NO:91) for mIgG1 and a light chain comprising the sequence of ALTQPASVSANPGETVKIACSGGDYYSYYYGWYQQKAPGSALVTVIYSDDKRPSDIPSR FSGSASGSTATLTITGVRAEDEAVYYCGGYDYSTYANAFGAGTTLTVLGQPKSSPSVTL FPPSSEELETNKATLVCTITDFYPGVVTVDWKVDGTPVTQGMETTQPSKQSNNKYMASS YLTLTARAWERHSSYSCQVTHEGHTVEKSLSRADCS (SEQ ID NO:111). The sequence of the 4523 murine CpG oligonucleotide is tucgtcgtgacgtt-c3, where lower case indicates phosphothioate linkages, bold indicates iodo-uridine, and underlining indicates phosphotriester linker (SEQ ID NO:121). RENCA renal carcinoma cells (ATCC) were cultured in Complete RPMI 1640 (RPMI 1640+10% FBS (Gibco)) at 37° C. and 5% CO₂. Cells were detached with Trypsin 0.25% (Gibco) and washed twice with RPMI 1640 (Gibco). Cells were resuspended at 20E6/mL in RPMI 1640 and kept on ice until use. 100 uL of suspended cells were subcutaneously implanted into the right flank of 6-week-old female BALB/c mice (Charles River). Tumor size was measured and recorded twice a week with calipers starting 9 days post implantation until duration of the study, approximately 35 days later. Tumor volume was calculated using the following formula: (length×width×width)/2. Once tumors reached on average 75 mm³, approximately 3 days post implantation, mice were randomized by tumor size and treatments were initiated. The conjugates were administered intraperitoneally 3 doses every 3 days at 3 mg/kg or 10 mg/kg. Mice whose tumors exceeded 2,000 mm³ or exhibited any signs of distress at any time during the study were sacrificed humanely as per IACUC-approved animal protocols.

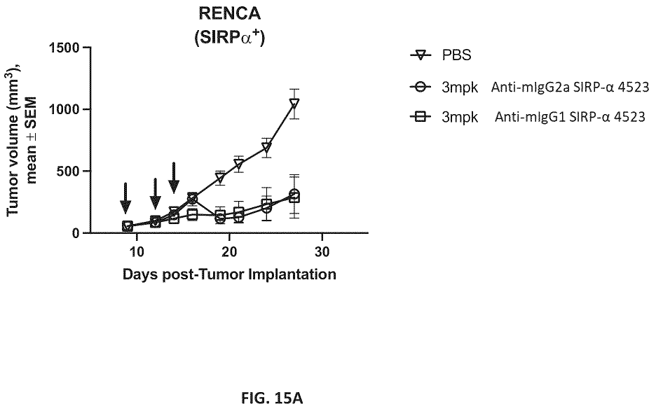

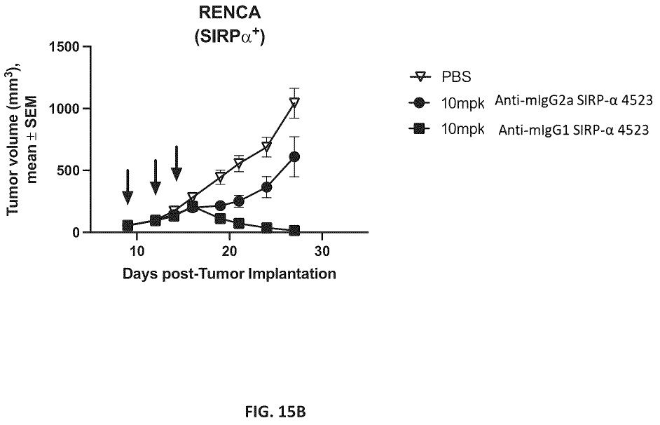

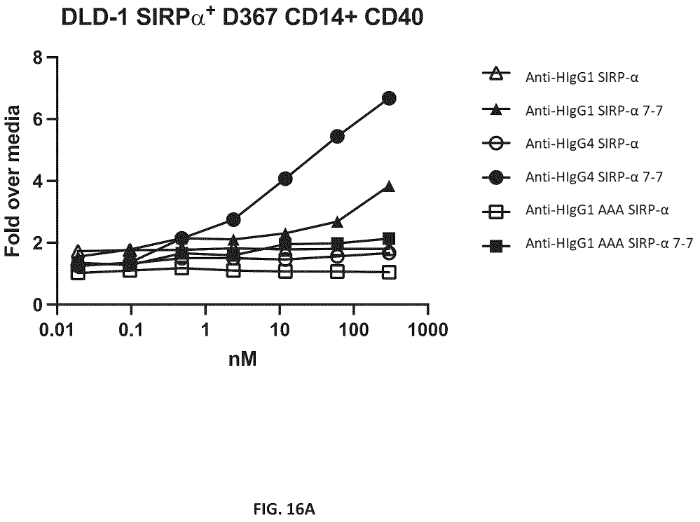

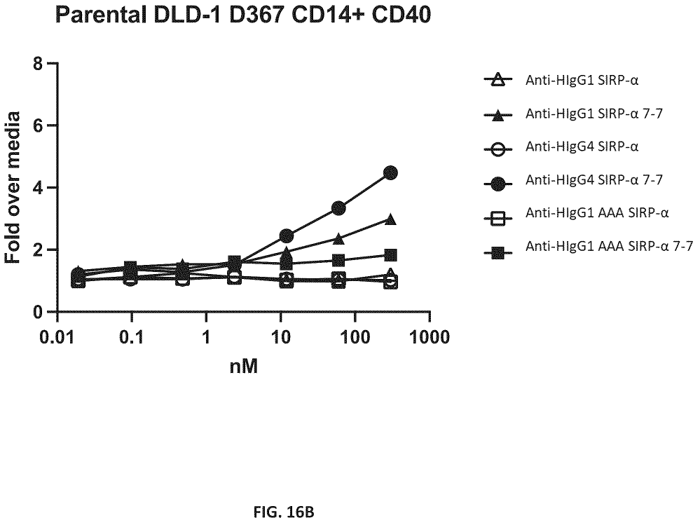

As shown in FIGS. 15A & 15B, anti-SIRP-α antibody-CpG oligonucleotide conjugates displayed equivalent anti-tumor response at the lower dosage of 3 mg/kg. However, at the higher dose level of 10 mg/kg, the conjugate with the mIgG2a Fc displayed reduced anti-tumor inhibition as compared to conjugate with the mIgG1 Fc domain. Anti-SIRP-α antibody-CpG oligonucleotide conjugates provide robust single agent activity against RENCA, a poorly immunogenic tumor type. Without wishing to be bound to theory, it is thought that the differential responses seen at a higher dose level of mIgG2a could be attributed to a scorpion effect (see, e.g., Kurlander R J. (1983) J Immunol. 131 (1): 140-7 and Voets, E., et al. (2019) j. immunotherapy cancer 7, 340), depletion of effector cell types, or a combination of both.

### Example 13: Monocyte Activation by Anti-SIRP-α Antibody-CpG Oligonucleotide Conjugates in PBMCs Co-Cultured with Tumor Cell Lines

Anti-SIRP-α antibody-CpG oligonucleotide conjugates were assessed for their ability to activate monocytes when co-cultured in the presence of a SIRP-α positive or SIRP-α-negative tumor cell lines.

Experiments using anti-SIRP-α antibody-CpG oligonucleotide conjugate used the 7-7 CpG oligo (SEQ ID NO:35) conjugated to anti-SIRP-α antibody comprising: (a) the heavy chain sequence of SEQ ID NO:68 and the light chain sequence of SEQ ID NO:75 (for anti-hIgG1), (b) the heavy chain sequence of SEQ ID NO:66 and the light chain sequence of SEQ ID NO: 75 (for anti-hIgG4), or (c) the heavy chain sequence of SEQ ID NO:67 and the light chain sequence of SEQ ID NO:75 (for anti-hIgG1 AAA). Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin (Gibco)). After one wash, PBMCs were resuspended in Complete RPMI (RPMI+10% FBS). PBMCs were immediately plated onto a 96-well format (0.5e6/well) in Complete RPMI. Tumor cells (0.025E6/well) from DLD-1 parental CFSE labeled, or DLD-1 transduced to overexpress SIRP-α+ and GFP were added to PBMC at 20:1 (effector to target). Anti-SIRP-α antibody-CpG oligonucleotide conjugates and unconjugated anti-SIRP-α antibodies were added to the cells from 300 nM to 0.41 nM at 37° C. under 5% CO₂ for 48 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3⁻CD19⁻CD14⁺ cells, dendritic cells were identified as CD3⁻CD19⁻CD56⁻CD14⁻HLADR⁺CD11c⁺. CD14⁺ monocyte activation levels of activation marker were assessed by median fluorescent intensity.

FIGS. 16A & 16B show data from three SIRP-α-CpG oligonucleotide conjugates, each conjugated to a different Fc domain (human IgG4, human IgG1 and human IgG1-AAA). The hIgG4 conjugate showed the strongest induction of CD40 in monocytes when co-cultured with DLD-1 transduced to overexpress SIRP-α and in non-SIRP-α expressing parental DLD-1 as compared to the other conjugates and unconjugated antibodies. The induction of CD40 in monocytes in the presence of DLD-1 irrespective of SIRP-α expression demonstrates that myeloid activation can be achieved via direct engagement of SIRP-α expressed on CD14 monocyte population and/or other modulatory mechanisms.

### Example 14: Anti-SIRP-α Antibody-CpG Oligonucleotide Conjugate is a TLR9 Agonist that Activates Myeloid Cells and Promotes Anti-Tumor Immunity

Novel therapies engaging both innate and adaptive immune responses may engender more robust and durable anti-cancer immunity (Kobold et al. (2019) Proc. Natl. Acad. Sci. 116:1087-1088). Activation of toll-like receptor 9 (TLR9) by unmethylated CpG oligonucleotides (ODNs) promotes innate inflammatory responses and the induction of adaptive immunity (Dowling et al. (2016) Clin. Transl. Immunol. 5:e85-10). Several CpG-ODNs have demonstrated clinical benefit in melanoma patients by intra-tumoral injection (Hamid et al. (2019) The Oncol. 25:343-359). Signal regulatory protein a (SIRPa) is a myeloid inhibitory receptor that suppresses immune activation following binding of its ligand CD47 (Kuo et al. (2020) J. Hematol. Oncol. 13:160). Blockade of CD47-SIRPα myeloid checkpoint pathway has demonstrated clinical benefit in patients with solid tumors (Lee et al. ALX148, a CD47 blocker in combination with standard chemotherapy and antibody regimens in patients with gastric/gastroesophageal junction (GC) cancer and head and neck squamous cell carcinoma (HNSCC); ASPEN-01. SITC 2020).

Examples 14-22 provide preclinical data demonstrating that anti-SIRP-α antibody-CpG oligonucleotide conjugate delivers a differentiated TLR-9 agonist (T-CpG) to myeloid cells via SIRP-α and FcγR engagement, triggering TLR9 signaling, cell activation and immune modulation resulting in robust anti-tumor efficacy. The anti-SIRP-α antibody-CpG oligonucleotide conjugate provides activity significantly above either component alone (i.e., only the anti-SIRP-α antibody or only the selected CpG oligonucleotide), demonstrating the suprising and unexpected benefit of combining these selected components.

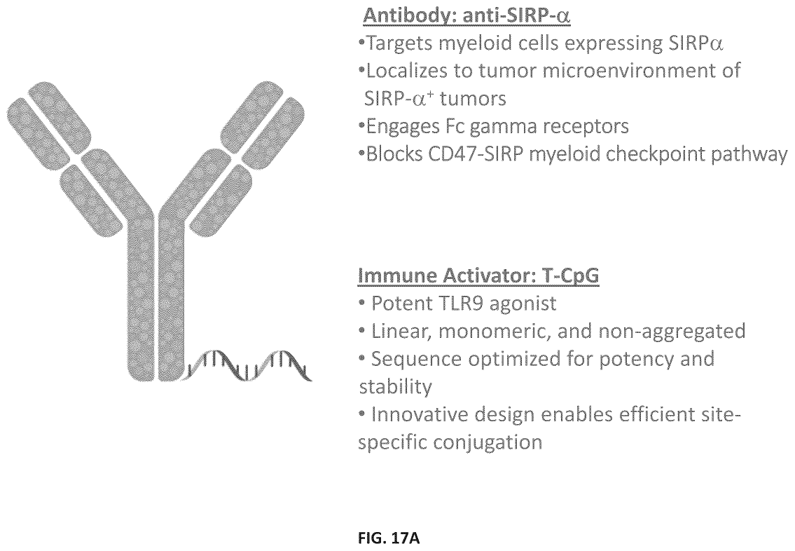

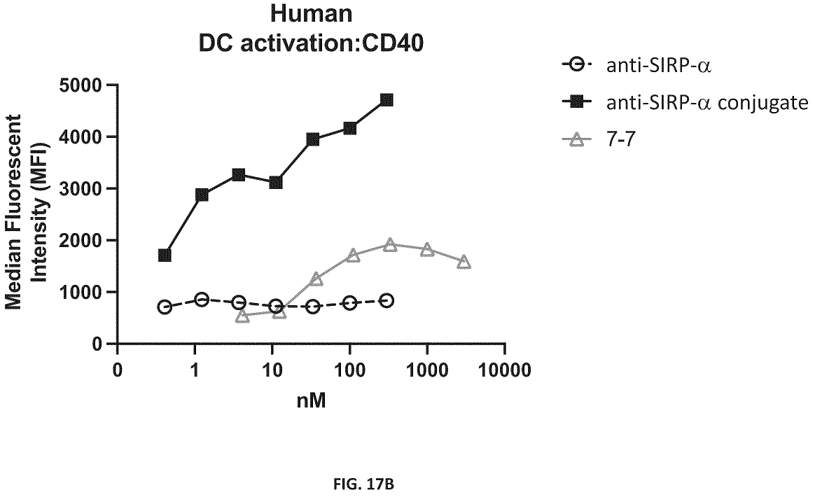

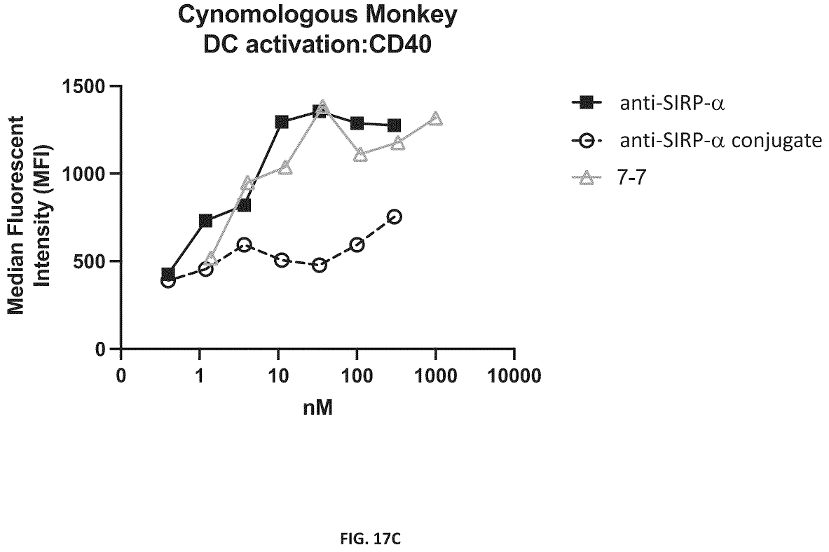

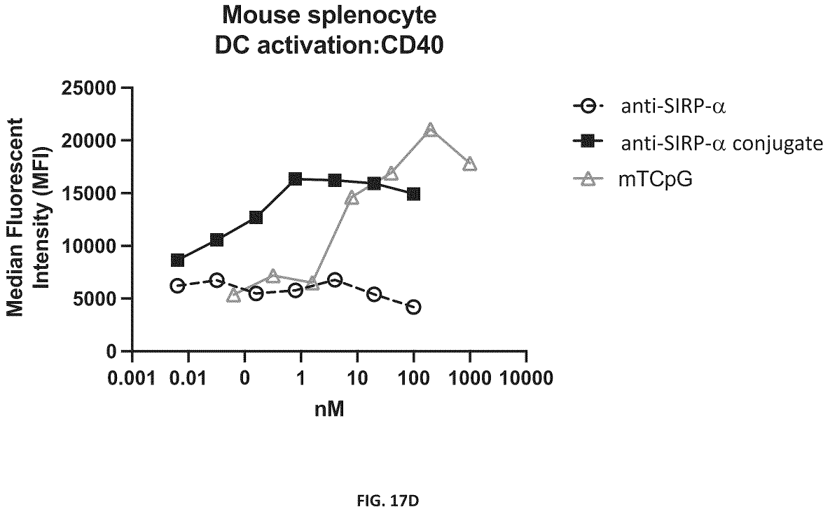

Anti-SIRP-α antibody-CpG oligonucleotide conjugates were comprised of a differentiated TLR-9 agonist (T-CpG) conjugated to an antibody against SIRPα (FIG. 17A). Human SIRP-α is expressed on dendritic cells and myeloid cells but not B cells, whereas TLR9 is expressed on dendritic cells, myeloid cells, and B cells.

### Example 15: T-CpG is a Potent TLR9 Agonist with Targeted Activation Across Species

### Materials and Methods

Trima residuals were received from Vitalant and diluted 1:3 with Phosphate Buffered Saline (PBS, Gibco). Diluted blood was underplayed with 15 mL Ficoll-Paque (GE Healthcare). Tubes were centrifuged for 30 minutes at 400×g with break off. PBMCs were collected from the interface, resuspended and washed in FACS buffer (PBS with 0.5% Bovine Serum Albumin (Gibco)). After one wash, PBMCs were resuspended in Complete RPMI (RPMI, 10% FBS, 1× Pen/Strep, 1× Glutamax). PBMCs were immediately plated onto a 96-well format (1e6/well) in Complete RPMI. Three-fold serial dilutions were added to the cells from 300 nM to 0.4 nM for anti-SIRP-α antibody (heavy chain comprising the sequence of SEQ ID NO:66 and light chain comprising the sequence of SEQ ID NO:75) and anti-SIRP-α conjugate (heavy chain comprising the sequence of SEQ ID NO:66 and a light chain comprising the sequence of SEQ ID NO:75, conjugated to CpG oligo 7-7 (SEQ ID NO:35)). Free CpG 7-7 (SEQ ID NO:35) was titrated from 3 μM to 4.12 nM. Cells were incubated at 37° C. under 5% CO₂ for 24 hours. Cells were pelleted by centrifugation for five minutes at 400×g and stained at 4° C. in Fixable Viability Dye eFluor 780 (Thermo Fisher) diluted 1:5000 in PBS. Cells were centrifuged and stained at 4° C. in FACS buffer for 60 minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86, anti-CD304, anti-CD1c, and anti-CD141 (Thermo Fisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3⁻CD19⁻CD14⁺ cells. Dendritic cells were identified as CD3-CD19−CD56−CD14−HLADR+CD11c+. Conventional DCs were identified as CD3⁻CD19⁻CD56⁻ CD14⁻HLADR⁺CD11c⁺CD1c⁺ and Plasmacytoid DCs were identified as CD3⁻CD19⁻CD56⁻ CD14⁻CD11c⁻HLADR⁺CD304⁺. Levels of activation marker were assessed by median fluorescent intensity.

Cynomolgus whole blood was received from BioIVT and diluted 1:3 with Phosphate Buffered Saline (PBS, Gibco). Diluted blood was underplayed with 15 mL 90% Ficoll-Paque (GE Healthcare) in PBS solution. Tubes were centrifuged for 30 minutes at 400×g with break off. Cells were collected from the interface, resuspended and washed in FACS buffer (PBS with 0.5% Bovine Serum Albumin (Gibco)). After one wash, cells were resuspended in ACK lysis buffer (Gibco) for 15 min at room temperature, washed in FACs buffer and centrifuged before repeating. Purified PBMCs were resuspended in Complete RPMI (RPMI, 10% FBS, 1× Pen/Strep, 1× Glutamax). PBMCs were immediately plated onto a 96-well format (0.3e6/well) in Complete RPMI. Three-fold serial dilutions were added to the cells from 1 μM to 1.4 nM of CpG oligonucleotide and 300 nM to 0.4 nM of anti-SIRP-α antibody (heavy chain comprising the sequence of SEQ ID NO:66 and light chain comprising the sequence of SEQ ID NO:75) and the corresponding antibody:CpG conjugate (heavy chain comprising the sequence of SEQ ID NO: 66 and light chain comprising the sequence of SEQ ID NO:75, conjugated to CpG oligo 7-7 (SEQ ID NO:35)) at 37° C. under 5% CO₂ for 48 hours. Cells were pelleted by centrifugation for five minutes at 400×g and stained at 4° C. in Fixable Viability Dye eFluor 780 (Thermo Fisher) diluted 1:5000 in PBS. Cells were centrifuged and stained at 4° C. in FACS buffer for 60 minutes containing FcR Blocking Reagent (Miltenyi Biotec), anti-CD14, anti-CD11c, anti-CD123, anti-CD3, anti-CD20, anti-CD16, anti-CD8, anti-HLADR, anti-CD40, anti-CD69 and anti-CD86 (Thermo Fisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Dendritic cells were identified as CD3⁻CD20⁻ CD16⁻CD14⁻CD8⁻CD123⁻HLADR⁺CD11c⁺. Levels of activation marker were assessed by median fluorescent intensity.

Mouse spleens were processed into single-cell suspension in ice-cold PBS, lysed with ACK lysis buffer (Gibco), washed twice and re-suspended in PBS supplemented with 2% FBS. Aliquots of 1-2 10⁶ cells stimulated for 48 hrs in Complete RPMI (RPMI+10% FBS, 1× Pen/Strep, 1× Glutamax). Splenocytes were plated onto a 96-well format (1e6/well) in the presence 100 nM to 6.4 pM of of anti-SIRP-α antibody comprising heavy chain comprising the sequence of SEQ ID NO:67 and light chain comprising the sequence of SEQ ID NO: 73; anti-SIRP-α antibody-CpG oligonucleotide conjugate comprising heavy chain comprising the sequence of SEQ ID NO:67 and light chain comprising the sequence of SEQ ID NO:73, conjugated to CpG oligo 4523 (SEQ ID NO: 121); or 1 uM to 64 pM of mT-CpG 4523 titrated 1:5. Cells were then incubated at 37° C. under 5% CO₂ for 48 hours. Cells were pelleted by centrifugation for five minutes at 400×g and stained at 4° C. in Fixable Viability Dye eFluor 780 (Thermo Fisher) diluted 1:5000 in PBS, followed by mouse Fc-block (Biolegend) and subsequently stained with the following antibodies at least 30 min at 4° C.: anti-SIGLEC H anti-CCR7, anti-CD86, anti-MHCII, anti-GR-1, anti-33D1 clone 33D1, anti-CD11b, anti-CD11c. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). All flow antibodies were purchased from either Biolegend or Thermo Fisher. Dead cells were excluded by gating on the eFluor 780-negative population. Dendritic cells were identified as either CD11c⁺MHCII⁺33D1⁺ or CD11c⁺MHCII⁺CD8⁺. Levels of activation marker were assessed by median fluorescent intensity.

### Results

Human PBMCs (FIG. 17B), cynomolgus PBMCs (FIG. 17C), or mouse splenocytes (FIG. 17D) were stimulated with anti-hSIRP-α antibody, anti-hSIRP-α antibody-CpG oligonucleotide conjugate, CpG 7-7 oligo, or anti-mSIRP-α antibody-CpG oligonucleotide conjugate with murine reactive mT-CpG for 24 hr or 48 hrs and surface marker expression (CD40) on dendritic cells was assayed by flow cytometry. The results demonstrated that the CpG 7-7 oligo is a potent TLR9 agonist with targeted dendritic cell activation across species. Additionally, in human dendritic cells, the anti-hSIRP-α antibody-CpG oligonucleotide conjugate showed significantly increased activation as compared with the antibody or oligonucleotide alone.

### Example 16: Cell Type Specificity and Activation by Anti-SIRP-α Antibody Conjugates

Ability of anti-SIRP-α antibody conjugates to specifically activate human target cells that express both SIRPα and TLR9 was examined.

### Materials and Methods

Trima residuals were received from Vitalant and diluted 1:3 with Phosphate Buffered Saline (PBS, Gibco). Diluted blood was underplayed with 15 mL Ficoll-Paque (GE Healthcare). Tubes were centrifuged for 30 minutes at 400×g with break off. PBMCs were collected from the interface, resuspended and washed in FACS buffer (PBS with 0.5% Bovine Serum Albumin (Gibco)). After one wash, PBMCs were resuspended in Complete RPMI (RPMI, 10% FBS, 1× Pen/Strep, 1× Glutamax). PBMCs were immediately plated onto a 96-well format (1e6/well) in Complete RPMI. 100 nM of either anti-hIgG4 SIRP-α conjugate (anti-SIRP-α antibody-CpG oligonucleotide conjugate with a heavy chain comprising the sequence of SEQ ID NO:66 and a light chain comprising the sequence of SEQ ID NO:73, conjugated to CpG oligo 7-7 (SEQ ID NO: 35)) or anti-CD22 antibody-CpG oligonucleotide conjugate were added to the cells. The anti-CD22 antibody had a heavy chain comprising a VH domain comprising the sequence of QVQLLESGGGVVQPGGSLRLSCAASGFAFSIYDMNWVRQAPGKGLEWVSAISSGGGTT YYADSVKGRFTISRDNAKNSLYLQMNSLRAEDTAVYYCARHSGYGTHWGVLFAYWG RGTLVTVSS (SEQ ID NO:112) with a human IgG1 Fc and a VL domain comprising the sequence of DIQMTQSPSSLSASVGDRVTITCRASQDIHGYLNWYQQKPGKAPKLLIYYTSILHSGVPS RFSGSGSGTDFTLTISSLQPEDFATYFCQQGSTLPWTFGQGTKLEIK (SEQ ID NO:113), and was conjugated to CpG oligo 7-7 (SEQ ID NO:35).

Cells were then incubated at 37° C. under 5% CO₂ for 24 hours. Cells were pelleted by centrifugation for five minutes at 400×g and stained at 4° C. in Fixable Viability Dye eFluor 780 (Thermo Fisher) diluted 1:5000 in PBS. Cells were centrifuged and stained at 4° C. in FACS buffer for 30-60 minutes containing FcR Blocking Reagent (Miltenyi Biotec), anti-CD14, anti-CD3, anti-CD19, and anti-CD86 (Thermo Fisher, Biolegend). Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Monocytes were identified as CD3⁻CD19⁻CD14⁺ cells. B cells were identified as CD3 CD19. Levels of activation marker were assessed by median fluorescent intensity.

### Results

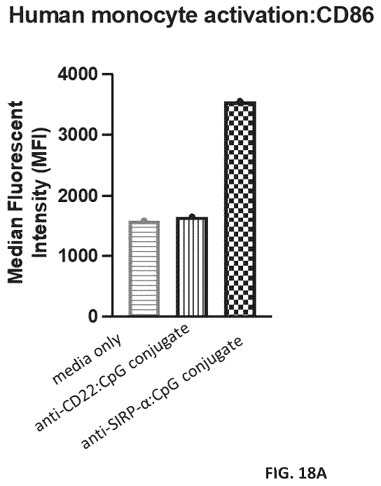

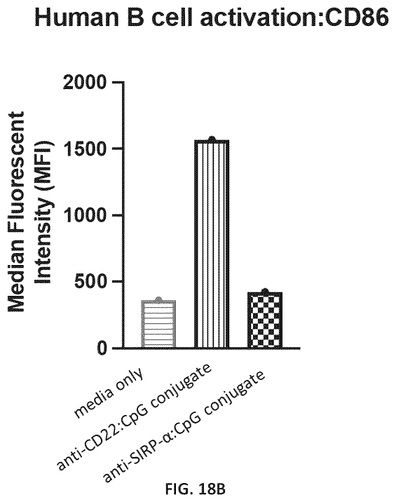

Ability of antibody conjugates to specifically activate human target cells was examined. Human PBMCs were stimulated with anti-SIRP-α antibody-CpG oligonucleotide conjugate or anti-CD22 antibody-CpG oligonucleotide conjugate for 24 hr and CD86 surface marker expression on monocytes (FIG. 18A) and B cells (FIG. 18B) was assayed by flow cytometry. The anti-SIRP-α antibody-CpG oligonucleotide conjugate specifically targeted and activated SIRP-α-expressing monocytes but not B-cells which do not express SIRP-α. In contrast, the B-cell targeting anti-CD22 antibody-CpG oligonucleotide demonstrates efficient activation of B-cells.

### Example 17: Ability of Anti-SIRP-α Antibody Conjugates to Activate Dendritic Cells Co-Cultured with SIRP-α-Positive or SIRP-α-Negative Tumor Cells

### Materials and Methods

Trima residuals were received from Vitalant and diluted 1:2 with Phosphate Buffered Saline (PBS, Gibco). Diluted blood was split into two tubes and underplayed with 15 mL Ficoll-Paque (GE Healthcare). Tubes were centrifuged for 30 minutes at 400×g. PBMCs were collected from the interface, resuspended and washed in FACS buffer (PBS with 0.5% Bovine Serum Albumin (Gibco)). After one wash, PBMCs were resuspended in Complete RPMI (RPMI, 10% FBS, 1× Pen/Strep, 1× Glutamax) PBMCs were immediately plated onto a 96-well format (0.5e6/well) in Complete RPMI. Tumor cells (0.025E6/well) from DLD-1 parental CFSE labeled, or DLD-1 transduced to overexpress SIRP-α+ and GFP were added to PBMC at 20:1 (effector to target). The antibodies and corresponding antibody-conjugated tested were: anti-SIRP-α hIgG4 antibody-CpG oligonucleotide conjugates (heavy chain comprising the sequence of SEQ ID NO:66 and light chain comprising the sequence of SEQ ID NO:73, conjugated to CpG oligo 7-7 (SEQ ID NO:35)) and unconjugated anti-SIRP-α hIgG4 (heavy chain comprising the sequence of SEQ ID NO:66 and light chain comprising the sequence of SEQ ID NO: 73), anti-SIRP-α hIgG1 antibody-CpG oligonucleotide conjugates (heavy chain comprising the sequence of SEQ ID NO:68 and light chain comprising the sequence of SEQ ID NO:75, conjugated to CpG oligo 7-7 (SEQ ID NO:35)) and unconjugated anti-SIRP-α hIgG1 (heavy chain comprising the sequence of SEQ ID NO:68 and light chain comprising the sequence of SEQ ID NO:75), and anti-SIRP-α hIgG1 AAA antibody-CpG oligonucleotide conjugates (heavy chain comprising the sequence of SEQ ID NO:67 and light chain comprising the sequence of SEQ ID NO:75, conjugated to CpG oligo 7-7 (SEQ ID NO:35)) and unconjugated anti-SIRP-α hIgG1 AAA (heavy chain comprising the sequence of SEQ ID NO:67 and light chain comprising the sequence of SEQ ID NO:75). The antibodies and corresponding antibody-oligocleotide conjugates were added to the cells from 300 nM to 0.41 nM at 37° C. under 5% CO₂ for 48 hours. Cells were pelleted by centrifugation for five minutes at 400×g and stained at 4° C. in Fixable Viability Dye eFluor 780 (Thermo Fisher) diluted 1:4000 in PBS. Cells were centrifuged and stained at 4° C. in FACS buffer for 30 minutes containing FcR Blocking Reagent (Miltenyi Biotec), anti-CD14, anti-CD11c, anti-CD3, anti-CD19, anti-CD56, anti-CD16, anti-HLADR, anti-CD40, anti-CD86. Cells were centrifuged and washed twice in FACS buffer and fixed in 0.5% paraformaldehyde. Cells were analyzed on Attune NxT Flow Cytometer (Thermo Fisher), with subsequent data analysis by Flowjo 10.7 (Treestar). Dead cells were excluded by gating on the eFluor 780-negative population. Dendritic cells were identified as CD3⁻CD19⁻CD56⁻CD14⁻ HLADR⁺CD11c⁺. Dendritic cell activation levels of activation marker were assessed by median fluorescent intensity.

### Results

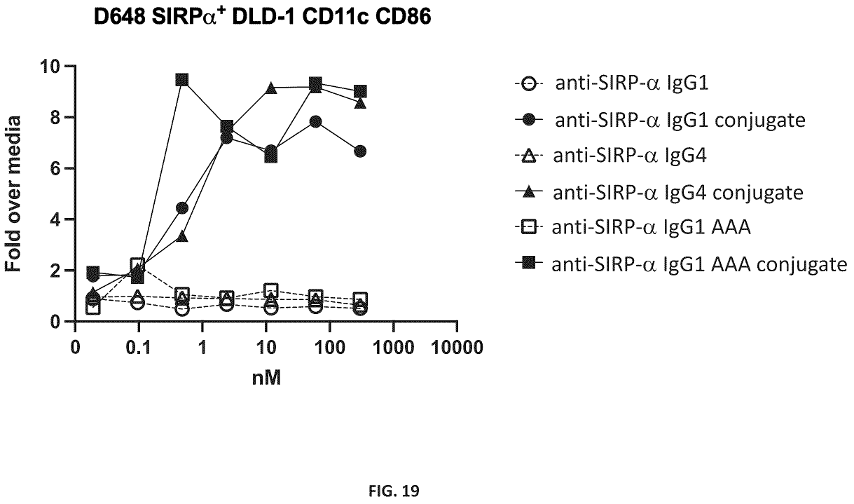

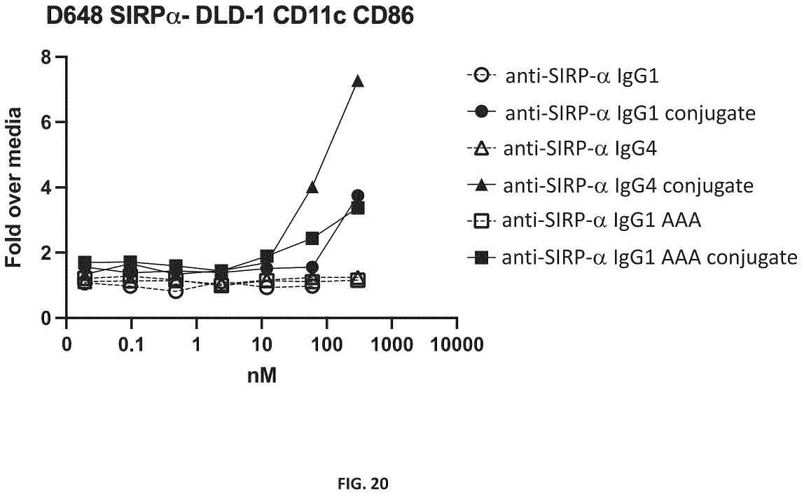

Human PBMCs were co-cultured either in presence of DLD-1 cells overexpressing SIRP-α (FIG. 19) or parental DLD-1 cells not expressing SIRP-α (FIG. 20) for 48 hr in the presence of respective anti-SIRP-α antibody-CpG oligonucleotide conjugates or anti-SIRP-α antibodies. Surface marker (CD86) expression was assayed by flow cytometry. These results demonstrated that expression of SIRPα on tumor cells significantly potentiated activation of myeloid cells (e.g., dendritic cells) by anti-SIRP-α antibody-CpG oligonucleotide conjugates at lower concentrations when compared to SIRP-α negative tumor cells in a co-culture assay. This was observed for all anti-SIRP-α antibody-CpG oligonucleotide conjugates tested and demonstrates that anti-SIRP-α antibody conjugates can offer lower threshold of myeloid cell activation in the context of SIRP-α-positive tumor cells versus that of SIRP-α-negative tumor cells.

### Example 18: Ability of Anti-SIRP-α Antibody Conjugates to Promote Phagocytosis of SIRP-α-Positive or SIRP-α-Negative Tumor Cells

### Materials and Methods

Human CD14⁺ cells were purified from Trima residuals (Vitalant) with Ficoll-Paque Plus and negative selection (Monocyte Isolation Kit II, Miltenyi Biotec) according to the manufacturers' protocols. Monocyte-derived macrophages (NPO) were made by seeding 10 million CD14 cells into 150 mm tissue culture dishes (Corning) in Complete RPMI growth medium supplemented with 10% human AB serum (Sigma) and 10 ng/ml MCSF for 3 days. On day 3, non-adherent cells were removed and incubated with growth media supplemented with 10% AB serum without MCSF for an additional 4 days. Adherent cells were detached from culture plates with TrypLE Select (Thermo Fisher Scientific). Target cells (DLD-1 or DLD-1 overexpressing SIRP-α) were labeled with the Celltrace CFSE Cell Proliferation kit (Thermo Fisher Scientific) according to the manufacturer's instructions. 100,000 target cells and 50,000 NPOs were incubated in ultra-low attachment U-bottom 96-well plates (Corning) with a titration from 150 nM to 0.2 nM of unconjugated and conjugated anti-SIRP-α antibodies for 2 hr at 37° C. The antibodies and corresponding antibody-conjugates tested are anti-SIRP-α hIgG4 antibody-CpG oligonucleotide conjugates (heavy chain comprising the sequence of SEQ ID NO:66 and light chain comprising the sequence of SEQ ID NO:73, conjugated to CpG oligo 7-7 (SEQ ID NO: 35)) and unconjugated anti-SIRP-α hIgG4 (heavy chain comprising the sequence of SEQ ID NO: 66 and light chain comprising the sequence of SEQ ID NO:73), anti-SIRP-α hIgG1 antibody-CpG oligonucleotide conjugates (heavy chain comprising the sequence of SEQ ID NO: 68 and light chain comprising the sequence of SEQ ID NO:75, conjugated to CpG oligo 7-7 (SEQ ID NO:35)) and unconjugated anti-SIRP-α hIgG1 (heavy chain comprising the sequence of SEQ ID NO:68 and light chain comprising the sequence of SEQ ID NO:75), and anti-SIRP-α hIgG1 AAA antibody-CpG oligonucleotide conjugates (heavy chain comprising the sequence of SEQ ID NO:67 and light chain comprising the sequence of SEQ ID NO:75, conjugated to CpG oligo 7-7 (SEQ ID NO:35)) and unconjugated anti-SIRP-α hIgG1 AAA (heavy chain comprising the sequence of SEQ ID NO:67 and light chain comprising the sequence of SEQ ID NO:75). For flow cytometry, cells were incubated in human FcR blocking reagent (Miltenyi Biotec) and stained with fluorochrome-labeled antibodies against CD33. To eliminate macrophage/target cell adhesion from analyses, antibody against CD326 was included. Furthermore, a pulse geometry gate of forward scatter signal area vs height was used to select for single cells. Fixable viability dye (Thermo Fisher Scientific) was used to identify live cells. Cells were acquired on a FACS Canto II flow cytometer (BD Biosciences) with subsequent analysis using FlowJo software. Percent phagocytosis indicates the percentage of viable CD33 macrophages that stain negative for CD326 and positive for CFSE.

### Results

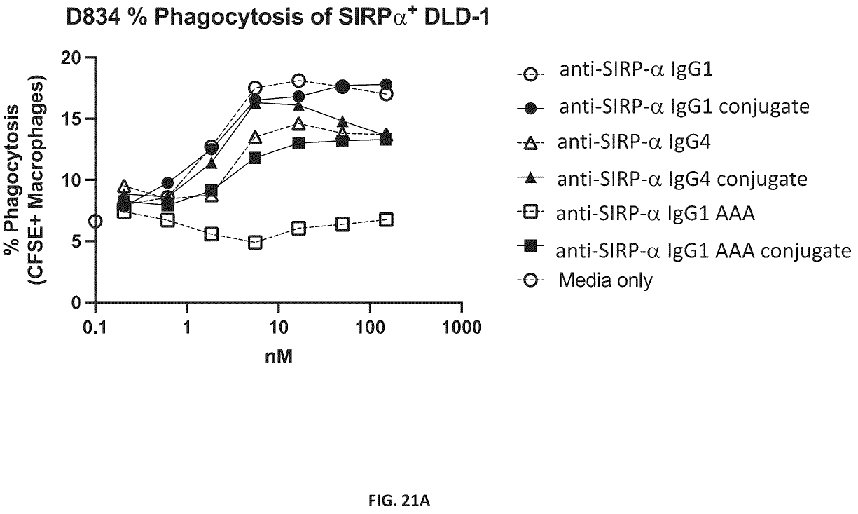

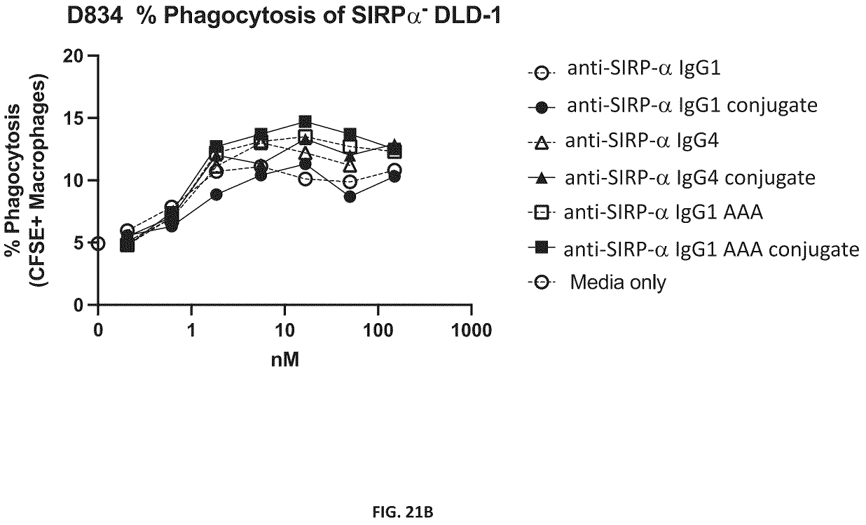

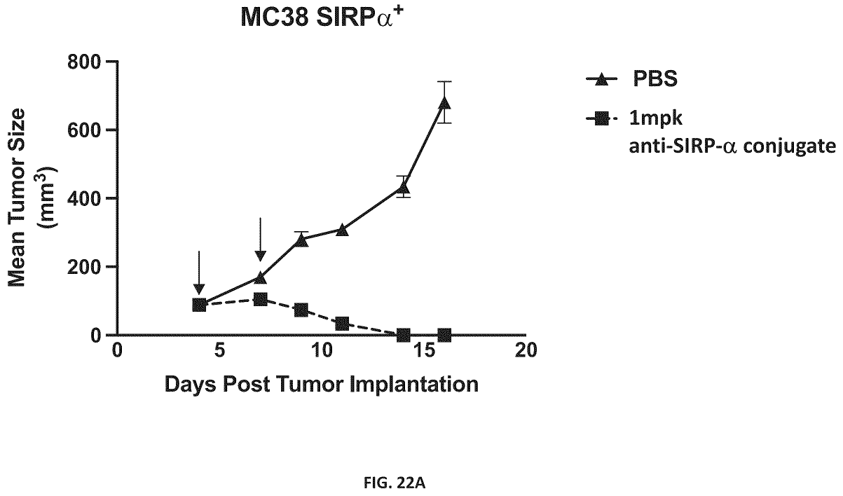

Human monocyte derived macrophages were co-incubated for 2 hrs with either DLD-1 overexpressing SIRP-α (FIG. 21A) or parental DLD-1 cells not expressing SIRP-α (FIG. 21B) in presence of various anti-SIRP-α antibody-CpG oligonucleotide conjugates or anti-SIRP-α antibodies. These results demonstrated that all tested anti-SIRP-α antibody-CpG oligonucleotide conjugates were able to promote phagocytosis of SIRP-α-positive and SIRP-α-negative tumor cells, compared to that of the corresponding unconjugated anti-SIRP-α antibodies.

### Example 19: Anti-Tumor Activity in a Mouse Syngeneic MC38 Model with SIRP-α-Positive and SIRP-α-Negative Tumor Cells

### Materials and Methods

Parental MC38 colon carcinoma cells (ATCC) and MC38 overexpressing mouse SIRPα were cultured in Complete Dulbecco's Modified Eagle's Medium (Gibco) (DMEM, 10% FBS, 1% Pen/Strep, 1% Glutmax, 1% sodium pyruvate) at 37° C. and 5% CO₂. Cells were detached with Trypsin 0.25% (Gibco) and washed twice with DMEM. Cells were resuspended at 20E6/mL in DMEM and kept on ice until use. 100 μL of suspended cells were subcutaneously implanted into the right flank of 6-week-old female C57/BL6 mice (Charles River). Tumor size was measured and recorded twice a week with calipers starting 4 days post implantation until duration of the study, approximately 17 days later. Tumor volume was calculated using the following formula: (length×width×width)/2. Once MC38 parental and MC38 SIRP-α tumors reached on average 94 mm³ to 88 mm³, respectively, approximately 4 days post implantation, mice were randomized by tumor size and treatments were initiated. The anti-SIRP-α mIgG1 conjugated to mTCpG (heavy chain comprising the sequence of SEQ ID NO:91 and light chain comprising the sequence of SEQ ID NO: 111, conjugated to mouse CpG oligo 4523 (SEQ ID NO: 121)) was administered intraperitoneally 2 doses every 3 days at 1 mg/kg. Mice whose tumors exceeded 2,000 mm³ or exhibited any signs of distress at any time during the study were sacrificed humanely as per IACUC-approved animal protocols.

### Results

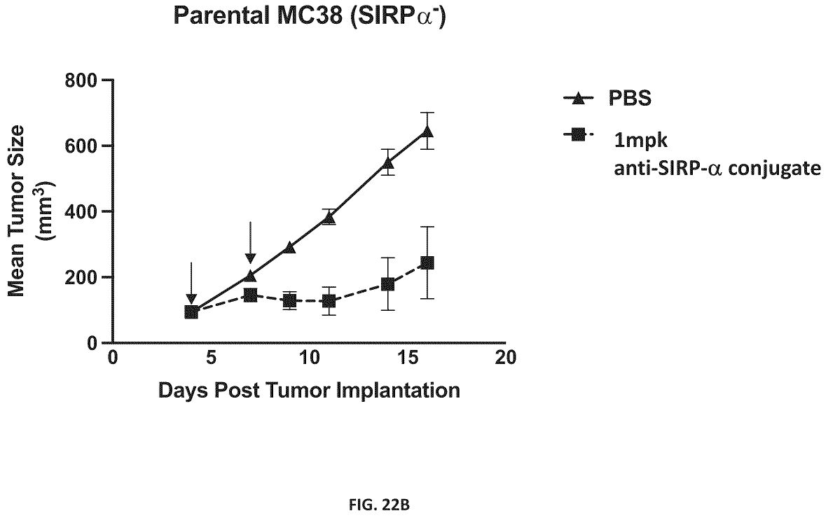

Mice bearing MC38 cells overexpressing SIRP-α (FIG. 22A) or parental MC38 cells (FIG. 22B) were dosed intraperitoneally (i.p.) twice, three days apart with anti-mSIRP-α antibody conjugated with murine reactive mT-CpG at 1 mg/kg (squares) or PBS control (triangles). The results demonstrated that localizing anti-SIRP-α antibody-CpG oligonucleotide conjugate to SIRP-α-positive tumor cells provided superior anti-tumor activity in this syngeneic tumor model when administered systemically, compared with parental MC38 cells not overexpressing SIRP-α.

### Example 20: Anti-Tumor Activity of Anti-SIRP-α Antibody Conjugates in RENCA, a Mouse Syngeneic Tumor Model Refractory to PD-1

### Materials and Methods

RENCA renal carcinoma cells line (ATCC) was cultured in Complete RPMI (RPMI, 10% FBS, 1× Pen/Strep, 1× Glutamax) at 37° C. and 5% CO₂. Cells were detached with Trypsin 0.25% (Gibco) and washed twice with RPMI 1640 (Gibco). Cells were resuspended at 20E6/mL in RPMI 1640 and kept on ice until use. 100 μL of suspended cells were subcutaneously implanted into the right flank of 6-week-old female BALB/c mice (Charles River). Tumor size was measured and recorded twice a week with calipers starting 9 days post implantation until duration of the study, approximately 27 days later. Tumor volume was calculated using the following formula: (length×width×width)/2. Once tumors reached on average 56 mm³, approximately 9 days post implantation, mice were randomized by tumor size and treatments were initiated. Anti-mSIRP-α conjugated to mT-CpG (heavy chain comprising the sequence of SEQ ID NO:91 and light chain comprising the sequence of SEQ ID NO:111, conjugated to mouse CpG oligo 4523 (SEQ ID NO:121)) was administered intraperitoneally 3 doses every 3 days at 10 mg/kg. In the anti-PD-1 RMP1-14 (BioXcell) treated cohort, dosing was initiated on day 10 post-tumor implantation once tumors reached on average 52 mm. A total of 3 doses 3 days apart at 10 mg/kg were administrated. Mice whose tumors exceeded 2,000 mm³ or exhibited any signs of distress at any time during the study were sacrificed humanely as per IACUC-approved animal protocols.

### Results

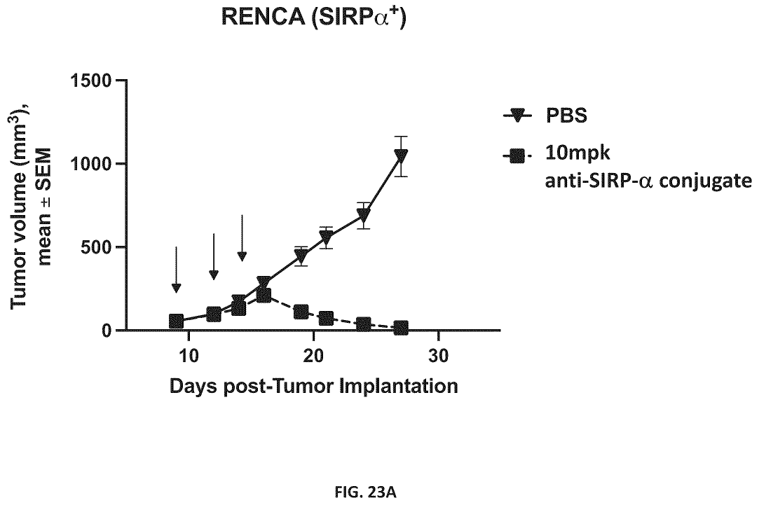

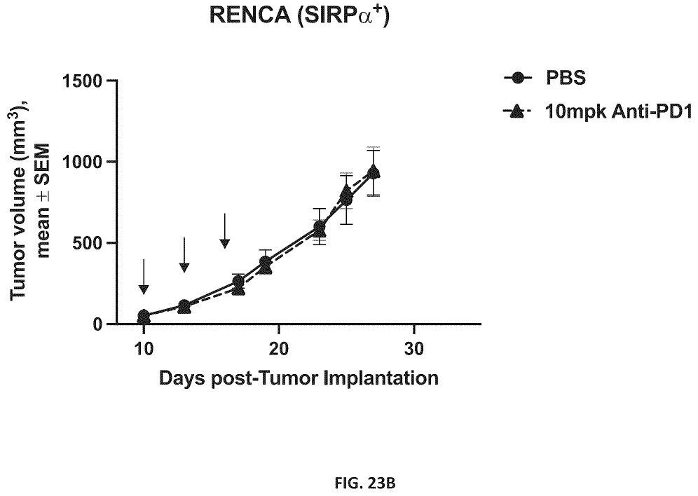

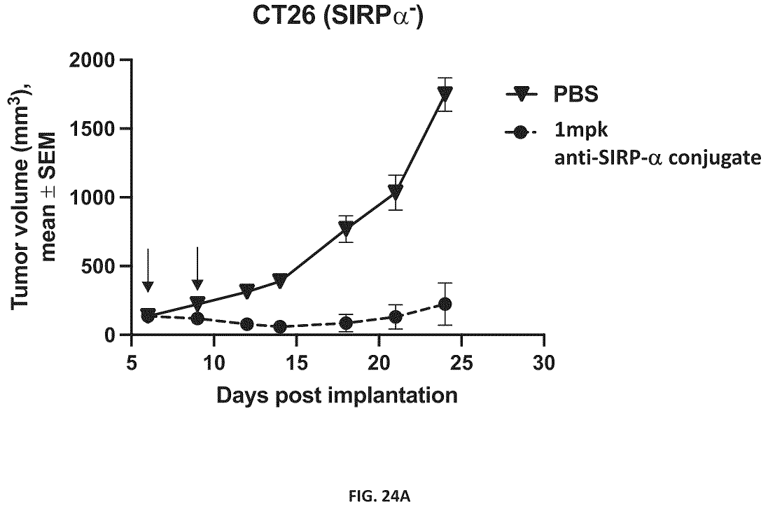

In FIG. 23A, mice bearing RENCA tumor cells, which are SIRP-α-positive and refractory to anti-PD-1 treatment, were dosed intraperitoneally (i.p.) three times, three days apart with anti-mSIRP-α antibody conjugated with murine reactive mT-CpG at 10 mg/kg (squares) or PBS (triangles). In FIG. 23B, a separate cohort was dosed with anti-PD-1 antibody RMP1-14 (BioXcell) at 10 mg/kg (closed triangles) three times, three days apart or PBS (empty triangles). These results demonstrated that anti-SIRP-α antibody-CpG oligonucleotide conjugate elicited a potent, single-agent anti-tumor response in this model, which is refractory to anti-PD-1 antibody-based single-agent treatment.

### Example 21: Anti-Tumor Activity of Anti-SIRP-α Antibody Conjugates in Combination with Anti-PD-1 Antibody

### Materials and Methods

CT26 mouse colon carcinoma cells (ATCC) were cultured in Complete RPMI (RPMI, 10% FBS, 1× Pen/Strep, 1× Glutamax) at 37° C. and 5% CO₂. Once cells were 80% confluent, cells were detached with Trypsin 0.25% (Gibco) and washed twice with RPMI 1640 (Gibco). Cells were resuspended at 20E6/mL in RPMI 1640 and kept on ice until use. 100 μL of suspended cells were subcutaneously implanted into the right flank of 6 week old female BALB/c mice (Charles River). Tumor size was measured and recorded twice a week with calipers starting 7 days post implantation until duration of the study, approximately 27 days later. Tumor volume was estimated using the following formula: (length×width×width)/2. Once tumors reached 110 mm³, approximately 9 days post implantation, mice were randomized by tumor size and treatments were initiated. Anti-mSIRP-α antibody-CpG oligonucleotide conjugate with a heavy chain comprising the sequence of SEQ ID NO:91 and a light chain comprising the sequence of SEQ ID NO: 111, conjugated to mouse CpG oligo 4523 (SEQ ID NO:121), was administered intraperitoneally at 1 mg/kg twice, 4 days apart, anti-PD-1 RMP14-1 (BioXcell) at 10 mg/kg twice, 4 days apart as a single agent or in combination with anti-mSIRP-α antibody-CpG oligonucleotide conjugate for a total of two doses 4 days apart and anti-PD-1 twice, two days post anti-mSIRP-α antibody-CpG oligonucleotide conjugate. Mice whose tumors exceeded 2,000 mm³ or exhibited any signs of distress at any time during the study were killed humanely as per IACUC-approved animal protocols.

### Results

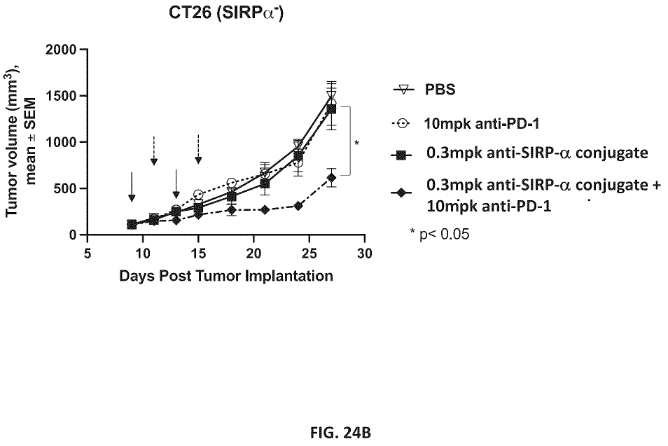

Anti-tumor activity of anti-SIRP-α antibody conjugates administered at a suboptimal dose in combination with anti-PD-1 antibody was tested in a mouse syngeneic tumor model. Mice bearing CT26 tumor cells were intraperitoneally (i.p.) treated with anti-mSIRPα antibody conjugated to murine reactive mT-CpG at 1 mg/kg (heavy chain comprising the sequence of SEQ ID NO: 91 and light chain comprising the sequence of SEQ ID NO: 111, conjugated to mouse CpG oligo 4523 (SEQ ID NO:121)) resulted in robust tumor growth inhibtion as a single agent (FIG. 24A). In a follow-up study in the same tumor model, mice were treated with anti-mSIRP-α antibody-CpG oligonucleotide conjugate at a suboptimal dose at 0.3 mg/kg, anti-PD-1 antibody at 10 mg/kg, both in combination, or PBS control (FIG. 24B). The results demonstrated that the combination of anti-SIRP-α antibody-CpG oligonucleotide conjugate and anti-PD-1 antibody elicited enhanced anti-tumor response in the CT26 syngeneic tumor model as compared to the anti-SIRP-α antibody-CpG oligonucleotide conjugate alone or the anti-PD-1 antibody alone. Surprisingly, the combination of anti-SIRP-α antibody-CpG oligonucleotide with the anti-PD-1 antibody provided an increased anti-tumor response even though the anti-PD-1 antibody when administered alone showed no anti-tumor activity in this model. Additionally, tumor growth inhibition attained with suboptimal dose of anti-mSIRP-α antibody-CpG oligonucleotide conjugate in combination with anti-PD-1 was similar to single agent anti-mSIRP-α antibody-CpG oligonucleotide conjugate at 1 mg/kg, further suggesting the combination approach enhanced anti-tumor response.

### Example 22: Anti-Tumor Activity of Anti-SIRP-α Antibody Conjugates in Combination with Anti-PD-L1 in B16F10 Tumor Model

### Materials and Methods

B16-F10 melanoma cell line (ATCC) was cultured in Complete Dulbecco's Modified Eagle's Medium (DMEM, 10% FBS, 1% Pen/Strep, 1% Glutamax, 1% Sodium Pyruvate) at 37° C. and 5% CO₂. Cells were detached with Trypsin 0.25% (Gibco) and washed twice with DMEM (Gibco). Cells were resuspended at 6E6/mL in DMEM and kept on ice until use. 100 μL of suspended cells were subcutaneously implanted into the right flank of 6-week-old female C₅₇BL/6 mice (Charles River). Tumor size was measured and recorded 2-3 times a week with calipers starting 7 days post-implantation until duration of the study, approximately 17 days later. Tumor volume was calculated using the following formula: (length×width×width)/2. Once tumors reached on average 55 mm³, approximately 7 days post-implantation, mice were randomized by tumor size and treatments were initiated. The conjugates were administered intraperitoneally 2 doses every 3 days at 30 mg/kg. Anti-PD-L1 was administered intraperitoneally 2 doses every 3 days at 10 mg/kg. Mice whose tumors exceeded 2,000 mm³ or exhibited any signs of distress at any time during the study were sacrificed humanely as per IACUC-approved animal protocols. The anti-mSIRP-α antibody conjugate comprised murine reactive mT-CpG oligonucleotide (SEQ ID NO: 121) conjugated to an anti-SIRP-α antibody comprising a heavy chain comprising the sequence of SEQ ID NO:91 and a light chain comprising the sequence of SEQ ID NO:111. The anti-PD-L1 antibody used was generated in-house and the heavy chain and light chain sequences corresponded to SEQ ID Nos: 116 and 117, respectively.

Anti-PD-L1 heavy chain
(SEQ ID NO: 116)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSAKTTPPSVYPLAPGSAAQTNSMVTLGCLV
KGYFPEPVTVTWNSGSLSSGVHTFPAVLQSDLYTLSSSVTVPSSTWPSE
TVTCNVAHPASSTKVDKKIVPRDCGCKPCICTVPEVSSVFIFPPKPKDV
LTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQV
YTIPPPKEQMAKDKVSLTCMITDFFPEDITVEWQWNGQPAENYKNTQPI
MDTDGSYFIYSKLNVQKSNWEAGNTFTCSVLHEGLHNHHTEKSLSHSPG
K
Anti-PD-L1 light chain
(SEQ ID NO: 117)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRADAAPTVSIFPPSSEQLTSGGASVVCFLNNFYPKDINVK
WKIDGSERQNGVLNSWTDQDSKDSTYSMSSTLTLTKDEYERHNSYTCEA
THKTSTSPIVKSFNRNEC

### Results

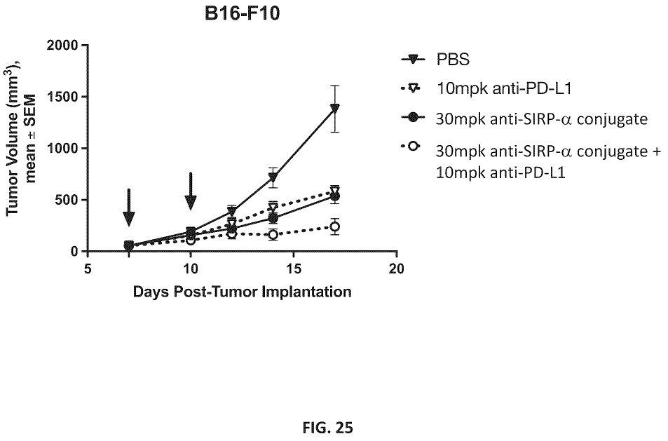

Anti-tumor activity of anti-SIRP-α antibody conjugates in combination with anti-PD-L1 antibody was tested in a mouse syngeneic tumor model. Mice bearing B16F10 tumor cells were intraperitoneally (i.p.) treated with either anti-SIRP-α antibody conjugated with murine reactive mT-CpG 4523 at 30 mg/kg, anti-PD-L1 antibody at 10 mg/kg, both in combination, or PBS control (FIG. 25). The results demonstrated that the combination of anti-SIRP-α antibody-CpG oligonucleotide conjugate and anti-PD-L1 antibody elicited enhanced anti-tumor response in the B16F10 syngeneic tumor model as compared to the anti-SIRP-α antibody-CpG oligonucleotide conjugate alone or the anti-PDL-1 antibody alone.

In conclusion, anti-SIRP-α antibody-CpG oligonucleotide conjugate was observed to specifically target myeloid cells and trigger TLR9 signaling (e.g., via SIRP-α and FcγR engagement), leading to robust cellular activation and cytokine induction in cultured PBMCs. Anti-SIRP-α antibody-CpG oligonucleotide conjugate potentiated activation of myeloid cells in the presence of SIRP-α-expressing tumor cells, and promoted phagocytosis of tumor cells independent of SIRP-α expression. Localization of anti-mSIRP-α antibody-mouse reactive CpG oligonucleotide conjugate to SIRP-α-positive tumors demonstrated robust and curative single-agent activity in multiple mouse models, including tumor types that are refractory to anti-PD-1 treatment. Anti-mSIRP-α antibody-mouse reactive CpG oligonucleotide conjugate enhanced tumor regression in combination with anti-PD-1 in a syngeneic tumor model.

### Example 23: Anti-SIRP-α Antibody Conjugate Inhibits Tumor Growth in CT26 Tumor-Bearing Mice Unresponsive to Prior Anti-PD-1 Therapy

### Materials and Methods

CT26 colon carcinoma cell line (ATCC) was cultured in Complete Roswell Park Memorial Institute-1640 medium (RPMI-1640 supplemented with 10% FBS (Gibco), 100 U/mL Penicillin and 100 μg/mL Streptomycin (Gibco), and 2 mM GlutaMAX (Gibco)) at 37° C. and 5% CO2. Cells were detached with Trypsin 0.25% (Gibco) and washed twice with RPMI-1640 (Gibco). Cells were resuspended at 206 cells/mL in RPMI-1640 and kept on ice until use. 100 μL of cell suspension, 2E6 CT26 cells, was subcutaneously implanted into the right flank of 6-week-old female BALB/c mice (Charles River). Tumor size was measured and recorded 2-3 times a week with calipers starting 6 days post-implantation until duration of the study, approximately 26 days later. Tumor volume was calculated using the following formula: (length×width×width)/2.

Once tumors reached on average 108 mm³, on day 6 post-implantation, mice were randomized by tumor size and treatment initiated with PBS or anti-PD-1 RMP1-14 (BioXcell) (10 mg/kg). Treatments were administered intraperitoneally twice 3 days apart. Two days after the second anti-PD-1 dose, tumor volumes were measured. Anti-PD-1-treated mice were considered non-responders if tumor measurement exceeded initial size and was greater than 250 mm³. Mice unresponsive to anti-PD-1 were re-randomized by tumor volume with an average size of 338 mm³ on day 11 into 3 new treatment cohorts. Beginning on day 11, mice received 1 mg/kg anti-SIRP-α antibody conjugate alone (cohort 1) or in combination with 10 mg/kg anti-PD-1 (cohort 2) or continued to receive 10 mg/kg anti-PD1 monotherapy (cohort 3). Treatments were administered intraperitoneally with a total of 2 doses every 3 days. Mice whose tumors exceeded 2,000 mm³ or exhibited any signs of distress at any time during the study were sacrificed humanely as per IACUC-approved animal protocols. The anti-mSIRP-α antibody conjugate comprised murine reactive mT-CpG oligonucleotide (SEQ ID NO: 121) conjugated to an anti-SIRP-α antibody comprising a heavy chain comprising the sequence of SEQ ID NO:91 and a light chain comprising the sequence of SEQ ID NO:111.

### Results

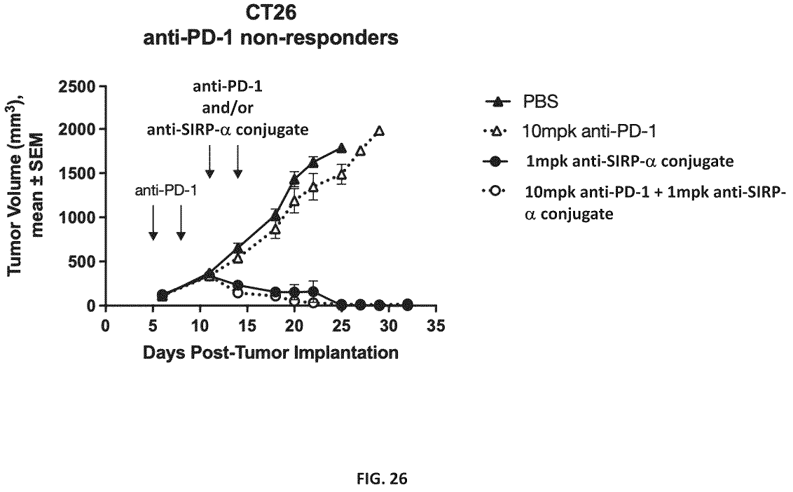

Activity of an anti-SIRP-α antibody-CpG oligonucleotide conjugate was tested in this CT26 tumor model in mice that were unresponsive to prior anti-PD-1 treatment as described above. As shown in FIG. 26, the anti-SIRP-α antibody-CpG oligonucleotide conjugate showed robust tumor growth inhibition at 1 mg/kg in CT26 tumor-bearing mice unresponsive to prior anti-PD-1 treatment. Tumor eradication was observed in 9 out of 9 mice with treatment of 1 mg/kg anti-SIRP-α antibody conjugate alone. Continued treatment with additional 2 doses of 10 mg/kg anti-PD-1 monotherapy resulted in only a slight delay in tumor growth in comparison to the PBS control cohort. The combination of 10 mg/kg anti-PD-1 with 1 mg/kg anti-SIRP-α antibody conjugate resulted in tumor eradication in 9 out of 10 mice and did not appear to further enhance tumor growth inhibition in comparison to 1 mg/kg anti-SIRP-α antibody conjugate treatment alone. These results demonstrate that treatment with anti-SIRP-α antibody-CpG oligonucleotide conjugate had potent anti-tumor activity even against tumors previously unresponsive to anti-PD-1 treatment.

### Example 24: Characteristics of Target Binding by Anti-SIRP-α Antibody Conjugate

### Materials and Methods

Binding of the anti-SIRP-α antibody conjugate was measured using a Biacore 8K high throughput, high-sensitivity SPR system (Cytiva, Global Life Sciences Solutions USA LLC, Marlborough, MA) equipped with S-type sensor chips. The interaction of the conjugate with SIRP-α was analyzed by flowing His-tagged SIRP-α over Protein A captured anti-SIRP-α antibody conjugate on a Biacore Series S Protein A Sensor Chip. The anti-SIRP-α antibody portion of the conjugate comprised a heavy chain comprising the sequence of SEQ ID NO:66 and a light chain comprising the sequence of SEQ ID NO:73 conjugated to CpG oligo 7-7 (SEQ ID NO: 35). The SIRP-α analytes used for the assay are shown in Table 18.

TABLE 18
SIRP-α analytes.

| Analyte | | |
|---|---|---|
| Ref. | SIRP-α analyte | Analyte Sequence |
| A | human SIRP-α V1 D1 | SEQ ID NO: 81 with 6-His |
| | domain-6His | tag at C-terminus |
| B | human SIRP-α V2 D1 | SEQ ID NO: 82 with 6-His |
| | domain-6His | tag at C-terminus |
| C | Cyno SIRP-α V4 D1 | SEQ ID NO: 84 followed |
| | domain-6His-Avi | by 6-His tag then Avi |
| | | peptide tag comprising the sequence |
| | | GLNDIFEAQKIEWHE |
| | | (SEQ ID NO: 114) at C- |
| | | terminus |
| D | Mouse SIRP-α normal | SEQ ID NO: 83 with 6-His |
| | D1 domain-6His | tag at C-terminus |
| E | Rat SIRP-α K32-N373- | SEQ ID NO: 115 with 6-His |
| | ECD-6His | tag at C-terminus |

Rat SIRPα-ECD:
(SEQ ID NO: 115)
KELKVTQADKSVSVAAGDSATLNCTVSSLTPVGPIKWFKGEGQNRSPIY
SFIGGEHFPRITNVSDATKRNNMDFSICISNVTPEDAGTYYCVKFQKGI
VEPDTEIKSGGGTTLYVLAKPSSPEVSGPDSRGSPGQTVNFTCKSYGFS
PRNITLKWLKDGKELSHLETTISSKSNVSYNISSTVSVKLSPEDIHSRV
ICEVAHVTLEGRPLNGTANFSNIIRVSPTLKITQQPLTPASQVNLTCQV
QKFYPKALQLNWLENGNLSRTDKPEHFTDNRDGTYNYTSLFLVNSSAHR
EDVVFTCQVEHDSQPAITENHTVRAFAHSSSGGSMETIPDNNAYYNWN

All SIRP-α analytes were injected in single cycle kinetics mode at nominal starting concentrations of 100 nM with 3-fold serial dilutions with the exception of rat SIRP-α. Higher starting concentration of 300 nM was used for assessing binding to rat SIRP-α at pH 7.4. Association times were monitored for 120 s, and dissociation times were monitored 600 s at 30 μL/min flow rate. The surfaces were regenerated with 75 mM phosphoric acid at pH1.65 for two pulses of 15 s at 30 μL/min flow rate. Experiments were done at either 25° C. or 37° C. and at pH 6.0 or 7.4. Assay buffers were 1×PBS 0.01% Tween-20 at pH 6.0 with and 1×HBS-EP+ (10 mM HEPES, pH7.4, 150 mM NaCl, 3 mM EDTA, 0.05% (v/v) Surfactant P20) at pH 7.4. All data points were collected in duplicate or greater.

The data were processed and analyzed with Biacore 8K Evaluation Software Version 4.0.8.20368 (Cytiva, Global Life Sciences Solutions USA LLC, Marlborough, MA). Reference responses from flow cell 1 were subtracted from the active responses from flow cell 2 to obtain the subtracted data (2-1). The responses from the nearest buffer blank injection were then subtracted from the reference subtracted data (2-1) to yield double-referenced data. These double-reference data were fit to a simple 1:1 Langmuir binding model with mass transport to determine the apparent association (ka) and dissociation rate constants (kd). The apparent equilibrium dissociation constant or affinity constant was then calculated based on their ratio as (K_{D}=kd/ka).

### Results

Results of the binding assays are shown in Table 19. The results indicated that the conjugate showed higher binding affinity for the SIRP-α from all species tested at the lower pH at both temperatures. Without wishing to be bound to theory, it is thought that these characteristics confer advantageous binding properties to the anti-SIRP-α antibody conjugate as an acidic microenvironment of tumors (pH 5.6 to pH 6.8) is a hallmark of malignant tumor cells.

TABLE 19
K_{D} measurements for anti-SIRP-α antibody conjugates.

| | | Analytes and K_{D} (nM) | | | | |
|---|---|---|---|---|---|---|
| pH | Temperature | A | B | C | D | E |
| 6.0 | 25° C. | <0.001 | <0.001 | 0.05 | <0.001 | 3.8 |
| | 37° C. | <0.001 | 0.01 | 0.10 | 0.07 | 8.5 |
| 7.4 | 25° C. | 0.10 | 0.23 | 0.45 | 0.69 | 61.4 |
| | 37° C. | 0.28 | 0.51 | 0.71 | 1.63 | 175 |

### Example 25: Potency of TLR9 Pathway Signaling Induced by Anti-SIRP-α Antibody Conjugate

### Materials and Methods

THP-1-Dual hTLR9 cell line (thpd-nfis, Invivogen) was derived from human THP-1 monocytes engineered to overexpress the human TLR9 (hTLR9) gene and featuring two reporter genes allowing for simultaneous assessment of the NF-kB pathway by monitoring the activity of an inducible secreted embryonic alkaline phosphatase (SEAP), and the interferon regulatory factor (IRF) pathway by monitoring the activity of an inducible secreted Lucia luciferase. Briefly, 250,000 cells in 100 uL of growth media (RPMI 1640 (Gibco), 2 mM L-glutamine (Gibco), 25 mM HEPES (Gibco), 10% FBS (Gibco), 100 U/mL penicillin (Gibco), 100 ug/mL streptomycin (Gibco), 100 ug/mL Normacin (Invivogen) were plated in 96-well flat-bottom plates (Nunc). An 8 step 2-fold serial dilution of anti-SIRP-α antibody or anti-SIRP-α antibody conjugate was added to cells in 50 μL for a final concentration range of 200 nM-1.56 nM. 10 μg/mL of a F(ab′)2 fragment goat anti-human, IgG Fcγ fragment specific (Jackson Immuno Research) was added in 50 μL of growth media as a cross-linking agent. Cells were incubated at 37 C for 24 hours. NF-kB and IRF induction from supernatant was detected according to manufacturer's protocol. EC₅₀ values were derived using three-parameter linear regression curve fir in Prism 9 (GraphPad), and data were presented as fold-over media control. The anti-SIRP-α antibody comprised a heavy chain comprising the sequence of SEQ ID NO:66 and a light chain comprising the sequence of SEQ ID NO:73. The anti-SIRP-α antibody conjugate tested comprised an anti-SIRP-α antibody with a heavy chain comprising the sequence of SEQ ID NO: 66 and a light chain comprising the sequence of SEQ ID NO:73, conjugated to CpG oligo 7-7 (SEQ ID NO:35). The naked CpG tested corresponded to CpG oligo 7-7 (SEQ ID NO:35).

### Results

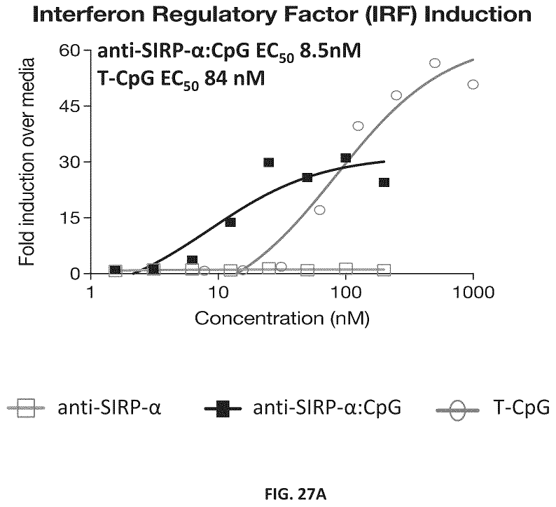

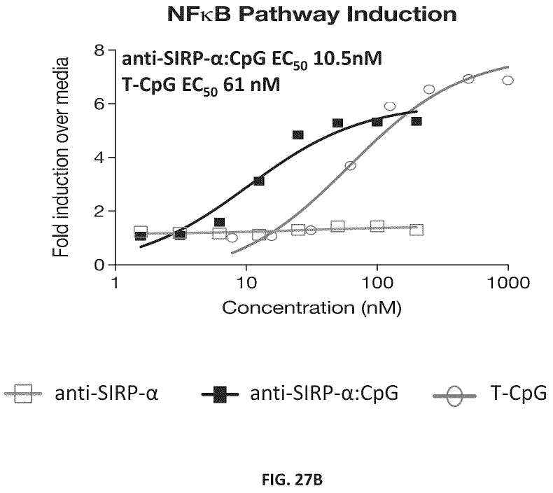

FIG. 27A demonstrates anti-SIRP-α antibody:CpG oligonucleotide conjugate elicited IRF induction with EC₅₀ of 8.5 nM, 10-fold more potently than T-CpG with EC₅₀ of 84 nM. Similarly, anti-SIRP-α antibody conjugate induced NF-kB with EC₅₀ of 10.5 nM, approximately 6-fold higher than free-CpG as presented in FIG. 27B. Unconjugated anti-SIRP-α antibody did not elicit a response.

Collectively, these data demonstrate robust induction of NF-kB and IRF pathways in response to TLR9 engagement by T-CpG that is potentiated by direct targeting of T-CpG via anti-SIRP-α antibody conjugate.

1. A conjugate comprising (i) an antibody or antigen-binding fragment thereof that specifically binds an extracellular domain of a human SIRP-α polypeptide and (ii) one or more immunomodulating oligonucleotides (P), wherein the antibody or antigen-binding fragment is linked to one or more Q-tag peptides (Q) comprising at least one glutamine residue, and wherein each immunomodulating oligonucleotide is linked to a Q-tag peptide via an amide bond with the glutamine residue of the Q-tag peptide and a linker (L) as shown in Formula (A): 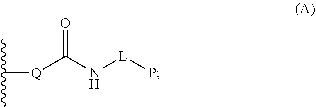
wherein [symbol] indicates the point of attachment of each Q to the antibody or antigen-binding fragment thereof (Ab);
wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H2 comprising the amino acid sequence of GISAGGSDTYYPASVKG (SEQ ID NO:57), and a CDR-H₃ comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO: 59), a CDR-L2 comprising the amino acid sequence of SDDKRPS (SEQ ID NO:60), and a CDR-L3 comprising the amino acid sequence of GGYDQSSYTNP (SEQ ID NO:61);
wherein each P is independently an immunomodulating oligonucleotide comprising the structure 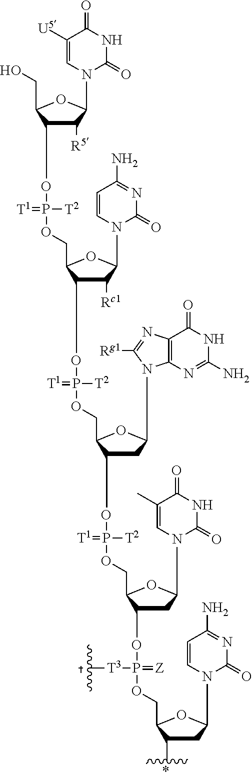 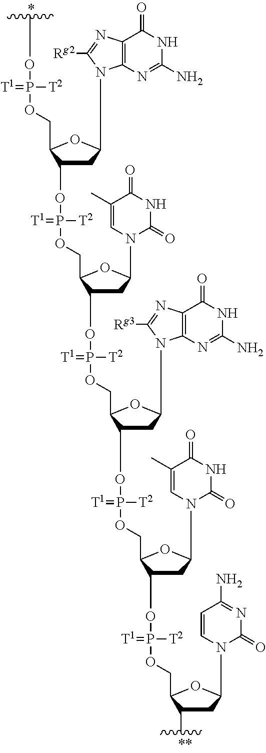 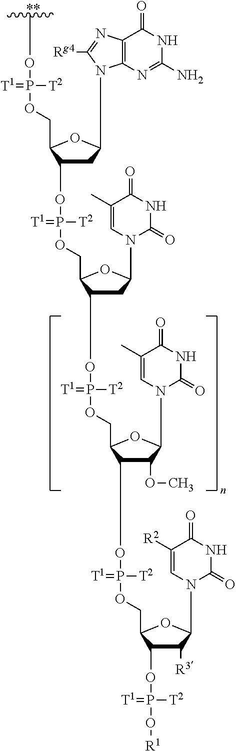
wherein [symbol]
* and [symbol] ** indicate the points of attachment within the oligonucleotide, and wherein [symbol] † indicates the point of attachment to the linker L;
each T¹ is independently O or S;
each T² is S⁻;
T³ is a group 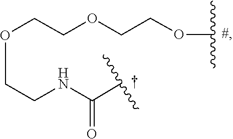 [SMILES: *OCCOCCOCCNC(*)=O]
wherein [symbol] † indicates the point of attachment to L and wherein [symbol] # indicates the point of attachment to the rest of the oligonucleotide;
Z is O or S;
U^{5′} is —H or halogen;
R^{5′} is —H or methoxy;
R^{c1} is —H or methoxy;
R^{g1}, R^{g2}, R^{g3}, and R^{g4} are H;
R^{3′} is methoxy;
R¹ is —(CH₂)₃—OH;
R² is —H or methyl; and
n is an integer from 0 to 2.

2. A conjugate comprising (i) an antibody or antigen-binding fragment thereof that specifically binds an extracellular domain of a human SIRP-α polypeptide and (ii) one or more immunomodulating oligonucleotides (P),
wherein the antibody or antigen-binding fragment is linked to one or more Q-tag peptides (Q) that comprise the amino acid sequence RPQGF (SEQ ID NO:47);
wherein the antibody or antigen-binding fragment thereof (Ab) comprises: (a) a heavy chain variable (VH) domain that comprises a CDR-H1 comprising the amino acid sequence of SNAMS (SEQ ID NO:56), a CDR-H₂ comprising the amino acid sequence of GISAGGSDTYYPASVKG (SEQ ID NO:57), and a CDR-H3 comprising the amino acid sequence of ETWNHLFDY (SEQ ID NO:58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61);
and wherein each immunomodulating oligonucleotide (P) is linked to a Q-tag peptide via an amide bond with the glutamine residue of the Q-tag peptide and a linker (L) as shown in Formula (A): 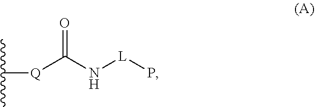
wherein [symbol] indicates the point of attachment of each Q to the antibody or antigen-binding fragment thereof (Ab).

3. The conjugate of claim 1, wherein
each P is independently an immunomodulating oligonucleotide comprising the structure

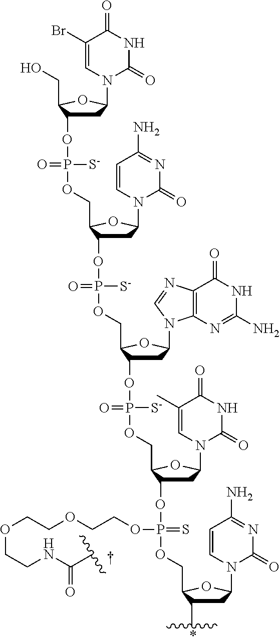

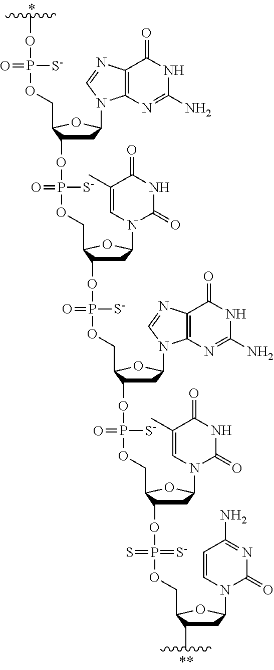

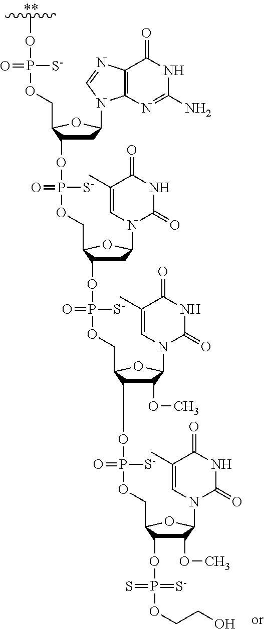

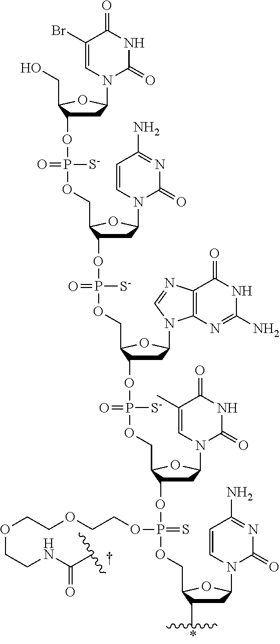

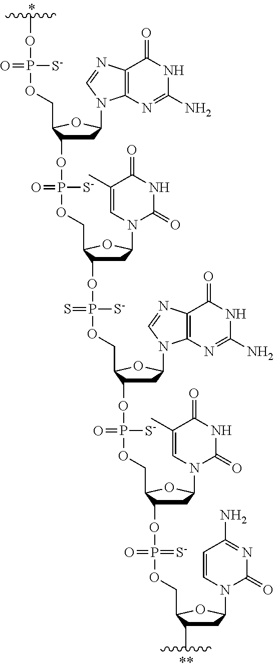

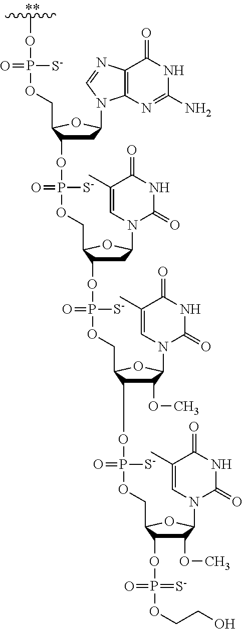

4-6. (canceled)

7. The conjugate of claim 1, wherein the VH domain comprises the amino acid sequence of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSS (SEQ ID NO:62), and wherein:
(a) the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDQSSYTNPFGGGTQLTVL (SEQ ID NO:63);
(b) the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDOSSYTNPFGGGTKLTVL (SEQ ID NO:64); or
(c) the VL domain comprises the amino acid sequence of SYELTQPPSVSVSPGQTARITCSGGSYSSYYYAWYQQKPGQAPVTLIYSDDKRPSNIPER FSGSSSGTTVTLTISGVQAEDEADYYCGGYDOSSYTNPFGGGTELTVL (SEQ ID NO:65).

8-9. (canceled)

10. The conjugate of claim 1, wherein the antibody or antigen-binding fragment is a monoclonal antibody, Fab, F(ab′)2, Fab′-SH, Fv, or scFv antibody or antibody fragment.

11-18. (canceled)

19. The conjugate of claim 1, wherein the antibody comprises an antibody heavy chain comprising an amino acid sequence selected from the group consisting of EVQLVESGGGVVQPGGSLRLSCAASGFTFSSNAMSWVRQAPGKGLEWVAGISAGGSDT YYPASVKGRFTISRDNSKNTLYLQMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEA AGAPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:88), EVQLVESGGGVVQPGGSLRLSCAASGFTFSSNAMSWVRQAPGKGLEWVAGISAGGSDT YYPASVKGRFTISRDNSKNTLYLOMNSLRAEDTAVYYCARETWNHLFDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLG (SEQ ID NO:87), and EVQLVESGGGVVQPGGSLRLSCAASGFTFSSNAMSWVRQAPGKGLEWVAGISAGGSDT YYPASVKGRFTISRDNSKNTLYLOMNSLRAEDTAVYYCARETWNHLFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHODWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTOKSLSLSPG (SEQ ID NO:89).

20-26. (canceled)

27. The conjugate of claim 1, wherein the antibody comprises an antibody light chain comprising an amino acid sequence selected from the group consisting of SEQ ID Nos: 72-80.

28. The conjugate of claim 1, wherein the antibody comprises:
(a) an antibody heavy chain, with C-terminal Q-tag peptide, comprising the amino acid sequence of SEQ ID NO:68 and an antibody light chain comprising the amino acid sequence of SEQ ID NO:79;
(b) an antibody heavy chain, with C-terminal Q-tag peptide, comprising the amino acid sequence of SEQ ID NO:67 and an antibody light chain comprising the amino acid sequence of SEQ ID NO:79;
(c) an antibody heavy chain, with C-terminal Q-tag peptide, comprising the amino acid sequence of SEQ ID NO:66 and an antibody light chain comprising the amino acid sequence of SEQ ID NO:79;
(d) an antibody heavy chain, with C-terminal Q-tag peptide, comprising the amino acid sequence of SEQ ID NO:68 and an antibody light chain comprising the amino acid sequence of SEQ ID NO:75;
(e) an antibody heavy chain, with C-terminal Q-tag peptide, comprising the amino acid sequence of SEQ ID NO:67 and an antibody light chain comprising the amino acid sequence of SEQ ID NO:75;
(f) an antibody heavy chain, with C-terminal Q-tag peptide, comprising the amino acid sequence of SEQ ID NO:66 and an antibody light chain comprising the amino acid sequence of SEQ ID NO:75;
(g) an antibody heavy chain, with C-terminal Q-tag peptide, comprising the amino acid sequence of SEQ ID NO:68 and an antibody light chain comprising the amino acid sequence of SEQ ID NO:73;
(h) an antibody heavy chain, with C-terminal Q-tag peptide, comprising the amino acid sequence of SEQ ID NO:67 and an antibody light chain comprising the amino acid sequence of SEQ ID NO:73; or
(i) an antibody heavy chain, with C-terminal Q-tag peptide, comprising the amino acid sequence of SEQ ID NO:66 and an antibody light chain comprising the amino acid sequence of SEQ ID NO:73.

29-30. (canceled)

31. The conjugate of claim 1, wherein each of the one or more Q-tag peptides comprises a sequence independently selected from the group consisting of SEQ ID NOs: 39-55.

32-43. (canceled)

44. The conjugate of claim 1, wherein the linker L is 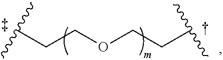
wherein m is an integer ranging from about 0 to about 50, and wherein [symbol] † indicates the point of attachment to T³, and [symbol] ‡ indicates the point of attachment to the rest of the conjugate.

45-74. (canceled)

75. The conjugate of claim 2, wherein each immunomodulating oligonucleotide P is independently 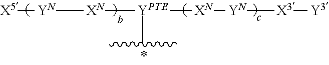
wherein
b and c are each independently an integer from 1 to 25; with the proviso that the sum of b and c is at least 5; [symbol]
* indicates the point of attachment of the immunomodulating oligonucleotide P to the rest of the conjugate;
X^{5′} is a 5′ terminal nucleoside comprising the structure 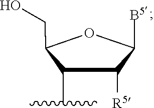
X^{3′} is a 3′ terminal nucleoside comprising the structure 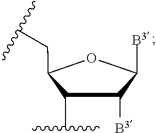
Y^{PTE} is an internucleoside phosphotriester comprising the structure 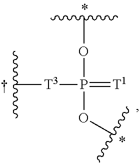
wherein * indicates the points of attachment to the rest of the oligonucleotide and [symbol] † indicates the point of attachment to the linker L, or, if L is absent, [symbol] † indicates the point of attachment to the Q tag peptide at the glutamine residue via an amide bond;
Y^{3′} is a terminal phosphotriester comprising the structure 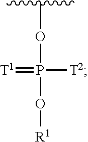
each X^{N} is independently a nucleoside comprising the structure 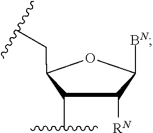
each Y^{N} is independently an internucleoside linker comprising the structure 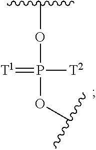
wherein each B^{N} is independently a modified or unmodified nucleobase;
each R^{N} is independently —H or —O—C₁₋₄-alkyl, wherein the C₁₋₄-alkyl of the —O—C₁₋₄-alkyl is optionally further substituted by —O—C₁-C₄-alkyl;
B^{5′} and B^{3′} are independently a modified or unmodified nucleobase;
R^{5′} and R^{3′} are independently —H or —O—C₁-C₄-alkyl, wherein the C₁₋₄-alkyl of the —O—C₁₋₄-alkyl is optionally further substituted by —O—C₁₋₄-alkyl;
each T₁ is independently O or S;
each T² is independently O⁻ or S⁻; and
T³ is a group comprising an oligoethylene glycol moiety; and
R¹ is C₁₋₄-alkylene-hydroxy.

76-91. (canceled)

92. The conjugate of claim 1, wherein the antibody comprises two antibody light chains, two antibody heavy chains, and two Q-tag peptides; wherein each of the Q-tag peptides is linked to the C-terminus of one of the antibody heavy chains; and wherein one of the Q-tag peptides is linked to an immunomodulating oligonucleotide (P) via an amide bond with the glutamine residue of the Q-tag peptide and linker (L).

93-112. (canceled)

113. A method for preparing a conjugate that comprises (i) an antibody or antigen-binding fragment thereof (Ab) that specifically binds human an extracellular domain of a human SIRP-α polypeptide and (ii) an immunomodulating oligonucleotide (P), comprising:
contacting the Ab with the oligonucleotide P in the presence of a transglutaminase;
wherein the antibody or antigen-binding fragment is linked to one or more Q-tag peptides (Q) comprising the amino acid sequence RPQGF (SEQ ID NO:47);
wherein each P independently comprises the following formula: 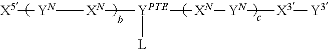
wherein
X^{5′} is a 5′ terminal nucleoside;
X^{3′} is a 3′ terminal nucleoside;
Y^{PTE} is an internucleoside phosphotriester;
Y^{3′} is a terminal phosphotriester;
each X^{N} is independently a nucleoside;
each Y^{N} is independently an internucleoside linker;
b and c are each independently an integer from 1 to 25; with the proviso that the sum of b and c is at least 5; and
L is a linker moiety comprising a terminal amine; and
wherein the antibody or antigen-binding fragment comprises: (a) a heavy chain variable (VH) domain that comprises a CDR-H1 comprising the amino acid sequence of SNAMS (SEQ ID NO: 56), a CDR-H₂ comprising the amino acid sequence of GISAGGSDTYYPASVKG (SEQ ID NO: 57), and a CDR-H3 comprising the amino acid sequence of ETWNHLFDY (SEQ ID NO: 58), and (b) a light chain variable (VL) domain that comprises a CDR-L1 comprising the amino acid sequence of SGGSYSSYYYA (SEQ ID NO:59), a CDR-L2 comprising the amino acid sequence of SDDKRPS (SEQ ID NO:60), and a CDR-L3 comprising the amino acid sequence of GGYDQSSYTNP (SEQ ID NO:61).

114-118. (canceled)

119. A pharmaceutical composition comprising the conjugate of claim 1 and a pharmaceutically acceptable carrier.

120. A method for treating cancer, comprising administering to an individual an effective amount of the conjugate according to claim 1.

121. The method of claim 120, wherein the cancer is lung cancer, intrahepatic cholangiocarcinoma, squamous cell cancer, brain tumors, glioblastoma, head and neck cancer, hepatocellular cancer, colorectal cancer, skin cancer, lung cancer, endometrial cancer, liver cancer, bladder cancer, gastric or stomach cancer, pancreatic cancer, cervical cancer, ovarian cancer, cancer of the urinary tract, urothelial cancer, breast cancer, peritoneal cancer, uterine cancer, salivary gland cancer, kidney or renal cancer, prostate cancer, vulval cancer, thyroid cancer, anal carcinoma, penile carcinoma, testis or testicular cancer, melanoma, multiple myeloma and B-cell lymphoma, non-Hodgkin's lymphoma (NHL), acute lymphoblastic leukemia (ALL), chronic lymphocytic leukemia (CLL), acute myeloid leukemia (AML), Merkel cell carcinoma, hairy cell leukemia, or chronic myeloblastic leukemia (CML), including metastases thereof.

122. (canceled)

123. The method of claim 120, wherein the cancer is predicted to be non-responsive to an inhibitor of PD-L1 or PD-1 or the individual does not achieve a significant therapeutic response to an inhibitor of PD-L1 or PD-1.

124-127. (canceled)

128. The method of claim 120, further comprising administering to the individual an additional therapeutic agent.

129-140. (canceled)

141. A method for activating myeloid cells, comprising administering to an individual in need thereof an effective amount of the conjugate according to claim 1.

142. A method for inducing TLR9 signaling in myeloid cells, comprising administering to an individual in need thereof an effective amount of the conjugate according to claim 1.

143-150. (canceled)
